(19) United States
(12) Patent Application Publication
(10) Pub. No.: US 2007/0225292 A1
(43) Pub. Date: Sep. 27, 2007

(54) Therapeutic Compounds: Pyridine as Scaffold

(76) Inventors: Kosrat Amin, Molndal (SE); Johan Broddefalk, Molndal (SE); Helene Desfosses, Montreal (CA); Emma Evertsson, Molndal (SE); Ziping Liu, Montreal (CA); Claire Milburn, Thousand Oaks, CA (US); Karolina Nilsson, Molndal (SE); Maxime Tremblay, Montreal (CA); Christopher Walpole, Montreal (CA); Zhong-yong Wei, Montreal (CA); Hua Yang, Montreal (CA)

Correspondence Address: WHITE & CASE LLP;PATENT DEPARTMENT, 1155 AVENUE OF THE AMERICAS, NEW YORK, NY, 10036, US

(21) Appl. No.: 11/569,315

(22) PCT Filed: May 20, 2005
(86) PCT No.: PCT/SE05/00753

§ 371 Date: Nov. 17, 2006

(30) Foreign Application Priority Data
May 25, 2004 (SE) 0401345-4

(51) Int. Cl. A61K 31/44; A61K 31/4965; C07D 213/81; C07D 241/24

(52) U.S. Cl. 514/252.1; 514/352; 544/336; 546/309

(57) ABSTRACT

Compounds of formula I or pharmaceutically acceptable salts thereof: 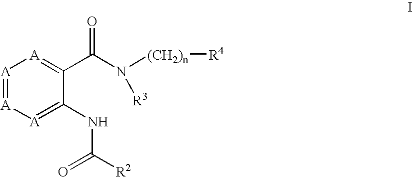
wherein R¹, R², R³, R⁴, n and A are as defined in the specification as well as salts and pharmaceutical compositions including the compounds are prepared. They are useful in therapy, in particular in the management of pain.

### BACKGROUND OF THE INVENTION

1. Field of the Invention

The invention is related to therapeutic compounds, pharmaceutical compositions containing these compounds, manufacturing processes thereof and uses thereof. Particularly, the present invention is related to compounds that may be effective in treating pain, cancer, multiple sclerosis, Parkinson's disease, Huntington's chorea, Alzheimer's disease, anxiety disorders, gastrointestinal disorders and/or cardiavascular disorders.

2. Discussion of Relevant Technology

Pain management has been an important field of study for many years. It has been well known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located predominately in the central nervous system, whereas CB₂ receptors are located primarily in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side effects, e.g., psychoactive side effects, the abuse potential, drug dependence and tolerance, etc. These undesired side effects are known to be mediated by the CB₁ receptors located in CNS. There are lines of evidence, however, suggesting that CB1 agonists acting at peripheral sites or with limited CNS exposure can manage pain in humans or animals with much improved overall in vivo profile.

Therefore, there is a need for new CB₁ receptor ligands such as agonists that may be useful in managing pain or treating other related symptoms or diseases with reduced or minimal undesirable CNS side effects.

### DESCRIPTION OF THE EMBODIMENTS

The present invention provides CB₁ receptor ligands which may be useful in treating pain and/or other related symptoms or diseases.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

“CB₁/CB₂ receptors” means CB₁ and/or CB₂ receptors.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms. Unless otherwise specified, “alkyl” general includes both saturated alkyl and unsaturated alkyl.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring.

The term “non-aromatic group” or “non-aromatic” used alone, as suffix or as prefix, refers to a chemical group or radical that does not contain a ring having aromatic character (e.g., 4n+2 delocalized electrons).

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to link two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alone or as a suffix or prefix, refers to a radical formed as a result of replacing one or more carbon atom of an alkyl with one or more heteroatoms selected from N, O, P and S.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a radical derived from a heterocycle by removing at least one hydrogen from a carbon of a ring of the heterocycle.

The term “heterocyclylene” used alone or as a suffix or prefix, refers to a divalent radical derived from a heterocycle by removing two hydrogens therefrom, which serves to link two structures together.

The term “heteroaryl” used alone or as a suffix or prefix, refers to a heterocyclyl having aromatic character, wherein the radical of the heterocyclyl is located on a carbon of an aromatic ring of the heterocyclyl.

The term “heterocycloalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (—NR), thio (═S), and oximino (═N—OR), wherein each “R” is a C₁₋₁₂hydrocarbyl. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanithene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein —R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “aryloxy” used alone or as suffix or prefix, refers to radicals of the general formula —O—Ar, wherein —Ar is an aryl.

The term “heteroaryloxy” used alone or as suffix or prefix, refers to radicals of the general formula —O—Ar′, wherein —Ar′ is a heteroaryl.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means —C(═O)—R, wherein —R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

Halogen includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least two atoms therebetween.

“Link,” “linked,” or “linking,” unless otherwise specified, means covalently linked or bonded.

When a first group, structure, or atom is “directly connected” to a second group, structure or atom, at least one atom of the first group, structure or atom forms a chemical bond with at least one atom of the second group, structure or atom.

“Saturated carbon” means a carbon atom in a structure, molecule or group wherein all the bonds connected to this carbon atom are single bond. In other words, there is no double or triple bonds connected to this carbon atom and this carbon atom generally adopts an sp³ atomic orbital hybridization.

“Unsaturated carbon” means a carbon atom in a structure, molecule or group wherein at least one bond connected to this carbon atom is not a single bond. In other words, there is at least one double or triple bond connected to this carbon atom and this carbon atom generally adopts a sp or Sp² atomic orbital hybridization. “RT”, “r.t.” or “rt” means room temperature.

“DMF” refers to dimethyl formamide.

“DIPEA” refers to N,N-diisopropylethylamine.

“HATU” refers to 2-(7-Aza-1H-benzotriazole-1-yl)-1,1,3,3-tetramethyluronium hexafluorophosphate.

One aspect of the invention is a compound of formula IC, a pharmaceutically acceptable salt thereof, diastereomers, enantiomers, or mixtures thereof: 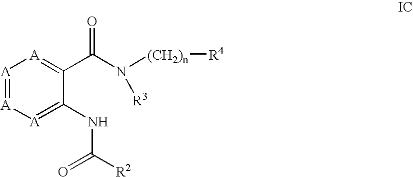
wherein:

A is selected from N and CR¹; and

R¹ is independently selected from hydrogen, halogen, cyano, amino, acetylamino, hydroxyl, alkoxy, ally, haloalkoxy, alkylene, haloalkyl, haloalkenyl and NR⁵R⁶;
each of R⁵ and R⁶ is independently selected from hydrogen, C₁₋₆alkyl, C₂₋₆alkenyl, alkoxyC₁₋₆ alkyl; C₁₋₆ alkylcarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocylcyl-C₁₋₆alkyl; wherein said C₁₋₆alkyl, C₂₋₆alkenyl, alkoxyC₁₋₆ alkyl; C₁₋₆ alkylcarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocylcyl-C₁₋₆alkyl used in defining R⁵ and R⁶ are optionally substituted by one or more groups selected from halogen, cyano, nitro, C₁₋₆ alkoxy, C₁₋₆ alkyl and hydroxy; and

and

R¹ is selected from aryl and heterocyclyl; wherein said aryl and heterocyclyl used in defining R² is optionally substituted by one or more groups selected from halogen, halo substituted alkyl, alkyl, cyano, nitro, alkoxy, hydroxy, hydroxy-alkyl, carbonyl, amino, alkyl-aryl, alkoxy, alkoxy-alkyl, alkylcarbonyl, alkoxycarbonyl, alkylamino, amino-alkyl, cycloalkyl, heteroaryl, heteroarylalkyl, aryl, aryl-alkyl and —NR⁵R⁶;

R³ is selected from hydrogen and alkyl;

R⁴ is selected from alkyl, cycloalkyl, cycloalkenyl, aryl and heterocyclyl; wherein said alkyl, cycloalkyl, cycloalkenyl, aryl and heterocyclyl used in defining R⁴ is optionally substituted by one or more groups selected from halogen, halo substituted alkyl, carbonyl, alkyl, cyano, nitro, amino, amino-alkyl, alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl and —NR⁵R⁶; and

n is selected from 0, 1, 2, 3, 4 and 5;

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halo substituted alkyl, carbonyl, alkyl, cyano, nitro, amino, amino-alkyl, alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶.

Particularly, the compounds of the present invention are those of formula IC, wherein

R¹ is independently selected from halogen, hydroxyl, cyano, C₁₋₆ alkoxy, C₁₋₆ alkyl, amino, C₁₋₄ haloalkoxy, C₂₋₆ alkylene, C₁₋₄ haloalkyl, C₂₋₆ haloalkenyl and NR⁵R⁶;
each of R⁵ and R⁶ is independently selected from hydrogen, C₁₋₆alkyl, C₂₋₆alkenyl, C₁₋₆ alkylalkoxy; C₁₋₆ alkylhydroxy, C₁₋₆ alkoxy, C₃₋₆cycloalkyl, C₃₋₆cycloalkyl-C₁₋₆alkyl, C₁₋₆alkylcarbonyl, C₁₋₄alkylcarbonyl, C₃₋₆heterocyclyl and C₃₋₆heterocylcyl-C₁₋₆alkyl; wherein said C₁₋₆alkyl, C₂₋₆alkenyl, C₁₋₆ alkylalkoxy; C₁₋₆ alkylhydroxy, C₁₋₆ alkoxy, C₃₋₆cycloalkyl, C₃₋₆cycloalkyl-C₁₋₆alkyl, C₁₋₆alkylcarbonyl, C₁₋₄alkylcarbonyl, C₃₋₆heterocyclyl and C₃₋₆heterocylcyl-C₁₋₆alkyl used in defining R⁵ and R⁶ are optionally substituted by one or more groups selected from halogen, cyano, nitro, C₁₋₃ alkoxy, C₁₋₃ alkyl, and hydroxy; and

A is selected from N and CR¹; and

R² is selected from aryl and C₂₋₆ heterocyclyl; wherein said aryl and C₂₋₆ heterocyclyl used in defining R² is optionally substituted by one or more groups selected from halogen, halo substituted C₁₋₆ alkyl, alkyl, cyano, nitro, C₁₋₆ alkoxy, hydroxy, hydroxy-C₁₋₆ alkyl, carbonyl, amino, C₁₋₆ alkoxy-alkyl, C₁₋₆ alkyl-carbonyl, aryl, aryl-C₁₋₆ alkyl and —NR⁵R⁶; and

R³ is selected from hydrogen and C₁₋₆ alkyl; and

R⁴ is selected from aryl and C₂₋₁₀ heterocyclyl; wherein said aryl and C₂₋₁₀ heterocyclyl used in defining R² is optionally substituted by one or more groups selected from halogen, halo substituted C₁₋₁₀ alkyl, carbonyl, alkyl, cyano, nitro, amino, amino-alkyl, alkoxy, hydroxy, alkoxy-alkyl, C₁₋₁₀ alkoxy-aryl, C₁₋₁₀ alkoxy-carbonyl, heterocyclic moiety, C₃₋₁₀ aryl, aryl-alkyl, heterocyclic-alkyl, hydroxy-alkyl, heteroaryl, alkyl-heteroaryl, aryl-alkyl and —NR⁵R⁶; and

n is selected from 0, 1, 2, 3 and 4; and

R³ and R⁴ together with the nitrogen atom to which they are attached may form a group selected from selected from azepanyl, pyrrolyl, pyrrolinyl, pyrrolidinyl, imidazolyl, imidazolidinyl, pyrazolyl, pyrazolinyl, pyrazolidinyl, isoxazolyl, isothiazolyl, isoxazolidinyl, oxadiazolyl, trazolyl, thiadiaxolyl, morpholinyl, piperidinyl, pyridinyl, thiomorpholinyl, pyridazinyl, pyrimidinyl, pyrazinyl, piperazinyl, triazinyl, tetrahydrofuranyl, tetrahydrofuranyl-methyl, tetrahydrofuranyl-ethyl, tetrahydropyranyl, tetrahydropyranylmethyl, tetrahydropyranylethyl or 1,4-dioxa-8-azaspiro[4,5]decan-8-yl; wherein said azepanyl, pyrrolyl, pyrrolinyl, pyrrolidinyl, imidazolyl, imidazolidinyl, pyrazolyl, pyrazolinyl, pyrazolidinyl, isoxazolyl, isothiazolyl, isoxazolidinyl, oxadiazolyl, trazolyl, thiadiaxolyl, morpholinyl, piperidinyl, pyridinyl, thiomorpholinyl, pyridazinyl, pyrimidinyl, pyrazinyl, piperazinyl, triazinyl, tetrahydrofuranyl, tetrahydrofuranyl-methyl, tetrahydrofuranyl-ethyl, tetrahydropyranyl, tetrahydropyranylmethyl, tetrahydropyranylethyl or 1,4-dioxa-8-azaspiro[4,5]decan-8-yl used in defining R³ and R⁴ are optionally substituted by one or more groups selected from halogen, fluoro substituted alkyl, C₁₋₆alkyl, cyano, nitro, hydroxy, amino, amino-C₁₋₄alkyl, hydroxy-C₁₋₄alkyl, C₁₋₄alkoxy, C₁₋₄alkoxy-C₁₋₄alkyl, C₁₋₄alkoxy-aryl, C₁₋₄alkoxycarbonyl, heterocyclic moiety, heterocyclic-C₁₋₄alkyl, aryl and aryl-C₁₋₄alkyl, and —NR⁵R⁶.

More particularly, the compounds of the present invention are those of formula IC, wherein

R¹ is independently selected from halogen, hydroxyl, C₁₋₆ alkoxy, C₁₋₆ alkyl, C₂₋₆ alkylene, NH₂, and NR⁵R⁶;
each of R⁵ and R⁶ is independently selected from hydrogen, C₁₋₆alkyl, C₂₋₆alkenyl, C₁₋₆ alkylalkoxy; C₁₋₆ alkylhydroxy, C₁₋₄alkylcarbonyl, C₁₋₄ alkoxy, C₃₋₆cycloalkyl, C₃₋₆cycloalkyl-C₁₋₆alkyl, C₃₋₆heterocyclyl and C₃₋₆heterocylcyl-C₁₋₆alkyl; wherein said C₁₋₆alkyl, C₂₋₆alkenyl, C₁₋₆ alkylalkoxy; C₁₋₆ alkylhydroxy, C₁₋₄alkylcarbonyl, C₁₋₄ alkoxy, C₃₋₆cycloalkyl, C₃₋₆cycloalkyl-C₁₋₆alkyl, C₃₋₆heterocyclyl and C₃₋₆heterocylcyl-C₁₋₆alkyl used in defining R⁵ and R¹ are optionally substituted by one or more groups selected from halogen, cyano, nitro, methoxy, ethoxy, methyl, ethyl, and hydroxy;

A is selected from N and CR¹; and

R² is selected from phenyl, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl, indolyl, indolinyl, quinolinyl, tetrahydroquinolinyl, isoquinolinyl, tetrahydroisoquinolinyl, 1,4-benzodioxanyl, coumarin, dihydrocoumarinyl, 2,3-dihydrobenzofuranyl, 1,2-benzisoxazolyl, 1,3-benzodioxolyl, 2,3-dihydro-1,4-benzodioxinyl, 3,4-dihydro-2H-1,5-benzodioxepinyl, 4H-1,3-benzodioxinyl, benzofuranyl, benzothiophenyl, benzoxazolyl, benzthiazolyl, benzimidazolyl, benztriazolyl, thioxanthinyl, carbazolyl, carbolinyl, acridinyl, pyrolizidinyl, naphthalenyl or quinolizidinyl; wherein said phenyl, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl, indolyl, indolinyl, quinolinyl, tetrahydroquinolinyl, isoquinolinyl, tetrahydroisoquinolinyl, 1,4-benzodioxanyl, coumarin, dihydrocoumarinyl, 2,3-dihydrobenzofuranyl, 1,2-benzisoxazolyl, 1,3-benzodioxolyl, 2,3-dihydro-1,4-benzodioxinyl, 3,4-dihydro-2H-1,5-benzodioxepinyl, 4H-1,3-benzodioxinyl, benzofuranyl, benzothiophenyl, benzoxazolyl, benzthiazolyl, benzimidazolyl, benztriazolyl, thioxanthinyl, carbazolyl, carbolinyl, acridinyl, pyrolizidinyl, naphthalenyl or quinolizidinyl used in defining R² is optionally substituted by one or more groups selected from hydrogen, halogen, hydroxy, C₁₋₄alkyl, amino, trifluoromethyl, C₁₋₄alkyl-aryl, C₁₋₄alkyl-heteroaryl, C₁₋₄alkoxy, C₁₋₆alkoxy-C₁₋₄alkyl, C₁₋₆alkamino, amino-C₁₋₄alkyl, C₃₋₈ aryl and heteroaryl, N,N-dimethylmethylamino, methylmethoxy, methyl-diazolyl, methyl-triazolyl, methyl-tetrazolyl, and —NR⁵R⁶; and

R³ is selected from hydrogen and C₁₋₆ alkyl; and

R⁴ is selected from amino, amino-C₁₋₆alkyl, hydroxy, hydroxy-C₁₋₆alkyl, C₁₋₆ alkyl, C₂₋₆alkenyl, C₂₋₆alkynyl, C₁₋₆alkoxy, C₁₋₆alkoxy-C₁₋₆alkyl, C₁₋₆alkoxy-aryl, C₁₋₆alkoxycarbonyl, C₁₋₆alkcarbonyl, C₃₋₁₀cycloalkyl, C₃₋₁₀cycloalkyl-C₁₋₆alkyl, C₄₋₈cycloalkenyl-C₁₋₆alkyl, C₄₋₈cycloalkenyl, C₃₋₁₀cycloalkoxy, C₃₋₁₀ aryl, aryl-C₁₋₆alkyl, amino-carbonyl-C₁₋₆alkyl, heterocyclic moiety, heterocyclic-C₁₋₆alkyl or heterocyclic-carbonyl-C₁₋₆alkyl wherein said amino, amino-C₁₋₆alkyl, hydroxy, hydroxy-C₁₋₆alkyl, C₁₋₁₀allyl, C₂₋₁₀alkenyl, C₂₋₁₀alkynyl, C₁₋₁₀alkoxy, C₁₋₁₀alkoxy-C₁₋₆alkyl, C₁₋₁₀alkoxy-aryl, C₁₋₁₀alkoxycarbonyl, C₁₋₁₀alkcarbonyl, C₃₋₁₀cycloalkyl, C₃₋₁₀cycloalkyl-C₁₋₆alkyl, C₄₋₈cycloalkenyl-C₁₋₆alkyl, C₄₋₉cycloalkenyl, C₃₋₁₀cycloalkoxy, C₃₋₁₀ aryl, aryl-C₁₋₆alkyl, amino-carbonyl-C₁₋₆alkyl, heterocyclic moiety, heterocyclic-C₁₋₆alkyl or heterocyclic-carbonyl-C₁₋₆alkyl used in defining R⁴ is optionally substituted with one or more substituents selected from halogen, hydroxy, hydroxy-C₁₋₆alkyl, cyano, carbonyl, nitro, amino, C₁₋₆alkyl, C₁₋₆alkoxy, C₁₋₆alkoxy-C₁₋₆alkyl, C₁₋₆alkcarbonyl, C₁₋₆alkoxycarbonyl, C₁₋₆alkamino, amino-C₁₋₆alkyl, C₃₋₆cycloalkyl, C₃₋₆ aryl-C₁₋₄ alkyl, C₃₋₆ aryl and —NR⁵R⁶; and

n is selected from 0, 1, 2, and 3; and

R³ and R⁴ together with the nitrogen atom to which they are attached may form a group selected from azepanyl, isoxazolidinyl, morpholinyl, piperazinyl, piperidinyl, pyrrolidinyl, tetrahydrofuranyl, tetrahydrofuranyl-methyl, tetrahydrofuranyl-ethyl, tetrahydropyranyl, tetrahydropyranylmethyl, tetrahydropyranylethyl or 1,4-dioxa-8-azaspiro[4,5]decan-8-yl with one or more substituents selected from halogen, cyano, nitro, methyl, ethyl, hydroxy, hydroxy-methyl, hydroxy-ethyl, amino-methyl, amino-ethyl, methoxy-methyl, methoxy-phenyl, ethoxycarbonyl, tert-butoxycarbonyl, diphenyl-methyl, morpholinyl-eth-2-yl, piperidinyl-methyl and pyridinyl.

Most particularly, the compounds of the present invention are those of formula IC, wherein

R¹ is independently selected from halogen, hydroxyl, C₁₋₃ alkoxy, C₁₋₆ alkyl, NH₂, C₂₋₆ alkylene and NR⁵R⁶;
each of R⁵ and R⁶ is independently selected from hydrogen, C₁₋₄alkyl, C₂₋₄alkenyl, C₁₋₄ alkylalkoxy; C₁₋₄ alkylhydroxy, alkoxy, C₃₋₆cycloalkyl, C₃₋₆cycloalkyl-C₁₋₄alkyl, methylcarbonyl, ethylcarbonyl, C₃₋₆heterocyclyl and C₃₋₆heterocylcyl-C₁₋₆alkyl; wherein said C₁₋₄alkyl, C₂₋₄alkenyl, C₁₋₄ alkylalkoxy; C₁₋₄ alkylhydroxy, alkoxy, C₃₋₆cycloalkyl, C₃₋₆cycloalkyl-C₁₋₄alkyl, methylcarbonyl, ethylcarbonyl, C₃₋₆heterocyclyl and C₃₋₆heterocylcyl-C₁₋₆alkyl used in defining R⁵ and R⁶ are optionally substituted by one or more groups selected from halogen, cyano, nitro, methoxy, ethoxy, methyl, ethyl, and hydroxy; and

A is selected from N and CR¹; and

R² is selected from 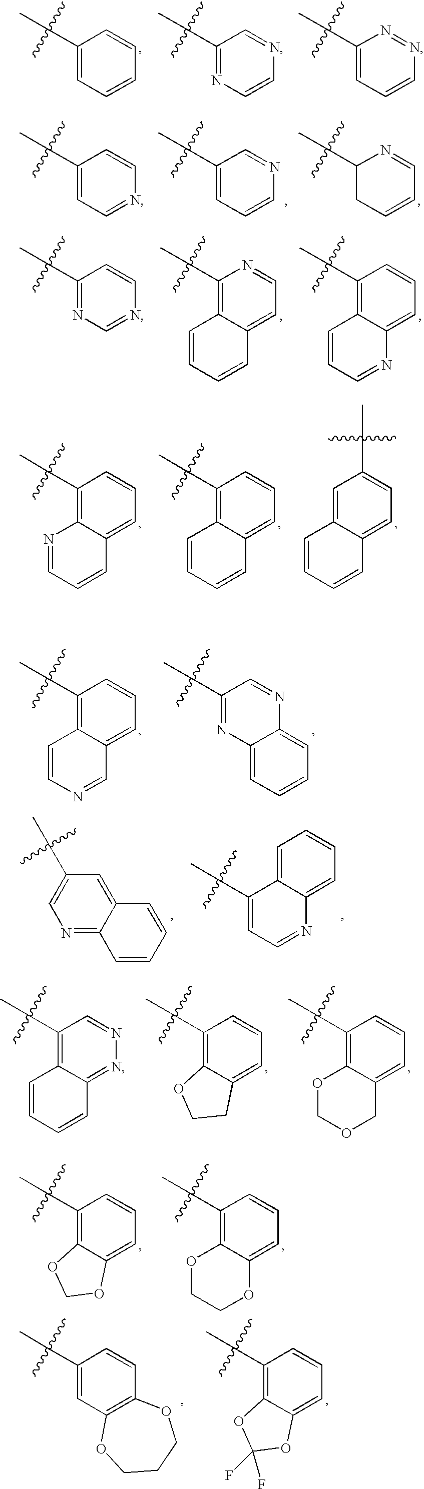
wherein above-identified groups are optionally substituted by one or more groups selected from Cl, Br, F, hydroxy, ethoxy, methoxy, trifluoromethyl, C₁₋₆ alkyl, cyano, nitro, and phenyl optionally substituted by one or more groups selected from methyl and ethyl; and

R³ is selected from hydrogen, methyl and ethyl; and

R⁴ is selected from 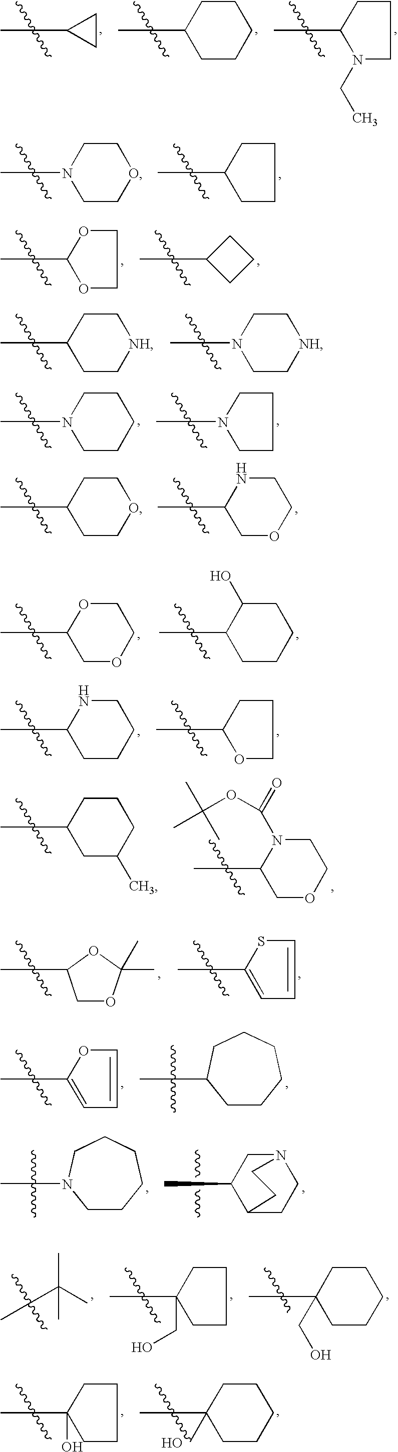
alkenyl, hydroxy, C₁₋₆ alkoxy, —CR⁵R⁶; and —NR⁵R⁶; wherein the groups used in defining R⁴ are optionally substituted by one or more groups selected from halogen, hydroxy, C₁₋₄ alkoxy, halo substituted alkyl, C₁₋₄ alkyl, cyano, nitro, —NR⁵R⁶; and phenyl optionally substituted by one or more selected from methyl and ethyl; and

n is selected from 0, 1, 2 and 3; and

R³ and R⁴ together with the nitrogen atom to which they are attached may form a group selected from

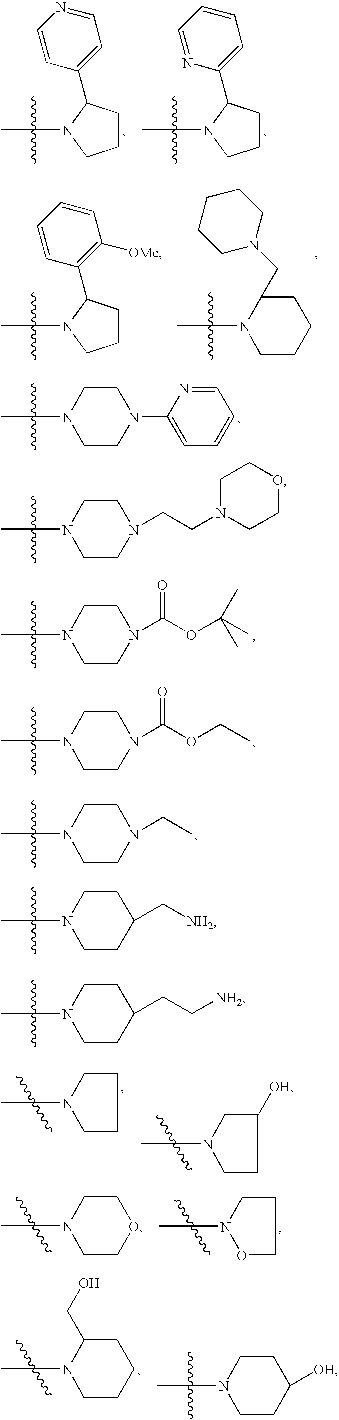

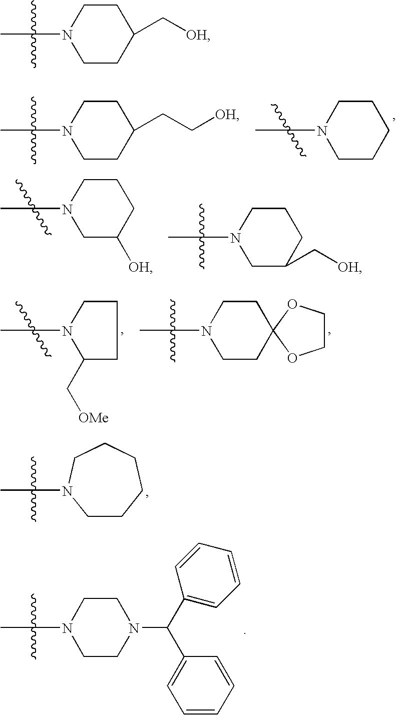

Another aspect of the invention is a compound of formula I, a pharmaceutically acceptable salt thereof, diastereomers, enantiomers, or mixtures thereof: 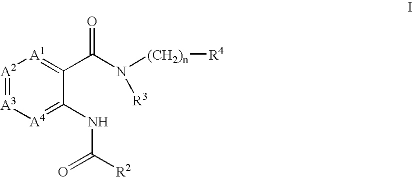
wherein:

one of A¹, A², A³ or A⁴ is N and the remaining are each and independently CR¹; and

R¹ is independently selected from hydrogen, halogen, cyano, amino, acetylamino, hydroxyl, alkoxy, alkyl, halogenated alkoxy, alkylene, halogenated alkyl, halogenated alkenyl and NR⁵R⁶;

R² is selected from 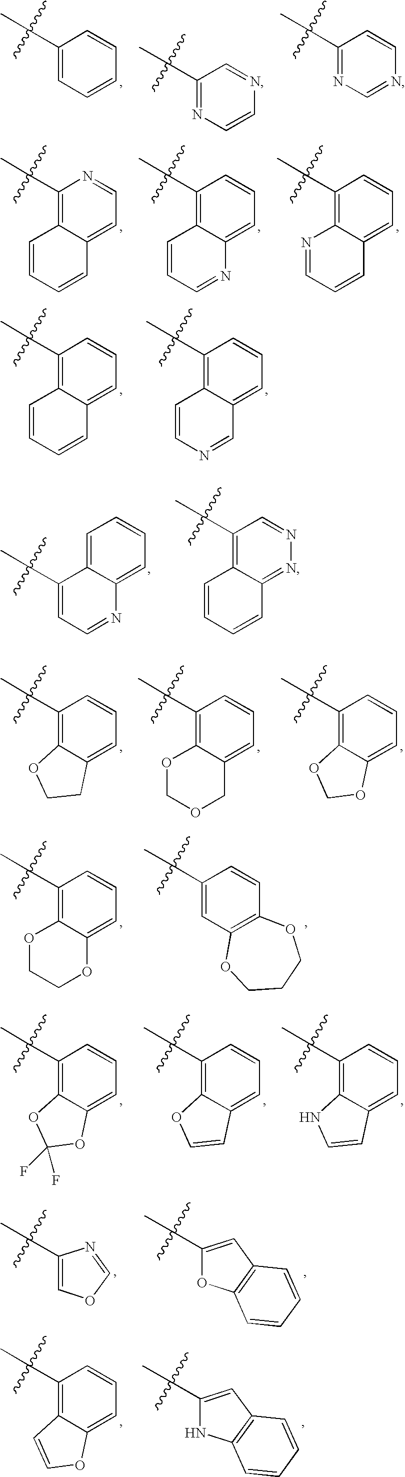 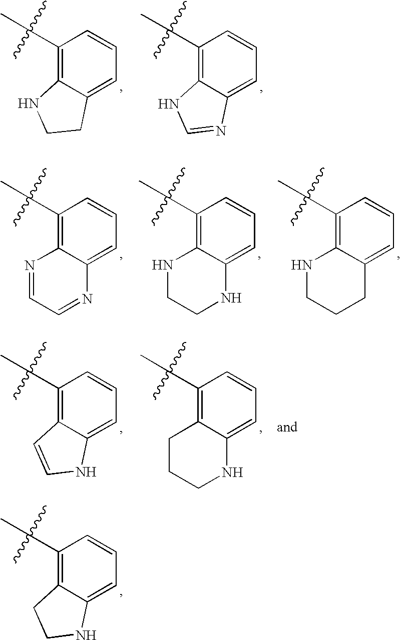
wherein said group used in defining R² is optionally substituted by one or more groups selected from halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkylenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, cyano, nitro, C₁₋₆ alkoxy, C₁₋₆ alkyl and hydroxy;

with a proviso that when n=0 then R⁴ is not thiazolyl or 5-chloropyridinyl;

with a further proviso that when R² is phenyl then n=0 and R⁴ is not unsubstituted methyl, C₃ alkyl or unsubstituted C₄ alkyl; and

with a further proviso that said compound of formula I is not any one of 3-(benzoylamino)-N-benzylpyridine-2-carboxamide;
3-(benzoylamino)-N-pyridin-3-ylpyridine-2-carboxamide;
3-(benzoylamino)-N-phenylpyridine-2-carboxamide;
3-(benzoylamino)-N-(3-nitrophenyl)pyridine-2-carboxamide;
3-(benzoylamino)-N-(4-methoxyphenyl)pyridine-2-carboxamide;
3-(benzoylamino)-N-[4-(dimethylamino)phenyl]pyridine-2-carboxamide;
N-(2-hydroxyethyl)-4-(2-naphthoylamino)nicotinamide;
4-(benzoylamino)-N-(2-hydroxyethyl)nicotinamide;
3-(benzoylamino)-2,6-dimethyl-N-phenylisonicotinamide;
3-(benzoylamino)-2,6-dimethyl-N-(3-nitrophenyl)isonicotinamide;
2-(benzoylamino)-N-[cyano(2-thienyl)methyl]nicotinamide; and
2-(benzoylamino)-N-[cyano(phenyl)methyl]nicotinamide.

In another embodiment, certain compounds of the present invention are those of formula I as defined above, wherein

R¹ is independently selected from hydrogen, halogen, hydroxyl, alkoxy, alkyl, halogenated alkoxy, and halogenated alkyl; and

R² is selected from 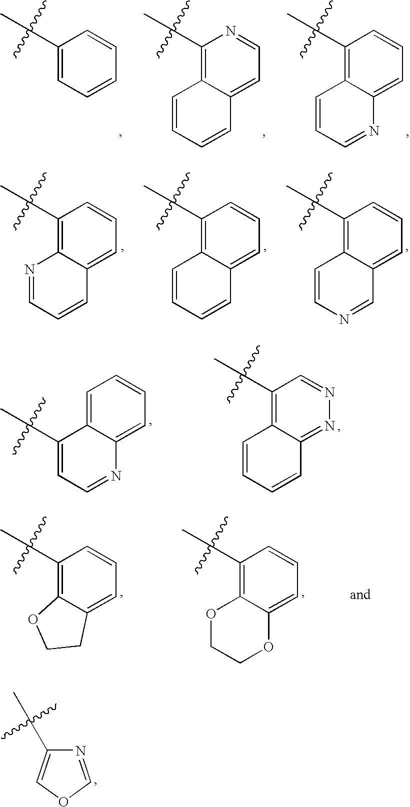
wherein said group used in defining R² is optionally substituted by one or more groups selected from halogen, halogenated alkyl, alkyl, halogenated alkoxy, cyano, nitro, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl-, aryl-alkyl and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkyl-carbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, cyano, nitro, C₁₋₆ alkoxy, C₁₋₆ alkyl and hydroxy.

In a further embodiment, certain compounds of the present invention are those of formula I, wherein

R¹ is independently selected from hydrogen, fluoro, chloro, hydroxyl, alkoxy, alkyl, halogenated alkoxy, and halogenated alkyl; and

R² is selected from 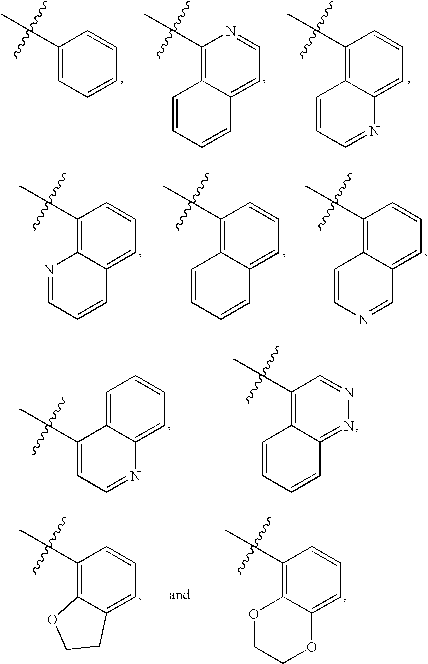
wherein said group used in defining R² is optionally substituted by one or more groups selected from halogen, halogenated alkyl, alkyl, halogenated alkoxy, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl- and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, C₁₋₆ alkoxy, C₁₋₆ alkyl and hydroxy.

In another embodiment, certain compounds of the present invention are those of formula IA or a pharmaceutically acceptable salt thereof, diastereomers, enantiomers, or mixtures thereof: 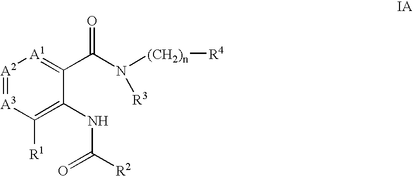
wherein:

one of A¹, A² or A³ is N and the remaining are each and independently CR¹; and

R¹ is independently selected from hydrogen, halogen, cyano, amino, acetylamino, hydroxyl, alkoxy, alkyl, halogenated alkoxy, alkylene, halogenated alkyl, halogenated alkenyl and NR⁵R⁶;

R²is selected from 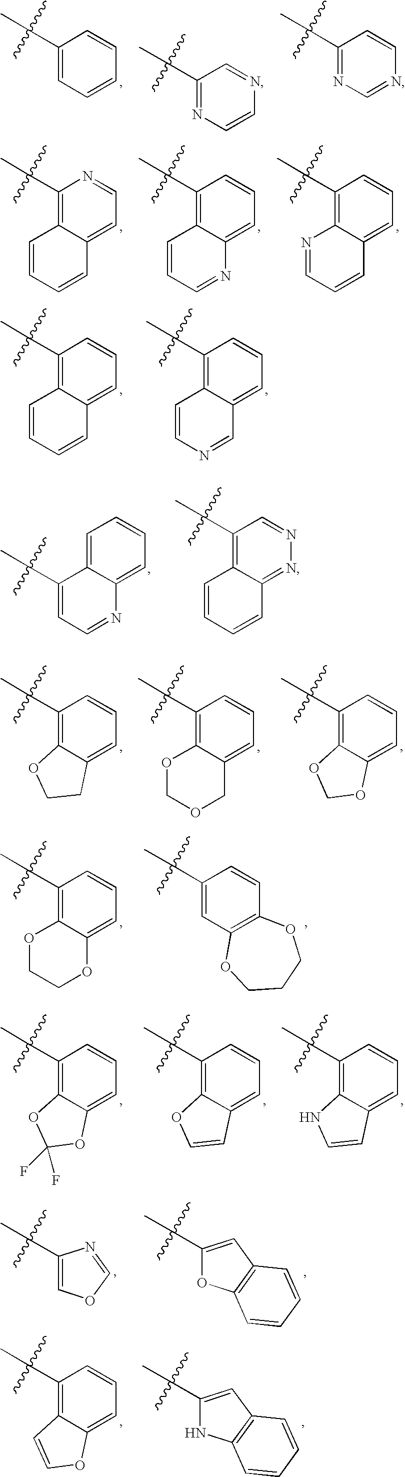 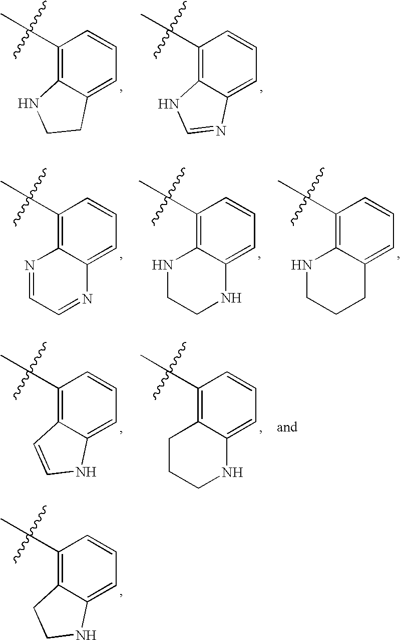
wherein said group used in defining R² is optionally substituted by one or more groups selected from halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl, wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, cyano, nitro, C₁₋₆ alkoxy, C₁₋₆ alkyl and hydroxy;

with a proviso that when n=0 then R⁴ is not thiazolyl or 5-chloropyridinyl;

with a further proviso that when R² is phenyl then n=0 and R⁴ is not unsubstituted methyl, C₃ alkyl or unsubstituted C₄ alkyl; and

with a further proviso that said compound of formula IA is not any one of
3-(benzoylamino)-N-benzylpyridine-2-carboxamide;
3-(benzoylamino)-N-pyridin-3-ylpyridine-2-carboxamide;
3-(benzoylamino)-N-phenylpyridine-2-carboxamide;
3-(benzoylamino)-N-(3-nitrophenyl)pyridine-2-carboxamide;
3-(benzoylamino)-N-(4-methoxyphenyl)pyridine-2-carboxamide;
3-(benzoylamino)-N-[4-(dimethylamino)phenyl]pyridine-2-carboxamide;
N-(2-hydroxyethyl)-4-(2-naphthoylamino)nicotinamide;
4-(benzoylamino)-N-(2-hydroxyethyl)nicotinamide;
3-(benzoylamino)-2,6-dimethyl-N-phenylisonicotinamide; and
3-(benzoylamino)-2,6-dimethyl-N-(3-nitrophenyl)isonicotinamide

In another embodiment, certain compounds of the present invention are those of formula IA as defined above, wherein

R¹ is independently selected from hydrogen, halogen, hydroxyl, alkoxy, alkyl, halogenated alkoxy, and halogenated alkyl; and

R² is selected from 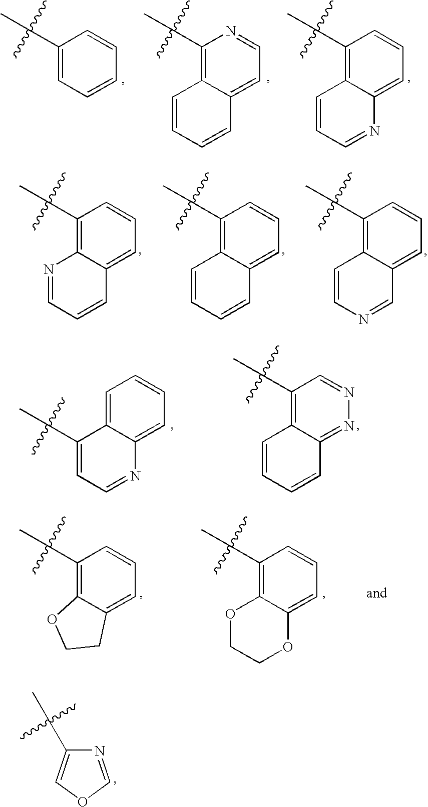
wherein said group used in defining R² is optionally substituted by one or more groups selected from halogen, halogenated alkyl, alkyl, halogenated alkoxy, cyano, nitro, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl-, aryl-alkyl and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkyl-carbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, cyano, nitro, C₁₋₆ alkoxy, C₁₋₆ alkyl and hydroxy.

In a further embodiment, certain compounds of the present invention are those of formula IA, wherein

R¹ is independently selected from hydrogen, fluoro, chloro, hydroxyl, alkoxy, alkyl, halogenated alkoxy, and halogenated alkyl; and

R¹ is selected from 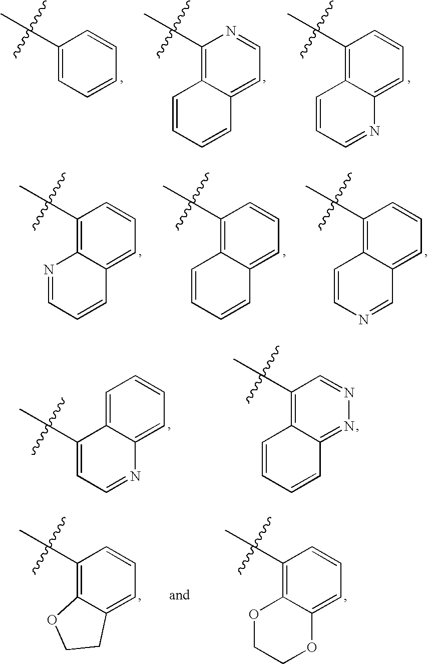
wherein said group used in defining R¹ is optionally substituted by one or more groups selected from halogen, halogenated alkyl, alkyl, halogenated alkoxy, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl- and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ allyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, C₁₋₆ alkoxy, C₁₋₆ alkyl and hydroxy.

In another embodiment, certain compounds of the present invention are those of formula IB or a pharmaceutically acceptable salt thereof, diastereomers, enantiomers, or mixtures thereof: 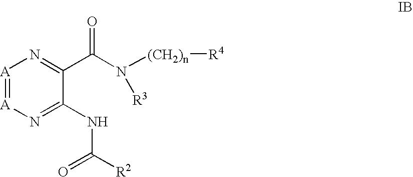
wherein:

A is each and independently CR¹; and

R¹ is independently selected from hydrogen, halogen, cyano, amino, acetylamino, hydroxyl, alkoxy, alkyl, halogenated alkoxy, alkylene, halogenated alkyl, halogenated alkenyl and NR⁵R⁶;

R² is selected from 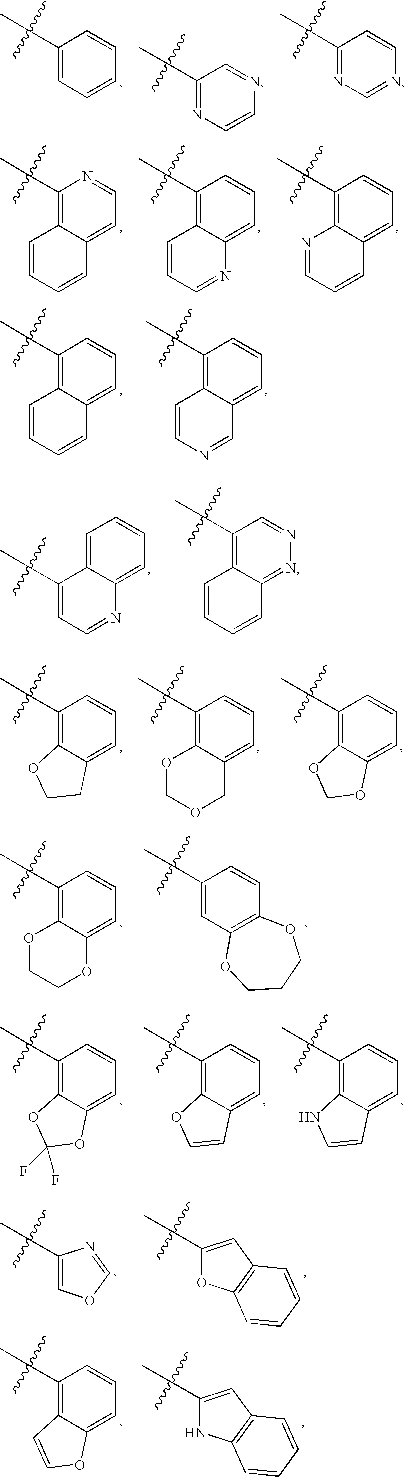 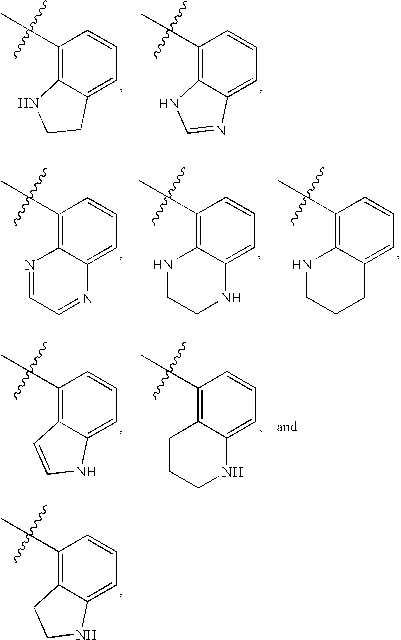
wherein said group used in defining R² is optionally substituted by one or more groups selected from halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, cyano, nitro, C₁₋₆ alkoxy, C₁₋₆ alkyl and hydroxy;

with a proviso that said compound of formula IB is not any one of 3-[(4-tert-butylbenzoyl)amino]-N-(5-chloro-pyridin-2-yl)pyrazine-2-carboxamide; N-[2-(1H-imidazol-2-yl)ethyl]-3-[[4-(1,1-dimethylethyl)benzoyl]amino]-2-pyrazinecarboxamide and 3-(benzoylamino)-N-(methoxycarbonylmethyl)pyrazine-2-carboxamide.

In a further embodiment, certain compounds of the invention are those formula IB, or a pharmaceutically acceptable salt thereof, diastereomers, enantiomers, or mixtures thereof, wherein

A is each and individually CR¹;

R¹ is independently selected from hydrogen, halogen, hydroxyl, alkoxy, alkyl, halogenated alkoxy, and halogenated alkyl;

R¹ is selected from 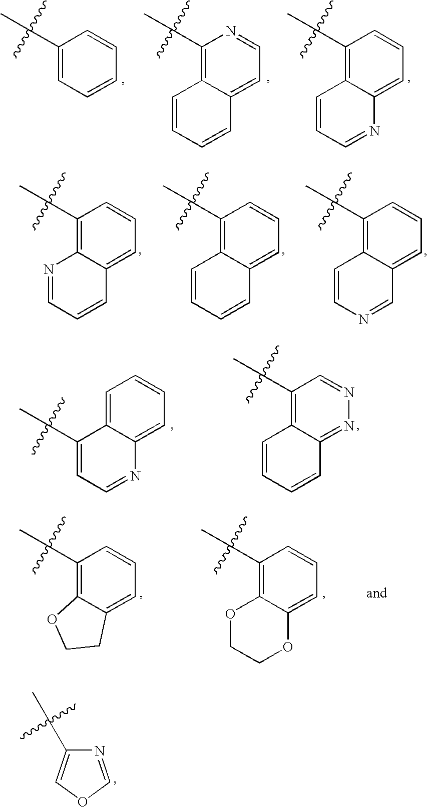
wherein said group used in defining R² is optionally substituted by one or more groups selected from halogen, halogenated alkyl, alkyl, halogenated alkoxy, cyano, nitro, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl-, aryl-alkyl and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkyl-carbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, cyano, nitro, C₁₋₆ alkoxy, C₁₋₆ alkyl and hydroxy.

In an even further embodiment, certain compounds of the invention are those of formula IB, or a pharmaceutically acceptable salt thereof, diastereomers, enantiomers, or mixtures thereof,

wherein

A is each and individually CR¹;

R¹ is independently selected from hydrogen, fluoro, chloro, hydroxyl, alkoxy, alkyl, halogenated alkoxy, and halogenated alkyl;

R² is selected from 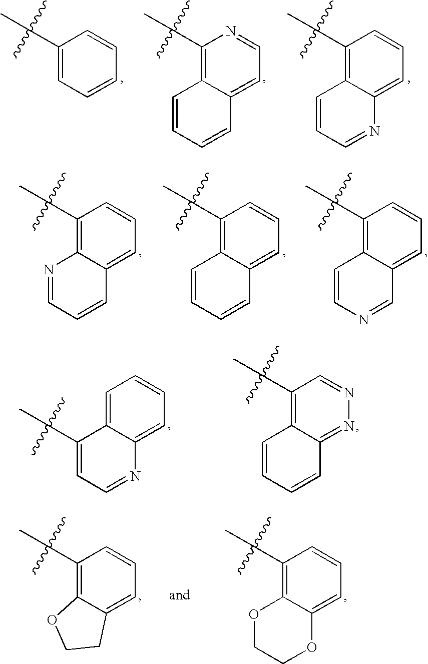
wherein said group used in defining R² is optionally substituted by one or more groups selected from halogen, halogenated alkyl, alkyl, halogenated alkoxy, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl- and —NR⁵R⁶;

R³ is selected from hydrogen and alkyl;

R⁴ is selected from alkyl, alkenyl, cycloalkyl, cycloalkenyl, alkoxy, aryl, heteroaryl and heterocyclyl; wherein said alkyl, alkenyl, cycloalkyl, cycloalkenyl, alkoxy, aryl, heteroaryl and heterocyclyl used in defining R⁴ is optionally substituted by one or more groups selected from halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and

n is selected from 0, 1, 2, 3, 4 and 5; or

R³ and R⁴ together with the nitrogen atom to which they are attached may form a group selected from heterocyclyl which is optionally fused with a five or six membered ring containing one or more heteroatoms; wherein said heterocyclyl which is optionally fused with a five or six membered ring containing one or more heteroatoms used in defining R³ and R⁴ is optionally substituted by one or more groups selected from halogen, halogenated alkyl, alkyl, cyano, nitro, amino, amino-alkyl, alkoxy, halogenated alkoxy, hydroxy, alkoxy-allyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶,

wherein each of R⁵ and R⁶ is independently selected from hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl; wherein said C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl used in defining R⁵ and R⁶ are optionally substituted by one or more groups selected from halogen, C₁₋₆ alkoxy, C₁₋₆ alkyl and hydroxy.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I, IA, IB or IC. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I, IA, IB or IC. It will further be understood that the present invention encompasses tautomers of the compounds of the Formula I, IA, IB or IC.

It will also be understood that certain compounds of the present invention may exist in solvated, for example hydrated, as well as unsolvated forms. It will further be understood that the present invention encompasses all such solvated forms of the compounds of the Formula I, IA, IB or IC.

Within the scope of the invention are also salts of the compounds of the Formula I, IA, IB or IC. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I, IA, IB or IC above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB₁ receptors. More particularly, the compounds of the invention exhibit activity as agonist of the CB₁ receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of CB₁ receptors is present or implicated. Furthermore, the compounds of the invention may be used to treat cancer, multiple sclerosis, Parkinson's disease, Huntington's chorea, Alzheimer's disease, anxiety disorders, gastrointestinal disorders and cardiovascular disorders.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of cannabinoid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastrointestinal disorders, e.g. gastroesophageal reflux disease, constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

In another aspect of the invention is the use of a compound according to Formula I, IA, IB or IC for inhibition of transient lower esophageal sphincter relaxations (TLESRs) and thus for treatment or prevention of gastroesophageal reflux disorder (GERD). The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. In further embodiments, the compounds according to the present invention are useful for the prevention of reflux, treatment or prevention of regurgitation, treatment or prevention of asthma, treatment or prevention of laryngitis, treatment or prevention of lung disease and for the management of failure to thrive.

A further aspect of the invention is the use of a compound according to Formula I, IA, IB or IC, for the manufacture of a medicament for the inhibition of transient lower esophageal sphincter relaxations, for the treatment or prevention of GERD, for the prevention of reflux, for the treatment or prevention of regurgitation, treatment or prevention of asthma, treatment or prevention of laryngitis, treatment or prevention of lung disease and for the management of failure to thrive.

Another aspect of the invention is the use of a compound according to Formula I, IA, IB or IC for the manufacture of a medicament for the treatment or prevention of functional gastrointestinal disorders, such as functional dyspepsia (FD). Yet another aspect of the invention is the use of a compound according to Formula I, IA, IB or IC for the manufacture of a medicament for the treatment or prevention of irritable bowel syndrome (IBS), such as constipation predominant IBS, diarrhea predominant IBS or alternating bowel movement predominant IBS. Exemplary irritable bowel syndrome (IBS) and functional gastrointestinal disorders, such as functional dyspepsia, are illustrated in Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders Diagnosis, Pathophysiology and Treatment. 2 ed. McLean, Va.: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl. 2), II1-II81.9-1-1999.

Also within the scope of the invention is the use of any of the compounds according to the Formula I, IA, IB or IC above, for the manufacture of a medicament for the treatment of any of the conditions discussed above.

A further aspect of the invention is a method for the treatment of a subject suffering from any of the conditions discussed above, whereby an effective amount of a compound according to the Formula I, IA, IB or IC above, is administered to a patient in need of such treatment.

Thus, the invention provides a compound of Formula I, IA, IB or IC, or pharmaceutically acceptable salt or solvate thereof, as hereinbefore defined for use in therapy.

In a further aspect, the present invention provides the use of a compound of Formula I, IA, IB or IC, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I, IA, IB or IC as defined above for the manufacture of a medicament.

Also within the scope of the invention is the use of any compound of Formula I, IA, IB or IC for the manufacture of a medicament for the therapy of pain.

Additionally provided is the use of any compound according to Formula I, IA, IB or IC for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I, IA, IB or IC above, is administered to a patient in need of such therapy.

Additionally, there is provided a pharmaceutical composition comprising a compound of Formula I, IA, IB or IC, or a pharmaceutically acceptable salt thereof, in association with a pharmaceutically acceptable carrier.

Particularly, there is provided a pharmaceutical composition comprising a compound of Formula I, IA, IB or IC, or a pharmaceutically acceptable salt thereof, in association with a pharmaceutically acceptable carrier for therapy, more particularly for therapy of pain.

Further, there is provided a pharmaceutical composition comprising a compound of Formula I, IA, IB or IC, or a pharmaceutically acceptable salt thereof, in association with a pharmaceutically acceptable carrier use in any of the conditions discussed above.

Another aspect of the invention is a method of preparing the compounds of the present invention.

One embodiment of the invention provides a method for preparing a compound of formula I, 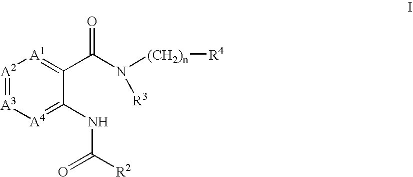
comprising the step of reacting a compound of formula II, 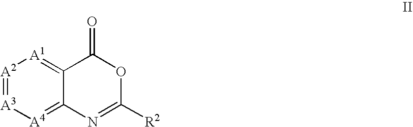
with a compound of R³(CH₂)ₙR⁴NH, in the presence of a base, such as an DIPEA, a solvent such as DMF,
wherein A¹, A², A³, A⁴, R², R³, R⁴ and n are as defined above.

Another embodiment of the invention provides a method for preparing a compound of formula IA, 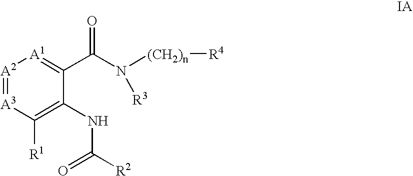
comprising the step of reacting a compound of formula IIA, 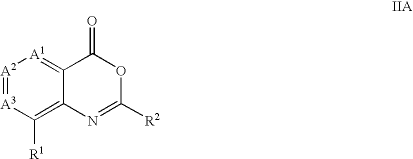
with a compound of R³(CH₂)ₙR⁴NH, in the presence of a base, such as an DIPEA, a solvent such as DMF,
wherein A¹, A², A³, R², R³, R⁴ and n are as defined above.

Another embodiment of the invention provides a method for preparing a compound of formula IB, 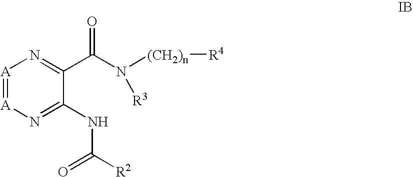
comprising the step of reacting a compound of formula IIB, 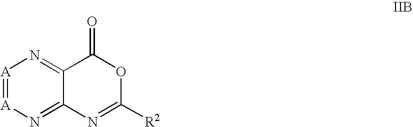
with a compound of R³(CH₂)ₙR⁴NH, in the presence of a base, such as an DIPEA, a solvent such as DMF,
wherein A, R², R³, R⁴ and n are as defined above.

Compounds of the present invention may also be prepared according to the synthetic routes as depicted in Schemes 1-5. 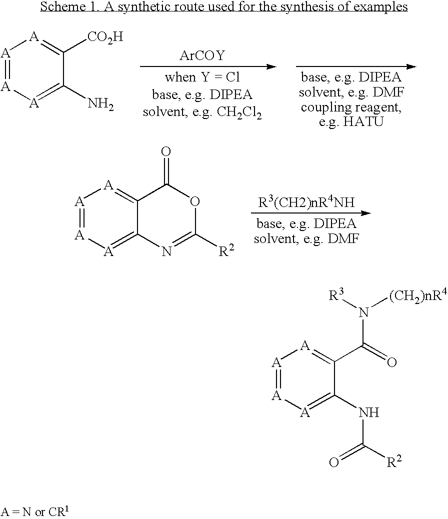 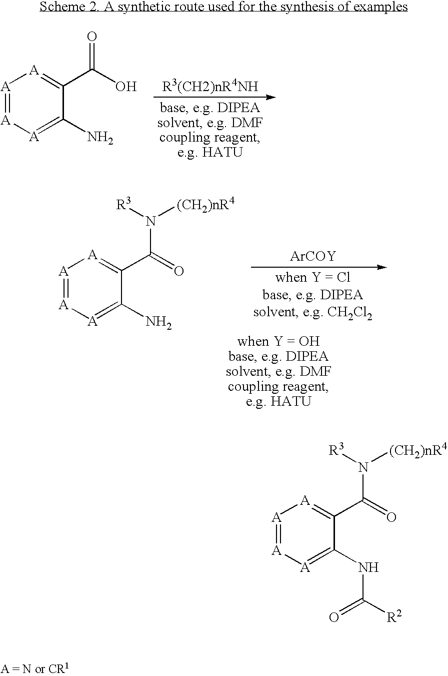 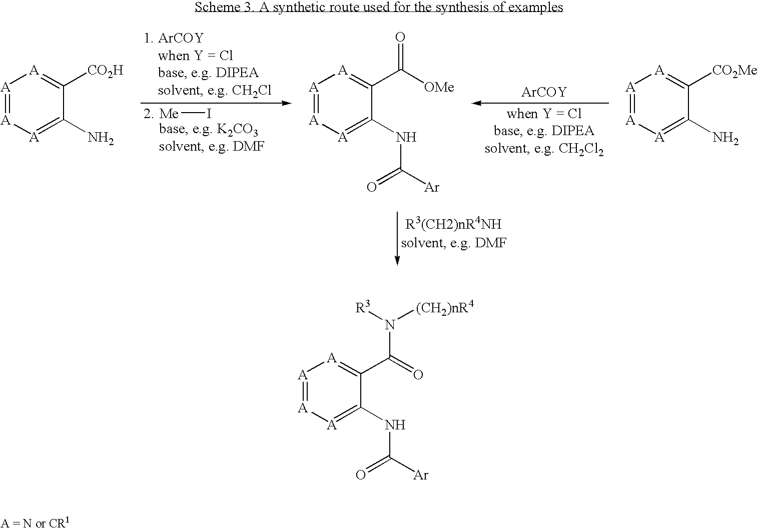 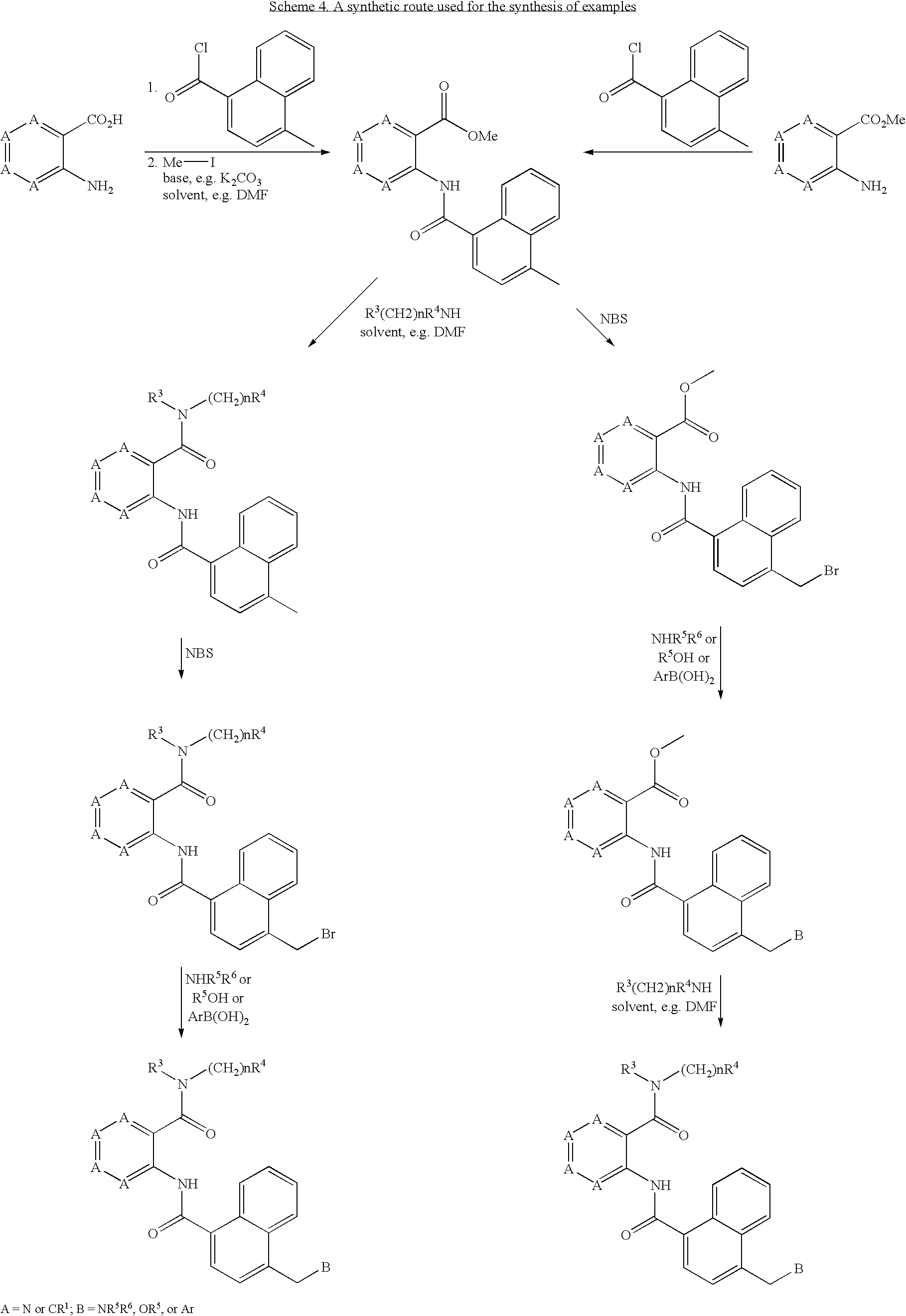 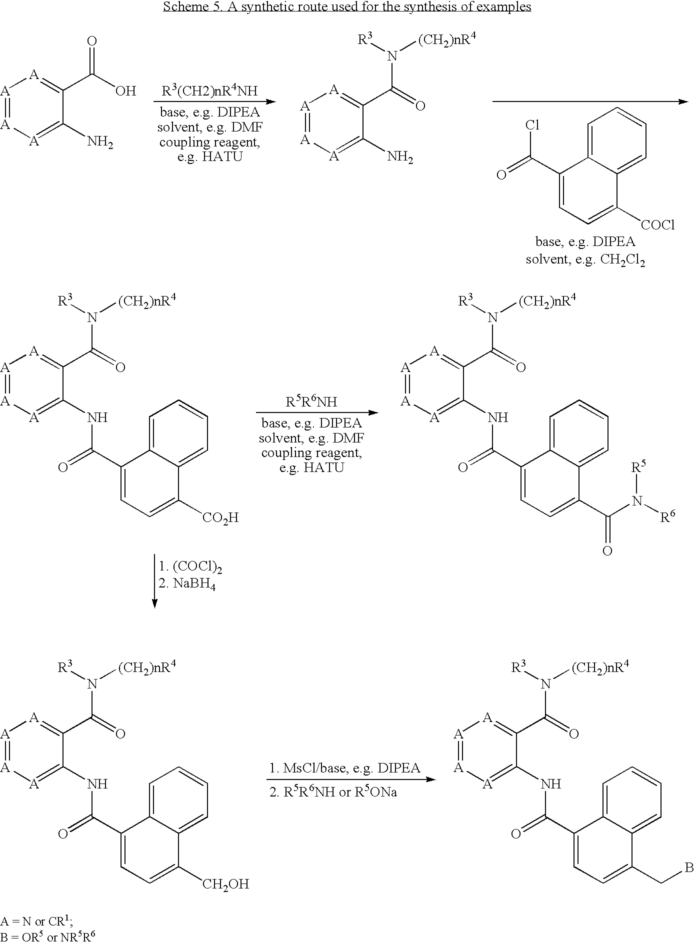
Biological Evaluation
hCB₁ and hCB₂ Receptor Binding

Human CB₁ receptor from Receptor Biology (hCB₁) or human CB₂ receptor from BioSignal (hCB₂) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ and hCB₂ GTPγS Binding

Human CB₁ receptor from Receptor Biology (hCB₁) or human CB₂ receptor membranes (BioSignal) are thawed at 37° C., passed 3 times through a 25-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the invention are evaluated from 10-point dose-response curves done in 300 μl with the appropriate amount of membrane protein and 100000-130000 dpm of GTPg³⁵S per well (0.11-0.14 nM). The basal and maximal stimulated binding is determined in absence and presence of 1 μM (hCB₂) or 10 μM (hCB₁) Win 55,212-2 respectively. The membranes are pre-incubated for 5 minutes with 56.25 μM (hCB2) or 1112.5 μM (hCB₁) GDP prior to distribution in plates (15 μM (hCB₂) or 30 μM (hCB₁) GDP final). The plates are vortexed and incubated for 60 minutes at room temperature, filtered on Unifilters GF/B (presoaked in water) with the Tomtec or Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) after adding 65 μl/well of MS-20 scintillation liquid. Antagonist reversal studies are done in the same way except that (a) an agonist dose-response curve is done in the presence of a constant concentration of antagonist, or (b) an antagonist dose-response curve is done in the presence of a constant concentration of agonist.

Based on the above assays, the dissociation constant (Ki) for a particular compound of the invention towards a particular receptor is determined using the following equation:
Ki=IC₅₀/(1+[rad]/Kd),

Wherein IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Using the above-mentioned assays, the Ki towards human CB₁ receptors for certain compounds of the invention is measured to be in the range of 0.2-5000 nM. The Ki towards human CB₂ receptors for certain compounds of the invention is measured to be in the range of about 4.5-4970 nM. The EC₅₀ towards human CB₁ receptors for certain compounds of the invention is measured to be in the range of about 1.5-2220 nM. The Eₘₐₓ towards human CB₁ receptors for certain compounds of the invention is measured to be in the range of about 20-130%.

The following table shows certain biological activities for some of the exemplified compounds.

| | | Ki (hCB1) | EC5O (hCB1) | Emax (hCB1) |
|---|---|---|---|---|
| COMPOUND | Structures | (nM) | (nM) | (%) |
| Example 80 | 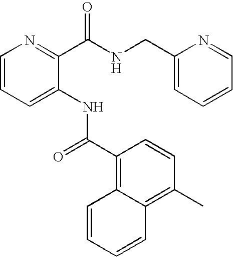 | 221 | 1227 | 75 |
| Example 171 | 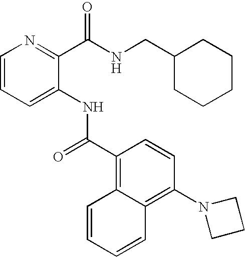 | 57 | 217 | 71 |
| Example 13 | 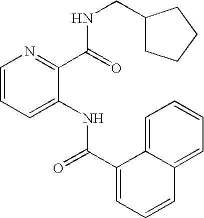 | 12 | 46 | 105 |
| Example 43 | 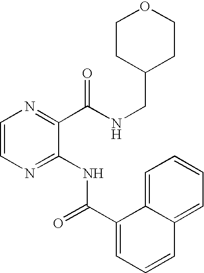 | 181 | 907 | 112 |

### EXAMPLES

The invention will further be described in more detail by the following Examples which describe methods whereby compounds of the present invention may be prepared, purified, analyzed and biologically tested, and which are not to be construed as limiting the invention.

### Example 1

### N-(Cyclobutylmethyl)-3-[(1-naphthalenylcarbonyl)amino]-2-pyridinecarboxamide

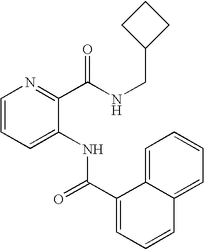

### Step A. N-(Cyclobutylmethyl)-3-[(1-naphthalenylcarbonyl)amino]-2-pyridinecarboxamide

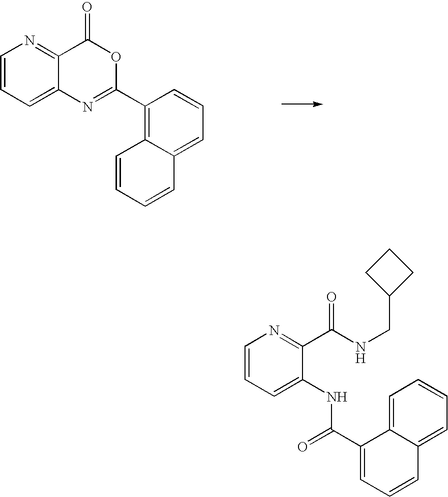

A solution of 2-(1-naphthalenyl)-H-pyrido[3,2-d][1,3]oxazin-4-one (100 mg, 0.365 mmol, see Step B for its preparation) in DMF (2 mL) was treated with cyclobutane methylamine (0.1 mL, 5.3 Min MeOH, 0.53 mmol) at 0° C. The mixture was stirred for 18 h at room temperature. After evaporation of the solvents, the residue was purified by MPLC using Hex/EtOAc (9:1) to provide the title compound (156 mg, 83%). ¹H NMR (400 MHz, CDCl₃) δ 1.69-1.78 (m, 2H), 1.81-1.91 (m, 2H), 1.99-2.07 (m, 2H), 2.51-2.62 (m, 1H), 3.34 (d, J=7.03 Hz, 2H), 7.52-7.59 (m, 4H), 7.87-7.89 (m, 1H), 7.92-7.96 (m, 1H), 8.03-8.05 (m, 1H), 8.30-8.35 (m, 1H), 8.42-8.45 (m, 1H), 9.27 (dd, J=8.59, 1.17 Hz, 1H). MS (ESI) (M+H)⁺ 360.0. Anal. (C, H, N) calcd for C₂₂H₂₁N₃O₂+0.30CH₃OH: C, 72.58; H, 6.06; N, 11.39. found C, 72.58; H, 5.86; N, 11.30.

### Step B. 2-(1-naphthalenyl)-H-pyrido[3,2-d][1,3]oxazin-4-one

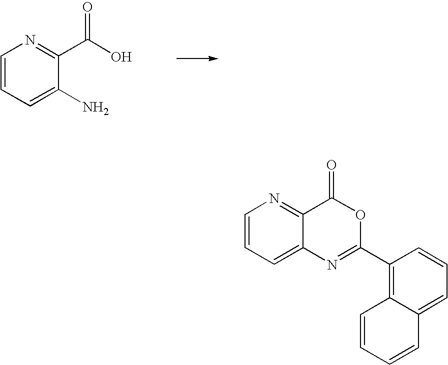

1-Naphthalenecarbonyl chloride (400 mg, 2.1 mmol) in CH₂Cl₂ (2 mL) was added into a solution of 3-amino-2-pyridinecarboxylic acid (277 mg, 2.0 mmol) and DIPEA (284 mg, 2.2 mmol) in DMF (10 mL) at 0° C. The reaction mixture was allowed to stir overnight at room temperature, and was then treated with DIPEA (284 mg, 2.2 mmol) and HATU (837 g, 2.2 mmol). After stirring for 1 h at room temperature, the reaction mixture was heated at 50° C. to provide the title compound which was used in Step A. MS (ESI) (M+H)⁺ 274.79.

### Example 2

### N-[2-(4-Morpholinyl)ethyl]-3-[(1-naphthalenylcarbonyl)amino]-2-pyridinecarboxamide

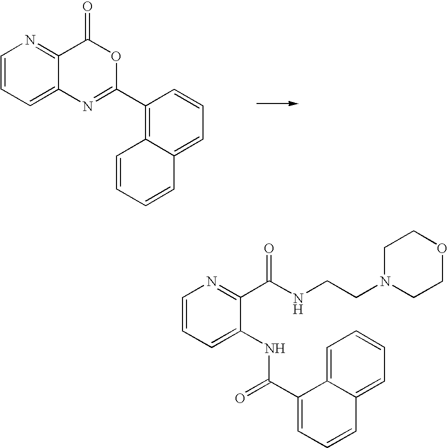

Following the procedure for Step A in Example 1, using DIPEA (0.67 mL, 3.8 mmol), 2-(1-naphthalenyl)-4H-pyrido[3,2-d][1,3]oxazin-4-one (100 mg, 0.36 mmol), and 4-morpholineethanamine (0.15 mL, 1.17 mmol) provided the title compound as its TFA salt after purification by reversed-phase HPLC (68 mg, 47%). ¹H NMR (400 MHz, CDCl₃) δ 2.47-2.54 (m, 4H), 2.60 (t, J=6.15 Hz, 2H), 3.46-3.55 (m, 2H), 3.73-3.75 (m, 4H), 7.51-7.60 (m, 4H), 7.89-7.92 (m, 2H), 7.97-7.99 (m, 1H), 8.31 (dd, J=4.39, 1.51 Hz, 1H), 8.53-8.55 (m, 1H), 8.72-8.78 (m, 1H), 9.41 (dd, J=8.59, 1.51 Hz, 1H), 12.80-12.86 (br s, 1H); MS (ESI) (M+H)⁺ 405.0; Anal. Calcd for C₂₃H₂₄N₄O₃+0.2 CH₃CN+0.6 CF₃CO₂H+0.7H₂O: C, 59.85; H, 5.43; N, 11.92. Found: C, 59.75; H, 5.35; N, 11.90.

### Example 3

### N-4-morpholinyl-3-[(1-naphthalenylcarbonyl)amino]-2-pyridinecarboxamide

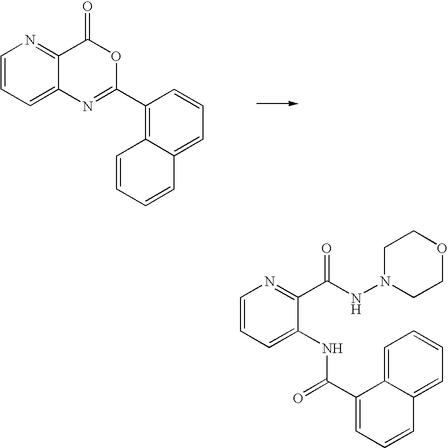

Following the procedure for Step A in Example 1, using DIPEA (0.67 mL, 3.8 mmol), 2-(1-naphthalenyl)-4H-pyrido[3,2-d][1,3]oxazin-4-one (100 mg, 0.36 mmol), and 4-morpholine amine (0.12 mL, 1.17 mmol) provided the title compound as its TFA salt after purification by reversed-phase HPLC (37 mg, 21%). ¹H NMR (400 MHz, CD₃OD) δ 2.87-2.89 (m, 4H), 3.74-3.77 (m, 4H), 7.54-7.64 (m, 4H), 7.90-7.92 (m, 1H), 7.95-7.97 (m, 1H), 8.05-8.07 (m, 1H), 8.37 (dd, J=4.49, 1.37 Hz, 1H), 8.42-8.44 (m, 1H), 9.28 (dd, J=8.59, 1.37 Hz, 1H), 12.65 (br s, 1H); MS (ESI) (M+H)⁺ 377.0; Anal. Calcd for C₂₁H₂₀N₄O₃+0.2H₂O: C, 66.37; H, 5.41; N, 14.74. Found: C, 66.46; H, 5.35; N, 14.63.

### Example 4

### 3-[(1-Naphthalenylcarbonyl)amino]-N-[(tetrahydro-2H-pyran-4-yl)methyl]-2-pyridinecarboxamide

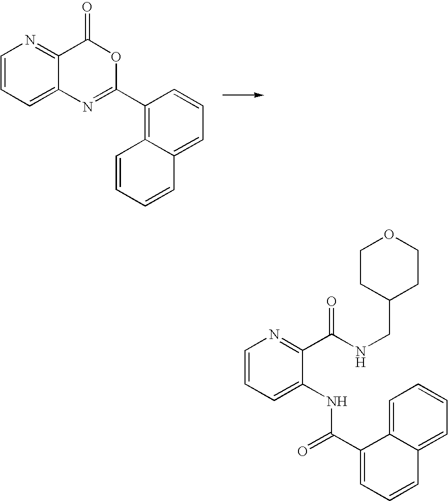

Following the procedure for Step A in Example 1, using 2-(1-naphthalenyl)-4H-pyrido[3,2-d][1,3]oxazin-4-one (122 mg, 0.446 mmol) and tetrahydro-2H-pyran-4-methanamine (62 mg, 0.535 mmol) provided the title compound (139 mg, 90%). ¹H NMR (400 MHz, CDCl₃) δ 0.98 (m, 2H), 1.23 (m, 3H), 1.56 (m, 1H), 1.76 (m, 5H), 3.25 (t, J=6.4 Hz, 2H), 7.54 (m, 4H), 7.90 (m, 2H), 7.98 (d, J=8.0 Hz, 1H), 8.28 (dd, J=8.4, 1.6 Hz, 1H), 8.53 (m, 2H), 9.41 (dd, J=8.4, 0.8 Hz, 1H), 12.87 (s, 1H); MS (ESI) (M+H)⁺=390.2; Anal. Calcd for C₂₃H₂₃N₃O₃: C, 70.93; H, 5.95; N, 10.79. Found: C, 70.82; H, 5.92; N, 10.64.

### Example 5

### N-Cyclohexyl-3-[(1-naphthalenylcarbonyl)amino]-2-pyridinecarboxamide

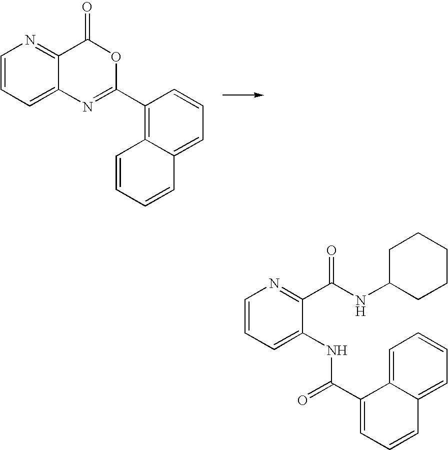

Following the procedure for Step A in Example 1, using DIPEA (1.02 mL, 5.8 mmol), 2-(1-naphthalenyl)-4H-pyrido[3,2-d][1,3]oxazin-4-one (150 mg, 0.55 mmol), and cyclohexylamine (0.19 mL, 1.65 mmol) provided the title compound as its TFA salt after purification by reversed-phase HPLC (68 mg, 33%). ¹H NMR (400 MHz, CD₃OD) δ 1.18-1.43 (m, 5H), 1.59-1.66 (m, 1H), 1.74-1.90 (m, 4H), 3.74-3.81 (m, 1H), 7.54-7.61 (m, 4H), 7.89-7.91 (m, 1H), 7.94-7.97 (m, 1H), 8.05-8.07 (m, 1H), 8.35 (dd, J=4.49, 1.46 Hz, 1H), 8.43-8.45 (m, 1H), 9.29 (dd, J=8.59, 1.46 Hz, 1H); MS (ESI) (M+H)⁺ 374.0; Anal. Calcd for C₂₃H₂₃N₃O₂: C, 73.97; H, 6.21; N, 11.25. Found: C, 74.14; H, 6.30; N, 11.33.

### Example 6

### N-(3-Methylcyclohexyl)-3-[(1-naphthalenylcarbonyl)amino]-2-pyridinecarboxamide

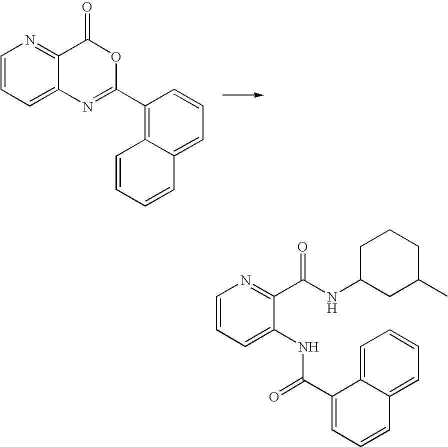

Following the procedure for Step A in Example 1, using 2-(1-naphthalenyl)-4H-pyrido[3,2-d][1,3]oxazin-4-one (100 mg, 0.36 mmol), and 3-methylcyclohexylamine (0.3 mL, 2.2 mmol) provided the title compound as its TFA salt after purification by reversed-phase HPLC (24 mg, 13%). ¹H NMR (400 MHz, CD₃OD) δ 0.82-1.04 (m, 5H), 1.19-1.79 (m, 6H), 1.87-1.92 (m, 1H), 3.74-3.81 (m, 1H), 7.54-7.63 (m, 4H), 7.91 (dd, J=7.03, 1.17 Hz, 1H), 7.94-7.98 (m, 1H), 8.05-8.08 (m, 1H), 8.34-8.37 (m, 1H), 8.43-8.45 (m, 1H), 9.27-9.31 (m, 1H); MS (ESI) (M+H)⁺ 388.0; Anal. Calcd for C₂₄H₂₅N₃O₂+0.2CH₃OH+0.1H₂O: C, 73.46; H, 6.62; N, 10.62. Found: C, 73.47; H, 6.46; N, 10.48.

### Example 7

### N-Cyclobutyl-3-[(1-naphthalenylcarbonyl)amino]-2-pyridinecarboxamide

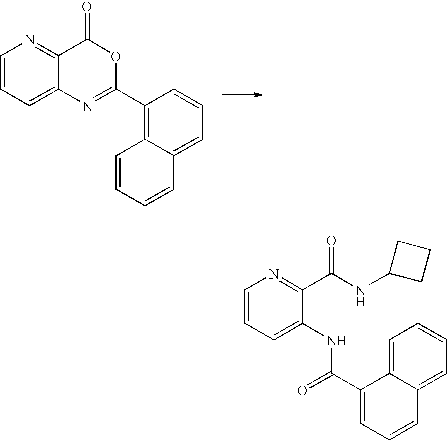

Following the procedure for Step A in Example 1, using 2-(1-naphthalenyl)-4H-pyrido[3,2-d][1,3]oxazin-4-one (100 mg, 0.36 mmol), and cyclobutylamine (0.2 mL, 2.16 mmol) provided the title compound as its TFA salt after purification by reversed-phase HPLC (20 mg, 12%). ¹H NMR (400 MHz, CD₃OD) δ 1.71-1.80 (m, 2H), 2.07-2.18 (m, 2H), 2.27-2.34 (m, 2H), 4.38-4.47 (m, 1H), 7.54-7.63 (m, 4H), 7.89-7.91 (m, 1H), 7.94-7.98 (m, 1H), 8.06-8.08 (m, 1H), 8.38 (dd, J=4.49, 1.32 Hz, 1H), 8.42-8.44 (m, 1H), 9.29 (dd, J=8.49, 1.32 Hz, 1H); MS (ESI) (M+H)⁺ 346.0; Anal. Calcd for C₂₁H₁₉N₃O₂+0.1H₂O: C, 72.65; H, 5.57; N, 12.10. Found: C, 72.63; H, 5.65; N, 12.02.

### Example 8

### N-(Cyclohexylmethyl)-3-[(1-naphthalenylcarbonyl)amino]-2-pyridinecarboxamide

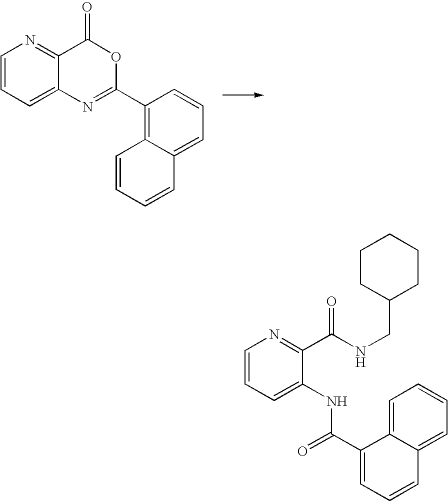

Following the procedure for Step A in Example 1, using 2-(1-naphthalenyl)-4H-pyrido[3,2-d][1,3]oxazin-4-one (129 mg, 0.47 mmol), and cyclohexanemethylamine (261 mg, 2.3 mmol) provided the title compound (172 mg, 95%). ¹H NMR (400 MHz, CD₃OD) δ 0.90-1.00 (m, 2H), 1.13-1.28 (m, 3H), 1.52-1.75 (m, 6H), 3.16 (d, J=6.83 Hz, 2H), 7.55-7.61 (m, 4H), 7.88-7.90 (m, 1H), 7.94-7.96 (m, 1H), 8.05-8.07 (m, 1H), 8.36 (dd, J=4.49, 1.56 Hz, 1H), 8.41-8.43 (m, 1H), 9.29 (dd, J=8.59, 1.37 Hz, 1H). MS (ESI) (M+H)⁺=388.0

### Example 9

### 3-[(1-Naphthalenylcarbonyl)amino]-N-(tetrahydro-2H-pyran-4-yl)-2-pyridinecarboxamide

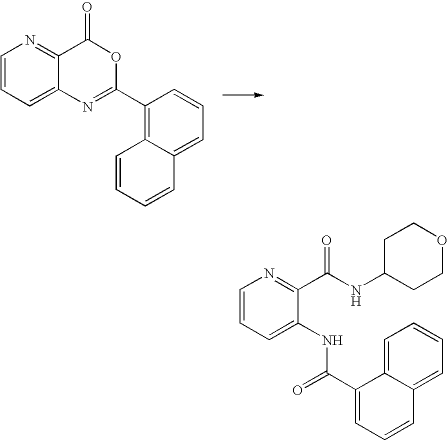

Following the procedure for Step A in Example 1, using DIPEA (0.2 mL, 1.08 mmol), 2-(1-naphthalenyl)-4H-pyrido[3,2-d][1,3]oxazin-4-one (100 mg, 0.36 mmol), and 4-tetrahydropyranamine (109 mg, 1.08 mmol) provided the title compound as its TFA salt after purification by reversed-phase HPLC (33 mg, 18%). ¹H NMR (400 MHz, CD₃OD) δ 1.63-1.73 (m, 2H), 1.81-1.88 (m, 2H), 3.44-3.50 (m, 2H), 3.90-3.96 (m, 2H), 3.98-4.07 (m, 1H), 7.56-7.62 (m, 3H), 7.88-7.90 (m, 1H), 7.93-7.97 (m, 1H), 8.05-8.07 (m, 1H), 8.36 (dd, J=4.49, 1.17 Hz, 1H), 8.40-8.45 (m, 1H), 9.28 (dd, J=8.59, 1.17 Hz, 1H); MS (ESI) (M+H)⁺ 376.3; Anal. Calcd for C₂₂H₂₁N₃O₃+0.2 CH₃OH: C, 69.83; H, 5.75; N, 11.00. Found: C, 69.87; H, 5.57; N, 10.93.

### Example 10

### 3-[(1-Naphthalenylcarbonyl)amino]-N-[2-(1-piperidinyl)ethyl]-2-pyridinecarboxamide

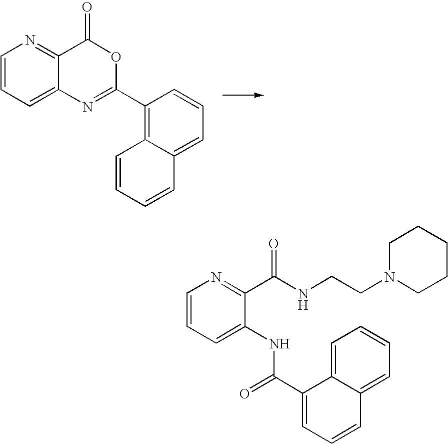

Following the procedure for Step A in Example 1, using DIPEA (0.4 mL, 2.2 mmol), 2-(1-naphthalenyl)-4H-pyrido[3,2-d][1,3]oxazin-4-one (200 mg, 0.73 mmol), and 1-(2-aminoethyl)piperidine (0.32 mL, 2.2 mmol) provided the title compound as its TFA salt after purification by reversed-phase HPLC (122 mg, 38%). ¹H NMR (400 MHz, CDCl₃) δ 1.22-1.85 (m, 7H), 2.81-2.96 (m, 2H), 3.53-3.78 (m, 5H), 7.49-7.66 (m, 4H), 7.86-7.94 (m, 2H), 8.04 (d, J=7.22 Hz, 1H), 8.34-8.41 (m, 2H), 9.20 (d, J=7.62 Hz, 1H); MS (ESI) (M+H)⁺ 403.3.

### Example 11

### N-(2-Hydroxypropyl)-3-[(1-naphthalenylcarbonyl)amino]-2-pyridinecarboxamide

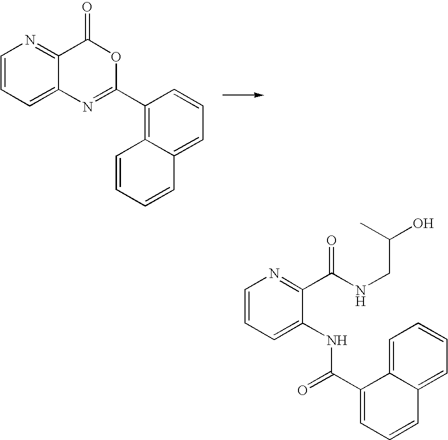

Following the procedure for Step A in Example 1, using DIPEA (0.1 mL, 1.1 mmol), 2-(1-naphthalenyl)-4H-pyrido[3,2-d][1,3]oxazin-4-one (100 mg, 0.36 mmol), and 1-amino-2-propanol (0.2 mL, 2.2 mmol) provided the title compound as its TFA salt after purification by reversed-phase HPLC (78 mg, 47%). ¹H NMR (400 MHz, CD₃OD) δ 1.14 (d, J=6.25 Hz, 3H), 3.23 (dd, J=13.57, 7.32 Hz, 1H), 3.40 (dd, J=13.57, 4.20 Hz, 1H), 3.86-3.92 (m, 1H), 7.52-7.61 (m, 4H), 7.89 (dd, J=7.03, 1.17 Hz, 1H), 7.92-7.96 (m, 1H), 8.05 (d, J=8.40 Hz, 1H), 8.35 (dd, J=4.49, 1.56 Hz, 1H), 8.41-8.43 (m, 1H), 9.28 (dd, J=8.59, 1.56 Hz, 1H), 12.90 (d, J=9.96 Hz, 1H); MS (ESI) (M+H)⁺ 350.3; Anal. Calcd for C₂₀H₁₉N₃O₃+0.1 CF₃COOH: C, 67.25; H, 5.34; N, 11.65. Found: C, 67.39; H, 5.45; N, 11.52.

### Example 12

### N-(2-Hydroxybutyl)-3-[(1-naphthalenylcarbonyl)amino]-2-pyridinecarboxamide

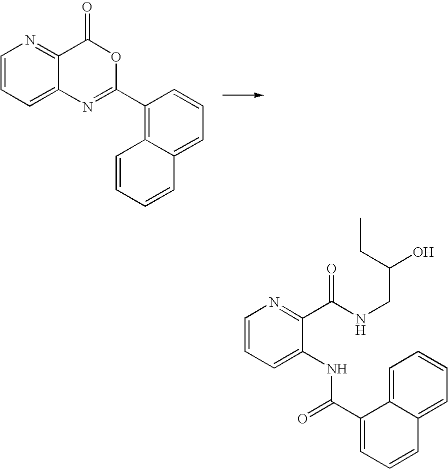

Following the procedure for Step A in Example 1, using DIPEA (0.1 mL, 1.1 mmol), 2-(1-naphthalenyl)-4H-pyrido[3,2-d][1,3]oxazin-4-one (100 mg, 0.36 mmol), and 1-amino-2-butanol (96 mg, 1.1 mmol) provided the title compound as its TFA salt after purification by reversed-phase HPLC (38 mg, 22%). ¹H NMR (400 MHz, CD₃OD) δ 0.93 (t, J=7.42 Hz, 3H), 1.38-1.53 (m, 2H), 3.22 (dd, J=13.67, 7.62 Hz, 1H), 3.46 (dd, J=13.67, 3.91 Hz, 1H), 3.58-3.64 (m, 1H), 7.52-7.61 (m, 4H), 7.88 (dd, J=7.03, 1.17 Hz, 1H), 7.92-7.95 (m, 1H), 8.04-8.06 (m, 1H), 8.35 (dd, J=4.49, 1.56 Hz, 1H), 8.40-8.43 (m, 1H), 9.28 (dd, J=8.59, 1.56 Hz, 1H); MS (ESI) (M+H)⁺ 364.2; Anal. Calcd for C₂₁H₂₁N₃O₃+0.4 CF₃COOH+0.1H₂O: C, 63.73; H, 5.30; N, 10.23. Found: C, 63.75; H, 5.25; N, 9.99.

### Example 13

### N-(Cyclopentylmethyl)-3-[(1-naphthalenylcarbonyl)amino]-2-pyridinecarboxamide

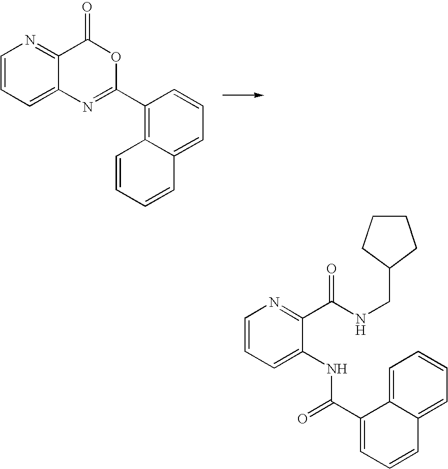

Following the procedure for Step A in Example 1, using DIPEA (0.2 mL, 1.1 mmol), 2-(1-naphthalenyl)-4H-pyrido[3,2-d][1,3]oxazin-4-one (100 mg, 0.36 mmol), and cyclopentanemethylamine (0.33 mL, 1.1 mmol) provided the title compound as its TFA salt after purification by reversed-phase HPLC (52 mg, 29%). ¹H NMR (400 MHz, CD₃OD) δ 1.16-1.24 (m, 2H), 1.45-1.63 (m, 4H), 1.66-1.74 (m, 2H), 2.05-2.17 (m, 1H), 3.20-3.23 (m, 2H), 7.49-7.56 (m, 4H), 7.86 (dd, J=7.03, 0.98 Hz, 1H), 7.89-7.93 (m, 1H), 8.00-8.02 (m, 1H), 8.29 (dd, J=4.49, 1.46 Hz, 1H), 8.40-8.44 (m, 1H), 9.01-9.07 (m, 1H), 9.23 (dd, J=8.59, 1.46 Hz, 1H), 12.89-12.93 (br.s, 1H); MS (ESI) (M+H)⁺ 374.2; Anal. Calcd for C₂₃H₂₃N₃O₂+0.2H₂O: C, 73.27; H, 6.26; N, 11.14. Found: C, 74.10; H, 6.19; N, 11.08.

### Example 14

### 3-[(1-Naphthalenylcarbonyl)amino]-N-(2-piperidinylmethyl)-2-pyridinecarboxamide

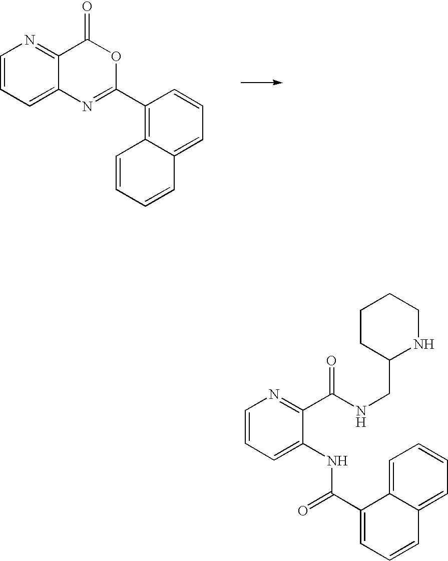

Following the procedure for Step A in Example 1, using 2-(1-naphthalenyl)-4H-pyrido[3,2-d][1,3]oxazin-4-one (100 mg, 0.36 mmol), and 2-(aminomethyl)piperidine (250 mg, 2.2 mmol) provided the title compound as its TFA salt after purification by reversed-phase HPLC (14 mg, 8%). ¹H NMR (400 MHz, CD₃OD) δ 1.43-1.64 (m, 3H), 1.73-1.95 (m, 3H), 2.80-2.86 (m, 1H), 3.20-3.22 (m, 2H), 3.48-3.67 (m, 2H), 7.53-7.58 (m, 3H), 7.63 (dd, J=8.59, 4.49 Hz, 1H), 7.88 (dd, J=7.23, 1.17 Hz, 1H), 7.93-7.97 (m, 1H), 8.04-8.06 (m, 1H), 8.39 (dd, J=4.49, 1.37 Hz, 1H), 8.40-8.43 (d, 1H), 9.27 (dd, J=8.59, 1.37 Hz, 1H); MS (ESI) (M+H)⁺ 389.2.

### Example 15

### N-(2,2-Dimethylpropyl)-3-(1-naphthoylamino)pyridine-2-carboxamide

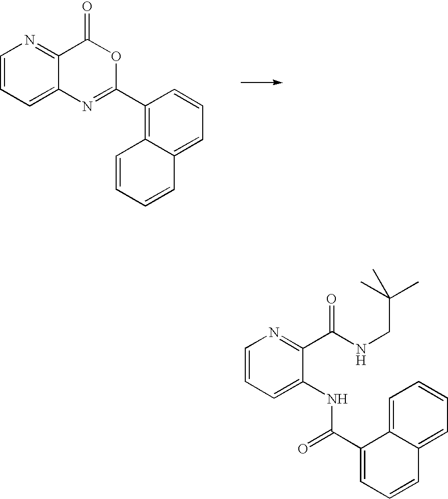

Following the procedure for Step A in Example 1, using 2-(1-naphthalenyl)-4H-pyrido[3,2-d][1,3]oxazin-4-one (100 mg, 0.36 mmol), and (2,2-dimethylpropyl)amine (174 mg, 2.0 mmol) provided the title compound as its TFA salt after purification by reversed-phase HPLC (49 mg, 29%). ¹H NMR (400 MHz, CD₃OD) δ 0.95 (s, 9H), 3.18 (s, 2H), 7.58 (m, 4H), 7.90 (d, J=7.2 Hz, 1H), 7.97 (m, 1H), 8.07 (d, J=8.4 Hz, 1H), 8.39 (m, 1H), 8.44 (m, 1H), 9.31 (d, J=8.8 Hz, 1H); MS (ESI) (M+H)⁺ 362.0.

### Example 16

### N-(2-Methoxy-1-methylethyl)-3-(1-naphthoylamino)pyridine-2-carboxamide

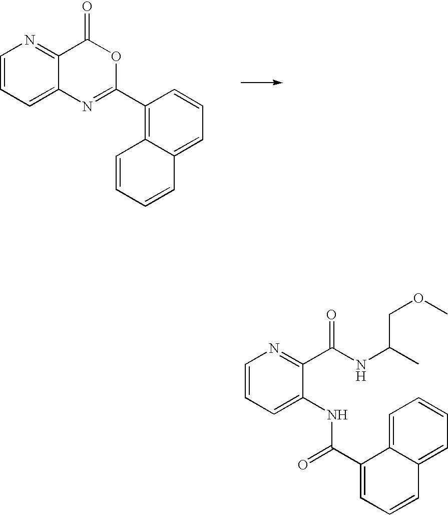

Following the procedure for Step A in Example 1, using 2-(1-naphthalenyl)-4H-pyrido[3,2-d][1,3]oxazin-4-one (100 mg, 0.36 mmol), and (2-methoxy-1-methylethyl)amine (178 mg, 2.0 mmol) provided the title compound as its TFA salt after purification by reversed-phase HPLC (56 mg, 33%). ¹H NMR (400 MHz, CDCl₃) δ 1.28 (d, J=6.8 Hz, 3H), 3.39 (s, 3H), 3.45 (m, 2H), 4.24 (m, 1H), 7.54 (m, 4H), 7.89 (m, 2H), 7.98 (d, J=8.4 Hz, 1H), 8.29 (m, 1H), 8.53 (m, 2H), 9.40 (dd, J=8.4, 1.2 Hz, 1H), 12.84 (s, 1H); MS (ESI) (M+H)⁺ 364.0.

### Example 17

### N-[(1-Hydroxycyclohexyl)methyl]-3-(1-naphthoylamino)pyridine-2-carboxamide

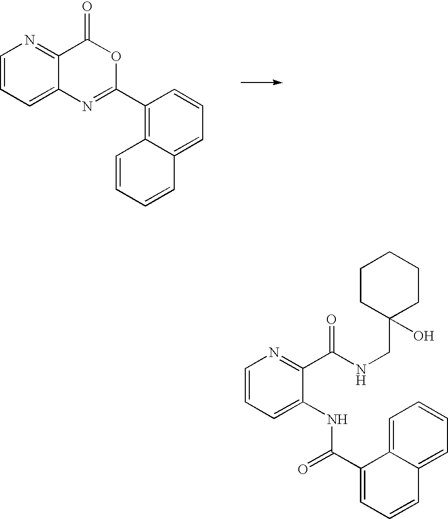

Following the procedure for Step A in Example 1, using DIPEA (129 mg, 1.0 mmol), 2-(1-naphthalenyl)-4H-pyrido[3,2-d][1,3]oxazin-4-one (100 mg, 0.36 mmol), and 1-(aminomethyl)cyclohexanol (129 mg, 1.0 mmol) provided the title compound as its TFA salt after purification by reversed-phase HPLC (29 mg, 16%). ¹H NMR (400 MHz, CD₃OD) δ 1.13-1.30 (m, 1H), 1.37 (d, J=10.15 Hz, 9H), 3.28 (s, 2H), 7.39-7.61 (m, 4H), 7.78-7.85 (m, 1H), 7.85-7.93 (m, 1H), 7.98 (d, J=8.20 Hz, 1H), 8.29 (dd, J=4.49, 1.37 Hz, 1H), 8.32-8.39 (m, 1H), 9.22 (dd, J=8.59, 1.37 Hz, 1H); MS (ESI) (M+H)⁺ 404.0.

### Example 18

### N-(Cyclobutylmethyl)-3-[[(4-methyl-1-naphthalenyl)carbonyl]amino]-2-pyridinecarboxamide

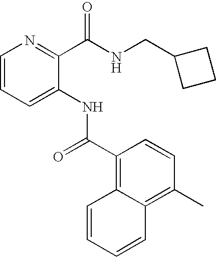

### Step A. N-(Cyclobutylmethyl)-3-[[(4-methyl-1-naphthalenyl)carbonyl]amino]-2-pyridinecarboxamide

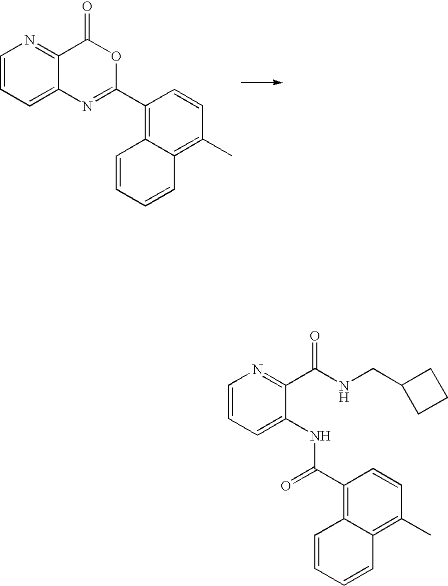

Following the procedure for Step A in Example 1, using 2-(4-methyl-1-naphthalenyl)-4H-pyrido[3,2-d][1,3]oxazin-4-one (130 mg, 0.45 mmol, see Step B for its preparation) and cyclobutylmethylamine (0.5 mL, 5.3 Min MeOH, 2.5 mmol) provided the title compound (105 mg, 72%). ¹H NMR (400 MHz, CD₃OD) δ 1.77 (m, 2H), 1.87 (m, 2H), 2.05 (m, 2H), 2.60 (m, 1H), 2.76 (s, 3H), 3.37 (d, J=7.03 Hz, 2H), 7.46 (d, J=7.23 Hz, 1H), 7.59 (m, 3H), 7.80 (d, J=7.23 Hz, 1H), 8.14 (m, 1H), 8.36 (dd, J=4.49, 1.37 Hz, 1H), 8.46 (m, 1H), 9.29 (dd, J=8.59, 1.37 Hz, 1H). MS (ESI) (M+H)⁺=374.0.

### Step B. 2-(4-Methyl-1-naphthalenyl)-4H-pyrido[3,2-d][1,3]oxazin-4-one

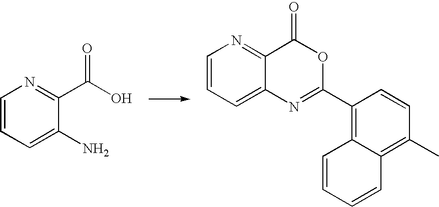

Following the procedure for Step B in Example 1, a suspension of 3-amino-2-pyridinecarboxylic acid (414 mg, 3.0 mmol) in CH₂Cl₂ (10 mL) and DIPEA (1.25 mL, 7.2 mmol) was treated with 4-methyl-1-naphthalenecarbonyl chloride, prepared from 4-methyl-1-naphthalenecarbonylic acid (590 mg, 3.17 mmol) with thionyl chloride (4.11 g, 35 mmol), and then with HATU (1.25 g, 3.3 mmol) in DMF (10 mL). The title compound was formed and directly used in Step A.

### Example 19

### 3-[[(4-Methyl-1-naphthalenyl)carbonyl]amino]-N-[(tetrahydro-2H-pyran-4-yl)methyl]-2-pyridinecarboxamide

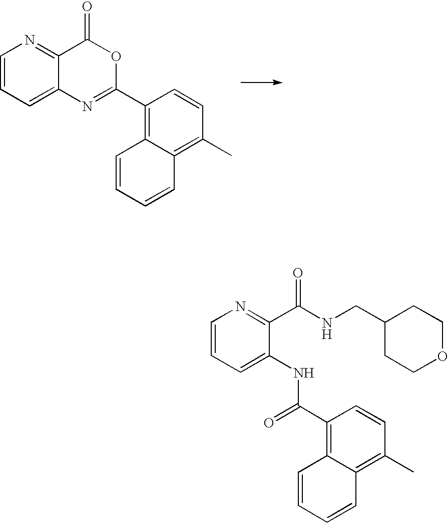

Following the procedure for Step A in Example 1, using 2-(4-methyl-1-naphthalenyl)-4H-pyrido[3,2-d][1,3]oxazin-4-one (108 mg, 0.375 mmol) and tetrahydro-2H-pyran-4-methanamine (122 mg, 1.06 mmol) provided the title compound (75 mg, 49%). ¹H NMR (400 MHz, CD₃OD) δ 1.26 (dd, J=11.91, 4.49 Hz, 1H), 1.33 (dd, J=11.9, 4.5 Hz, 1H), 1.63 (m, 2H), 1.85 (m, 1H), 2.76 (s, 3H), 3.24 (d, J=7.03 Hz, 2H), 3.36 (m, 2H), 3.90 (dd, J=11.03, 3.22 Hz, 2H), 7.45 (m, 1H), 7.60 (m, 3H), 7.79 (d, J=7.23 Hz, 1H), 8.13 (m, 1H), 8.36 (dd, J=4.49, 1.37 Hz, 1H), 8.46 (m, 1H), 9.28 (dd, J=8.59, 1.37 Hz, 1H). MS (ESI) (M+H)⁺=404.0. Anal. (C, H, N) calcd for C₂₄H₂₅N₃O₃+0.1H₂O: C, 71.13; H, 6.27; N, 10.37. found C, 71.03; H, 6.04; N, 10.26.

### Example 20

### 3-[(4-Methyl-1-naphthoyl)amino]-N-(piperidin-2-ylmethyl)pyridine-2-carboxamide

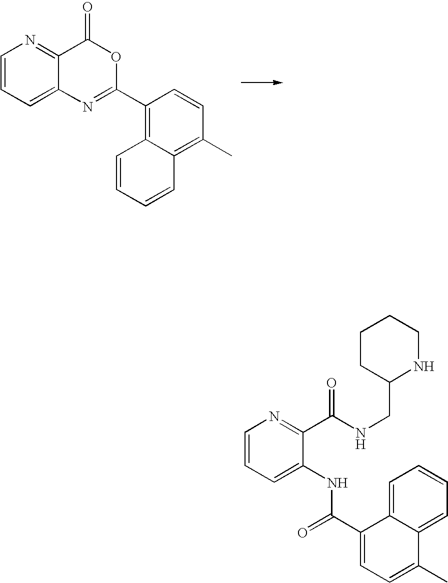

Following the procedure for Step A in Example 1, using 2-(4-methyl-1-naphthyl)-4H-pyrido[3,2-d][1,3]oxazin-4-one (288 mg, 1.0 mmol) and (piperidin-2-yl-methyl)amine (340 mg, 3.0 mmol) provided the title compound as its TFA salt after purification by reversed-phase HPLC (195 mg, 38%). ¹H NMR (400 MHz, CD₃OD) δ 1.58 (m, 3H), 1.88 (m, 3H), 2.77 (s, 3H), 2.86 (m, 1H), 3.29 (m, 2H), 3.58 (m, 2H), 7.43 (d, J=7.6 Hz, 1H), 7.61 (m, 3H), 7.80 (d, J=7.6 Hz, 1H), 8.15 (d, J=8.0 Hz, 1H), 8.41 (dd, J=4.4, 1.2 Hz, 1H), 8.46 (dd, J=8.0, 0.8 Hz, 1H), 9.28 (dd, J=8.8, 0.8 Hz, 1H); MS (ESI) (M+H)⁺ 403.3.

### Example 21

### N-(Cyclobutylmethyl)-3-[[(4-methoxy-1-naphthalenyl)carbonyl]amino]-2-pyridinecarboxamide

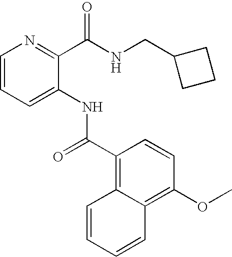

### Step A. N-(Cyclobutylmethyl)-3-[[(4-methoxy-1-naphthalenyl)carbonyl]amino]-2-pyridinecarboxamide

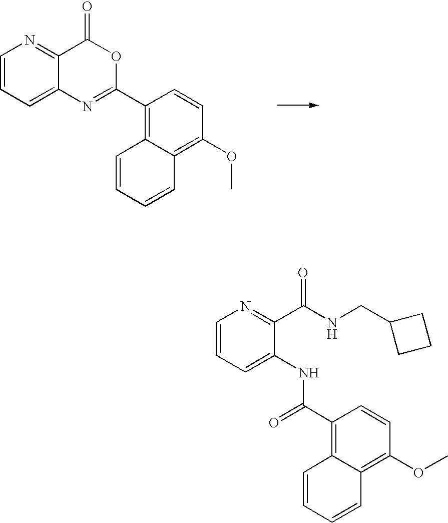

Following the procedure for Step A in Example 1, using 2-(4-methoxy-1-naphthalenyl)-4H-pyrido[3,2-d][1,3]oxazin-4-one (120 g, 0.40 mmol, see Step B for its preparation) and cyclobutylmethylamine (0.5 mL, 5.3 M in MeOH, 2.5 mmol) provided the title compound (87 mg, 56%). ¹H NMR (400 MHz, CD₃OD) δ 1.77 (m, 2H), 1.88 (m, 2H), 2.06 (m, 2H), 2.61 (m, 1H), 3.38 (d, J=7.23 Hz, 2H), 4.08 (s, 3H), 7.02 (d, J=8.20 Hz, 1H), 7.56 (m, 3H), 7.93 (d, J=8.01 Hz, 1H), 8.32 (m, 2H), 8.52 (m, 1H), 9.27 (dd, J=8.59, 1.37 Hz, 1H). MS (ESI) (M+H)⁺=390.0.

### Step B. 2-(4-Methoxy-1-naphthalenyl)-4H-pyrido[3,2-d][1,3]oxazin-4-one

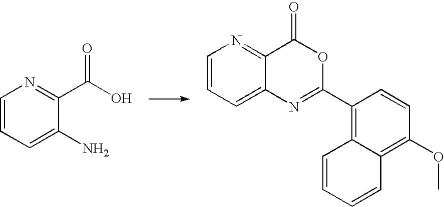

Following the procedure for Step B in Example 18, using 3-amino-2-pyridinecarboxylic acid (690 mg, 5.0 mmol), DIPEA (780 mg, 6.0 mmol), 4-methoxy-1-naphthalenecarbonyl chloride, prepared from 4-methoxy-1-naphthoic acid (1.0 g, 5.0 mmol) and oxalyl chloride (5 mL, 2.0 M in CH₂Cl₂, 10 mmol), and then HATU (2.28 g, 6.0 mmol) provided the title compound which was directly used in Step A.

### Example 22

### 3-[(4-Methoxy-1-naphthoyl)amino]-N-(tetrahydro-2H-pyran-4-ylmethyl)pyridine-2-carboxamide

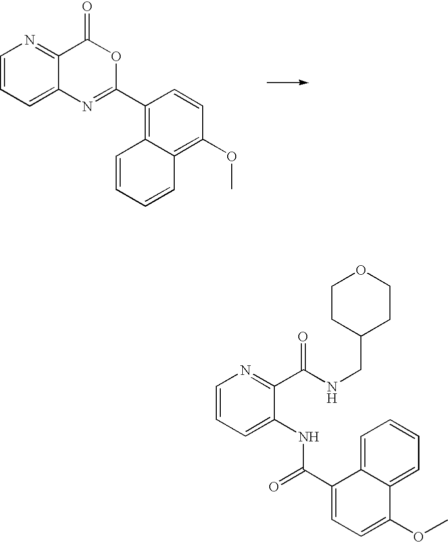

Following the procedure for Step A in Example 1, using 2-(4-methoxy-1-naphthyl)-4H-pyrido[3,2-d][1,3]oxazin-4-one (120 mg, 0.4 mmol), and tetrahydro-2H-pyran-4-methanamine (210 mg, 1.8 mmol) provided the title compound (81 mg, 48%). ¹H NMR (400 MHz, CD₃OD) δ 1.31 (m, 2H), 1.64 (dd, J=13.08, 1.17 Hz, 2H), 1.87 (m, J=7.62, 3.51 Hz, 1H), 3.26 (m, J=6.83 Hz, 2H), 3.36 (m, 2H), 3.91 (dd, J=11.72, 3.51 Hz, 2H), 4.08 (s, 3H), 7.01 (d, J=8.20 Hz, 1H), 7.56 (m, 3H), 7.93 (d, J=8.01 Hz, 1H), 8.33 (m, 2H), 8.51 (d, J=8.59 Hz, 1H), 9.26 (m, 1H). MS (ESI) (+H)⁺=420.0.

### Example 23

### N-(Cyclohexylmethyl)-3-[[[4-(dimethylamino)-1-naphthalenyl]carbonyl]amino]-2-pyridinecarboxamide

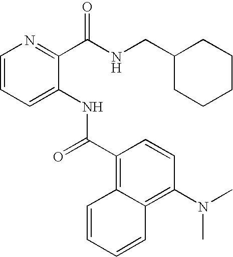

### Step A. N-(Cyclohexylmethyl)-3-[[[4-(dimethylamino)-1-naphthalenyl]carbonyl]amino]-2-pyridinecarboxamide

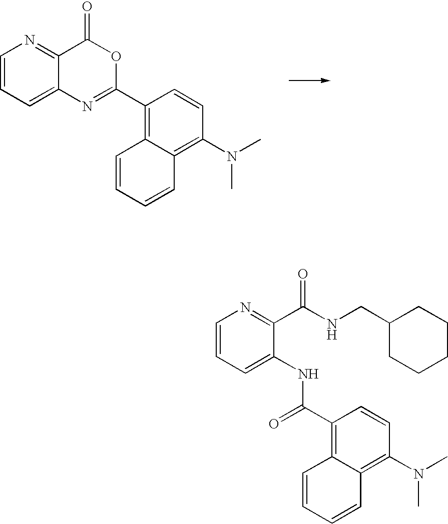

Following the procedure for Step A in Example 1, using 2-[4-(dimethylamino)-1-naphthalenyl]-4H-pyrido[3,2-d][1,3]oxazin-4-one (1.47 g, 4.64 mmol, see Step B for its preparation) and cyclohexanemethylamine (174 mmol, 1.54 mmol) provided the title compound as its TFA salt after purification by reversed-phase HPLC (15 mg, 2%). ¹H NMR (400 MHz, CDCl₃) δ 1.00 (m, 2H), 1.21 (m, 4H), 1.75 (m, 4H), 3.03 (s, 6H), 3.25 (t, J=6.64 Hz, 2H), 7.18 (d, J=7.81 Hz, 1H), 7.51 (m, 1H), 7.57 (m, 2H), 7.89 (d, J=7.81 Hz, 1H), 8.27 (m, 2H), 8.54 (t, J=5.86 Hz, 1H), 8.61 (m, 1H), 9.39 (dd, J=8.59, 1.37 Hz, 1H), 12.83 (s, 1H); MS (ESI) (M+H)⁺ 431.0.

### Step B. 2-[4-(Dimethylamino)-1-naphthalenyl]-4H-pyrido[3,2-d][1,3]oxazin-4-one

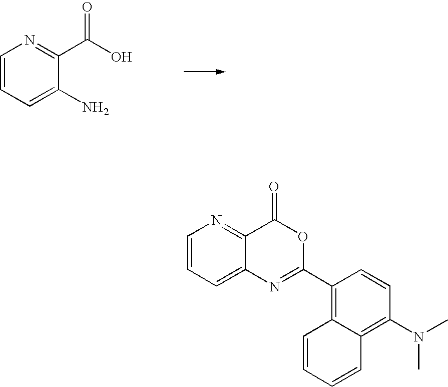

Following the procedure for Step B in Example 18, using 3-amino-2-pyridinecarboxylic acid (672 mg, 4.87 mmol), DIPEA (780 mg, 6.0 mmol), 4-dimethylamino-1-naphthalenecarbonyl chloride prepared from 4-dimethylamino-1-naphthoic acid (1.0 g, 4.64 mmol) and oxalyl chloride (3 mL, 2.0 M in CH₂Cl₂, 6 mmol), and then HATU (1.9 g, 5.0 mmol) provided the title compound which was directly used in Step A.

### Example 24

### 3-[[[4-(Dimethylamino)-1-naphthalenyl]carbonyl]amino]-N-[(tetrahydro-2H-pyran-4-yl)methyl]-2-pyridinecarboxamide

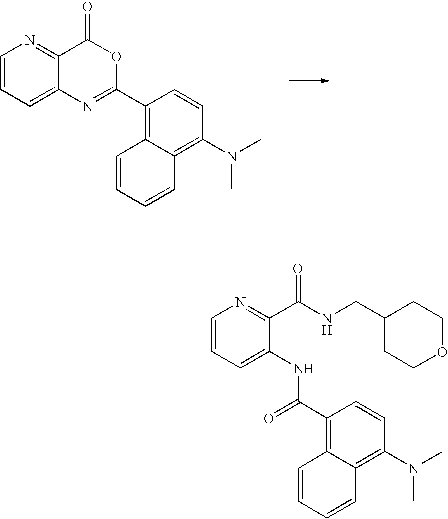

Following the procedure for Step A in Example 1, using 2-[4-(Dimethylamino)-1-naphthalenyl]-4H-pyrido[3,2-d][1,3]oxazin-4-one (1.47 g, 4.64 mmol) and 4-aminomethyltetrahydropyran (177 mg, 1.54 mmol) provided the title compound as its TFA salt after purification by reversed-phase HPLC (30 mg, 4%). ¹H NMR (400 MHz, CDCl₃) δ 1.40 (m, 1H), 1.68 (dd, J=12.79, 1.46 Hz, 2H), 3.00 (s, 6H), 3.32 (t, J=6.64 Hz, 2H), 3.38 (m, 2H), 3.99 (dd, J=11.42, 3.61 Hz, 2H), 7.13 (d, J=7.81 Hz, 1H), 7.54 (m, 3H), 7.88 (d, J=7.81 Hz, 1H), 8.26 (m, 2H), 8.60 (m, 2H), 9.40 (dd, J=8.49, 1.27 Hz, 1H), 12.73 (s, 1H); MS (ESI) (M+H)⁺ 433.0.

### Example 25

### N-(Cyclobutylmethyl)-3-[[[4-(dimethylamino)-1-naphthalenyl]carbonyl]amino]-2-pyridinecarboxamide

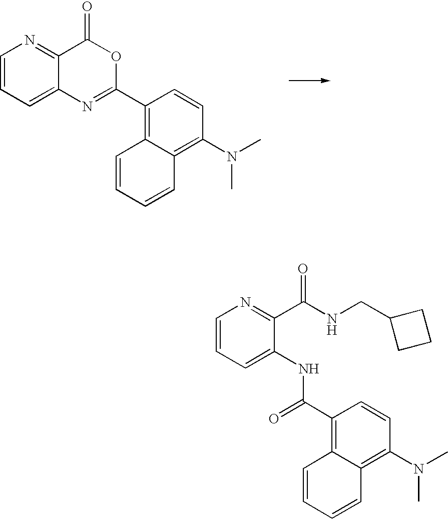

Following the procedure for Step A in Example 1, using 2-[4-(Dimethylamino)-1-naphthalenyl]-4H-pyrido[3,2-d][1,3]oxazin-4-one (1.47 g, 4.64 mmol) and cyclobutanemethylamine (393 mg, 4.62 mmol) provided the title compound as its TFA salt after purification by reversed-phase HPLC (18 mg, 2%). ¹H NMR (400 MHz, CDCl₃) δ 1.75 (m, 2H), 1.90 (m, 2H), 2.10 (m, 2H), 2.59 (m, 1H), 3.10 (s, 6H), 3.43 (dd, J=7.23, 6.25 Hz, 2H), 7.27 (d, J=2.73 Hz, 1H), 7.51 (m, 1H), 7.60 (m, 2H), 7.90 (d, J=8.01 Hz, 1H), 8.27 (dd, J=4.49, 1.56 Hz, 1H), 8.30 (m, 1H), 8.46 (t, J=5.57 Hz, 1H), 8.61 (m, 1H), 9.38 (dd, J=8.59, 1.37 Hz, 1H), 12.86 (s, 1H); MS (ESI) (M+H)⁺ 403.3.

### Example 26

### N-(Cyclobutyloxy)-3-[(1-naphthalenylcarbonyl)amino]-2-pyridinecarboxamide

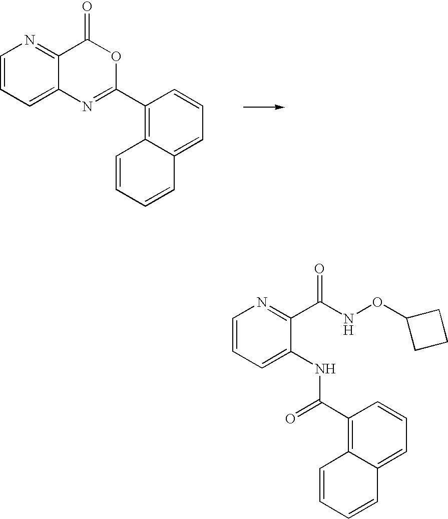

Following the procedure for Step A in Example 1, using 2-(1-naphthalenyl)-4H-pyrido[3,2-d][1,3]oxazin-4-one (55 mg, 0.2 mmol) and O-cyclobutylhydroxylamine (prepared as ref. A. Miyake et al J. Antibiot. 53 (10), 1071-1085, 2000) (38 mg, 0.44 mmol) provided the title compound as its TFA salt after purification by reversed-phase HPLC (41 mg, 43%). ¹H NMR (400 MHz, CD₃OD) δ 1.53 (m, 1H), 1.75 (m, 1H), 2.17 (m, 4H), 4.51 (m, 1H), 7.59 (m, 4H), 7.91 (dd, J=7.03, 0.98 Hz, 1H), 7.96 (m, 1H), 8.07 (d, J=8.20 Hz, 1H), 8.36 (dd, J=4.49, 1.37 Hz, 1H), 8.43 (m, 1H), 9.27 (dd, J=8.59, 1.37 Hz, 1H). MS (ESI) (M+H)⁺=362.0. Anal. Calcd for C₂₁H₁₉N₃O₃+3.0TFA+5.2MeCN+7.1H₂O: C, 42.99; H, 5.00; N, 10.99. Found: C, 43.01; H, 5.00; N, 11.00.

### Example 27

### N-(Cyclopentyloxy)-3-[(1-naphthalenylcarbonyl)amino]-2-pyridinecarboxamide

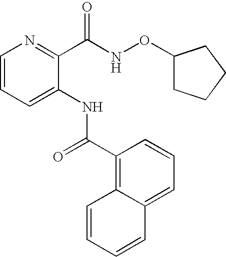

### Step A. N-(cyclopentyloxy)-3-[(1-naphthalenylcarbonyl)amino]-2-pyridinecarboxamide

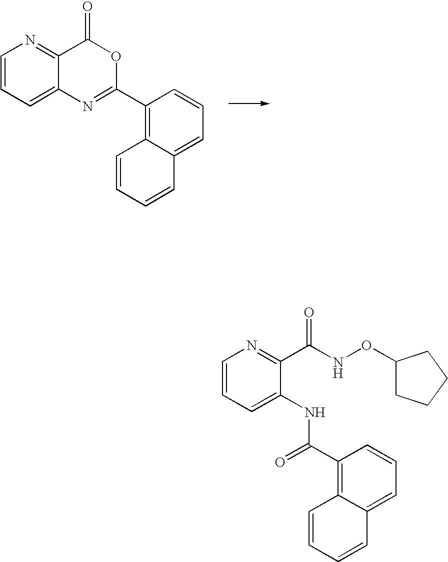

Following the procedure for Step A in Example 1, using 2-(1-naphthalenyl)-4H-pyrido[3,2-d][1,3]oxazin-4-one (55 mg, 0.2 mmol), O-cyclopentylhydroxylamine hydrochloride (66 mg, 0.48 mmol, see Step B & C for its preparation) and DIPEA (67 mg, 0.52 mmol) provided the title compound as its TFA salt after purification by reversed-phase HPLC (52 mg, 67%). ¹H NMR (400 MHz, CD₃OD) δ 1.57 (m, 2H), 1.74 (m, 4H), 1.89 (m, 2H), 4.58 (m, 1H), 7.59 (m, 4H), 7.91 (dd, J=7.13, 1.07 Hz, 1H), 7.96 (m, 1H), 8.07 (d, J=8.40 Hz, 1H), 8.36 (dd, J=4.49, 1.56 Hz, 1H), 8.43 (m, 1H) 9.27 (dd, J=8.59, 1.37 Hz, 1H). MS (ESI) (M+H)⁺=376.0. Anal. Calcd for C₂₂H₂₁N₃O₃+0.1TFA+0.1H₂O: C, 68.61; H, 5.52; N, 10.81. Found: C, 68.51; H, 5.45; N, 10.68.

### Step B. tert-Butyl(cyclopentyloxy)carbamate

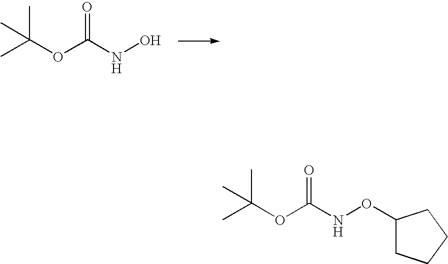

Sodium hydride (0.88 g, 23 mmol) was added to a solution of the N-Boc hydroxylamine (1.33 g, 10 mmol) in THF (60 mL) at 0° C. Stirring for 30 min., cyclopentyl bromide (1.49 g, 10 mmol) was added. The mixture was heated at reflux for 8 h, quenched with aqueous sodium biscarbonate, washed with brine, and dried over sodium sulphate. After evaporation of the solvent, the residue was purified by MPLC using hexane/EtOAc (4:1) on SiO₂ to give the title compound as a colorless oil (0.43 g, 21%). ¹H NMR (400 MHz, CDCl₃) δ 1.48 (s, 9H), 1.56 (m, 2H), 1.70 (m, 4H), 1.82 (m, 2H), 4.40 (m, 1H), 7.01 (s, 1H).

### Step C. O-Cyclopentylhydroxylamine

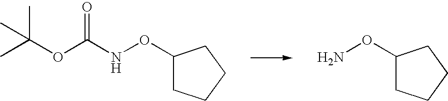

Hydrogen chloride in dioxane (3 mL, 4 M, 12 mmol) was added to a solution of the tert-butyl (cyclopentyloxy)carbamate (0.43 g, 2.14 mmol) in CH₂Cl₂ (1 mL) at room temperature. After stirring for 2 h, removal of solvents provided the title compound as its HCl salt (0.29 g, 100%). ¹H NMR (400 MHz, DMSO-D₆) δ 1.56 (m, 4H), 1.74 (m, 4H), 4.64 (m, 1H), 10.87 (s, 3H).

### Example 28

### N-(Cyclohexyloxy)-3-[(1-naphthalenylcarbonyl)amino]-2-pyridinecarboxamide

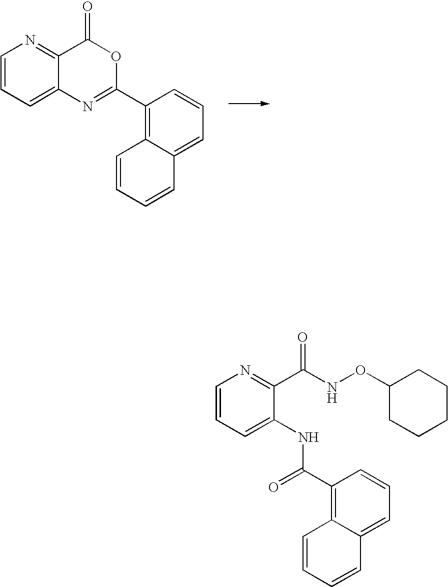

Following the procedure for Step A in Example 1, using 2-(1-naphthalenyl)-4H-pyrido[3,2-d][1,3]oxazin-4-one (55 mg, 0.2 mmol) and O-cyclohexylhydroxylamine (prepared as ref. A. Miyake et al J. Antibiot. 53 (10), 1071-1085, 2000) (51 mg, 0.44 mmol) provided the title compound as its TFA salt after purification by reversed-phase HPLC (64 mg, 78%). ¹H NMR (400 MHz, CD₃OD) δ 1.26 (m, 3H), 1.42 (m, 2H), 1.54 (m, 1H), 1.77 (m, 2H), 1.98 (m, 2H), 3.90 (m, 1H), 7.59 (m, 4H), 7.91 (dd, J=7.13, 1.07 Hz, 1H), 7.97 (m, 1H), 8.07 (d, J=8.40 Hz, 1H), 8.36 (dd, J=4.49, 1.56 Hz, 1H) 8.43 (m, 1H) 9.27 (dd, J=8.59, 1.37 Hz, 1H). MS (ESI) (M+H)⁺=390.0. Anal. Calcd for C₂₃H₂₃N₃O₃+0.2TFA: C, 68.18; H, 5.67; N, 10.19. Found: C, 68.41; H, 5.72; N, 10.18.

### Example 29

### N-(Cyclohexyloxy)-3-[(4-methoxy-1-naphthalenylcarbonyl)amino]-2-pyridinecarboxamide

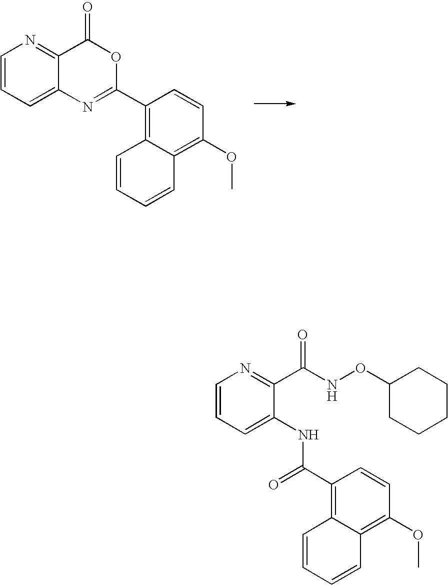

Following the procedure for Step A in Example 1, using 2-(4-methoxy 1-naphthalenyl)-H-pyrido[3,2-d][1,3]oxazin-4-one (120 mg, 0.4 mmol) and O-cyclohexylhydroxylamine (prepared as ref. A. Miyake et al J. Antibiot. 53 (10), 1071-1085, 2000) (205 mg, 1.8 mmol) provided the title compound as its TFA salt after purification by reversed-phase HPLC (91 mg, 54%). ¹H NMR (400 MHz, CD₃OD) δ 1.28 (m, 3H), 1.48 (m, 3H), 1.79 (m, 2H), 1.99 (m, 2H), 3.92 (m, 1H), 4.08 (s, 3H), 7.01 (d, J=8.20 Hz, 1H), 7.56 (m, 3H), 7.93 (d, J=8.01 Hz, 1H), 8.32 (m, 2H), 8.51 (d, J=8.20 Hz, 1H), 9.25 (dd, J=8.59, 1.37 Hz, 1H). MS (ESI) (M+H)⁺=420.0.

### Example 30

### N-(Cyclobutylmethyl)-3-[(2-methoxybenzoyl)amino]-2-pyridinecarboxamide

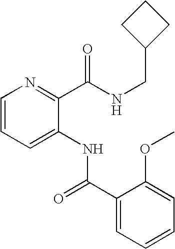

### Step A. N-(Cyclobutylmethyl)-3-[(2-methoxybenzoyl)amino]-2-pyridinecarboxamide

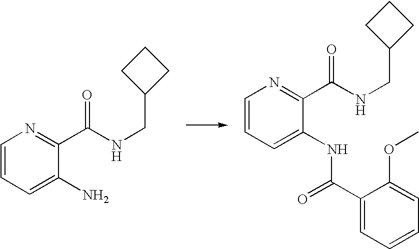

DIPEA (0.13 mL, 0.73 mmol) was added into a solution of 3-amino-N-(cyclobutylmethyl)-2-pyridinecarboxamide (87 mg, 0.43 mmol, see Step B for its preparation) and 2-methoxy-benzoic acid (79 mg, 0.52 mmol) in DMF (10 mL) at 0° C. After stirring for 20 min. HATU (179 mg, 0.47 mmol) was added. The reaction mixture was stirred for 24 h at room temperature, and was then quenched with H₂O (50 mL) and extracted with EtOAc (2×50 mL). The crude product was purified by reversed-phase HPLC to provide the title compound as its TFA salt (51 mg, 26%). ¹H NMR (400 MHz, CD₃OD) δ 1.77-1.97 (m, 4H), 2.06-2.14 (m, 2H), 2.59-2.70 (m, 1H), 3.43-3.47 (m, 2H), 4.07 (s, 3H), 7.06-7.10 (m, 1H), 7.19 (d, J=8.40 Hz, 1H), 7.51-7.57 (m, 2H), 8.00 (dd, J=7.81, 1.76 Hz, 1H), 8.30 (dd, J=4.39, 1.46 Hz, 1H), 8.89-8.96 (br. s., 1H), 9.24 (dd, J=8.59, 1.46 Hz, 1H), 12.93-13.02 (br. s., 1H); MS (ESI) (M+H)⁺ 340.3.

### Step B. 3-Amino-N-(cyclobutylmethyl)-2-pyridinecarboxamide

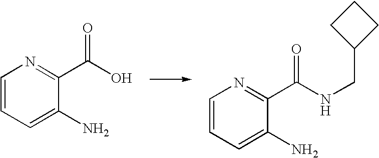

HATU (3.03 g, 7.96 mmol) was added to a solution of 3-aminopyridine-2-carboxylic acid (1.0 g, 7.24 mmol), cyclobutanemethylamine (2.7 mL, 5.3 M in MeOH, 14.5 mmol), and DIPEA (3.8 g, 30 mmol) in DMF (50 ml) at room temperature. After 24 hr, the reaction mixture was quenched with H₂O (100 mL), and extracted with EtOAc (2×100 mL). The combined organic phases were washed with brine, and condensed in vacuo to provide the title compound (1.22 g, 82%).

### Example 31

### N-[2-[[(Cyclobutylmethyl)amino]carbonyl]-3-pyridinyl]-4-quinolinecarboxamide

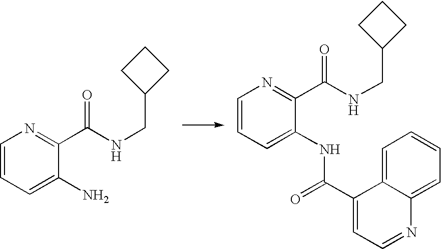

Following the procedure for Step A in Example 30, using DIPEA (0.07 mL, 0.42), 3-amino-N-(cyclobutylmethyl)-2-pyridinecarboxamide (50 mg, 0.24 mmol), quinoline-4-carboxylic acid (50 mg, 0.29 mmol), and HATU (110 mg, 0.29 mmol) provided the title compound as its TFA salt after purification by reversed-phase HPLC (9 mg, 8%). ¹H NMR (400 MHz, CD₃OD) δ 1.71-1.93 (m, 4H), 2.02-2.10 (m, 2H), 2.57-2.64 (m, 1H), 3.38 (d, J=7.23 Hz, 2H), 7.64 (m, 1H), 7.76-7.78 (m, 1H), 7.82-7.96 (m, 2H), 8.17-8.19 (d, 1H), 8.41 (dd, J=4.59, 1.51 Hz, 1H), 8.50-8.52 (m, 1H), 9.10 (d, J=4.69 Hz, 1H), 9.27 (dd, J=8.59, 1.51 Hz, 1H); MS (ESI) (M+H)⁺ 361.2.

### Example 32

### N-[2-[[(Cyclobutylmethyl)amino]carbonyl]-3-pyridinyl]-5-isoquinolinecarboxamide

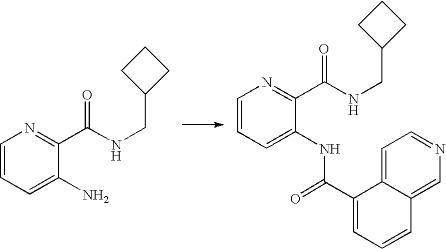

Following the procedure for Step A in Example 30, using DIPEA (0.17 mL, 0.97), 3-amino-N-(cyclobutylmethyl)-2-pyridinecarboxamide (100 mg, 0.49 mmol), isoquinoline-5-carboxylic acid (168 mg, 0.97 mmol), and HATU (369 mg, 0.97 mmol) provided the title compound as its TFA salt after purification by reversed-phase HPLC (97 mg, 42%). ¹H NMR (400 MHz, CD₃OD) δ 1.47-1.96 (m, 4H), 2.02-2.10 (m, 2H), 2.58-2.65 (m, 1H), 3.39 (d, J=7.23 Hz, 2H), 7.62 (dd, J=8.59, 4.59 Hz, 1H), 8.10 (dd, J=8.30, 7.32 Hz, 1H), 8.39 (dd, J=4.59, 1.41 Hz, 1H), 8.59-8.64 (m, 3H), 8.98-8.90 (m, 1H) 9.26 (dd, J=8.59, 1.41 Hz, 1H), 9.73-9.80 (br. s., 1H); MS (ESI) (M+H)⁺ 361.2.

### Example 33

### N-(Cyclobutylmethyl)-3-[[(2,3-dihydro-1,4-benzodioxin-5-yl)carbonyl]amino]-2-pyridinecarboxamide

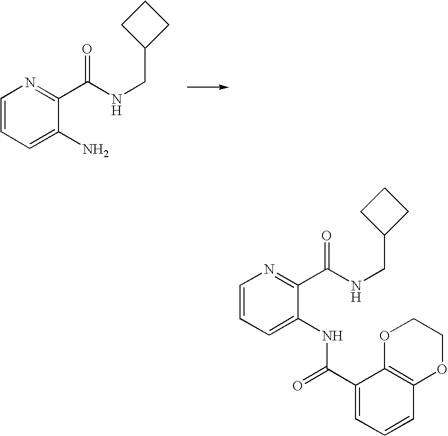

Following the procedure for Step A in Example 30, using DIPEA (0.17 mL, 0.97), 3-amino-N-(cyclobutylmethyl)-2-pyridinecarboxamide (100 mg, 0.49 mmol), 1,4-benzodioxan-5-carboxylic acid (175 mg, 0.97 mmol), and HATU (369 mg, 0.97 mmol) provided the title compound as its TFA salt after purification by reversed-phase HPLC (90 mg, 50%). ¹H NMR (400 MHz, DMSO-D₆) δ 1.70-1.85 (m, 4H), 1.93-2.01 (m, 2H), 2.53-2.62 (m, 1H), 3.32-3.36 (m, 2H), 4.33-3.45 (m, 4H), 6.95 (t, J=7.91 Hz, 1H), 7.07-7.10 (m, 1H), 7.42 (dd, J=7.81, 1.56 Hz, 1H), 7.62 (dd, J=8.59, 4.41 Hz, 1H), 8.34 (dd, J=4.41, 1.51 Hz, 1H), 9.04-9.07 (m, 1H), 9.17 (dd, J=8.59, 1.51 Hz, 1H), 12.87-12.91 (br. s., 1H); MS (ESI) (M+H)⁺ 368.3.

### Example 34

### N-(Cyclobutylmethyl)-3-[[(2,3-dihydro-7-benzofuranyl)carbonyl]amino]-2-pyridinecarboxamide

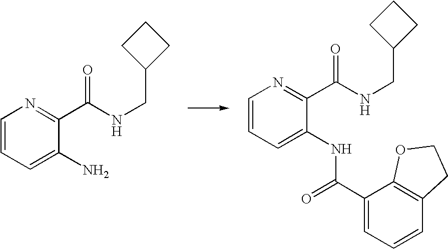

Following the procedure for Step A in Example 30, using DIPEA (0.17 mL, 0.97), 3-amino-N-(cyclobutylmethyl)-2-pyridinecarboxamide (100 mg, 0.49 mmol), 2,3-dihydrofuran-7-carboxylic acid (159 mg, 0.97 mmol), and HATU (369 mg, 0.97 mmol) provided the title compound as its TFA salt after purification by reversed-phase HPLC (92 mg, 38%). ¹H NMR (400 MHz, DMSO-D₆) δ 1.68-1.85 (m, 4H), 1.93-2.01 (m, 2H), 2.52-2.60 (m, 1H), 3.26-3.37 (m, 4H), 4.73 (t, J=8.79 Hz, 2H), 6.96-6.99 (m, 1H), 7.46 (dd, J=7.23, 1.17 Hz, 1H), 7.61 (dd, J=8.59, 4.49 Hz, 1H), 7.65 (dd, J=7.81, 1.17 Hz, 1H), 8.34 (dd, J=4.49, 1.46 Hz, 1H), 8.99-9.02 (m, 1H), 9.06 (dd, J=8.59, 1.46 Hz, 1H), 12.62 (s, 1H); MS (ESI) (M+H)⁺ 352.3.

### Example 35

### N-(Cyclobutylmethyl)-3-[(3-methoxy-2-methylbenzoyl)amino]-2-pyridinecarboxamide

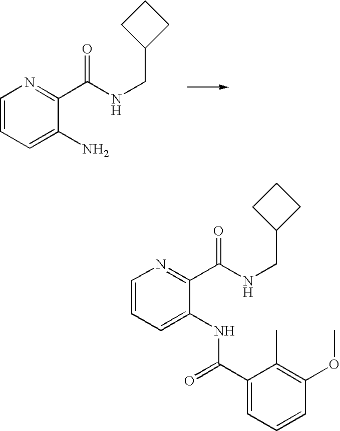

Following the procedure for Step A in Example 30, using DIPEA (0.17 mL, 0.97), 3-amino-N-(cyclobutylmethyl)-2-pyridinecarboxamide (100 mg, 0.49 mmol), 3-methoxy-2-methylbenzoic acid (161 mg, 0.97 mmol), and HATU (369 mg, 0.97 mmol) provided the title compound as its TFA salt after purification by reversed-phase HPLC (44 mg, 19%). ¹H NMR (400 MHz, CD₃OD) δ 1.72-1.97 (m, 4H), 2.01-2.10 (m, 2H), 2.31 (s, 3H), 2.55-2.64 (m, 1H), 3.37 (d, J=7.23 Hz, 2H), 3.87 (s, 3H), 7.09 (d, J=8.20 Hz, 1H), 7.14-7.16 (m, 1H), 7.28-7.32 (m, 1H), 7.56 (dd, J=8.59, 4.49 Hz, 1H), 8.33 (dd, J=4.49, 1.31 Hz, 1H), 9.18 (dd, J=8.59, 1.31 Hz, 1H); MS (ESI) (M+H)⁺ 354.2.

### Example 36

### N-(2-{[(Tetrahydro-2H-pyran-4-ylmethyl)amino]carbonyl}pyridin-3-yl)quinoline-4-carboxamide

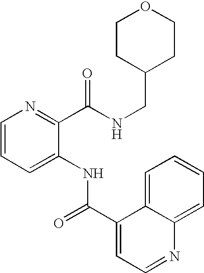

### Step A. N-(2-{[(Tetrahydro-2H-pyran-4-ylmethyl)amino]carbonyl}pyridin-3-yl)quinoline-4-carboxamide

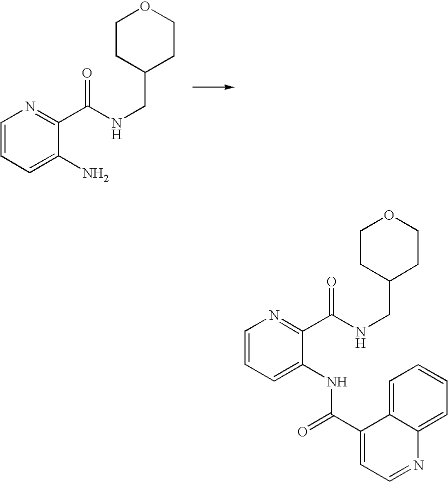

Following the procedure for Step A in Example 30, using DIPEA (65 mg, 0.5 mmol), 3-amino-N-(tetrahydro-2H-pyran-4-ylmethyl)pyridine-2-carboxamide (50 mg, 0.21 mmol, see Step B for its preparation), and quinoline-4-carboxylic acid (52 mg, 0.3 mmol), and HATU (114 mg, 0.3 mmol) provided the title compound as its TFA salt after purification by reversed-phase HPLC (24 mg, 23%). ¹H NMR (400 MHz, CD₃OD) δ 1.31 (m, 2H), 1.61 (m, 2H), 1.82 (m, 1H), 3.26 (m, 2H), 3.35 (m, 2H), 3.90 (m, 2H), 7.64 (m, 1H), 7.90 (m, 1H), 8.06 (m, 1H), 8.13 (m, 1H), 8.24 (d, J=8.8 Hz, 1H), 8.43 (dd, J=4.4, 1.6 Hz, 1H), 8.58 (d, J=8.0 Hz, 1H), 9.24 (dd, J=8.4, 1.6 Hz, 2H); MS (ESI) (M+H)⁺ 391.2.

### Step B. 3-Amino-N-(tetrahydro-2H-pyran-4-ylmethyl)pyridine-2-carboxamide

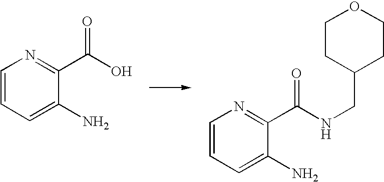

Following the procedure for Step B in Example 30, using HATU (1.52 g, 4.0 mmol), 3-aminopyridine-2-carboxylic acid (387 mg, 3.0 mmol), tetrahydro-2H-pyran-4-methanamine (456 mg, 4.0 mmol), and DIPEA (520 mg, 4.0 mmol) provided the title compound (650 mg, 92%).

### Example 37

### N-(2-{[(Tetrahydro-2H-pyran-4-ylmethyl)amino]carbonyl}pyridin-3-yl)isoquinoline-5-carboxamide

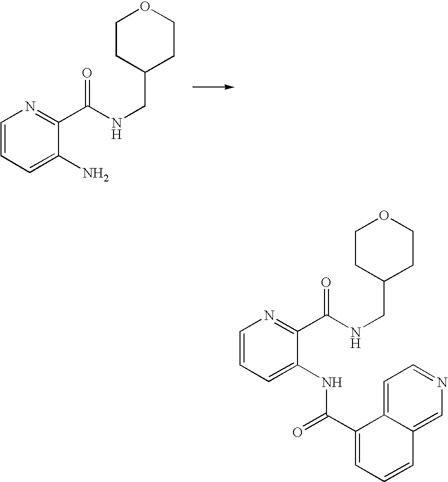

Following the procedure for Step A in Example 30, using DIPEA (65 mg, 0.5 mmol), 3-amino-N-(tetrahydro-2H-pyran-4-ylmethyl)pyridine-2-carboxamide (50 mg, 0.21 mmol), and isoquinoline-5-carboxylic acid (52 mg, 0.3 mmol), and HATU (114 mg, 0.3 mmol) provided the title compound as its TFA salt after purification by reversed-phase HPLC (25 mg, 24%). ¹H NMR (400 MHz, CD₃OD) δ 1.32 (m, 2H), 1.65 (m, 2H), 1.88 (m, 1H), 3.29 (m, 2H), 3.38 (m, 2H), 3.93 (m, 2H), 7.65 (m, 1H), 8.13 (m, 1H), 8.42 (d, J=4.4 Hz, 1H), 8.63 (m, 3H), 9.05 (m, 1H), 9.29 (d, J=4.4 Hz, 1H), 9.45 (m, 1H); MS (ESI) (M+H)⁺ 391.0.

### Example 38

### N-(2-{[(Tetrahydro-2H-pyran-4-ylmethyl)amino]carbonyl}pyridin-3-yl)quinoline-5-carboxamide

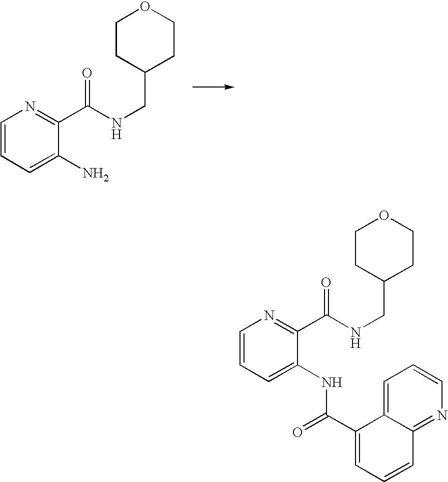

Following the procedure for Step A in Example 30, using DIPEA (65 mg, 0.5 mmol), 3-amino-N-(tetrahydro-2H-pyran-4-yl-methyl)pyridine-2-carboxamide (50 mg, 0.21 mmol), and quinoline-5-carboxylic acid (52 mg, 0.3 mmol), and HATU (114 mg, 0.3 mmol) provided the title compound as its TFA salt after purification by reversed-phase HPLC (30 mg, 28%). ¹H NMR (400 MHz, CD₃OD) δ 1.31 (m, 2H), 1.67 (m, 2H), 1.88 (m, 1H), 3.29 (m, 2H), 3.38 (m, 2H), 3.92 (m, 2H), 7.63 (dd, J=8.4, 4.4 Hz, 1H), 7.86 (dd, J=8.8, 4.8 Hz, 1H), 8.09 (m, 1H), 8.24 (d, J=7.6 Hz, 1H), 8.31 (d, J=8.8 Hz, 1H), 8.41 (d, J=4.4 Hz, 1H), 9.10 (m, 1H), 9.28 (dd, J=8.8, 1.6 Hz, 1H), 9.37 (d, J=8.0 Hz, 1H); MS (ESI) (M+H)⁺ 391.2.

### Example 39

### N-(Cyclohexylmethyl)-4-(1-naphthoylamino)nicotinamide

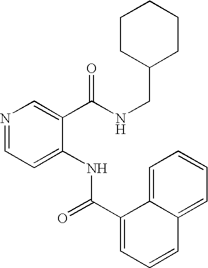

### Step A. N-(Cyclohexylmethyl)-4-(1-naphthoylamino)nicotinamide

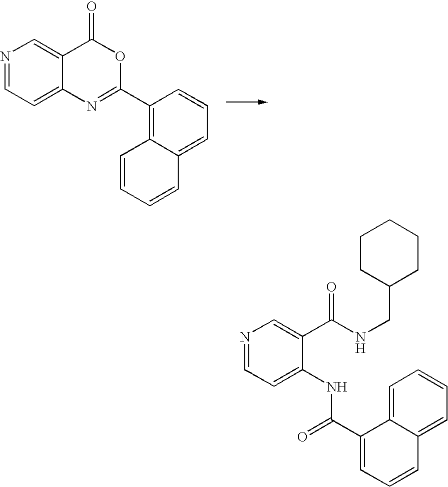

Following the procedure for Step A in Example 1, using 2-(1-naphthyl)-4H-pyrido[4,3-d][1,3]oxazin-4-one (137 mg, 0.5 mmol, see Step B for its preparation) and cyclohexylmethylamine (226 mg, 2.0 mmol) provided the title compound as its TFA salt after purification by reversed-phase HPLC (39 mg, 16%). ¹H NMR (400 MHz, CDCl₃) δ 0.99 (m, 2H), 1.23 (m, 3H), 1.63 (m, 1H), 1.76 (m, 5H), 3.22 (d, J=6.8 Hz, 2H), 7.61 (m, 3H), 7.98 (m, 2H), 8.14 (d, J=8.4 Hz, 1H), 8.53 (m, 1H), 8.72 (m, 1H), 9.05 (s, 1H), 9.22 (d, J=6.8 Hz, 1H); MS (ESI) (M+H)⁺ 388.0.

### Step B. 2-(1-Naphthyl)-4H-pyrido[4,3-d][1,3]oxazin-4-one

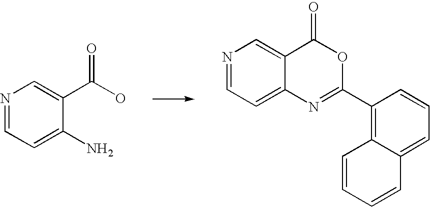

Following the procedure for Step B in Example 1, using 4-aminonicotinic acid (138 mg, 1.0 mmol), 1-naphthalenecarbonyl chloride (191 mg, 1.0 mmol), DIPEA (284 mg, 2.2 mmol), and then HATU (419 mg, 1.1 mmol) provided the title compound as a DMF (6 mL) solution which was used directly in Step A. MS (ESI) (M+H)⁺ 274.79.

### Example 40

### N-(Cyclobutylmethyl)-4-(1-naphthoylamino)nicotinamide

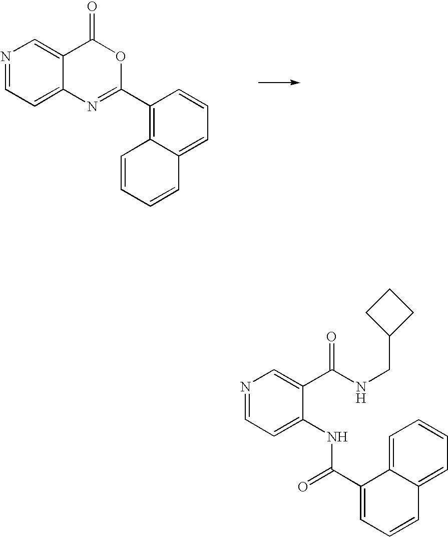

Following the procedure for Step A in Example 1, using 2-(1-naphthyl)-4H-pyrido[4,3-d][1,3]oxazin-4-one (137 mg, 0.5 mmol) and cyclobutylmethylamine (170 mg, 2.0 mmol) provided the title compound as its TFA salt after purification by reversed-phase HPLC (45 mg, 19%). ¹H NMR (400 MHz, CDCl₃) δ 1.74 (m, 2H), 1.88 (m, 2H), 2.08 (m, 2H), 2.61 (m, 1H), 3.46 (m, 2H), 7.62 (m, 3H), 7.94 (m, 2H), 8.09 (d, J=8.4 Hz, 1H), 8.39 (s, 1H), 8.55 (m, 2H), 9.34 (d, J=6.4 Hz, 1H), 9.39 (s, 1H), 13.10 (s, 1H); MS (ESI) (M+H)⁺ 360.0.

### Example 41

### N-(Cyclohexylmethyl)-3-(1-naphthoylamino)isonicotinamide

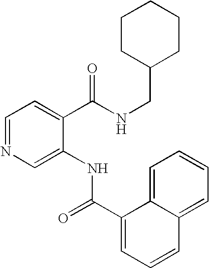

### Step A. N-(Cyclohexylmethyl)-3-(1-naphthoylamino)isonicotinamide

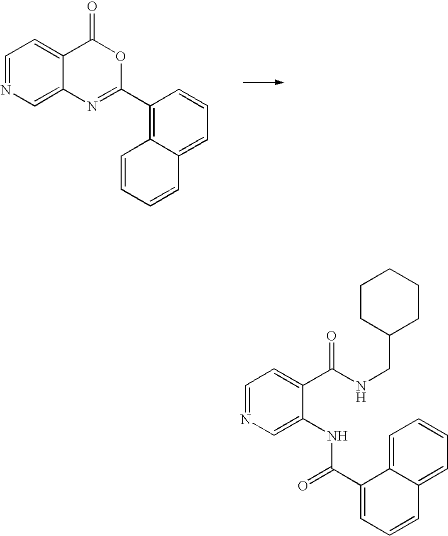

Following the procedure for Step A in Example 1, using 2-(1-naphthyl)-4H-pyrido[3,4-d][1,3]oxazin-4-one (137 mg, 0.5 mmol, see Step B for its preparation) and cyclohexylmethylamine (226 mg, 2.0 mmol) provided the title compound as its TFA salt after purification by reversed-phase HPLC (55 mg, 22%). ¹H NMR (400 MHz, CD₃OD) δ 0.99 (m, 2H), 1.22 (m, 3H), 1.70 (m, 6H), 3.22 (d, J=7.2 Hz, 2H), 7.59 (m, 3H), 7.90 (dd, J=7.2, 1.2 Hz, 1H), 7.96 (m, 1H), 7.99 (brs, 1H), 8.08 (d, J=8.4 Hz, 1H), 8.47 (m, 1H), 8.64 (brs, 1H), 10.08 (brs, 1H); MS (ESI) (M+H)⁺ 388.1.

### Step B. 2-(1-Naphthyl)-4H-pyrido[3,4-d][1,3]oxazin-4-one

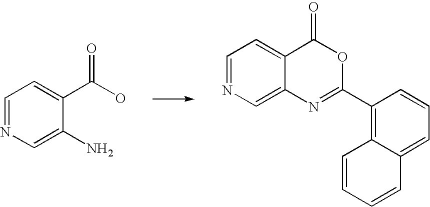

Following the procedure for Step B in Example 1, using 3-aminoisonicotinic acid (138 mg, 1.0 mmol), 1-naphthalenecarbonyl chloride (191 mg, 1.0 mmol), DIPEA (284 mg, 2.2 mmol), and then HATU (419 mg, 1.1 mmol) provided the title compound as a DMF (6 mL) solution which was used directly in Step A. MS (ESI) (M+H)⁺ 274.79.

### Example 42

### N-Cyclobutyl-3-(1-naphthoylamino)isonicotinamide

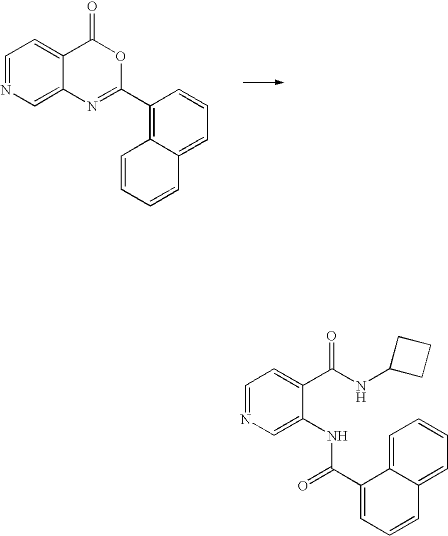

Following the procedure for Step A in Example 1, using 2-(1-naphthyl)-4H-pyrido[3,4-d][1,3]oxazin-4-one (137 mg, 0.5 mmol) and cyclobutylamine (142 mg, 2.0 mmol) provided the title compound as its TFA salt after purification by reversed-phase HPLC (43 mg, 19%). ¹H NMR (400 MHz, CD₃OD) δ 1.73 (m, 2H), 2.07 (m, 2H), 2.28 (m, 2H), 4.24 (m, 1H), 7.53 (m, 3H), 7.84 (dd, J=7.2, 1.2 Hz, 1H), 7.88 (m, 1H), 8.0 (d, J=8.0 Hz, 1H), 8.04 (m, 1H), 8.40 (m, 1H), 8.54 (brs, 1H), 9.90 (brs, 1H); MS (ESI) (M+H)⁺ 346.1.

### Example 43

### 3-(1-Naphthoylamino)-N-(tetrahydro-2H-pyran-4-ylmethyl)pyrazine-2-carboxamide

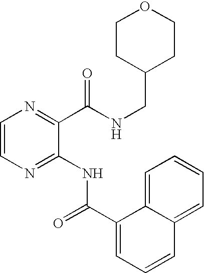

### Step A. 3-(1-Naphthoylamino)-N-(tetrahydro-2H-pyran-4-yl-methyl)pyrazine-2-carboxamide

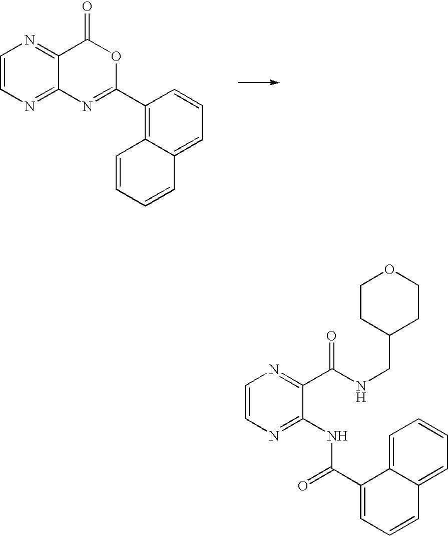

Following the procedure for Step A in Example 1, using 2-(1-naphthyl)-4H-pyrazino[2,3-d][1,3]oxazin-4-one (69 mg, 0.25 mmol, see Step B for its preparation), and tetrahydro-2H-pyran-4-methanamine (115 mg, 1.0 mmol) provided the title compound as its TFA salt after purification by reversed-phase HPLC (12 mg, 10%). ¹H NMR (400 MHz, CD₃OD) δ 1.27 (m, 2H), 1.62 (m, 2H), 1.88 (m, 1H), 3.29 (m, 4H), 3.91 (m, 2H), 7.59 (m, 3H), 7.95 (m, 2H), 8.10 (m, 1H), 8.43 (m, 1H), 8.48 (m, 1H), 8.59 (m, 1H); MS (ESI) (M+H)⁺ 391.0.

### Step B. 2-(1-Naphthyl)-4H-pyrazino[2,3-d][1,3]oxazin-4-one

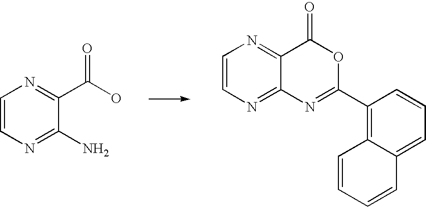

Following the procedure for Step B in Example 1, using 3-aminopyrazine-2-carboxylic acid (139 mg, 1.0 mmol), 1-naphthalenecarbonyl chloride (191 mg, 1.0 mmol), DIPEA (284 mg, 2.2 mmol), and HATU (419 mg, 1.1 mmol) provided the title compound as a DMF (6 mL) solution which was used directly in Step A. MS (ESI) (M+H)⁺ 275.82.

### Example 44

### N-(Cyclohexylmethyl)-3-(1-naphthoylamino)pyrazine-2-carboxamide

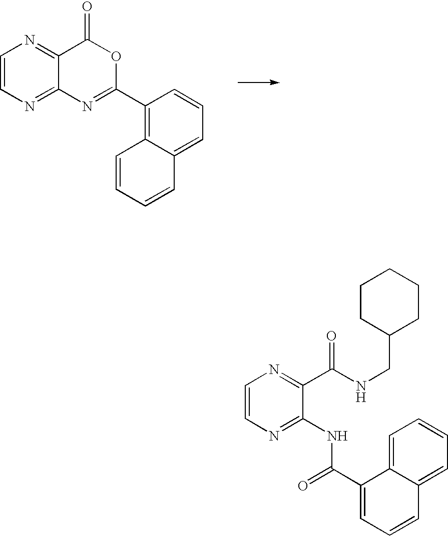

Following the procedure for Step A in Example 1, using 2-(1-naphthyl)-4H-pyrazino[2,3-d][1,3]oxazin-4-one (69 mg, 0.25 mmol), and cyclohexylmethylamine (113 mg, 1.0 mmol) provided the title compound as its TFA salt after purification by reversed-phase HPLC (6 mg, 5%). ¹H NMR (400 MHz, CD₃OD) δ 0.96 (m, 2H), 1.22 (m, 3H), 1.72 (m, 6H), 3.19 (m, 2H), 7.55 (m, 3H), 7.95 (m, 2H), 8.06 (m, 1H), 8.48 (m, 3H); MS (ESI) (M+H)⁺ 389.0.

### Example 45

### N-(Cyclobutylmethyl)-3-(1-naphthoylamino)pyrazine-2-carboxamide

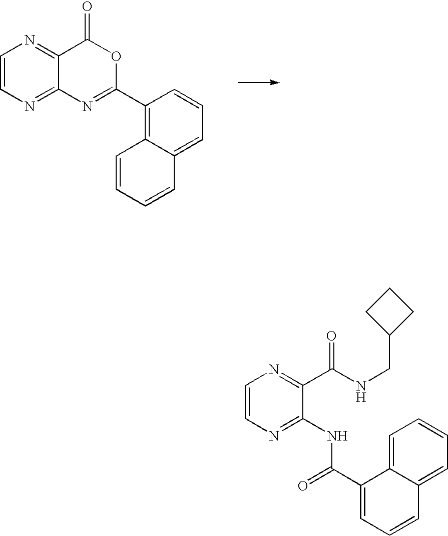

Following the procedure for Step A in Example 1, using 2-(1-naphthyl)-4H-pyrazino[2,3-d][1,3]oxazin-4-one (69 mg, 0.25 mmol), and cyclobutylmethylamine (85 mg, 1.0 mmol) provided the title compound as its TFA salt after purification by reversed-phase HPLC (8 mg, 7%). ¹H NMR (400 MHz, CD₃OD) δ 1.75 (m, 2H), 1.86 (m, 2H), 2.03 (m, 2H), 2.59 (m, 1H), 3.36 (m, 2H), 7.57 (m, 3H), 7.95 (m, 2H), 8.06 (m, 1H), 8.48 (m, 3H); MS (ESI) (M+H)⁺ 361.0.

### Example 46

### N-(Cyclopentylmethyl)-3-(1-naphthoylamino)pyrazine-2-carboxamide

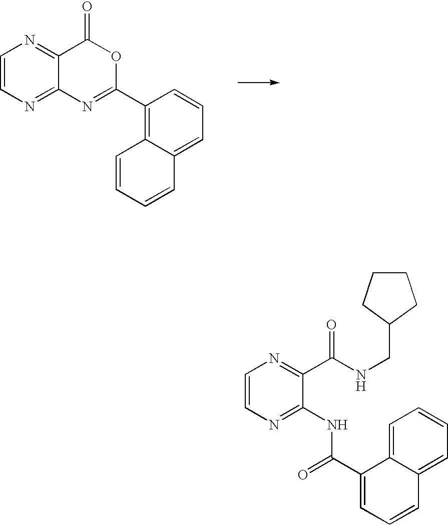

Following the procedure for Step A in Example 1, using 2-(1-naphthyl)-4H-pyrazino[2,3-d]L[1,3]oxazin-4-one (69 mg, 0.25 mmol), and cyclopentylmethylamine (99 mg, 1.0 mmol) provided the title compound as its TFA salt after purification by reversed-phase HPLC (9.5 mg, 8%). ¹H NMR (400 MHz, CD₃OD) δ 1.27 (m, 3H), 1.63 (m, 5H), 2.19 (m, 1H), 3.29 (m, 2H), 7.58 (m, 3H), 7.95 (m, 2H), 8.06 (m, 1H), 8.48 (m, 3H); MS (ESI) (M+H)⁺ 375.0.

### Example 47

### N-(2-Cyclohexylethyl)-3-(1-naphthoylamino)pyrazine-2-carboxamide

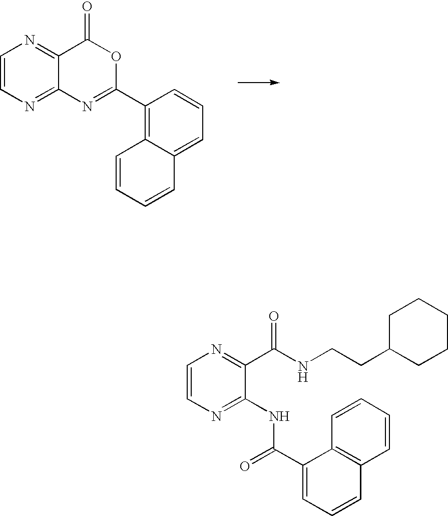

Following the procedure for Step A in Example 1, using 2-(1-naphthyl)-4H-pyrazino[2,3-d][1,3]oxazin-4-one (83 mg, 0.3 mmol), and (2-cyclohexylethyl)amine hydrochloride (164 mg, 1.0 mmol) provided the title compound as its TFA salt after purification by reversed-phase HPLC (48 mg, 31%). ¹H NMR (400 MHz, CDCl₃) δ 0.94 (m, 2H), 1.20 (m, 4H), 1.51 (m, 2H), 1.71 (m, 5H), 3.44 (m, 2H), 7.56 (m, 3H), 7.89 (d, J=8.0 Hz, 1H), 7.98 (m, 2H), 8.15 (m, 1H), 8.28 (s, 1H), 8.62 (d, J=8.0 Hz, 1H), 8.70 (s, 1H), 12.77 (s, 1H); MS (ESI) (M+H)⁺ 403.0.

### Example 48

### 3-[(4-Methyl-1-naphthoyl)amino]-N-pentylpyrazine-2-carboxamide

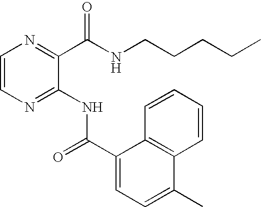

### Step A: 3-[(4-Methyl-1-naphthoyl)amino]-N-pentylpyrazine-2-carboxamide

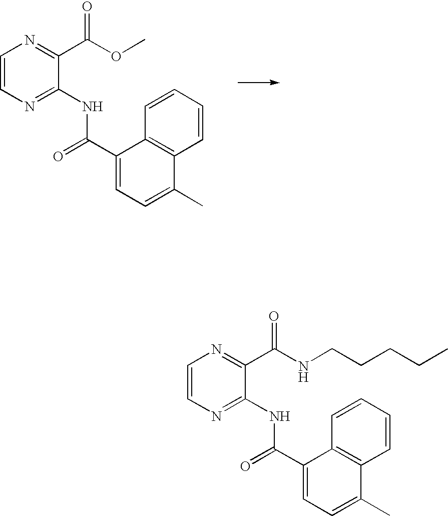

A solution of methyl 3-[(4-methyl-1-naphthoyl)amino]pyrazine-2-carboxylate (257 mg, 0.8 mmol) and pentan-1-amine (174 mg, 2.0 mmol) in 15 mL MeCN was heated at 100° C. for 2 hr. After removal of solvents, the residue was purified by reversed-phase HPLC to give the title compound as its TFA salt (225 mg, 57%). ¹H NMR (400 MHz, CD₃OD) δ 0.86 (t, J=7.6 Hz, 3H), 1.29 (m, 4H), 1.55 (m, 2H), 2.71 (s, 3H), 3.30 (t, J=7.6 Hz, 2H), 7.40 (d, J=8.0 Hz, 1H), 7.56 (m, 2H), 7.83 (d, J=8.0 Hz, 1H), 8.08 (m, 1H), 8.35 (s, 1H), 8.52 (m, 2H). MS (ESI) (M+H)⁺ 377.0.

### Step B: Methyl 3-[(4-methyl-1-naphthoyl)amino]pyrazine-2-carboxylate

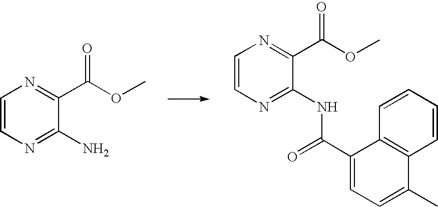

At 90° C. a solution of 4-methyl-1-naphthalenecarbonyl chloride (12 mmol) in CH₂ClCH₂Cl (20 mL) was slowly added into a solution of methyl 3-aminopyrazine-2-carboxylate (1.53 g, 10.0 mmol) and DMAP (100 mg) in CH₂ClCH₂Cl (100 mL) and pyridine (10 mL) during a period of six hours. The resulting reaction mixture was stirred at the same temperature overnight, and was then condensed, and extracted by EtOAc, washed by brine, dried over MgSO₄. Removal of solvents provided a crude product, which was purified by flash silica gel column using heptane/EtOAc (10:0 to 0:10) to give the title product as a solid (1.5 g, 47%): ¹H NMR (400 MHz, CDCl₃) δ 1H NMR (400 MHz, CD₃OD) 2.77 (s, 3H), 3.94 (s, 3H), 7.46 (d, J=8.0 Hz, 1H), 7.60 (m, 2H), 7.79 (d, J=8.0 Hz, 1H), 8.14 (d, J=8.0 Hz, 1H), 8.42 (m, 1H), 8.50 (m, 1H), 8.64 (m, 1H).

### Example 49

### N-(3-Methylbutyl)-3-[(4-methyl-1-naphthoyl)amino]pyrazine-2-carboxamide

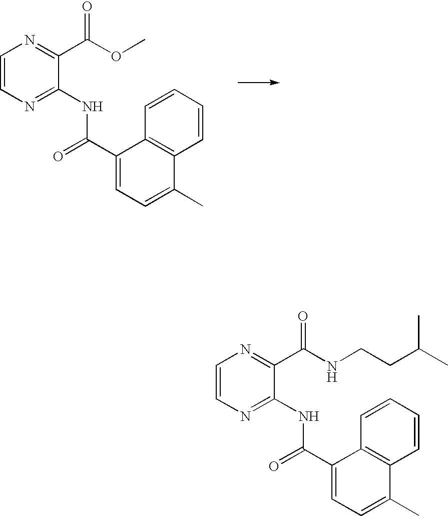

Following the procedure for Step A in Example 48, using methyl 3-[(4-methyl-1-naphthoyl)amino]pyrazine-2-carboxylate (129 mg, 0.4 mmol) and 3-methylbutan-1-amine (87 mg, 1.0 mmol) provided the title compound as its TFA salt after purification by reversed-phase HPLC (85 mg, 43%). ¹H NMR (400 MHz, CD₃OD) δ 0.87 (d, J=7.6 Hz, 6H), 1.42 (m, 2H), 1.56 (m, 1H), 2.68 (s, 3H), 3.31 (dd, J=7.6, 4.0 Hz, 2H), 7.38 (d, J=8.0 Hz, 1H), 7.54 (m, 2H), 7.81 (d, J=8.0 Hz, 1H), 8.05 (m, 1H), 8.32 (s, 1H), 8.52 (m, 2H); MS (ESI) (M+H)⁺ 377.0.

### Example 50

### N-(Cyclobutylmethyl)-3-[(4-methyl-1-naphthoyl)amino]pyrazine-2-carboxamide

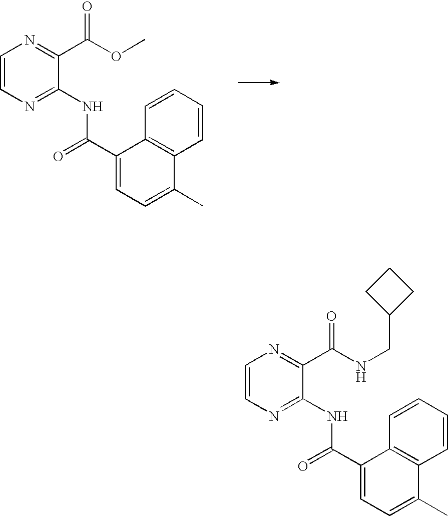

Following the procedure for Step A in Example 48, using methyl 3-[(4-methyl-1-naphthoyl)amino]pyrazine-2-carboxylate (1.6 g, 5.0 mmol) and (cyclobutylmethyl)amine (0.84 g, 10.0 mmol) provided the title compound after purification by silica gel column (720 mg, 39%). ¹H NMR (400 MHz, CD₃OD) δ 1.75 (m, 2H), 1.86 (m, 2H), 2.04 (m, 2H), 2.59 (m, 1H), 2.75 (s, 3H), 3.37 (d, J=7.6 Hz, 2H), 7.44 (d, J=8.0 Hz, 1H), 7.59 (m, 2H), 7.85 (d, J=8.0 Hz, 1H), 8.12 (dd, J=8.0 Hz, 1H), 8.38 (d, J=2.4 Hz, 1H), 8.54 (m, 1H), 8.55 (m, 1H); MS (ESI) (M+H)⁺ 375.0.

### Example 51

### 3-[(4-Methyl-1-naphthoyl)amino]-N-(tetrahydro-2H-pyran-4-ylmethyl)pyrazine-2-carboxamide

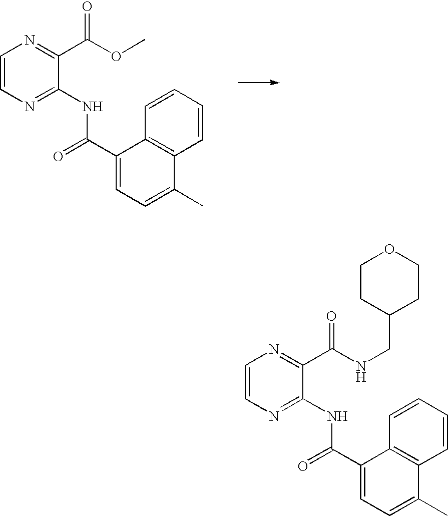

Following the procedure for Step A in Example 48, using methyl 3-[(4-methyl-1-naphthoyl)amino]pyrazine-2-carboxylate (64 mg, 0.2 mmol) and (tetrahydro-2H-pyran-4-ylmethyl)amine (34 mg, 0.4 mmol) provided the title compound as its TFA salt after purification by reversed-phase HPLC (28 mg, 27%). ¹H NMR (400 MHz, CD₃OD) δ 1.30 (m, 2H), 1.63 (m, 2H), 1.86 (m, 1H), 2.75 (s, 3H), 3.24 (m, 2H), 3.34 (m, 2H), 3.89 (m, 2H), 7.44 (d, J=8.0 Hz, 1H), 7.59 (m, 2H), 7.85 (d, J=8.0 Hz, 1H), 8.12 (dd, J=8.0 Hz, 1H), 8.38 (d, J=2.4 Hz, 1H), 8.54 (m, 1H), 8.55 (m, 1H); MS (ESI) (M+H)⁺ 405.0.

### Example 52

### N-(Cyclobutylmethyl)-3-[(4-ethyl-1-naphthoyl)amino]pyrazine-2-carboxamide

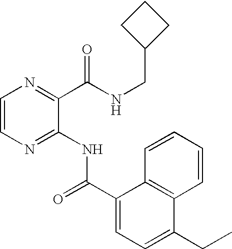

### Step A: N-(Cyclobutylmethyl)-3-[(4-ethyl-1-naphthoyl)amino]pyrazine-2-carboxamide

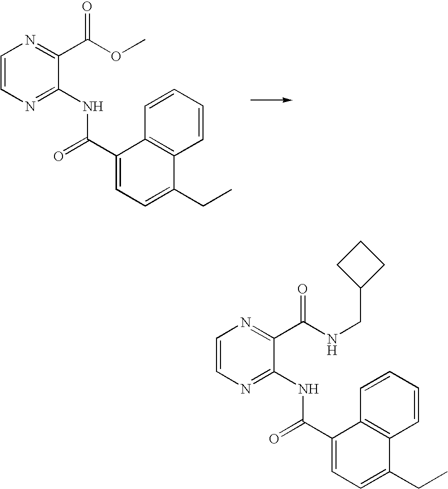

Following the procedure for Step A in Example 48, using methyl 3-[(4-ethyl-1-naphthoyl)amino]pyrazine-2-carboxylate (0.3 mmol) and (cyclobutylmethyl)amine (85 mg, 1.0 mmol) provided the title compound as its TFA salt after purification by reversed-phase HPLC (52 mg, 35%). ¹H NMR (400 MHz, CD₃OD) δ 1.38 (t, J=7.6 Hz, 3H), 1.75 (m, 2H), 1.85 (m, 2H), 2.02 (m, 2H), 2.58 (m, 1H), 3.16 (d, J=7.6 Hz, 2H), 3.36 (q, J=7.2 Hz, 2H), 7.45 (d, J=8.0 Hz, 1H), 7.57 (m, 2H), 7.87 (d, J=8.0 Hz, 1H), 8.16 (dd, J=8.0 Hz, 1H), 8.37 (d, J=2.4 Hz, 1H), 8.52 (m, 1H), 8.54 (d, J=2.4 Hz, 1H); MS (ESI) (M+H)⁺ 389.0.

### Step B: Methyl 3-[(4-ethyl-1-naphthoyl)amino]pyrazine-2-carboxylate

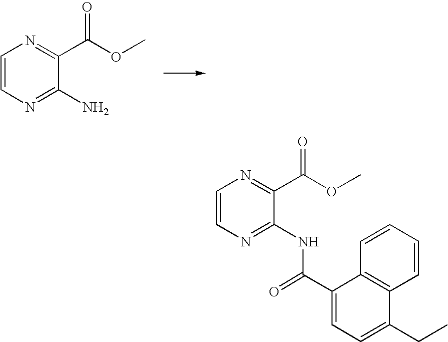

Following the procedure for Step B in Example 48, using 4-ethyl-1-naphthalenecarbonyl chloride (0.45 mmol) and methyl 3-aminopyrazine-2-carboxylate (46 mg, 0.3 mmol) provided a crude methyl 3-[(4-ethyl-1-naphthoyl)amino]pyrazine-2-carboxylate, which was used directly in the Step A.

### Example 53

### N-(Cyclohexylmethyl)-3-[(4-ethyl-1-naphthoyl)amino]pyrazine-2-carboxamide

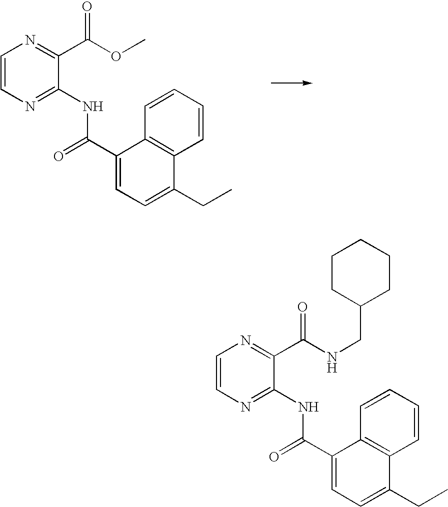

Following the procedure for Step A in Example 48, using methyl 3-[(4-ethyl-1-naphthoyl)amino]pyrazine-2-carboxylate (101 mg, 0.3 mmol) and (cyclohexylmethyl)amine (113 mg, 1.0 mmol) provided the title compound as its TFA salt after purification by reversed-phase HPLC (94 mg, 59%). ¹H NMR (400 MHz, CD₃OD) δ 0.96 (m, 2H), 1.21 (m, 4H), 1.39 (t, J=7.6 Hz, 3H), 1.60 (m, 1H), 1.71 (m, 4H), 3.17 (d, J=7.6 Hz, 2H), 3.18 (q, J=7.6 Hz, 2H), 7.47 (d, J=8.6 Hz, 1H), 7.57 (m, 2H), 7.88 (d, J=7.6 Hz, 1H), 8.19 (dd, J=8.0, 1.6 Hz, 1H), 8.42 (m, 1H), 8.52 (m, 1H), 8.59 (m, 1H); MS (ESI) (M+H)⁺ 417.0.

### Example 54

### 3-[(4-Ethyl-1-naphthoyl)amino]-N-(tetrahydro-2H-pyran-4-ylmethyl)pyrazine-2-carboxamide

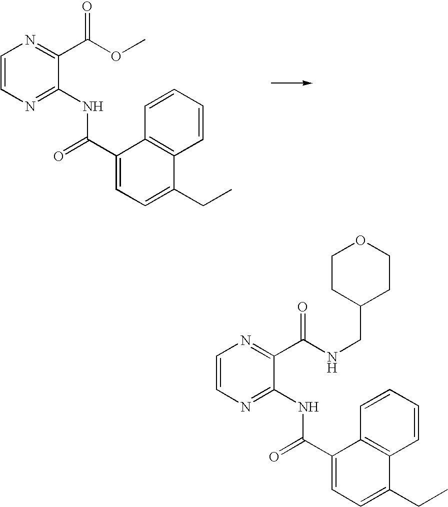

Following the procedure for Step A in Example 48, using methyl 3-[(4-ethyl-1-naphthoyl)amino]pyrazine-2-carboxylate (101 mg, 0.3 mmol) and (tetrahydro-2H-pyran-4-ylmethyl)amine (51 mg, 0.6 mmol) provided the title compound as its TFA salt after purification by reversed-phase HPLC (32 mg, 20%). ¹H NMR (400 MHz, CD₃OD) δ 1.30 (m, 2H), 1.39 (t, J=7.6 Hz, 3H), 1.63 (m, 2H), 1.86 (m, 1H), 3.18 (q, J=7.6 Hz, 2H), 3.24 (m, 2H), 3.34 (m, 2H), 3.89 (m, 2H), 7.47 (d, J=8.6 Hz, 1H), 7.59 (m, 2H), 7.88 (d, J=7.6 Hz, 1H), 8.19 (dd, J=8.0, 1.6 Hz, 1H), 8.42 (m, 1H), 8.52 (m, 1H), 8.59 (m, 1H); MS (ESI) (M+H)⁺ 419.0.

### Example 55

### N-(Cyclobutylmethyl)-3-{[4-(1H-1,2,3-triazol-1-ylmethyl)-1-naphthoyl]amino}pyrazine-2-carboxamide

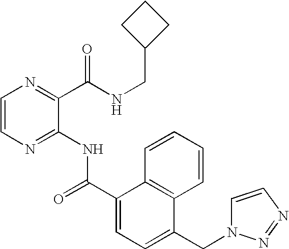

### Step A: N-(Cyclobutylmethyl)-3-{[4-(1H-1,2,3-triazol-1-ylmethyl)-1-naphthoyl]amino}pyrazine-2-carboxamide

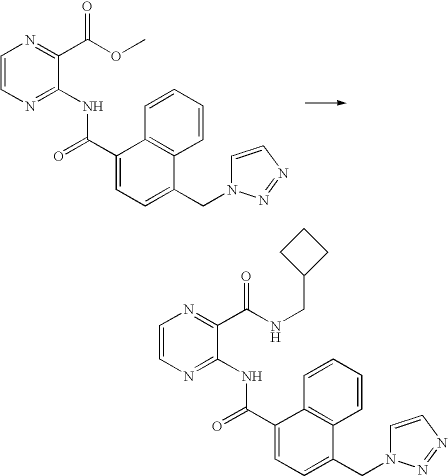

Following the procedure for Step A in Example 48, using methyl 3-{[4-(1H-1,2,3-triazol-1-ylmethyl)-1-naphthoyl]amino}pyrazine-2-carboxylate (34 mg, 0.09 mmol) and (cyclobutylmethyl)amine (85 mg, 1.0 mmol) provided the title compound as its TFA salt after purification by reversed-phase HPLC (17 mg, 35%). ¹H NMR (400 MHz, CD₃OD) δ 1.76 (m, 2H), 1.90 (m, 2H), 2.05 (m, 2H), 2.61 (m, 1H), 3.38 (m, 2H), 6.22 (s, 2H), 7.47 (d, J=8.0 Hz, 1H), 7.64 (m, 2H), 7.76 (s, 1H), 7.94 (d, J=8.0 Hz, 1H), 7.99 (s, 1H), 8.24 (d, J=8.0 Hz, 1H), 8.43 (m, 1H), 8.51 (m, 1H), 8.56 (m, 1H); MS (ESI) (M+H)⁺ 442.4.

### Step B: Methyl 3-{[4-(1H-1,2,3-triazol-1-ylmethyl)-1-naphthoyl]amino}pyrazine-2-carboxylate

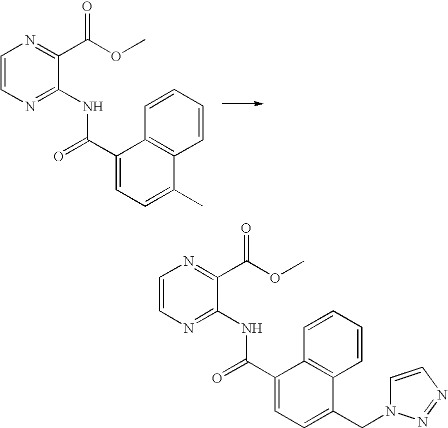

To a stirring solution of methyl 3-[(4-methyl-1-naphthoyl)amino]pyrazine-2-carboxylate (210 mg, 0.65 mmol) and NBS (462 mg, 2.6 mmol) in 20 mL of ClCH₂CH₂Cl at r.t was added 1,1′-azobis(cyclohexanecarbonitrile (5 mg). The solution was heated at 110° C. for 2 hr, and was then cooled to r.t. After removal of solvents (<20° C.), the residue was dissolved in 10 mL DMF, and followed by addition of 1,2,3-triazole (690 mg, 10 mmol). The resulting solution was then stirred for 4 hr at r.t. After removal of solvents, the residue was purified by MPLC (EtOAc) to give methyl 3-{[4-(1H-1,2,3-triazol-1-ylmethyl)-1-naphthoyl]amino}pyrazine-2-carboxylate (102 mg, 40%). MS (ESI) (M)⁺ 388.91.

### Example 56

### N-(Cyclohexylmethyl)-3-{[4-(1H-1,2,3-triazol-1-ylmethyl)-1-naphthoyl]amino}pyrazine-2-carboxamide

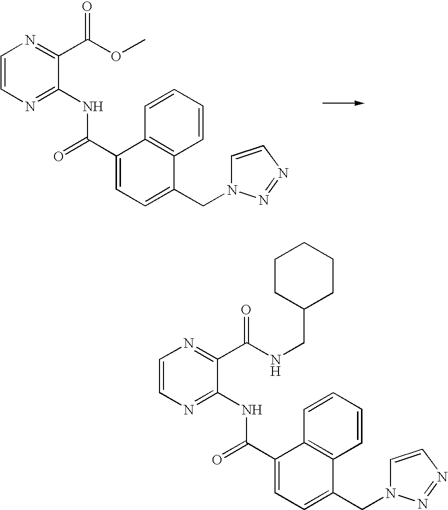

Following the procedure for Step A in Example 48, using methyl 3-{[4-(1H-1,2,3-triazol-1-ylmethyl)-1-naphthoyl]amino}pyrazine-2-carboxylate (34 mg, 0.09 mmol) and (cyclohexylmethyl)amine (113 mg, 1.0 mmol) provided the title compound as its TFA salt after purification by reversed-phase HPLC (16 mg, 33%). ¹H NMR (400 MHz, CD₃OD) δ 0.98 (m, 2H), 1.21 (m, 3H), 1.73 (m, 6H), 3.19 (m, 2H), 6.22 (s, 2H), 7.48 (d, J=8.0 Hz, 1H), 7.65 (m, 2H), 7.77 (s, 1H), 7.95 (d, J=8.0 Hz, 1H), 7.99 (s, 1H), 8.24 (d, J=8.0 Hz, 1H), 8.43 (m, 1H), 8.54 (m, 1H), 8.58 (m, 1H); MS (ESI) (M+H)⁺ 470.0.

### Example 57

### N-(Tetrahydro-2H-pyran-4-ylmethyl)-3-{[4-(1H-1,2,3-triazol-1-ylmethyl)-1-naphthoyl]amino}pyrazine-2-carboxamide

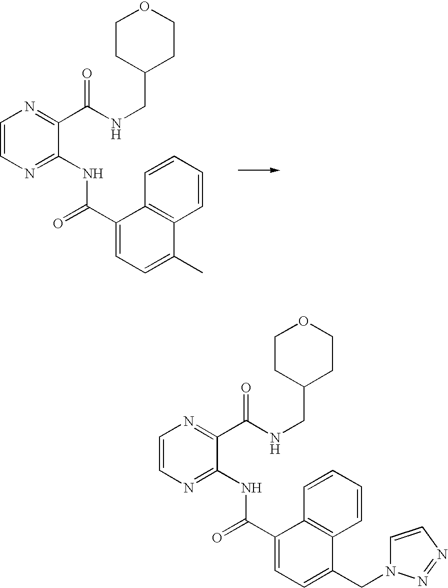

Following the procedure for Step B in Example 55, using 3-[(4-methyl-1-naphthoyl)amino]-N-(tetrahydro-2H-pyran-4-ylmethyl)pyrazine-2-carboxamide (50 mg, 0.12 mmol) and 1,2,3-triazole (69 mg, 1.0 mmol) provided the title compound as its TFA salt after purification by reversed-phase HPLC (14 mg, 20%). ¹H NMR (400 MHz, CD₃OD) δ 1.30 (m, 2H), 1.63 (m, 2H), 1.89 (m, 1H), 3.24 (m, 2H), 3.36 (m, 2H), 3.88 (m, 2H), 6.21 (s, 2H), 7.45 (d, J=7.6 Hz, 1H), 7.64 (m, 2H), 7.76 (s, 1H), 7.93 (d, J=7.6 Hz, 1H), 7.98 (s, 1H), 8.24 (d, J=8.0 Hz, 1H), 8.42 (m, 1H), 8.52 (m, 1H), 8.59 (m, 1H); MS (ESI) (M+H)⁺ 472.0.

### Example 58

### N-(3-Methylbutyl)-3-{[4-(1H-1,2,3-triazol-1-ylmethyl)-1-naphthoyl]amino}pyrazine-2-carboxamide

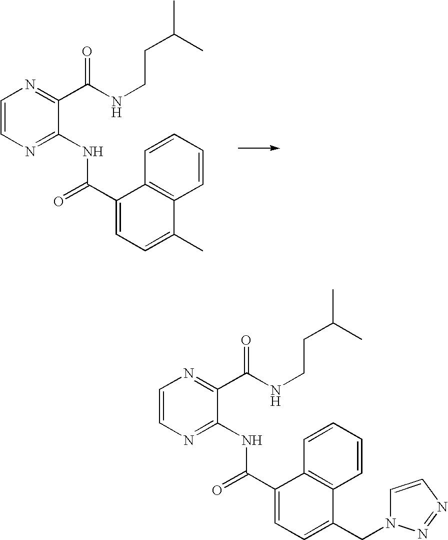

Following the procedure for Step B in Example 55, using N-(3-methylbutyl)-3-[(4-methyl-1-naphthoyl)amino]pyrazine-2-carboxamide (40 mg TFA salt, 0.08 mmol) and 1,2,3-triazole (69 mg, 1.0 mmol) provided the title compound as its TFA salt after purification by reversed-phase HPLC (13 mg, 30%). ¹H NMR (400 MHz, CD₃OD) δ 0.87 (d, J=7.6 Hz, 6H), 1.42 (m, 2H), 1.56 (m, 1H), 3.31 (m, 2H), 6.22 (s, 2H), 7.47 (d, J=8.0 Hz, 1H), 7.64 (m, 2H), 7.76 (s, 1H), 7.94 (d, J=8.0 Hz, 1H), 7.99 (s, 1H), 8.24 (d, J=8.0 Hz, 1H), 8.43 (m, 1H), 8.51 (m, 1H), 8.56 (m, 1H); MS (ESI) (M+H)⁺ 444.0.

### Example 59

### 3-{[4-(Methoxymethyl)-1-naphthoyl]amino}-N-(tetrahydro-2H-pyran-4-ylmethyl)pyrazine-2-carboxamide

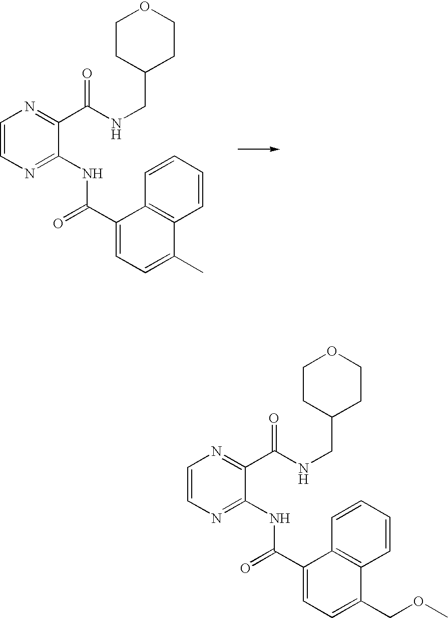

To a stirring solution of 3-[(4-methyl-1-naphthoyl)amino]-N-(tetrahydro-2H-pyran-4-ylmethyl)pyrazine-2-carboxamide (50 mg, 0.12 mmol) and NBS (266 mg, 1.5 mmol) in 20 mL of ClCH₂CH₂Cl at r.t was added 1,1′-azobis(cyclohexanecarbonitrile (5 mg). The solution was heated at 110° C. for 3 hr, and was then cooled to r.t. After removal of solvents (<20° C.), the residue was dissolved in 10 mL MeOH, and followed by addition of NaOMe solution (2 mL, 10% in MeOH). The resulting solution was then stirred for 4 hr at r.t. After standard workup, the residue was purified by reversed-phase HPLC to give the title compound as its TFA salt (6 mg, 9%). ¹H NMR (400 MHz, CD₃OD) δ 1.30 (m, 2H), 1.634 (m, 2H), 1.87 (m, 1H), 3.26 (m, 2H), 3.36 (m, 2H), 3.49 (s, 3H), 3.91 (m, 2H), 4.98 (s, 2H), 7.61 (m, 2H), 7.66 (d, J=7.6 Hz, 1H), 7.92 (d, J=7.6 Hz, 1H), 8.19 (d, J=8.0 Hz, 1H), 8.40 (m, 1H), 8.51 (m, 1H), 8.59 & 9.24 (m, 1H); MS (ESI) (M+H)⁺ 435.0.

### Example 60

### N-(Cyclobutylmethyl)-3-{[4-(methoxymethyl)-1-naphthoyl]amino}pyrazine-2-carboxamide

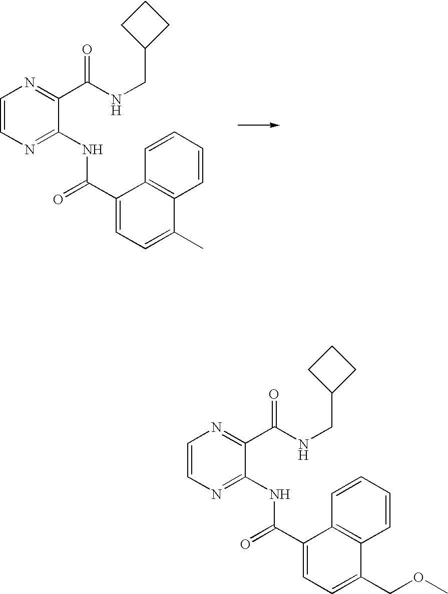

Following the procedure in Example 59, using N-(cyclobutylmethyl)-3-[(4-methyl-1-naphthoyl)amino]pyrazine-2-carboxamide (50 mg, 0.13 mmol) provided the title compound as its TFA salt after purification by reversed-phase HPLC (20 mg, 29%). ¹H NMR (400 MHz, CD₃OD) δ 1.76 (m, 2H), 1.90 (m, 2H), 2.05 (m, 2H), 2.61 (m, 1H), 3.38 (m, 2H), 3.49 (s, 3H), 3.91 (m, 2H), 4.99 (s, 2H), 7.62 (m, 2H), 7.66 (d, J=7.6 Hz, 1H), 7.93 (d, J=7.6 Hz, 1H), 8.20 (d, J=8.0 Hz, 1H), 8.40 (m, 1H), 8.50 (m, 1H), 8.59 (m, 1H); MS (ESI) (M+H)⁺ 405.0.

### Example 61

### N-(Cyclohexylmethyl)-3-[(4-methoxy-1-naphthoyl)amino]pyrazine-2-carboxamide

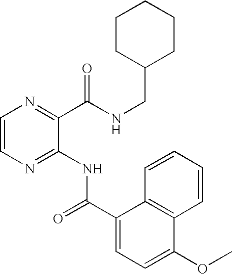

### Step A: N-(Cyclohexylmethyl)-3-[(4-methoxy-1-naphthoyl)amino]pyrazine-2-carboxamide

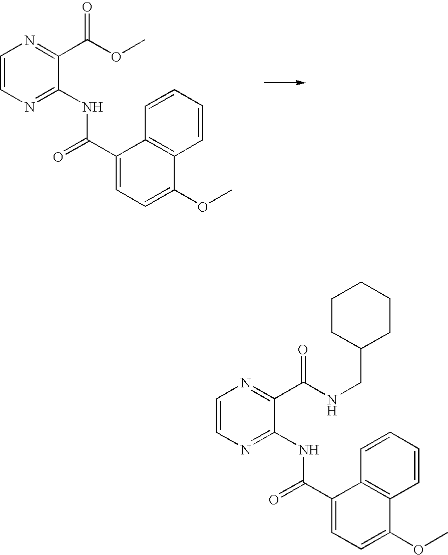

Following the procedure for Step A in Example 48, using methyl 3-[(4-methoxy-1-naphthoyl)amino]pyrazine-2-carboxylate (169 mg, 0.5 mmol) and (cyclohexylmethyl)amine (113 mg, 1.0 mmol) provided the title compound as its TFA salt after purification by reversed-phase HPLC (122 mg, 46%). ¹H NMR (400 MHz, CD₃OD) δ 0.87 (m, 2H), 1.10 (m, 3H), 1.64 (m, 6H), 3.09 (d, J=7.6 Hz, 2H), 3.94 (s, 3H), 6.85 (d, J=8.0 Hz, 1H), 7.43 (m, 1H), 7.51 (m, 1H), 7.91 (d, J=8.0 Hz, 1H), 8.20 (d, J=8.0 Hz, 1H), 8.25 (s, 1H), 8.45 (s, 1H), 9.58 (d, J=8.0 Hz, 1H); MS (ESI) (M+H)⁺ 419.0.

### Step B: Methyl 3-[(4-Methoxy-1-naphthoyl)amino]pyrazine-2-carboxylate

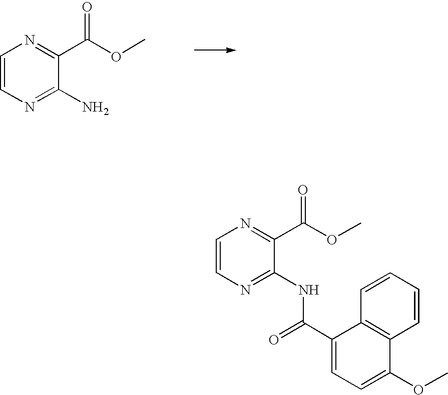

Following the procedure for Step B in Example 48, using 4-methoxy-1-naphthalenecarbonyl chloride (3.0 mmol and methyl 3-aminopyrazine-2-carboxylate (459 mg, 3.0 mmol) provided the title compound after purification (584 mg, 58%).

### Example 62

### 3-{[5-Bromo-4-(1H-1,2,3-triazol-1-ylmethyl)-1-naphthoyl]amino}-N-(cyclohexylmethyl)pyrazine-2-carboxamide

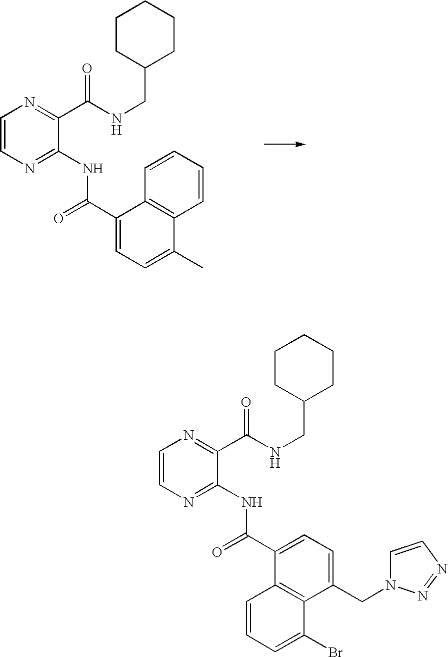

To a stirring solution of N-(cyclohexylmethyl)-3-[(4-methyl-1-naphthoyl)amino]pyrazine-2-carboxamide (100 mg, 0.25 mmol) and NBS (231 mg, 1.3 mmol) in 20 mL of ClCH₂CH₂Cl at r.t was added 1,1′-azobis(cyclohexanecarbonitrile (5 mg). The solution was heated at 110° C. for 24 hr, and was then cooled to r.t. After removal of solvents (<20° C.), the residue was dissolved in 10 mL MeCN, and followed by addition of 1,2,3-triazole (345 mg, 5 mmol). The resulting solution was then stirred for 4 hr at r.t. After condensation, the residue was purified to provide the title compound as its TFA salt by reversed-phase HPLC (35 mg, 21%). ¹H NMR (400 MHz, CD₃OD) δ 0.88 (m, 2H), 1.12 (m, 3H), 1.64 (m, 6H), 3.09 (m, 2H), 4.79 (s, 2H), 7.38 (d, J=8.0 Hz, 1H), 7.55 (m, 1H), 7.66 (s, 1H), 7.84 (d, J=8.0 Hz, 1H), 7.88 (s, 1H), 8.14 (d, J=8.0 Hz, 1H), 8.42 (d, J=8.0 Hz, 1H), 8.60 (s, 1H), 8.93 (m, 1H); MS (ESI) (M+H)⁺ 547.7.

### Example 63

### 3-[(4-Methoxy-1-naphthoyl)amino]-N-(tetrahydrofuran-2-ylmethyl)pyridine-2-carboxamide

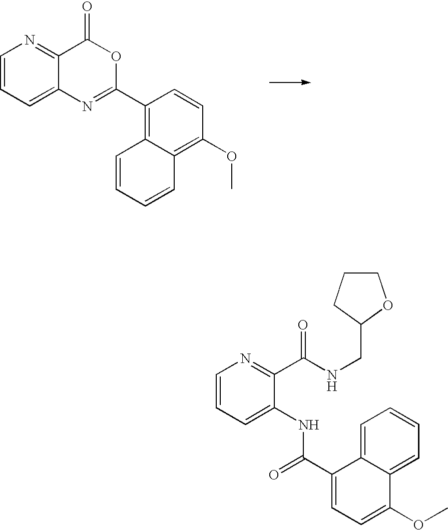

Following the procedure for Step A in Example 1, using 2-(4-methoxy-1-naphthyl)-4H-pyrido[3,2-d][1,3]oxazin-4-one (12 mg, 0.04 mmol), and (tetrahydrofuran-2-ylmethyl)amine (20 mg, 0.2 mmol) provided the title compound (4.5 mg, 28%). MS (ESI) (M+H)⁺=406.2.

### Example 64

### N-(1,4-Dioxan-2-ylmethyl)-3-[(4-methoxy-1-naphthoyl)amino]pyridine-2-carboxamide

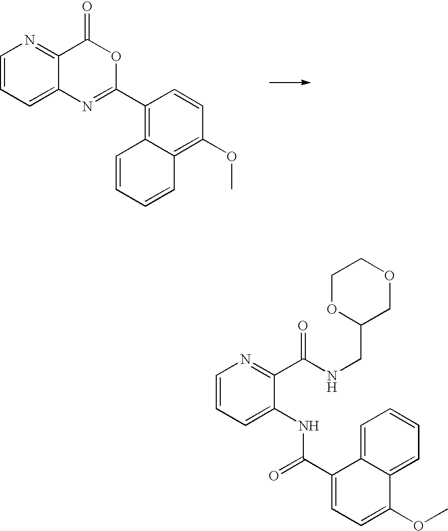

Following the procedure for Step A in Example 1, using 2-(4-methoxy-1-naphthyl)-4H-pyrido[3,2-d][1,3]oxazin-4-one (12 mg, 0.04 mmol), and (1,4-dioxan-2-ylmethyl)amine (23 mg, 0.2 mmol) provided the title compound (4.5 mg, 28%). MS (ESI) (M+H)⁺=422.2.

### Example 65

### 3-[(4-Methoxy-1-naphthoyl)amino]-N-(tetrahydro-2H-pyran-4-yl)pyridine-2-carboxamide

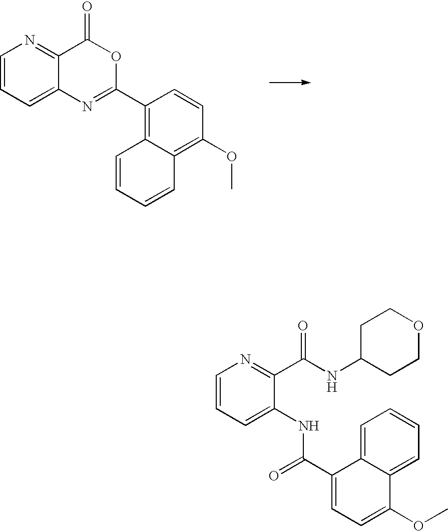

Following the procedure for Step A in Example 1, using 2-(4-methoxy-1-naphthyl)-4H-pyrido[3,2-d][1,3]oxazin-4-one (100 mg, 0.33 mmol), and tetrahydro-2H-pyran-4-amine (101 mg, 1.0 mmol) provided the title compound as its TFA salt after purification by reversed-phase HPLC (34 mg, 20%). ¹H NMR (400 MHz, CD₃OD) δ 1.71 (m, 2H), 1.85 (m, 2H), 3.27 (m, 2H), 3.49 (m, 2H), 3.93 (m, 2H), 4.05 (m, 1H), 4.08 (s, 3H), 7.02 (d, J=8.4 Hz, 1H), 7.59 (m, 3H), 7.92 (d, J=8.0 Hz, 1H), 8.34 (m, 2H), 8.53 (d, J=8.0 Hz, 1H), 9.26 (d, J=8.0 Hz, 1H); MS (ESI) (M+H)⁺=406.0.

### Example 66

### 3-[(4-Methoxy-1-naphthoyl)amino]-N-[2-(tetrahydro-2H-pyran-4-yl)ethyl]pyridine-2-carboxamide

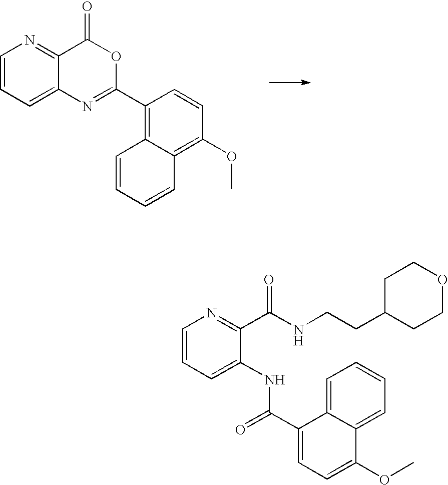

Following the procedure for Step A in Example 1, using 2-(4-methoxy-1-naphthyl)-4H-pyrido[3,2-d][1,3]oxazin-4-one (100 mg, 0.33 mmol), and [2-(tetrahydro-2H-pyran-4-yl)ethyl]amine (129 mg, 1.0 mmol) provided the title compound as its TFA salt after purification by reversed-phase HPLC (34 mg, 19%). ¹H NMR (400 MHz, CDCl₃) δ 1.35 (m, 2H), 1.63 (m, 5H), 3.38 (m, 2H), 3.46 (m, 2H), 3.95 (m, 2H), 4.06 (s, 3H), 6.88 (d, J=8.0 Hz, 1H), 7.52 (m, 2H), 7.60 (m, 1H), 7.93 (d, J=−8.0 Hz, 1H), 8.26 (d, J=4.4 Hz, 1H), 8.35 (d, J=8.0 Hz, 1H), 8.42 (brs, 1H), 8.64 (d, J=8.0 Hz, 1H), 9.39 (dd, J=8.4, 1.2 Hz, 1H), 12.75 (brs, 1H); MS (ESI) (M+H)⁺=434.0.

### Example 67

### 3-[(4-Methoxy-1-naphthoyl)amino]-N-[(2R)-piperidin-2-ylmethyl]pyridine-2-carboxamide

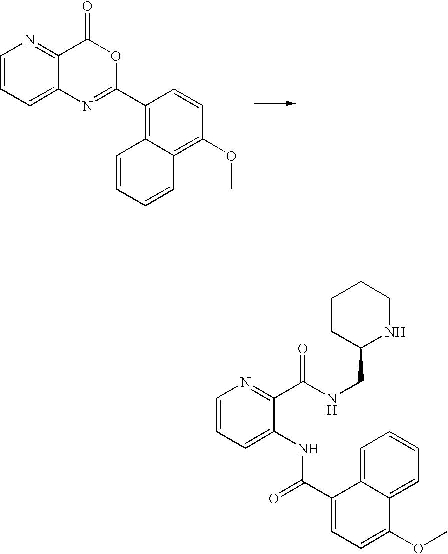

Following the procedure for Step A in Example 1, using 2-(4-methoxy-1-naphthyl)-4H-pyrido[3,2-d][1,3]oxazin-4-one (100 mg, 0.33 mmol), and [(2R)-piperidin-2-ylmethyl]amine (114 mg, 1.0 mmol) provided the title compound as its TFA salt after purification by reversed-phase HPLC (58 mg, 33%). ¹H NMR (400 MHz, CD₃OD) δ 1.54 (m, 3H), 1.83 (m, 3H), 2.85 (m, 1H), 3.27 (m, 2H), 3.59 (m, 2H), 4.07 (s, 3H), 6.96 (d, J=8.0 Hz, 1H), 7.62 (m, 3H), 7.91 (d, J=8.0 Hz, 1H), 8.31 (d, J=4.4 Hz, 1H), 8.38 (d, J=8.0 Hz, 1H), 8.48 (d, J=8.0 Hz, 1H), 9.24 (d, J=8.0 Hz, 1H); MS (ESI) (M+H)⁺=419.0.

### Example 68

### 3-[(4-Methoxy-1-naphthoyl)amino]-N-(morpholin-3-ylmethyl)pyridine-2-carboxamide

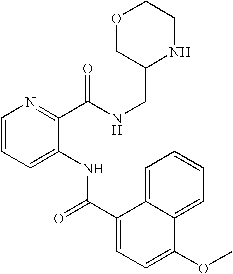

### Step A: 3-[(4-Methoxy-1-naphthoyl)amino]-N-(morpholin-3-ylmethyl)pyridine-2-carboxamide

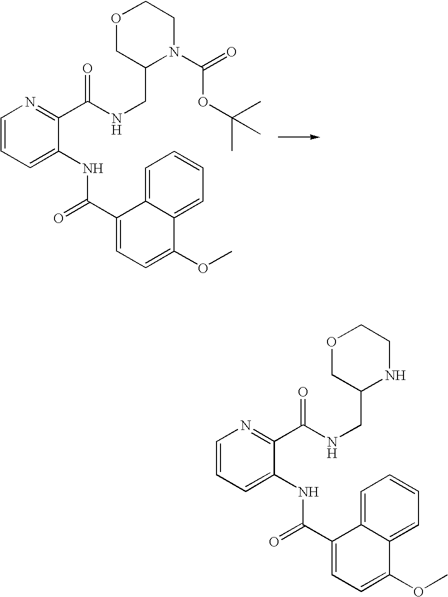

The crude tert-butyl 3-{[({3-[(4-methoxy-1-naphthoyl)amino]pyridin-2-yl}carbonyl)amino]methyl}morpholine-4-carboxylate from Step B was treated with 4 N HCl in dioxane for 1 hr at r.t. After evaporation, the residue was purified by reversed-phase HPLC to provide the title compound as its TFA salt (56 mg, 32% for two steps). ¹H NMR (400 MHz, CD₃OD) δ 3.02 (m, 1H), 3.21 (m, 2H), 3.47 (m, 2H), 3.59 (m, 2H), 3.82 (m, 1H), 3.90 (m, 1H), 4.07 (s, 3H), 6.97 (d, J=8.0 Hz, 1H), 7.56 (m, 3H), 7.91 (d, J=8.0 Hz, 1H), 8.31(d, J=4.4 Hz, 1H), 8.38 (d, J=8.0 Hz, 1H), 8.48 (d, J=8.0 Hz, 1H), 9.25 (d, J=8.0 Hz, 1H); MS (ESI) (M+H)⁺=421.0.

### Step B: tert-Butyl 3-{[({3-[(4-methoxy-1-naphthoyl)amino]pyridin-2-yl}carbonyl)amino]methyl}morpholine-4-carboxylate

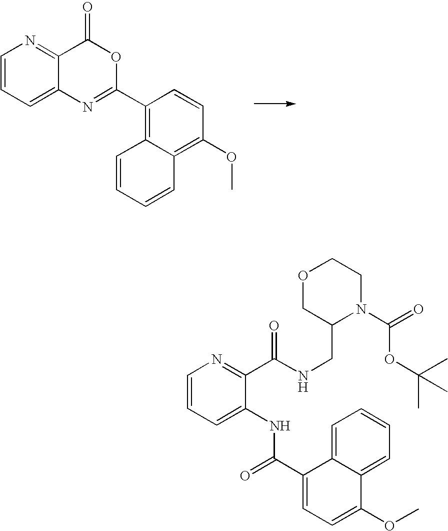

Following the procedure for Step A in Example 1, using 2-(4-methoxy-1-naphthyl)-4H-pyrido[3,2-d][1,3]oxazin-4-one (100 mg, 0.33 mmol), and tert-butyl 3-(aminomethyl)morpholine-4-carboxylate (216 mg, 1.0 mmol) provided crude tert-butyl 3-{[({3-[(4-methoxy-1-naphthoyl)amino]pyridin-2-yl}carbonyl)amino]methyl}morpholine-4-carboxylate, which was used directly in Step A.

### Example 69

### N-[(1-Hydroxycyclohexyl)methyl]-3-[(4-methoxy-1-naphthoyl)amino]pyridine-2-carboxamide

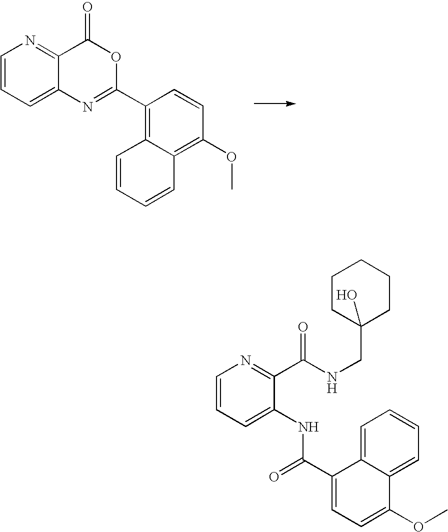

Following the procedure for Step A in Example 1, using 2-(4-methoxy-1-naphthyl)-4H-pyrido[3,2-d][1,3]oxazin-4-one (100 mg, 0.33 mmol), 1-(aminomethyl)cyclohexanol hydrochloride (165 mg, 1.0 mmol), and DIPEA (1 mL) provided the title compound as its TFA salt after purification by reversed-phase HPLC (58 mg, 32%). ¹H NMR (400 MHz, CDCl₃) δ 1.28 (m, 2H), 1.58 (m, 8H), 2.07 (brs, 1H), 3.45 (d, J=6.4 Hz, 2H), 4.06 (s, 3H), 6.87 (d, J=8.0 Hz, 1H), 7.53 (m, 2H), 7.59 (m, 1H), 7.92 (d, J=8.0 Hz, 1H), 8.27(m, 1H), 8.32 (d, J=8.0 Hz, 1H), 8.64 (d, J=8.0 Hz, 1H), 8.79 (s, 1H), 9.39 (d, J=8.0 Hz, 1H), 12.69 (s, 1H); MS (ESI) (M+H)⁺=434.0.

### Example 70

### N-(Cyclohexylmethyl)-3-[(4-ethoxy-1-naphthoyl)amino]pyridine-2-carboxamide

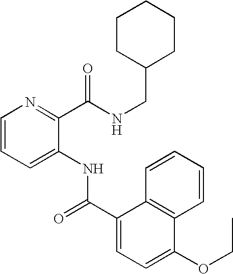

### Step A: N-(Cyclohexylmethyl)-3-[(4-ethoxy-1-naphthoyl)amino]pyridine-2-carboxamide

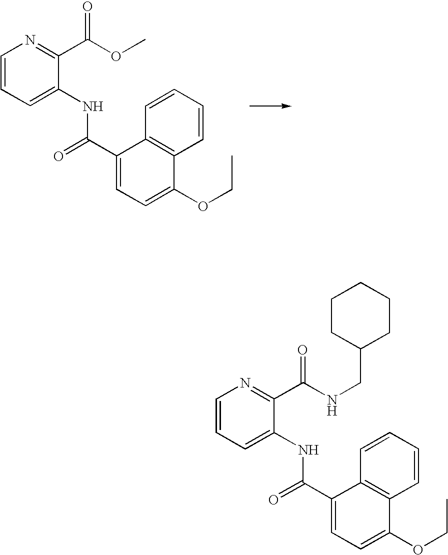

Following the procedure for Step A in Example 48, using methyl 3-[(4-ethoxy-1-naphthoyl)amino]pyridine-2-carboxylate (100 mg, 0.29 mmol) and (cyclohexylmethyl)amine (113 mg, 1.0 mmol) provided the title compound as its TFA salt after purification by reversed-phase HPLC (36 mg, 23%). ¹H NMR (400 MHz, CDCl₃) δ 1.0 (m, 2H), 1.23 (m, 3H), 1.59 (m, 5H), 1.76 (m, 4H), 3.25 (m, 2H), 4.26 (m, 2H), 6.85 (d, J=8.0 Hz, 1H), 7.52 (m, 3H), 7.92 (d, J=8.0 Hz, 1H), 8.25 (s, 1H), 8.37 (d, J=8.0 Hz, 1H), 8.59 (s, 1H), 8.60 (d, J=8.0 Hz, 1H), 9.38 (d, J=8.0, Hz, 1H), 12.8 (s, 1H); MS (ESI) (M+H)⁺ 432.0.

### Step B: Methyl 3-[(4-ethoxy-1-naphthoyl)amino]pyridine-2-carboxylate

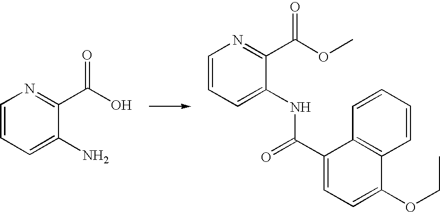

4-Ethoxy-1-naphthoic acid (7.0 mmol) in 50 mL CH₂Cl₂ was treated with oxalyl chloride (10 mL, 2.0 M in CH₂Cl₂, 20 mmol) at r.t for 1 hr, and then heated to 50° C. for 1 hr. The reaction mixture was then condensed to afford 4-ethoxy-1-naphthalenecarbonyl chloride, which was added into a solution of 3-amino-2-pyridinecarboxylic acid (7.0 mmol) and DIPEA (14 mmol) in DMF (40 mL) at 0° C. After stirred for 1 hr at r.t, and for 1 hr at 50° C., K₂CO₃ (1.86 g, 14 mmol) was added into the reaction mixture, and followed by addition of MeI (3.1 mL, 50 mmol) in portion at r.t. After stirred overnight, the reaction mixture was condensed, and extracted by EtOAc, washed by brine, dried over MgSO₄. Removal of solvents provided a crude methyl 3-[(4-ethoxy-1-naphthoyl)amino]pyridine-2-carboxylate as a solid (2.25 g, 92%), which was used directly in Step A.

### Example 71

### 3-[(4-Ethoxy-1-naphthoyl)amino]-N-pentylpyridine-2-carboxamide

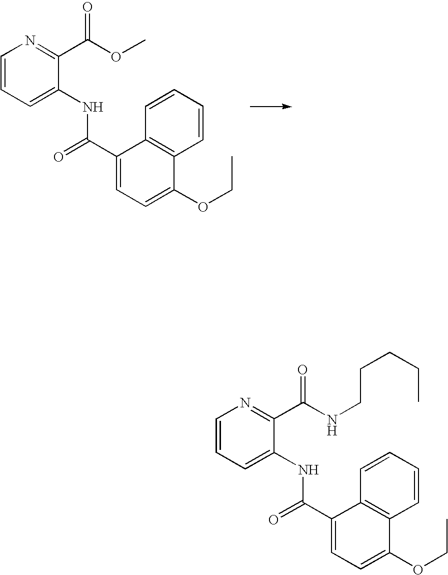

Following the procedure for Step A in Example 48, using methyl 3-[(4-ethoxy-1-naphthoyl)amino]pyridine-2-carboxylate (100 mg, 0.29 mmol) and pentan-1-amine (130 mg, 1.5 mmol) provided the title compound as its TFA salt after purification by reversed-phase HPLC (16 mg, 11%). ¹H NMR (400 MHz, CDCl₃) δ 0.91 (t, J=7.6 Hz, 3H), 1.37 (m, 4H), 1.59 (m, 5H), 3.41 (m, 2H), 4.27 (m, 2H), 6.85 (d, J=8.0 Hz, 1H), 7.52 (m, 3H), 7.92 (d, J=8.0 Hz, 1H), 8.25 (s, 1H), 8.37 (d, J=8.0 Hz, 1H), 8.48 (s, 1H), 8.63 (d, J=8.0 Hz, 1H), 9.38 (d, J=8.0, Hz, 1H), 12.8 (s, 1H); MS (ESI) (M+H)⁺ 406.0.

### Example 72

### 3-[(4-Ethoxy-1-naphthoyl)amino]-N-(tetrahydro-2H-pyran-4-ylmethyl)pyridine-2-carboxamide

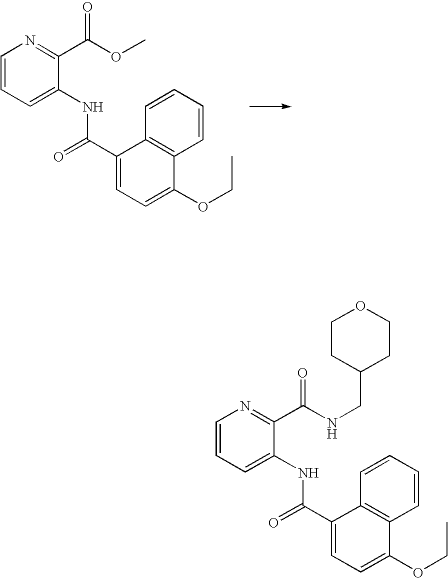

Following the procedure for Step A in Example 48, using methyl 3-[(4-ethoxy-1-naphthoyl)amino]pyridine-2-carboxylate (100 mg, 0.29 mmol) and (tetrahydro-2H-pyran-4-ylmethyl)amine (172 mg, 1.5 mmol) provided the title compound as its TFA salt after purification by reversed-phase HPLC (18 mg, 12%). ¹H NMR (400 MHz, CDCl₃) δ 1.41 (m, 2H), 1.59 (m, 3H), 1.68 (m, 2H), 1.82 (m, 1H), 3.34 (m, 2H), 3.44 (m, 2H), 4.05 (m, 2H), 4.28 (m, 2H), 6.85 (d, J=8.0 Hz, 1H), 7.55 (m, 3H), 7.90 (d, J=8.0 Hz, 1H), 8.27 (d, J=4.0 Hz, 1H), 8.37 (d, J=8.0 Hz, 1H), 8.57 (d, J=8.0 Hz, 1H), 8.62 (s, 1H), 9.38 (d, J=8.0, Hz, 1H), 12.7 (s, 1H); MS (ESI) (M+H)⁺ 434.0.

### Example 73

### N-(Cyclopentylmethyl)-3-[(4-ethoxy-1-naphthoyl)amino]pyridine-2-carboxamide

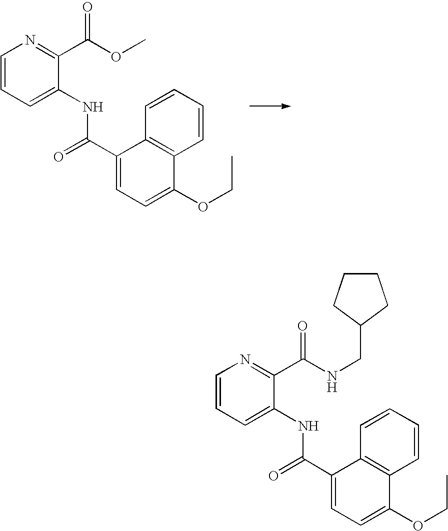

Following the procedure for Step A in Example 48, using methyl 3-[(4-ethoxy-1-naphthoyl)amino]pyridine-2-carboxylate (100 mg, 0.29 mmol) and (cyclopentylmethyl)amine (149 mg, 1.5 mmol) provided the title compound as its TFA salt after purification by reversed-phase HPLC (36 mg, 24%). ¹H NMR (400 MHz, CDCl₃) δ 1.25 (m, 2H), 1.59 (m, 7H), 1.82 (m, 2H), 2.18 (m, 1H), 3.35 (m, 2H), 4.27 (m, 2H), 6.85 (d, J=8.0 Hz, 1H), 7.52 (m, 3H), 7.92 (d, J=8.0 Hz, 1H), 8.35 (s, 1H), 8.37 (d, J=8.0 Hz, 1H), 8.56 (s, 1H), 8.61 (d, J=8.0 Hz, 1H), 9.38 (d, J=8.0, Hz, 1H), 12.8 (s, 1H); MS (ESI) (M+H)⁺ 418.0.

### Example 74

### 3-[(4-Ethoxy-1-naphthoyl)amino]-N-[2-(tetrahydro-2H-pyran-4-yl)ethyl]pyridine-2-carboxamide

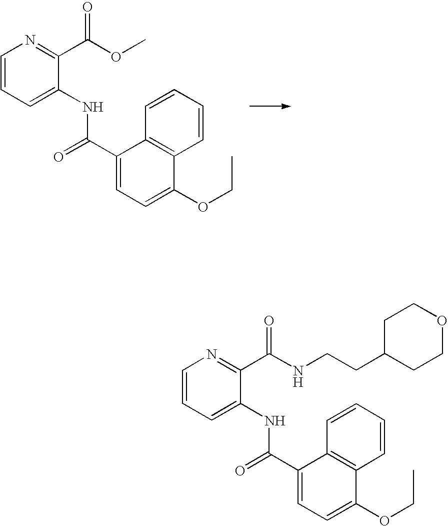

Following the procedure for Step A in Example 48, using methyl 3-[(4-ethoxy-1-naphthoyl)amino]pyridine-2-carboxylate (100 mg, 0.29 mmol) and 2-(tetrahydro-2H-pyran-4-yl)ethanamine (194 mg, 1.5 mmol) provided the title compound as its TFA salt after purification by reversed-phase HPLC (84 mg, 52%). ¹H NMR (400 MHz, CDCl₃) δ 1.35 (m, 2H), 1.59 (m, 7H), 2.28 (m, 1H), 3.38 (m, 2H), 3.47 (m, 2H), 3.95 (m, 2H), 4.27 (m, 2H), 6.85 (d, J=8.0 Hz, 1H), 7.52 (m, 3H), 7.90 (d, J=8.0 Hz, 1H), 8.25 (d, J=4.0 Hz, 1H), 8.35 (d, J=8.0 Hz, 1H), 8.48 (s, 1H), 8.64 (d, J=8.0 Hz, 1H), 9.38 (d, J=8.0, Hz, 1H), 12.7 (s, 1H); MS (ESI) (M+H)⁺ 448.0

### Example 75

### N-(Cyclobutylmethyl)-3-[(4-ethoxy-1-naphthoyl)amino]pyridine-2-carboxamide

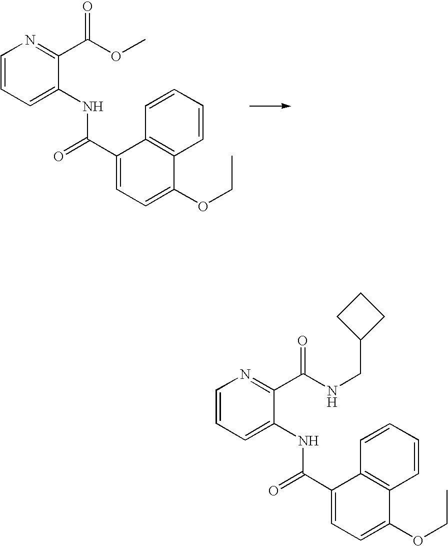

Following the procedure for Step A in Example 48, using methyl 3-[(4-ethoxy-1-naphthoyl)amino]pyridine-2-carboxylate (100 mg, 0.29 mmol) and (cyclobutylmethyl)amine (128 mg, 1.5 mmol) provided the title compound as its TFA salt after purification by reversed-phase HPLC (14 mg, 10%). ¹H NMR (400 MHz, CDCl₃) δ 1.60 (m, 3H), 1.69-1.78 (m, 2H), 1.81-1.91 (m, 2H), 1.99-2.07 (m, 2H), 2.51-2.62 (m, 1H), 3.34 (m, 2H), 4.27 (m, 2H), 6.85 (d, J=8.0 Hz, 1H), 7.52 (m, 3H), 7.92 (d, J=8.0 Hz, 1H), 8.35 (s, 1H), 8.37 (d, J=8.0 Hz, 1H), 8.56 (s, 1H), 8.61 (d, J=8.0 Hz, 1H), 9.38 (d, J=8.0, Hz, 1H), 12.8 (s, 1H); MS (ESI) (M+H)⁺ 404.0.

### Example 76

### N-Cyclobutyl-3-[(5-methyl-1-naphthoyl)amino]pyridine-2-carboxamide

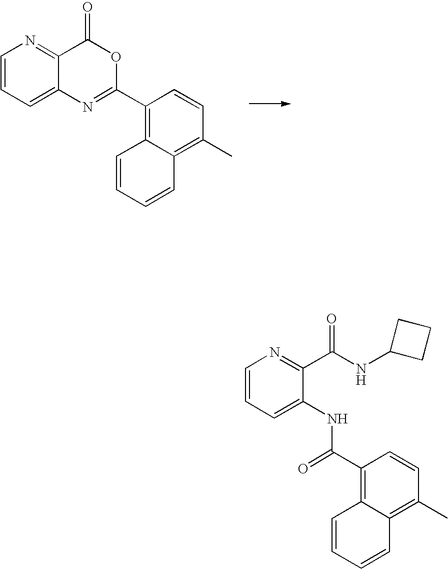

Following the procedure for Step A in Example 1, using 2-(4-methyl-1-naphthyl)-4H-pyrido[3,2-d][1,3]oxazin-4-one (193 mg, 0.67 mmol), and cyclobutylamine (200 mg, 2.81 mmol) provided the title compound after purification by MPLC on silica gel using hexane/EtOAc (4:1) (200 mg, 83%). ¹H NMR (400 MHz, CD₃OD) δ 1.71-1.85 (m, 2H), 2.05-2.20 (m, 2H), 2.22-2.41 (m, 2H), 2.76 (s, 3H), 4.34-4.51 (m, 1H), 7.45 (dd, J=7.32, 0.88 Hz, 1H), 7.52-7.66 (m, 3H), 7.78 (d, J=7.23 Hz, 1H), 8.08-8.20 (m, 1H), 8.37 (dd, J=4.49, 1.56 Hz, 1H), 8.42-8.48 (m, 1H), 9.28 (dd, J=8.49, 1.46 Hz, 1H). MS (ESI) (M+H)⁺ 360.0. Anal. Calcd for C₂₂H₂₁N₃O₂ (359.43): C, 73.52; H, 5.89; N, 11.69. Found: C, 73.44; H, 5.08; N, 11.48.

### Example 77

### 3-(1-Naphthoylamino)-N-[(2R)-piperidin-2-ylmethyl]pyridine-2-carboxamide

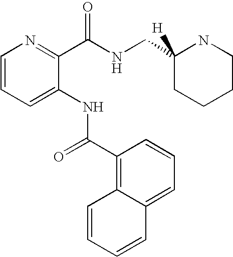

### Step A. 3-(1-Naphthoylamino)-N-[(2R)-piperidin-2-ylmethyl]pyridine-2-carboxamide

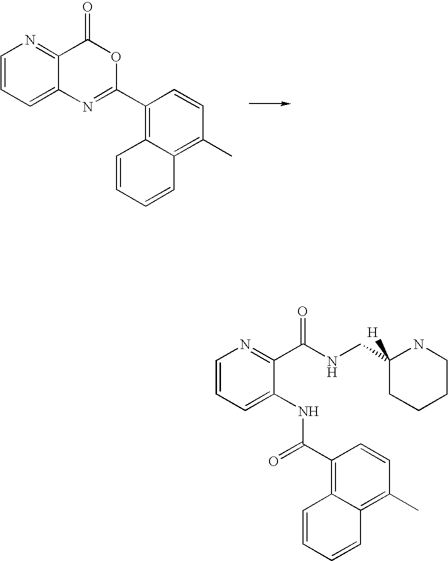

Following the procedure for Step A in Example 1, using 2-(4-methyl-1-Naphthalenyl)-H-pyrido[3,2-d][1,3]oxazin-4-one (260.0 mg, 0.9 mmol) and [(2R)-piperidin-2-ylmethyl]amine (for preparation, see following Steps B, C and D) (260.0 mg, 2.28 mmol) in DMF (8.0 mL) provided the title compound after purification by MPLC on silica gel using CH₂Cl₂/MeOH (20:1). (162 mg, 45%) as a white solid. [α]_{D}: +17.4° (c 0.265, EtOH). ¹H NMR (400 MHz, CD₃OD) δ 1.54 (m, 3H), 1.87 (m, 3H), 2.75 (s, 3H), 2.85 (m, 1H), 3.24 (m, 2H), 3.53 (dd, J=14.65, 3.71 Hz, 1H), 3.61 (dd, J=14.6, 7.6 Hz, 1H), 7.42 (d, J=7.23 Hz, 1H), 7.61 (m, 3H), 7.79 (d, J=7.23 Hz, 1H), 8.14 (m, 1H), 8.40 (dd, J=4.49, 1.56 Hz, 1H), 8.44 (dd, J=7.32, 1.46 Hz, 1H), 9.27 (dd, J=8.59, 1.37 Hz, 1H). MS (ESI) (M+H)⁺=403.0. Anal. Calcd for C₂₄H₂₆N₄O₂+1.40TFA+2.10H₂O: C, 53.65; H, 5.31; N, 9.34. Found: C, 53.61; H, 5.32; N, 9.49.

### Step B. tert-Butyl (2R)-2-(aminocarbonyl)piperidine-1-carboxylate

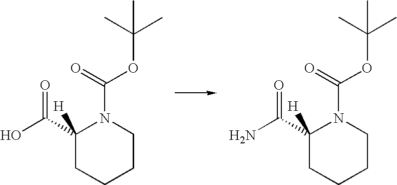

HATU (5.60 g, 14.7 mmol) was added to a mixture of the (2R)-1-(tert-butoxycarbonyl)piperidine-2-carboxylic acid (2.29 g, 10 mmol), ammonium chloride (3.21 g, 60 mmol) and DIPEA (3.88 g, 30 mmol) in DMF (70 mL) at 0° C. The mixture was stirred for 18 h at room temperature, diluted with H₂O (100 mL) and extracted with EtOAc (3×100 mL). The combined organic phases were washed with 10% Na₂CO₃ solution (2×30 mL), brine (2×30 mL) and dried with Na₂SO₄. After filtration and concentration, the title compound was purified by MPLC on silica gel using hexane/EtOAc (1:1) (2.28 g, 100%) as a white solid. ¹H NMR (400 MHz, CDCl₃) δ 1.46 (s, 9H), 1.63 (m, 2H), 2.22 (m, 1H), 2.91 (m, 1H), 3.06 (m, 3H), 4.01 (m, 1H), 4.71 (m, 1H), 6.46 (s broad, 2H). MS (ESI) (M+H)⁺=228.92

### Step C. (2R)-Piperidine-2-carboxamide

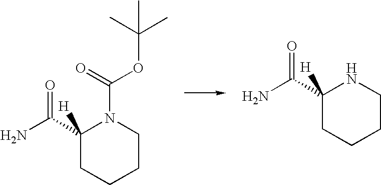

tert-Butyl (2R)-2-(aminocarbonyl)piperidine-1-carboxylate (2.28 g, 10 mmol) was treated with 4 N HCl in dioxane (60 mL, 240 mmol) for 4 h at room temperature. After evaporation of the solvent, the title compound washed with ether and dried in vacuo (HCl salt, 1.65 g, 100%). 1H NMR (400 MHz, DMSO-D6) δ 1.36-1.81 (m, 5H), 2.11 (m, 1H), 2.77-2.97 (m, 1H), 3.16 (m, 1H), 3.67 (m, 1H), 7.54 (s, 1H), 7.94 (s, 1H), 8.61 (s, 1H), 9.22 (s, 1H).

### Step D. [(2R)-Piperidin-2-ylmethyl]amine

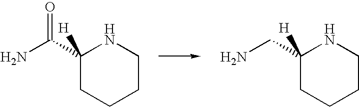

(2R)-piperidine-2-carboxamide (HCl salt, 1.65 g, 10 mmol) was treated with LAH (2.2 g, 58 mmol) in THF (150 mL) for 18 h at room temperature and 3 h at reflux. The mixture was cooled down, quenched with MeOH (10 mL) and H₂O (10 mL). Na₂SO₄ (100 g) was added. The resulting mixture was stirred for 2 h at room temperature. After filtration and evaporation of the solvent (1.14 g, 100%) of the title compound was obtained as a crude product, which was directly used for next step.

### Example 78

### 3-(1-Naphthoylamino)-N-[(2S)-piperidin-2-ylmethyl]pyridine-2-carboxamide

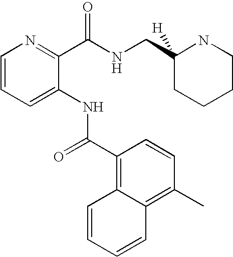

### Step A. 3-(1-Naphthoylamino)-N-[(2S)-piperidin-2-ylmethyl]pyridine-2-carboxamide

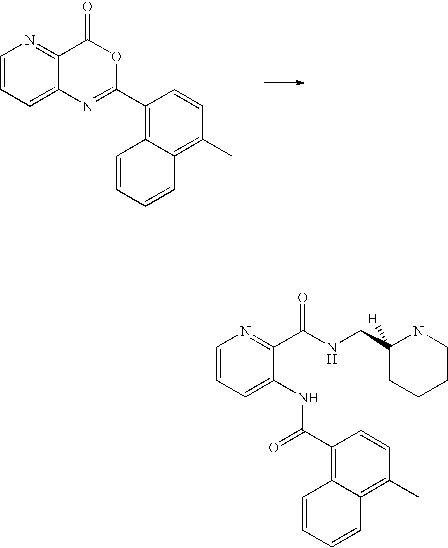

Following the procedure for Step A in Example 1, using 2-(4-methyl-1-Naphthalenyl)-H-pyrido[3,2-d][1,3]oxazin-4-one (110 mg, 0.38 mmol) and [(2S)-piperidin-2-ylmethyl]amine (110 mg, 0.96 mmol) (for preparation, see following Steps B, C and D) in DMF (8.0 mL) provided the title compound after purification by MPLC on silica gel using CH₂Cl₂/MeOH (20:1) (61.8 mg, 40%) as a white solid. [α]_{D}−14.2° (c 0.265, EtOH). 1H NMR (400 MHz, CD₃OD) δ 1.54 (m, 3H), 1.87 (m, 3H) 2.74 (s, 3H), 2.84 (m, 1H), 3.22 (m, 2H), 3.52 (dd, J=14.65, 3.71 Hz, 1H), 3.60 (m, 1H), 7.40 (d, J=7.23 Hz, 1H), 7.59 (m, 3H), 7.78 (d, J=7.22 Hz, 1H), 8.12 (d, J=8.01 Hz, 1H), 8.38 (d, J=3.51 Hz, 1H), 8.43 (m, 1H), 9.25 (d, J=8.01 Hz, 1H). MS (ESI) (M+H)⁺=403.3. Anal. Calcd for C₂₄H₂₆N₄O₂+1.20TFA+0.10H₂O: C, 58.60; H, 5.10; N, 10.35. Found: C, 58.52; H, 5.17; N, 10.36.

### Step B. tert-Butyl (2S)-2-(aminocarbonyl)piperidine-1-carboxylate

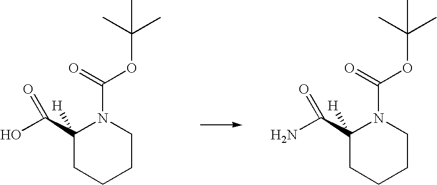

Following the procedure for Step B in example 77, using HATU (5.56 g, 14.6 mmol, (2S)-1-(tert-butoxycarbonyl)piperidine-2-carboxylic acid (2.29 g, 10 mmol), ammonium chloride (3.20 g, 60 mmol) and DIPEA (3.88 g, 30 mmol) in DMF (70 mL) provided the title compound after purification by MPLC on silica gel using hexane/EtOAc (1:1) (2.28 g, 100%) as a white solid. 1H NMR (400 MHz, CDCl₃) δ 1.47 (s, 9H), 1.52 (m, 3H), 1.64 (m, 3H), 2.89 (s broad, 2H), 4.04 (s broad, 1H), 6.06 (s broad, 1H), 6.21 (s broad, 1H). MS (ESI) (M+H)⁺=228.92

### Step C. (2S)-Piperidine-2-carboxamide

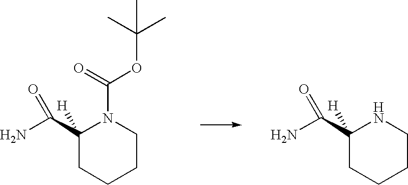

Following the procedure for Step C in example 77, using tert-Butyl (2S)-2-(aminocarbonyl)piperidine-1-carboxylate (2.28 g, 10 mmol) and 4 NHCl in dioxane (60 mL, 240 mmol) provided the title compound (HCl salt, 1.65 g, 100%). 1H NMR (400 MHz, DMSO-D6) δ 1.33-1.80 (m, 5H), 2.08 (m, 1H), 2.85 (m, 1H), 3.15 (m, 1H), 3.51-3.75 (m, 1H), 7.53 (s, 1H), 7.88 (s, 1H), 8.58 (s, 1H), 9.07 (s, 1H).

### Step D. [(2S)-Piperidin-2-ylmethyl]amine

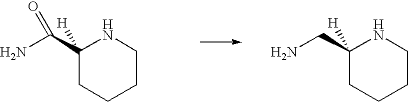

Following the procedure for Step D in example 77, using (2R)-piperidine-2-carboxamide (HCl salt, 1.65 g 10 mmol) and LAH (2.6 g, 68 mmol) in THF (150 mL) provided the title compound (1.14 g, 100%) as a crude product, which was directly used for next step.

### Example 79

### 3-(1-Naphthoylamino)-N-(pyridin-2-ylmethyl)pyridine-2-carboxamide

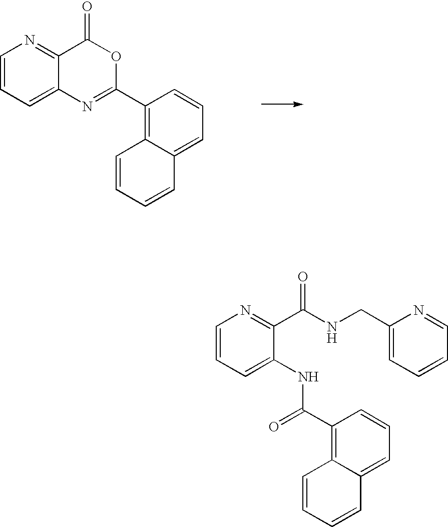

Following the procedure for Step A in Example 1, using 2-(1-Naphthalenyl)-H-pyrido[3,2-d][1,3]oxazin-4-one (54.9 mg, 0.2 mmol) and (pyridin-2-ylmethyl)amine (74.2 mg, 0.68 mmol) in DMF (2.0 mL) provided the title compound as a white solid. Yield: 56.3 mg (74%). 1H NMR (400 MHz, CD₃OD) δ 4.91 (s, 2H), 7.55 (m, 3H), 7.68 (dd, J=8.69, 4.59 Hz, 1H), 7.84 (dd, J=7.22, 1.17 Hz, 1H), 7.94 (m, 2H), 8.05 (dd, J=8.20, 3.71 Hz, 2H), 8.39 (dd, J=6.25, 3.71 Hz, 1H), 8.44 (dd, J=4.59, 1.46 Hz, 1H), 8.55 (t, J=8.01 Hz, 1H), 8.69 (d, J=6.05 Hz, 1H), 9.30 (m, 1H). MS (ESI) (M+H)⁺=383.0. Anal. Calcd for C₂₃H₁₈N₄O₂+2.10HCl+1.30H₂O: C, 57.27; H, 4.74; N, 11.61. Found: C, 57.35; H, 4.71; N, 11.65.

### Example 80

### 3-(4-Methyl 1-naphthoylamino)-N-(pyridin-2-ylmethyl)pyridine-2-carboxamide

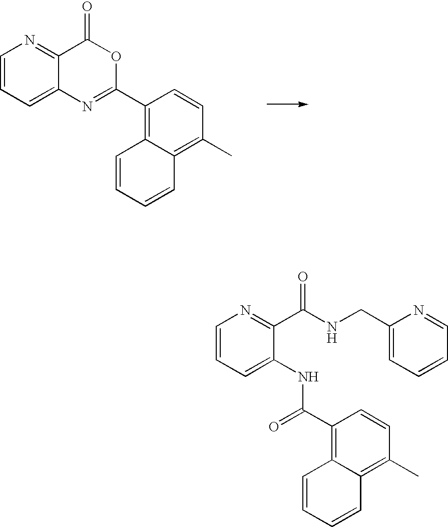

Following the procedure for Step A in Example 1, using 2-(4-Methyl-1-naphthalenyl)-H-pyrido[3,2-d][1,3]oxazin-4-one (86.5 mg, 0.3 mmol) and (pyridin-2-ylmethyl)amine (105.0 mg, 0.97 mmol) in DMF (3.0 mL) provided the title compound as its TFA salt after purification by reversed-phase HPLC using 10-85% MeCN/H₂O (54.9 mg, 36%). 1H NMR (400 MHz, CD₃OD) δ 2.74 (s, 3H), 4.71 (s, 2H), 7.41 (m, 2H), 7.57 (m, 3H), 7.64 (dd, J=8.59, 4.49 Hz, 1H), 7.77 (d, J=7.22 Hz, 1H), 7.92 (m, 1H), 8.12 (m, 1H), 8.40 (dd, J=4.49, 1.37 Hz, 1H), 8.46 (m, 1H), 8.51 (s, 1H), 9.30 (dd, J=8.59, 1.37 Hz, 1H). MS (ESI) (M+H)⁺=397.0. Anal. Calcd for C₂₄H₂₀N₄O₂+0.2TFA+0.20H₂O: C, 69.31; H, 4.91; N, 13.25. Found: C, 69.27; H, 4.96; N, 13.22.

### Example 81

### 3-[(4-Amino-1-naphthoyl)amino]-N-(cyclohexylmethyl)pyridine-2-carboxamide

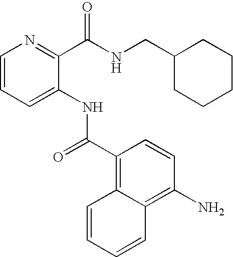

### Step A. 3-[(4-Amino-1-naphthoyl)amino]-N-(cyclohexylmethyl)pyridine-2-carboxamide

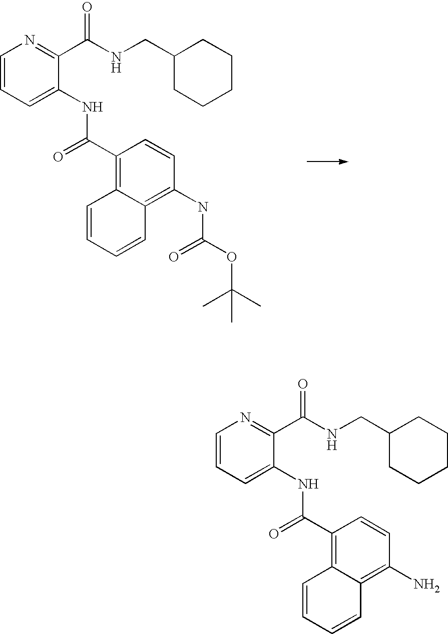

tert-Butyl (4-{[(2-{[(cyclohexylmethyl)amino]carbonyl}pyridin-3-yl)amino]carbonyl}-1-naphthyl)carbamate (14.2 mg, 0.028 mmol) in CH₂Cl₂ (1.5 mL) was treated with trifluoroacetic acid (1.5 mL). The reaction mixture was stirred for 3 h at room temperature. After concentration and lyophilization, the title compound was obtained as TFA salt (14.0 mg, 97%). 1H NMR (400 MHz, CD₃OD) δ 0.86-1.00 (m, 2H), 1.07-1.29 (m, 4H), 1.48-1.58 (m, 1H), 1.68 (m, 4H), 3.14 (d, J=6.83 Hz, 2H), 6.79 (d, J=8.01 Hz, 1H), 7.36-7.54 (m, 3H), 7.74 (d, J=8.01 Hz, 1H), 8.00 (dd, J=8.40, 0.78 Hz, 1H), 8.25 (dd, J=4.49, 1.17 Hz, 1H), 8.54 (d, J=8.20 Hz, 1H,) 9.18 (dd, J=8.59, 1.37 Hz, 1H). MS (ESI) (M+H)⁺=403.3. Anal. Calcd for C₂₄H₂₆N₄O₂+0.30TFA+0.50EtOAc+0.50H₂O (495.77): C, 65.66; H, 6.36; N, 11.30. Found: C, 65.54; H, 6.42; N, 11.34.

### Step B. tert-Butyl[4-(4-oxo-4H-pyrido[3,2-d][1,3]oxazin-2-yl)-1-naphthyl]carbamate

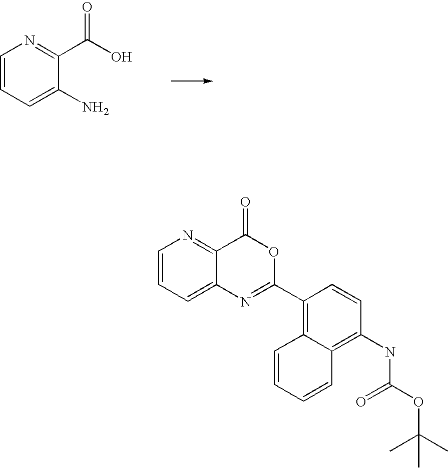

Oxalyl chloride (0.28 mL, 2.0M, 0.56 mmol) in CH₂Cl₂ was added to a solution of 4-[(tert-butoxycarbonyl)amino]-1-naphthoic acid (72.7 mg, 0.25 mmol) in CH₂Cl₂ (5 mL). Stirring for 4.5 h at room temperature and evaporation of the solvent, the residue was dissolved in CH₂Cl₂ (5 mL). 3-Amino-2-pyridinecarboxylic acid (34.5 mg, 0.25 mmol) and DIPEA (105 uL, 77.8 mg, 0.60 mmol) were added at 0° C. Stirring for 2 h at room temperature and evaporation of the solvent, DMF (5 mL), DIPEA (105 uL, 77.8 mg, 0.60 mmol) and then HATU (104.6 mg, 0.28 mmol) were added. The resulting mixture was stirred overnight at room temperature. The title compound was formed and directly used for next step.

### Step C. tert-Butyl (4-{[(2-{[(cyclohexylmethyl)amino]carbonyl}pyridin-3-yl)amino]carbonyl}-1-naphthyl)carbamate

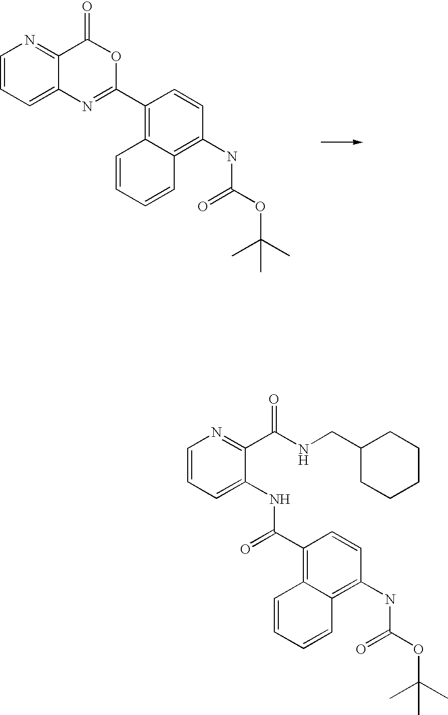

A solution of tert-Butyl[4-(4-oxo-4H-pyrido[3,2-d][1,3]oxazin-2-yl)-1-naphthyl]carbamate (0.25 mmol) in DMF (5 mL) (for preparation see following Step B) was treated with cyclohexane methylamine (160 uL, 139 mg, 0.1.2 mmol) at 0° C. The mixture was stirred for 18 h at room temperature. After evaporation of the solvent, the title compound was purified by MPLC on silica gel using hexane/EtOAc (4:1) (29.4 mg, 23%). 1H NMR (400 MHz, CD₃OD) δ 0.91-1.04 (m, 2H), 1.12-1.30 (m, 4H,) 1.56 (s, 9H), 1.59-1.80 (m, 5H), 3.19 (d, J=7.03 Hz, 2H,) 7.53-7.65 (m, 3H), 7.81-7.86 (m, 1H), 7.88-7.94 (m, 1H), 8.14 (dd, J=6.74, 3.22 Hz, 1H), 8.36 (dd, J=4.39, 1.27 Hz, 1H), 8.46-8.55 (m, 1H), 9.28 (dd, J=8.49, 1.27 Hz, 1H). MS (ESI) (M+H)⁺=503.3. Anal. Calcd for C₂₉H₃₄N₄O₄+0.5HCl+0.3H₂O (526.25): C, 66.19; H, 6.72; N, 10.65. Found: C, 66.14; H, 6.73; N, 10.24.

### Example 82

### N-(Cyclohexylmethyl)-3-[(4-methyl-1-naphthalenylcarbonyl)amino]-2-pyridinecarboxamide

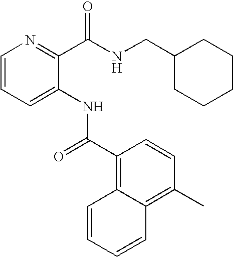

### Step A. N-(Cyclohexylmethyl)-3-[(4-methyl-1-naphthalenylcarbonyl)amino]-2-pyridinecarboxamide

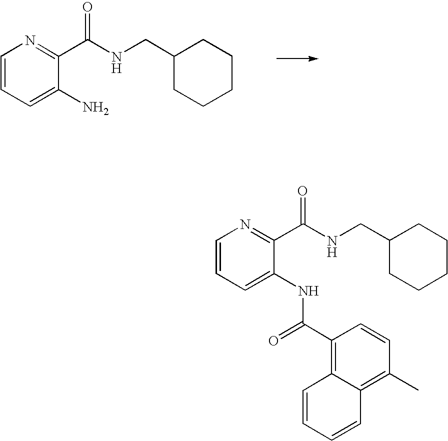

4-Methyl-1-naphthalenecarbonyl chloride (80 mg, 0.39 mmol) was added to a solution of 3-amino-N-(cyclohexylmethyl)pyridine-2-carboxamide (61 mg, 0.26 mmol) (for preparation see following step B) and DMAP (64 mg, 0.52 mmol) in CH₂Cl₂ (10 mL) at 0° C. The mixture was stirred overnight at room temperature, quenched with saturated NaHCO₃ solution (5 mL), and extracted with EtOAc (3×50 mL). The combined organic phases were washed with brine (2×10 mL) and dried with Na₂SO₄. After filtration and concentration, the title compound was purified by MPLC on silica gel using hexane/EtOAc (4:1) (96 mg, 92%). ¹H NMR (400 MHz, CD₃OD) δ 0.88-1.05 (m, 2H), 1.09-1.34 (m, 3H), 1.52-1.68 (m, 2H), 1.68-1.81 (m, 4H), 2.76 (s, 3H), 3.18 (d, J=6.83 Hz, 2H), 7.39-7.50 (m, 1H), 7.54-7.65 (m, 3H), 7.80 (d, J=7.23 Hz, 1H), 8.06-8.18 (m, 1H), 8.36 (dd, J=4.49, 1.56 Hz, 1H), 8.43-8.50 (m, 1H), 9.29 (dd, J=8.59, 1.56 Hz, 1H). MS (ESI) (M+H)⁺ 402.0. Anal. Calcd for C₂₅H₂₇N₃O₂+0.10H₂O (403.31): C, 74.45; H, 6.80; N, 10.42. Found: C, 74.42; H, 6.89; N, 10.13.

### Step B. 3-Amino-N-(cyclohexylmethyl)pyridine-2-carboxamide

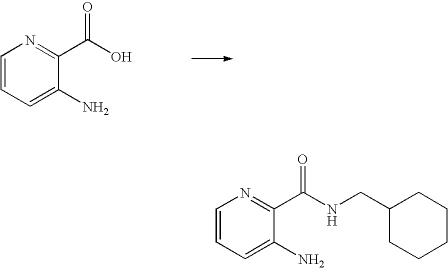

3-Aminopyridine-2-carboxylic acid (138 mg, 1.0 mmol) was added to a solution of cyclohexane methylamine (226 mg, 2.0 mmol) and DIPEA (259 mg, 0.35 mmol) in DMF (5 mL). After stirring for 30 min, HATU (456 mg, 1.2 mmol) was added at 0° C. The resulting mixture was stirred overnight at room temperature, quenched with water (50 ml), and extracted with EtOAc (3×40 mL). The combined organic phases were washed with water (2×5 mL), brine (5 mL), and dried with Na₂SO₄. After filtration and concentration, the title compound was purified by MPLC on silica gel using hexane/EtOAc (1:1) (124 mg, 53%). ¹H NMR (400 MHz, CDCl₃) δ 0.93-1.07 (m, 2H), 1.13-1.32 (m, 3H), 1.51-1.70 (m, 2H), 1.70-1.86 (m, 4H), 3.26 (t, J=6.64 Hz, 2H), 6.00 (s, 2H), 7.00 (dd, J=8.40, 1.37 Hz, 1H), 7.15 (dd, J=8.40, 4.30 Hz, 1H), 7.85 (dd, J=4.30, 1.37 Hz, 1H), 8.22 (s, 1H). (MS (ESI) (M+H)⁺ 233.89.

### Example 83

### N-(Cyclohexylmethyl)-3-[(2,2-dimethylbutanoyl)amino]pyridine-2-carboxamide

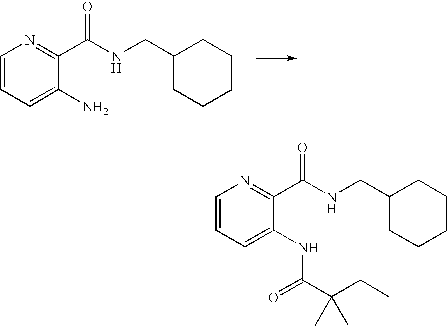

Following the procedure for Step A in Example 82, using 2,2-Dimethylbutanoyl chloride (30.0 mg, 0.223 mmol), 3-amino-N-(cyclohexylmethyl)pyridine-2-carboxamide (24.3 mg, 0.104 mmol) and DMAP (30.0 mg, 0.246 mmol) in CH₂Cl₂ (5 mL) provided the title compound after purification by MPLC on silica gel using hexane/EtOAc (9:1) (31.2 mg, 91%). ¹H NMR (400 MHz, CD₃OD) δ 0.88 (t, J=7.52 Hz, 3H), 0.94-1.08 (m, 2H), 1.16-1.25 (m, 2H), 1.28 (s, 6H,) 1.28-1.35 (m, 2H), 1.56-1.64 (m, 1H), 1.68 (q, J=7.42 Hz, 2H), 1.72-1.82 (m, 4H), 3.24 (d, J=6.83 Hz, 2H), 7.48 (dd, J=8.59, 4.49 Hz, 1H), 8.27 (dd, J=4.49, 1.37 Hz, 1H), 9.04 (dd, J=8.59, 1.37 Hz, 1H). MS(ESI) (M+H)⁺ 332.0. Anal. Calcd for C₁₉H₂₉N₃O₂+0.1H₂O (333.26): C, 68.48; H, 8.81; N, 12.61. Found: C, 68.61; H, 8.92; N, 12.28.

### Example 84

### 3-[(4-Amino-1-naphthoyl)amino]-N-(tetrahydro-2H-pyran-4-ylmethyl)pyridine-2-carboxamide

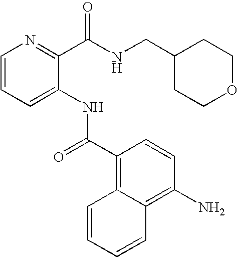

### Step A. 3-[(4-Amino-1-naphthoyl)amino]-N-(tetrahydro-2H-pyran-4-ylmethyl)pyridine-2-carboxamide

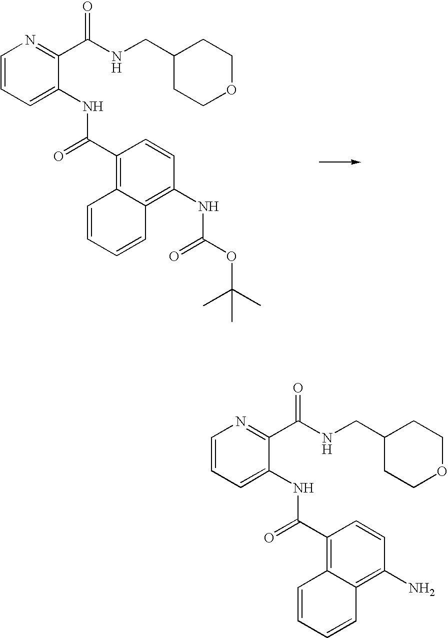

tert-Butyl (4-{[(2-{[(tetrahydro-2H-pyran-4-ylmethyl)amino]carbonyl}pyridin-3-yl)amino]carbonyl}-1-naphthyl)carbamate (377.0 mg, 0.747 mmol) in CH₂Cl₂ (5 mL) was treated with 4N HCl/dioxane (5 mL). The reaction mixture was stirred for 4 h at room temperature. After concentration and dried in vacuo, the title compound was obtained as a white solid (374.7 mg, 100%). 1H NMR (400 MHz, CD₃OD) δ 1.20-1.38 (m, 2H), 1.64 (m, 2H), 1.77-1.95 (m, 1H), 3.25 (d, J=7.03 Hz, 2H), 3.31-3.41 (m, 2H), 3.83-3.98 (m, 2H), 7.55-7.64 (m, 1H), 7.66-7.75 (m, 3H), 7.93 (d, J=7.81 Hz, 1H), 8.01-8.12 (m, 1H), 8.37 (d, J=2.73 Hz, 1H), 8.53-8.65 (m, 1H), 9.27 (d, J=8.59 Hz, 1H). MS (ESI) (M+H)⁺=405.0. Anal. Calcd for C₂₃H₂₄N₄O₃+1.70HCl (466.46): C, 59.22; H, 5.55; N, 12.01. Found: C, 59.28; H, 5.45; N, 11.87.

### Step B. tert-Butyl (4-{[(2-{[(tetrahydro-2H-pyran-4-ylmethyl)amino]carbonyl}pyridin-3-yl)amino]carbonyl}-1-naphthyl)carbamate

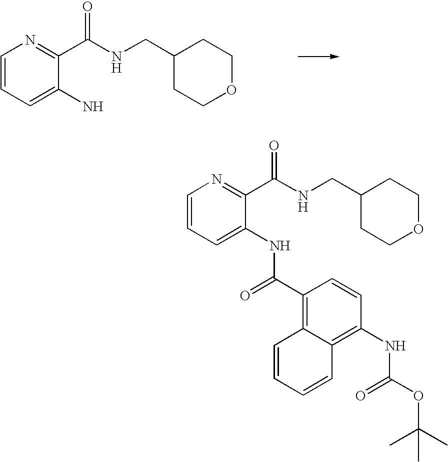

Oxalyl chloride (3.8 mL, 2.0M, 7.6 mmol) in CH₂Cl₂ was added to a solution of 4-[(tert-butoxycarbonyl)amino]-1-naphthoic acid (985.8 mg, 3.42 mmol) and DMAP (459.6 mg, 3.76 mmol) in CH₂Cl₂ (70 mL) at 0° C. Stirring for 2 h at room temperature and evaporation of the solvent and excess oxalyl chloride, the residue was dissolved in CH₂Cl₂ (70 mL). A solution of 3-amino-N-(tetrahydro-2H-pyran-4-ylmethyl)pyridine-2-carboxamide (807.2 mg, 3.42 mmol) and DMAP (459.6 mg, 3.76 mmol) in (10 mL) was added. The resulting mixture was stirred overnight at room temperature, washed with saturated NaHCO₃ solution (2×10 mL) and dried over Na₂SO₄. The title compound was purified by MPLC on silica gel using hexane/EtOAc (1:1) (377.0 mg, 22%) as a white solid. ¹H NMR (400 MHz, CD₃OD) δ1.22-1.39 (m, 2H), 1.56 (s, 9H), 1.59-1.69 (m, 2H), 1.79-1.95 (m, 1H), 3.25 (d, J=7.03 Hz, 2H), 3.32-3.44 (m, 2H), 3.90 (dd, J=11.42, 3.03 Hz, 2H,) 7.53-7.66 (m, 3H), 7.79-7.87 (m, 1H), 7.88-7.96 (m, 1H), 8.14 (dd, J=6.54, 3.42 Hz, 1H), 8.36 (dd, J=4.49, 1.37 Hz, 1H), 8.50 (dd, J=6.54, 3.03 Hz, 1H), 9.27 (dd, J=8.59, 1.56 Hz, 1H). MS (ESI) (+H)⁺ 505.0. Anal. Calcd for C₂₈H₃₂N₄O₅+0.50MeOH (520.01): C, 65.75; H, 6.58; N, 10.76. Found: C, 65.76; H, 6.51; N, 10.65.

### Example 85

### 3-{[4-(Acetylamino)-1-naphthoyl]amino}-N-(tetrahydro-2H-pyran-4-ylmethyl)pyridine-2-carboxamide

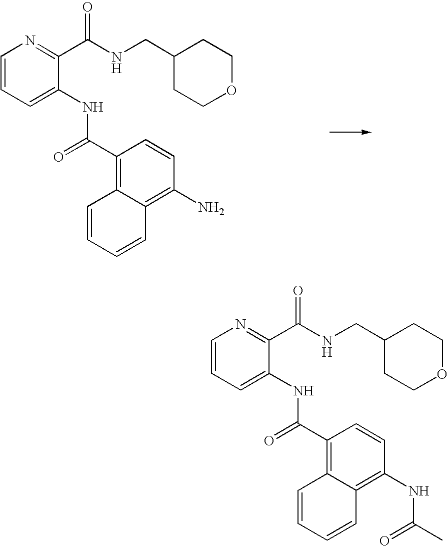

Acetyl chloride (7.7 mg, 0.099 mmol) was added to a solution of 3-[(4-Amino-1-naphthoyl)amino]-N-(tetrahydro-2H-pyran-4-ylmethyl)pyridine-2-carboxamide hydrochloride (33.4 mg, 0.076 mmol) and DMAP (23.2 mg, 0.19 mmol) in CH₂Cl₂ (5 mL). The reaction mixture was stirred overnight at room temperature, diluted with CH₂Cl₂ (100 mL), washed with saturated NaHCO₃ solution (2×10 mL) and dried over Na₂SO₄. After filtration and concentration, the title compound was purified by MPLC on silica gel using hexane/EtOAc (1:1) (27.3 mg, 81%). 1H NMR (400 MHz, CD₃OD) δ 1.22-1.39 (m, 2H), 1.63 (m, 2H), 1.78-1.93 (m, 1H), 2.30 (s, 3H), 3.24 (d, J=6.83 Hz, 2H), 3.31-3.41 (m, 2H), 3.90 (m, 2H), 7.56-7.65 (m, 3H), 7.83 (d, J=8.01 Hz, 1H), 7.90-7.94 (m, 1H), 8.08-8.21 (m, 1H), 8.37 (dd, J=4.49, 1.37 Hz, 1H), 8.45-8.56 (m, 1H), 9.28 (dd, J=8.59, 1.56 Hz, 1H). MS (ESI) (M+H)⁺ 447.0. Anal. Calcd for C₂₅H₂₆N₄O₄+0.20HCl+0.40EtOAc (499.25): C, 64.96; H, 6.06; N, 11.22. Found: C, 65.05; H, 6.03; N, 11.16.

### Example 86

### 3-[(4-{[(Methylamino)carbonyl]amino}-1-naphthoyl)amino]-N-(tetrahydro-2H-pyran-4-ylmethyl)pyridine-2-carboxamide

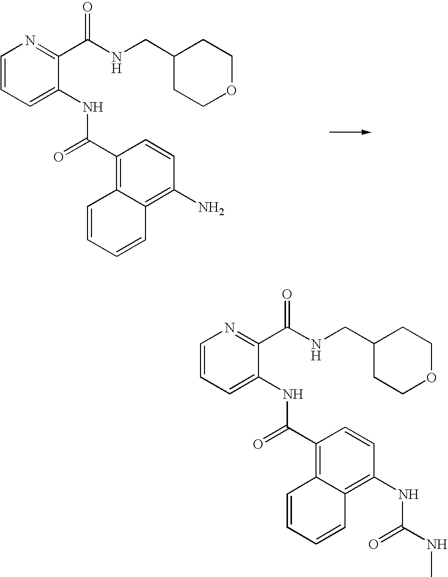

DIPEA (12.6 mg, 17 μL, 0.0976 mmol) was added to a suspension of 3-[(4-amino-1-naphthoyl)amino]-N-(tetrahydro-2H-pyran-4-ylmethyl)pyridine-2-carboxamide hydrochloride (36.0 mg, 0.0816 mmol) in 1,2-dichloroethane (3 mL). Stirring for 10 min. a clear solution was formed. Methylisocyanate (20 μL) was added. The reaction mixture was heated for 3 days at 60° C., diluted with CH₂Cl₂ (100 mL), washed with brine (2×10 mL) and dried over Na₂SO₄. After filtration and concentration, the title compound was purified by MPLC on silica gel using hexane/EtOAc (1:1) (23.4 mg, 62%). 1H NMR (400 MHz, CD₃OD) δ 1.22-1.38 (m, 2H), 1.64 (m, 2H), 1.78-1.95 (m, 1H), 2.84 (s, 3H), 3.25 (d, J=6.83 Hz, 2H,) 3.32-3.42 (m, 2H), 3.91 (m, 2H), 7.55-7.64 (m, 3H), 7.86-7.92 (m, 1H), 7.95-8.01 (m, 1H), 8.12 (dd, J=6.74, 3.03 Hz, 1H), 8.35 (dd, J=4.49, 1.56 Hz, 1H), 8.49-8.57 (m, 1H), 9.27 (dd, J=8.49, 1.46 Hz, 1H). MS (ESI) (M+H)⁺ 462.0. Anal. Calcd for C₂₅H₂₇N₅O₄+0.6MeOH: C, 63.96; H, 6.16; N, 14.57. Found: C, 64.17; H, 6.17; N, 14.30.

### Example 87

### Methyl (4-{[(2-{[(tetrahydro-2H-pyran-4-ylmethyl)amino]carbonyl}pyridin-3-yl)amino]carbonyl}-1-naphthyl)carbamate

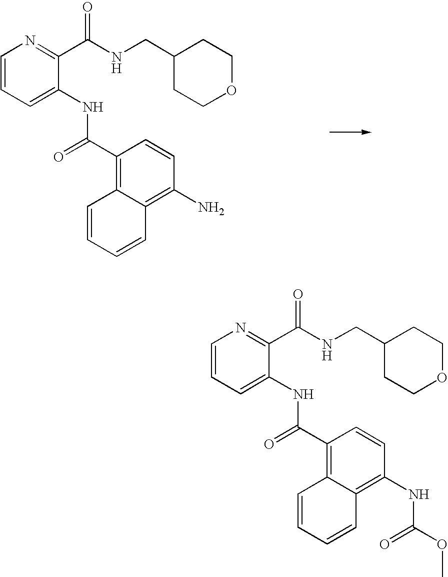

A solution of 3-[(4-amino-1-naphthoyl)amino]-N-(tetrahydro-2H-pyran-4-ylmethyl)pyridine-2-carboxamide (45.9 mg, 0.114 mmol), DMAP (56.0 mg, 0.458 mmol) and methyl chloroformate (122 mg, 100 μL, 1.29 mmol) in MeCN (5 mL) was heated at 100° C. in a Personal Chemistry SmithSynthesizer microwave instrument for 1 h. After concentration, the title compound was purified by MPLC on silica gel using hexane/EtOAc (1:1) (18.3 mg, 38%). 1H NMR (400 MHz, CD₃OD) δ 1.20-1.44 (m, 2H), 1.64 (m, 2H), 1.76-2.03 (m, 1H), 3.26 (m, 2H) 3.32-3.46 (m, 2H), 3.83 (s, 3H), 3.91 (m, 2H), 7.45-7.72 (m, 3H), 7.83-7.99 (m, 2H), 8.08-8.22 (m, 1H), 8.38 (dd, J=4.49, 1.37 Hz, 1H), 8.47-8.60 (m, 1H), 9.29 (dd, J=8.59, 1.56 Hz, 1H). MS (ESI) (M+H)⁺ 463.0. Anal. Calcd for C₂₅H₂₆N₄O₅+0.1HCl+0.9MeCN+0.3H₂O (508.51): C, 63.30; H, 5.83; N, 13.50. Found: C, 63.20; H, 5.83; N, 13.45.

### Example 88

### N-(Cyclohexyloxy)-3-[(4-methyl-1-naphthoyl)amino]pyridine-2-carboxamide

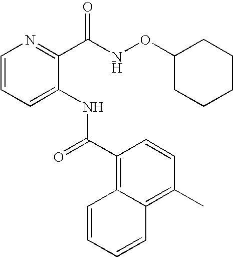

### Step A. N-(Cyclohexyloxy)-3-[(4-methyl-1-naphthoyl)amino]pyridine-2-carboxamide

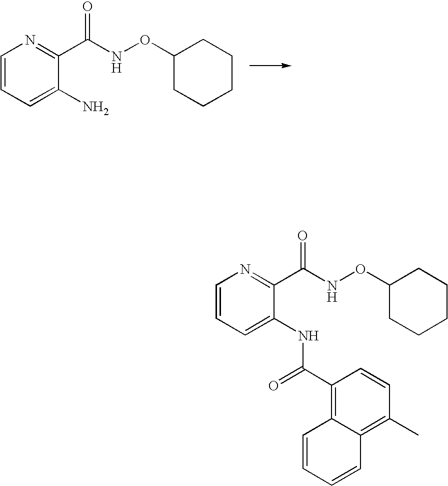

4-Methyl-1-naphthoyl chloride (126.6 mg, 0.62 mmol) was added to a solution of 3-amino-N-(cyclohexyloxy)pyridine-2-carboxamide (97.0 mg, 0.41 mmol) (for preparation see following step B) and DMAP (100.2 mg, 82 mmol) in CH₂Cl₂ (10 mL) at 0° C. The mixture was stirred overnight at room temperature, quenched with saturated NaHCO₃ solution (5 mL), and extracted with EtOAc (3×50 mL). The combined organic phases were washed with brine (10 mL) and dried over Na₂SO₄. After evaporation of the solvent, the title compound was purified by MPLC on silica gel using hexane/EtOAc (1:1) (30.5 mg, 18%). 1H NMR (400 MHz, CD₃OD) δ 1.14-1.36 (m, 3H), 1.38-1.59 (m, 3H), 1.72-1.82 (m, 2H), 1.93-2.04 (m, 2H), 2.76 (s, 3H), 3.82-3.97 (m, 1H), 7.45 (d, J=7.23 Hz, 1H), 7.53-7.67 (m, 3H), 7.80 (d, J=7.23 Hz, 1H), 8.07-8.17 (m, 1H), 8.35 (dd, J=4.49, 1.37 Hz, 1H), 8.43-8.48 (m, 1H), 9.26 (dd, J=8.59, 1.37 Hz, 1H). MS (ESI) (M+H)⁺=404.0. Anal. Calcd for C₂₄H₂₅N₃O₃+0.20TFA+0.3H₂O (431.69): C, 67.89; H, 6.02; N, 9.73. Found: C, 67.98; H, 6.04; N, 9.54.

### Step B. 3-Amino-N-(cyclohexyloxy)pyridine-2-carboxamide

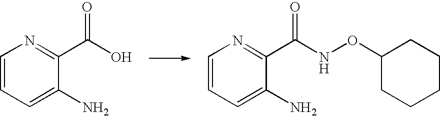

HATU (2.32 g, 6.10 mmol) was added to a solution of O-cyclohexylhydroxylamine (prepared as ref. A. Miyake et al J. Antibiot. 53 (10), 1071-1085, 2000) (0.86 g, 7.50 mmol), DIPEA (1.29 g, 10.0 mmol) and 3-aminopyridine-2-carboxylic acid (0.69 g, 5.00 mmol) in DMF (20 mL) at 0° C. The mixture was stirred overnight at room temperature, diluted with EtOAc (200 mL), washed with H₂O (2×10 mL), brine (10 mL) and dried over Na₂SO₄. After evaporation of the solvent, the title compound was purified by MPLC on silica gel using hexane/EtOAc (1:1) (1.35 g, 100%) as a white solid. 1H NMR (400 MHz, CDCl₃) δ 1.30 (m, 2H), 1.52 (m, 4H), 1.80 (m, 2H), 2.06 (m, 2H), 3.96 (m, 1H), 5.93 (s, 2H), 7.00 (dd, J=8.40, 1.37 Hz, 1H), 7.17 (dd, J=8.40, 4.30 Hz, 1H), 7.82 (dd, J=4.30, 1.37 Hz, 1H), 10.12 (s, 1H).

### Example 89

### 3-[(4-Methyl-1-naphthoyl)amino]-N-[(1-methylpiperidin-2-yl)methyl]pyridine-2-carboxamide

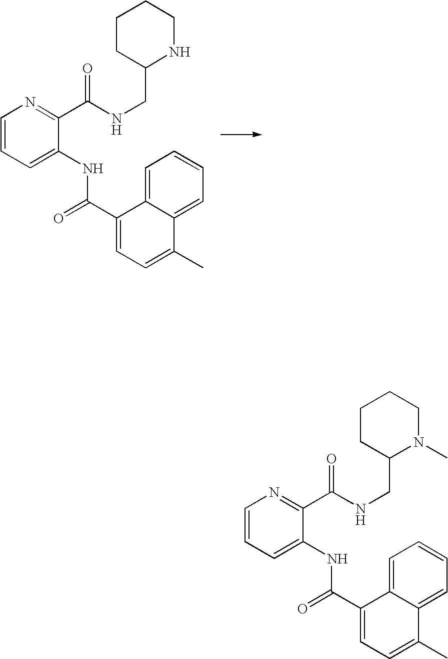

To a solution of 3-[(4-methyl-1-naphthoyl)amino]-N-(piperidin-2-ylmethyl)pyridine-2-carboxamide (TFA salt, 161 mg) and formaldehyde (37% in H₂O, 100 mg) in CH₂Cl₂ (15 mL) at r.t, was added NaBH(OAc)₃ (300 mg) in one portion. The reaction mixture was stirred at r.t for 3 hours, and then concentrated. The residue was dissolved in EtOAc, washed with aqueous NH₄Cl, dried (Na₂SO₄), filtered and concentrated. Purification by reversed-phase HPLC provided the title compound as its TFA salt (34 mg, 20%). ¹H NMR (400 MHz, CD₃OD) δ 1.60 (m, 3H), 1.84 (m, 2H), 2.06 (m, 1H), 2.77 (s, 3H), 2.86 (m, 1H), 3.01 (s, 3H), 3.02 (m, 1H), 3.25 (m, 1H), 3.42 (m, 1H), 3.58 (m, 1H), 3.98 (m, 1H), 7.43 (d, J=7.6 Hz, 1H), 7.61 (m, 3H), 7.80 (d, J=7.6 Hz, 1H), 8.15 (d, J=8.0 Hz, 1H), 8.41 (dd, J=4.4, 1.2 Hz, 1H), 8.46 (dd, J=8.0, 0.8 Hz, 1H), 9.28 (dd, J=8.8, 0.8 Hz, 1H); MS (ESI) (M+H)⁺ 417.3.

### Example 90

### 3-[(4-Ethyl-1-naphthoyl)amino]-N-(tetrahydro-2H-pyran-4-ylmethyl)pyridine-2-carboxamide

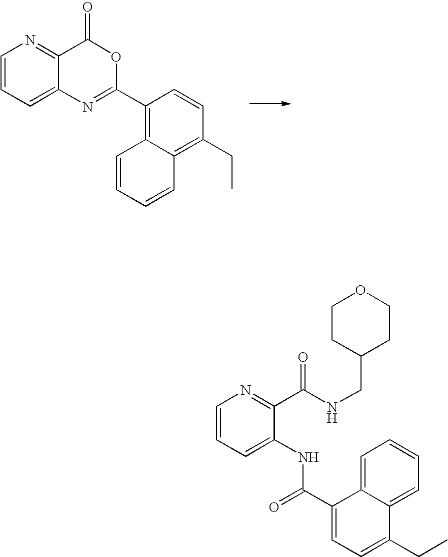

Following the procedure for Step A in Example 1, using 2-(4-ethyl-1-naphthyl)-4H-pyrido[3,2-d][1,3]oxazin-4-one (76 mg, 0.25 mmol), and tetrahydro-2H-pyran-4-methanamine (115 mg, 1.0 mmol) provided the title compound after purification by silica gel column (18 mg, 17%). ¹H NMR (400 MHz, CD₃OD) δ 1.30 (m, 2H), 1.39 (t, J=7.6 Hz, 3H), 1.62 (m, 2H), 1.87 (m, 1H), 3.18 (q, J=7.6 Hz, 2H), 3.23 (m, 2H), 3.34 (m, 2H), 3.88 (m, 2H), 7.46 (d, J=7.6 Hz, 1H), 7.60 (m, 3H), 7.81 (d, J=7.6 Hz, 1H), 8.18 (d, J=8.0 Hz, 1H), 8.35 (d, J=4.4 Hz, 1H), 8.46 (d, J=8.0 Hz, 1H), 9.27 (d, J=8.0 Hz, 1H); MS (ESI) (M+H)⁺=418.0.

### Example 91

### 3-[(4-Ethyl-1-naphthoyl)amino]-N-(piperidin-2-ylmethyl)pyridine-2-carboxamide

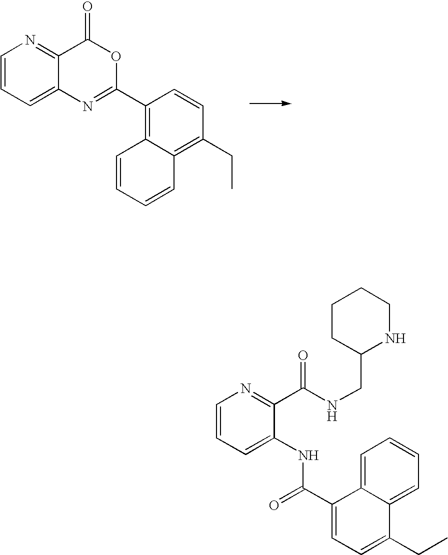

Following the procedure for Step A in Example 1, using 2-(4-ethyl-1-naphthyl)-4H-pyrido[3,2-d][1,3]oxazin-4-one (76 mg, 0.25 mmol) and (piperidin-2-yl-methyl)amine (114 mg, 1.0 mmol) provided the title compound as its TFA salt after purification by reversed-phase HPLC (16 mg, 12%). ¹H NMR (400 MHz, CD₃OD) δ 1.38 (t, J=7.6 Hz, 3H), 1.55 (m, 3H), 1.85 (m, 3H), 2.84 (m, 1H), 3.18 (q, J=7.6 Hz, 2H), 3.29 (m, 2H), 3.56 (m, 2H), 7.43 (d, J=7.6 Hz, 1H), 7.62 (m, 3H), 7.81 (d, J=7.6 Hz, 1H), 8.18 (d, J=8.0 Hz, 1H), 8.39 (d, J=4.4 Hz, 1H), 8.44 (d, J=8.0 Hz, 1H), 9.26 (d, J=8.0 Hz, 1H); MS (ESI) (M+H)⁺ 417.0.

### Example 92

### 3-[(4-Isopropyl-1-naphthoyl)amino]-N-(tetrahydro-2H-pyran-4-ylmethyl)pyridine-2-carboxamide

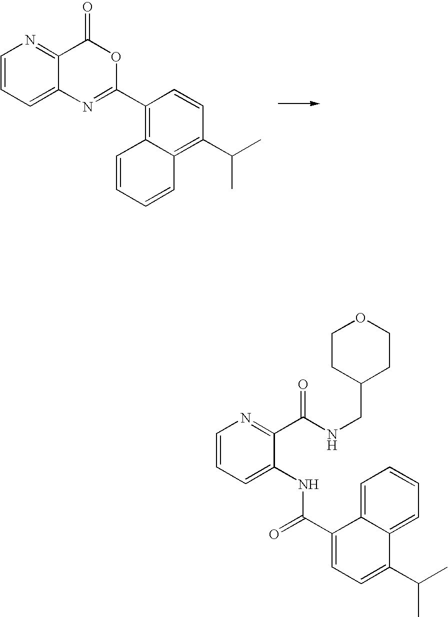

Following the procedure for Step A in Example 1, using 2-(4-isopropyl-1-naphthyl)-4H-pyrido[3,2-d][1,3]oxazin-4-one (79 mg, 0.25 mmol), and tetrahydro-2H-pyran-4-methanamine (115 mg, 1.0 mmol) provided the title compound (32 mg, 30%). ¹H NMR (400 MHz, CD₃OD) δ 1.30 (m, 2H), 1.33 (d, J=6.8 Hz, 6H), 1.67 (m, 2H), 1.87 (m, 1H), 3.06 (m, 1H), 3.30 (m, 2H), 3.38 (m, 2H), 3.92 (m, 2H), 7.49 (m, 2H), 7.70 (brs, 1H), 7.91 (m, 2H), 7.98 (dd, J=8.0, 4.0 Hz, 1H), 8.28 (d, J=4.0 Hz, 1H), 8.45 (brs, 1H), 9.18 (d, J=8.0 Hz, 1H); MS (ESI) (M+H)⁺=432.2.

### Example 93

### N-(2-Hydroxyethyl)-3-(1-naphthoylamino)pyridine-2-carboxamide

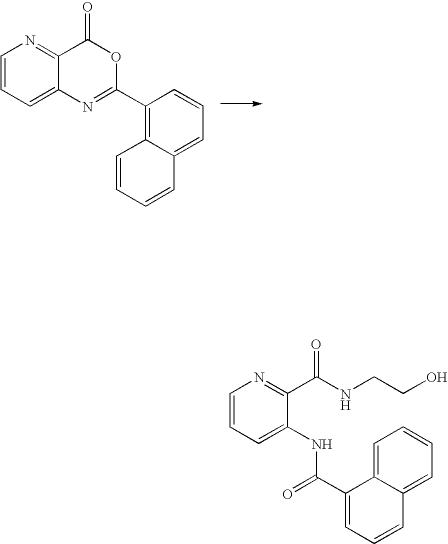

Following the procedure for Step A in Example 1, using 2-(1-naphthalenyl)-4H-pyrido[3,2-d][1,3]oxazin-4-one (100 mg, 0.36 mmol), and 2-aminoethanol (122 mg, 2.0 mmol) provided the title compound as its TFA salt after purification by reversed-phase HPLC (75 mg, 46%). MS (ESI) (+H)⁺ 336.0.

### Example 94

### 3-[(4-Isopropyl-1-naphthoyl)amino]-N-(piperidin-2-ylmethyl)pyridine-2-carboxamide

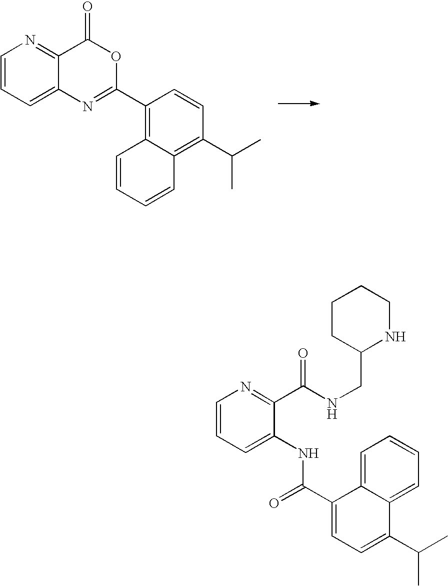

Following the procedure for Step A in Example 1, using 2-(4-isopropyl-1-naphthyl)-4H-pyrido[3,2-d][1,3]oxazin-4-one (79 mg, 0.25 mmol), and (piperidin-2-yl-methyl)amine (114 mg, 1.0 mmol) provided the title compound as its TFA salt after purification by reversed-phase HPLC (25 mg, 18%). ¹H NMR (400 MHz, CD₃OD) δ 1.35 (d, J=6.8 Hz, 6H), 1.60 (m, 3H), 1.90 (m, 3H), 2.87 (m, 1H), 3.11 (m, 1H), 3.33 (m, 2H), 3.66 (m, 2H); 7.54 (dd, J=8.0, 4.0 Hz, 1H), 7.60 (m, 1H), 7.76 (brs, 1H), 7.94 (m, 2H), 8.02 (dd, J=8.0, 4.0 Hz, 1H), 8.36 (d, J=4.0 Hz, 1H), 8.51 (brs, 1H), 9.24 (d, J=8.0 Hz, 1H); MS (ESI) (M+H)⁺ 431.3.

### Example 95

### 3-{[4-(Methoxymethyl)-1-naphthoyl]amino}-N-(piperidin-2-ylmethyl)pyridine-2-carboxamide

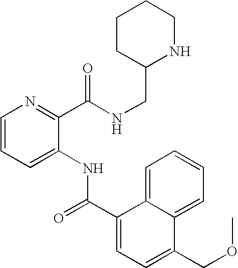

### Step A. 3-{[4-(Methoxymethyl)-1-naphthoyl]amino}-N-(piperidin-2-ylmethyl)pyridine-2-carboxamide

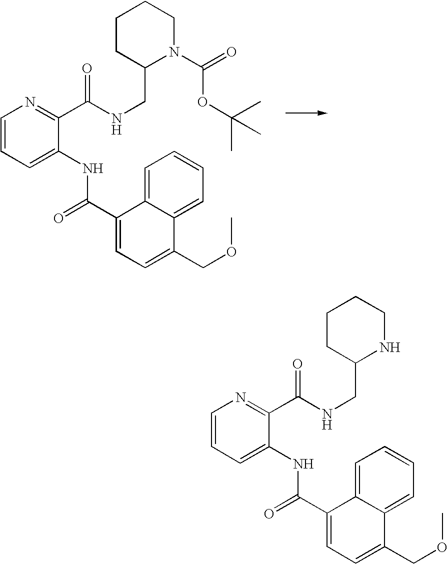

The crude tert-butyl 2-({[(3-{[4-(methoxymethyl)-1-naphthoyl]amino}pyridin-2-yl)carbonyl]amino}methyl)piperidine-1-carboxylate (crude, 0.3 mmol) from Step D was treated with TFA in CH₂Cl₂ (1:1) for 2 hrs at r.t. Removal of solvents gave a residue which was purified by reversed-phase HPLC to provide the title compound as its TFA salt (38 mg, 23%). ¹H NMR (400 MHz, CD₃OD) δ 1.55 (m, 3H), 1.88 (m, 3H), 2.85 (m, 1H), 3.23 (m, 2H), 3.49 (s, 3H), 3.55 (m, 2H), 4.97 (s, 2H), 7.61 (m, 3H), 7.66 (dd, J=8.0, 4.0 Hz, 1H), 7.86 (d, J=8.0 Hz, 1H), 8.18 (d, J=8.0 Hz, 1H), 8.41 (d, J=4.0 Hz, 1H), 8.44 (d, J=8.0 Hz, 1H), 9.28 (dd, J=8.0 Hz, 1H); MS (ESI) (M+H)⁺ 433.0.

### Step B. tert-Butyl 2-({[(3-aminopyridin-2-yl)carbonyl]amino}methyl)piperidine-1-carboxylate

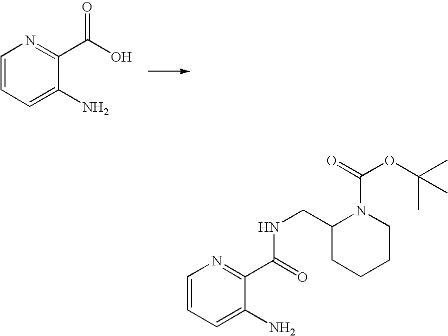

To a solution of 3-aminopyridine-2-carboxylic acid (552 mg, 4.0 mmol), tert-butyl 2-(aminomethyl)piperidine-1-carboxylate (1.28 g, 6.0 mmol,) and DIPEA (6.0 mmol) in DMF (25 mL)/THF (10 mL), was added HATU (2.3 g, 6.0 mmol) in one portion. The solution was stirred for 1 hr at r.t and for 1 hr at 50° C., and then concentrated. The residue was dissolved in EtOAc, washed with brine, dried (Na₂SO₄), filtered and concentrated. Purification by MPLC afforded the title compound (1.05 g, 79%).

### Step C. tert-Butyl 2-({[(3-{[4-(bromomethyl)-1-naphthoyl]amino}pyridin-2-yl)carbonyl]amino}methyl)piperidine-1-carboxylate

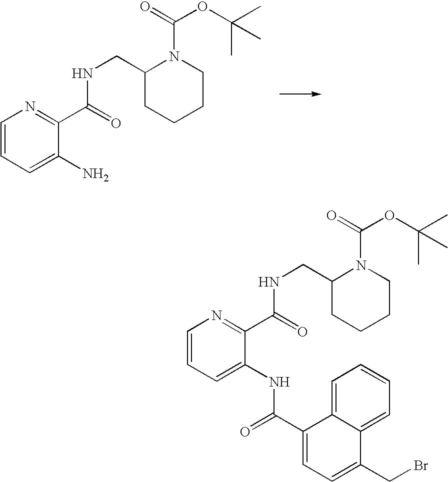

To a suspension of 4-(bromomethyl)-1-naphthoic acid (100 mg, 0.38 mmol) in CH₂Cl₂ (5 mL) at room temperature, was added oxalyl chloride (0.5 mL, 1.0 mmol) drop wise. The solution was stirred at room temperature for 10 minutes, and then heated at 50° C. for 30 minutes. After removal of solvents, the residue was added to a solution of tert-Butyl 2-({[(3-aminopyridin-2-yl)carbonyl]amino}methyl)piperidine-1-carboxylate (100 mg, 0.3 mmol) and DIPEA (1.0 mmol) in CH₂Cl₂ (10 mL) at 0° C. The reaction mixture was stirred at r.t for 2 hr, and was then concentrated. The residue was used directly in Step D.

### Step D. tert-Butyl 2-({[(3-{[4-(methoxymethyl)-1-naphthoyl]amino}pyridin-2-yl)carbonyl]amino}methyl)piperidine-1-carboxylate

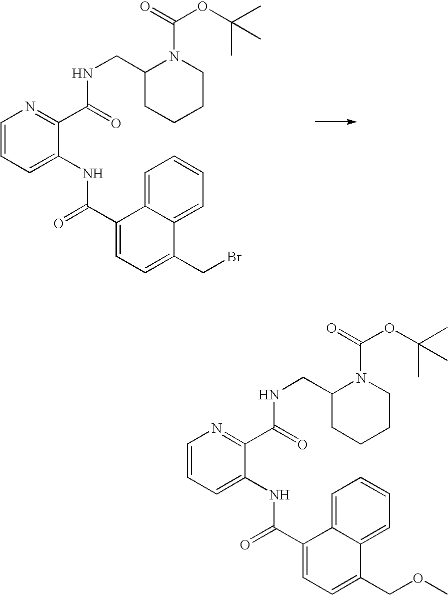

To a solution of tert-butyl 2-({[(3-{[4-(bromomethyl)-1-naphthoyl]amino}pyridin-2-yl)carbonyl]amino}methyl)piperidine-1-carboxylate (crude, 0.3 mmol) in MeOH (10 mL) was added NaOMe (30% in MeOH, 1.0 mL) at 0° C. The solution was stirred at room temperature for 1 hr, and was then concentrated. The residue was dissolved in EtOAc, washed with brine, and dried (Na₂SO₄). Removal of solvents afforded the crude title compound, which was used directly in Step A.

### Example 96

### 3-{[4-(Ethoxymethyl)-1-naphthoyl]amino}-N-(piperidin-2-ylmethyl)pyridine-2-carboxamide

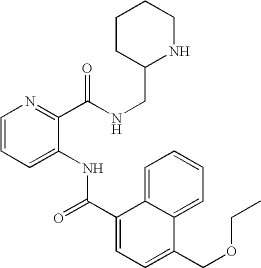

### Step A. 3-{[4-(Ethoxymethyl)-1-naphthoyl]amino}-N-(piperidin-2-ylmethyl)pyridine-2-carboxamide

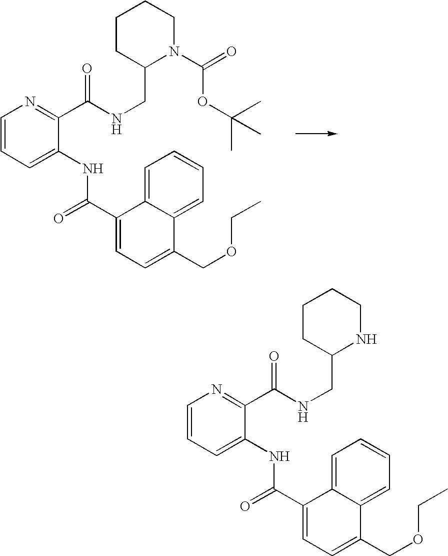

The crude tert-butyl 2-({[(3-{[4-(ethoxymethyl)-1-naphthoyl]amino}pyridin-2-yl)carbonyl]amino}methyl)piperidine-1-carboxylate from Step B was treated with TFA in CH₂Cl₂ (1:1) for 2 hrs at r.t. Removal of solvents gave a residue which was purified by reversed-phase HPLC to provide the title compound as its TFA salt (55 mg, 57%). MS (ESI) (M+H)⁺ 447.0.

### Step B. tert-Butyl 2-({[(3-{[4-(ethoxymethyl)-1-naphthoyl]amino}pyridin-2-yl)carbonyl]amino}methyl)piperidine-1-carboxylate

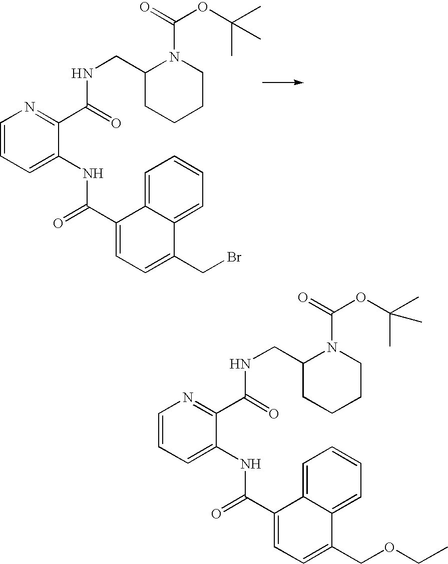

To a solution of tert-butyl 2-({[(3-{[4-(bromomethyl)-1-naphthoyl]amino}pyridin-2-yl)carbonyl]amino}methyl)piperidine-1-carboxylate (100 mg) in EtOH (5 mL) was added NaOEt (100 mg) at 0° C. The solution was stirred at room temperature for 1 hr, and was then concentrated. The residue was dissolved in EtOAc, washed with brine, and dried (Na₂SO₄). Removal of solvents afforded the crude title compound, which was used directly in Step A.

### Examples 97

### N-(piperidin-2-ylmethyl)-3-{[4-(1H-1,2,4-triazol-1-ylmethyl)-1-naphthoyl]amino}pyridine-2-carboxamide

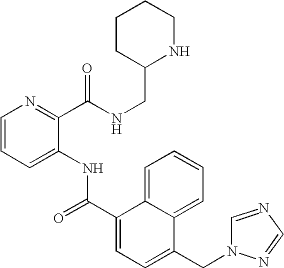

### Step A. N-(piperidin-2-ylmethyl)-3-{[4-(1H-1,2,4-triazol-1-ylmethyl)-1-naphthoyl]amino}pyridine-2-carboxamide

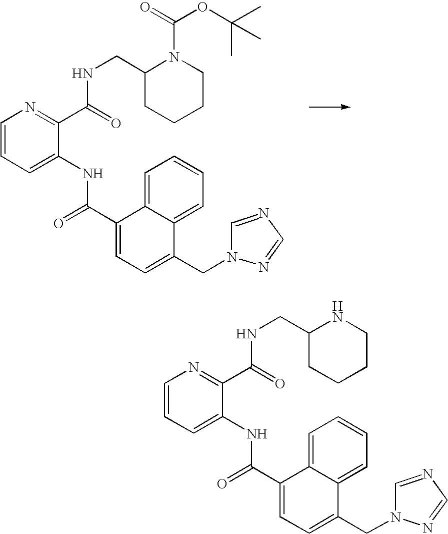

The crude products from Step B were treated with TFA in CH₂Cl₂ (1:1) for 2 hrs at r.t. Removal of solvents gave a residue, which was purified by reversed-phase HPLC to provide N-(piperidin-2-ylmethyl)-3-{[4-(1H-1,2,4-triazol-1-ylmethyl)-1-naphthoyl]amino}pyridine-2-carboxamide as its TFA salt (25 mg, 21%). ¹H NMR (400 MHz, CD₃OD) δ 1.54 (m, 3H), 1.88 (m, 3H), 2.84 (m, 1H), 3.22 (m, 2H), 3.56 (m, 2H), 6.02 (s, 2H), 7.34 (d, J=8.0 Hz, 1H), 7.66 (m, 3H), 7.87 (d, J=8.0 Hz, 1H), 8.05 (s, 1H), 8.25 (d, J=8.0 Hz, 1H), 8.41 (dd, J=4.0 Hz, 1H), 8.48 (d, J=8.0 Hz, 1H), 8.63 (s, 1H), 9.28 (d, J=8.0 Hz, 1H); MS (ESI) (M+H)⁺ 470.0.

### Step B. tert-Butyl 2-({[(3-{[4-(1H-1,2,4-triazol-1-ylmethyl)-1-naphthoyl]amino}pyridin-2-yl)carbonyl]amino}methyl)piperidine-1-carboxylate

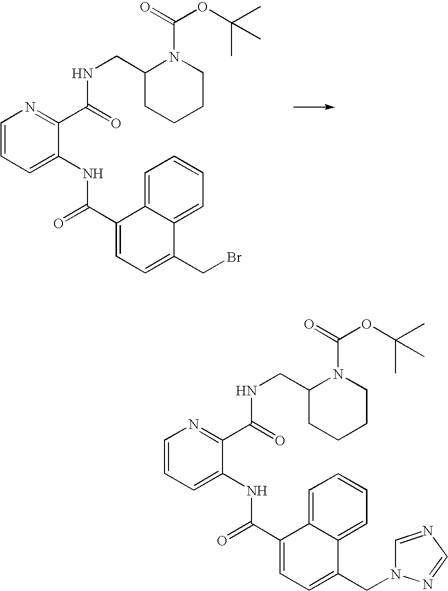

To a solution of tert-butyl 2-({[(3-{[4-(bromomethyl)-1-naphthoyl]amino}pyridin-2-yl)carbonyl]amino}methyl)piperidine-1-carboxylate (100 mg) in DMF (5 mL) was added 1,2,4-triazole (300 mg, 4.3 mmol) at r.t. The solution was stirred at 90° C. for 2 hr, and was then concentrated. The residue was dissolved in EtOAc, washed with brine, and dried (Na₂SO₄). Removal of solvents afforded crude products, which were used directly in Step A.

### Examples 98 & 99

### N-(Piperidin-2-ylmethyl)-3-{[4-(1H-1,2,3-triazol-1-ylmethyl)-1-naphthoyl]amino}pyridine-2-carboxamide

### N-(Piperidin-2-ylmethyl)-3-{[4-(2H-1,2,3-triazol-2-ylmethyl)-1-naphthoyl]amino}pyridine-2-carboxamide

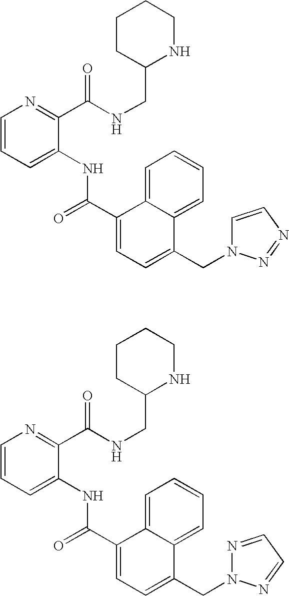

### Step A. N-(Piperidin-2-ylmethyl)-3-{[4-(1H-1,2,3-triazol-1-ylmethyl)-1-naphthoyl]amino}pyridine-2-carboxamide

### And N-(Piperidin-2-ylmethyl)-3-{[4-(2H-1,2,3-triazol-2-ylmethyl)-1-naphthoyl]amino}pyridine-2-carboxamide

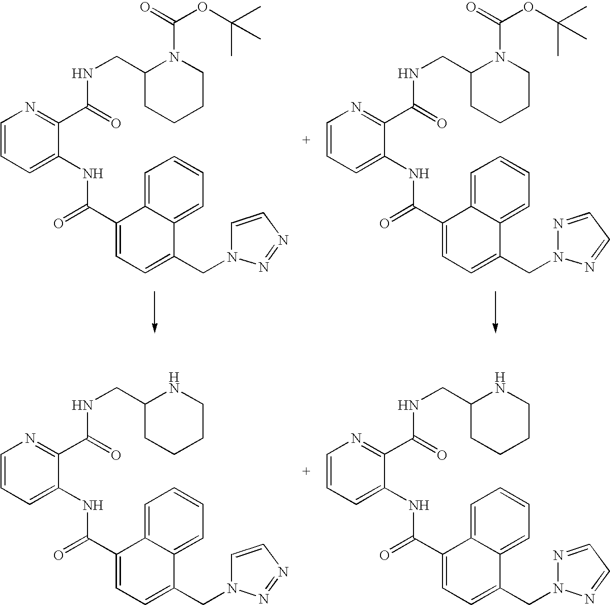

The crude products from Step B were treated with TFA in CH₂Cl₂ (1:1) for 2 hrs at r.t. Removal of solvents gave a residue, which was purified by reversed-phase HPLC to provide N-(piperidin-2-ylmethyl)-3-{[4-(1H-1,2,3-triazol-1-ylmethyl)-1-naphthoyl]amino}pyridine-2-carboxamide as its TFA salt (58 mg, 32%). ¹H NMR (400 MHz, CD₃OD) δ 1.54 (m, 3H), 1.88 (m, 3H), 2.84 (m, 1H), 3.22 (m, 2H), 3.56 (m, 2H), 6.21 (s, 2H), 7.35 (d, J=8.0 Hz, 1H), 7.64 (m, 3H), 7.77 (s, 1H), 7.87 (d, J=8.0 Hz, 1H), 8.02 (s, 1H), 8.26 (d, J=8.0 Hz, 1H), 8.41 (d, J=4.0 Hz, 1H), 8.47 (d, J=8.0 Hz, 1H), 9.27 (d, J=8.0 Hz, 1H); MS (ESI) (M+H)⁺ 470.0.

And N-(piperidin-2-ylmethyl)-3-{[4-(2H-1,2,3-triazol-2-ylmethyl)-1-naphthoyl]amino}pyridine-2-carboxamide as its TFA salt (12 mg, 7%). ¹H NMR (400 MHz, CD₃OD) δ 1.54 (m, 3H), 1.88 (m, 3H), 2.84 (m, 1H), 3.24 (m, 2H), 3.56 (m, 2H), 6.18 (s, 2H), 7.32 (d, J=8.0 Hz, 1H), 7.63 (m, 3H), 7.73 (s, 2H), 7.85 (d, J=8.0 Hz, 1H), 8.30 (d, J=8.0 Hz, 1H), 8.41 (dd, J=4.4, 1.2 Hz, 1H), 8.45 (d, J=8.0 Hz, 1H), 9.27 (d, J=8.0 Hz, 1H); MS (ESI) (M+H)⁺ 470.0.

### Step B. tert-Butyl 2-({[(3-{[4-(1H-1,2,3-triazol-1-ylmethyl)-1-naphthoyl]amino}pyridin-2-yl)carbonyl]amino}methyl)piperidine-1-carboxylate

### and tert-Butyl 2-({[(3-{[4-(2H-1,2,3-triazol-2-ylmethyl)-1-naphthoyl]amino}pyridin-2-yl)carbonyl]amino}methyl)piperidine-1-carboxylate

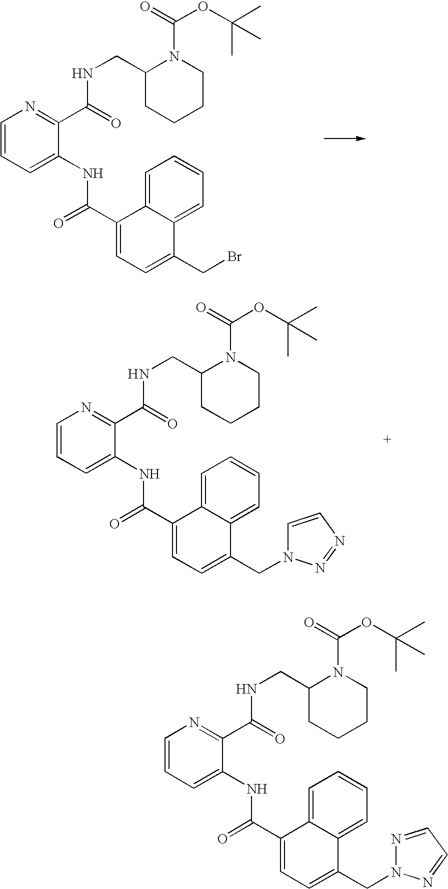

To a solution of tert-butyl 2-({[(3-{[4-(bromomethyl)-1-naphthoyl]amino}pyridin-2-yl)carbonyl]amino}methyl)piperidine-1-carboxylate (150 mg) in DMF (5 mL) was added 1,2,4-triazole (500 mg, 7.2 mmol) at r.t. The solution was stirred at 90° C. for 2 hr, and was then concentrated. The residue was dissolved in EtOAc, washed with brine, and dried (Na₂SO₄). Removal of solvents afforded crude products, which were used directly in Step A.

### Example 100

### 3-[(4-Methyl-1-naphthoyl)amino]-N-[2-(tetrahydro-2H-pyran-4-yl)ethyl]pyridine-2-carboxamide

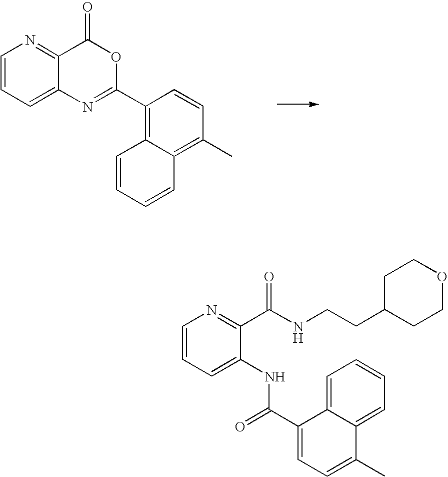

Following the procedure for Step A in Example 1, using 2-(4-methyl-1-naphthyl)-4H-pyrido[3,2-d][1,3]oxazin-4-one (960 mg, 3.3 mmol), and [2-(tetrahydro-2H-pyran-4-yl)ethyl]amine (1.29 g, 10.0 mmol) provided the title compound as its TFA salt after purification by reversed-phase HPLC (584 mg, 33%). ¹H NMR (400 MHz, CDCl₃) δ 1.35 (m, 2H), 1.63 (m, 5H), 2.75 (s 3H), 3.40 (m, 4H), 3.97 (m, 2H), 7.40 (d, J=8.0 Hz, 1H), 7.53 (dd, J=8.0, 4.0 Hz, 1H), 7.58 (m, 1H), 7.81 (d, J=8.0 Hz, 1H), 8.06 (m, 1H), 8.27 (d, J=4.0 Hz, 1H), 8.44 (m, 1H), 8.58 (m, 1H), 9.40 (dd, J=8.0, 1.2 Hz, 1H), 12.78 (brs, 1H); MS (ESI) (M+H)⁺ 418.0.

### Example 101

### 3-{[4-(Methoxymethyl)-1-naphthoyl]amino}-N-[2-(tetrahydro-2H-pyran-4-yl)ethyl]pyridine-2-carboxamide

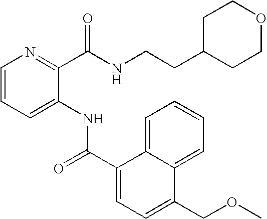

### Step A. 3-{[4-(Methoxymethyl)-1-naphthoyl]amino}-N-[2-(tetrahydro-2H-pyran-4-yl)ethyl]pyridine-2-carboxamide

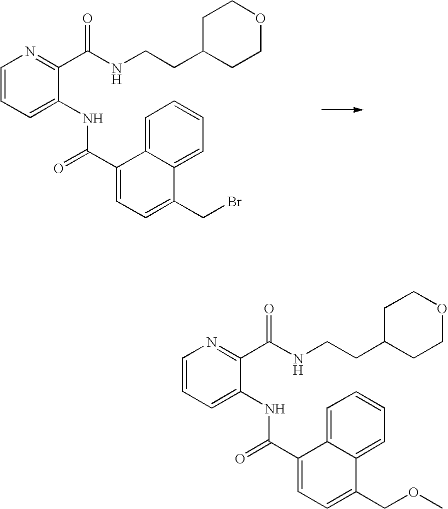

To a solution of 3-{[4-(bromomethyl)-1-naphthoyl]amino}-N-[2-(tetrahydro-2H-pyran-4-yl)ethyl]pyridine-2-carboxamide from Step B in MeOH (10 mL) was added NaOMe (30% in MeOH, 1.0 mL) at 0° C. The solution was stirred at room temperature for 1 hr, and was then concentrated. The residue was dissolved in EtOAc, washed with brine, and dried (Na₂SO₄). Removal of solvents afforded a residue, which was purified by reversed-phase HPLC to provide the title compound as its TFA salt (9 mg, 7%). ¹H NMR (400 MHz, CDCl₃) δ 1.34 (m, 2H), 1.60 (m, 5H), 3.43 (m, 4H), 3.48 (s 3H), 3.95 (m, 2H), 4.96 (s, 2H), 7.54 (dd, J=8.0, 4.0 Hz, 1H), 7.59 (m, 3H), 7.87 (d, J=4.0 Hz, 1H), 8.14 (m, 1H), 8.28 (d, J=4.0 Hz, 1H), 8.43 (m, 1H), 8.56 (m, 1H), 9.41 (dd, J=8.0, 1.2 Hz, 1H), 12.82 (brs, 1H); MS (ESI) (M+H)⁺=448.0.

### Step B. 3-{[4-(Bromomethyl)-1-naphthoyl]amino}-N-[2-(tetrahydro-2H-pyran-4-yl)ethyl]pyridine-2-carboxamide

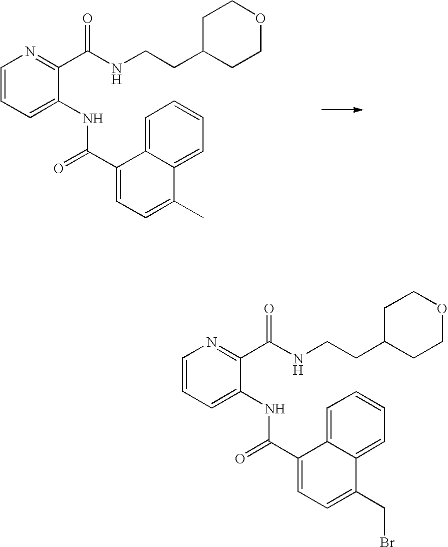

To a solution of 3-[(4-methyl-1-naphthoyl)amino]-N-[2-(tetrahydro-2H-pyran-4-yl)ethyl]pyridine-2-carboxamide (100 mg, 0.24 mmol) and NBS (150 mg, 0.8 mmol) in 1,2-C₂H₄Cl₂ (20 mL) at room temperature, was added 1,1′-azobis(cyclohexanecarbonitrile) (5 mg) in one portion. The solution was heated at 80° C. for 2.5 hours, cooled to room temperature, concentrated and the residue was used directly in Step A.

### Example 102

### 3-[(4-Methyl-1-naphthoyl)amino]-N-(morpholin-3-ylmethyl)pyridine-2-carboxamide

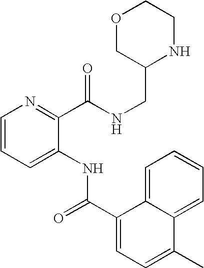

### Step A: 3-[(4-Methyl-1-naphthoyl)amino]-N-(morpholin-3-ylmethyl)pyridine-2-carboxamide

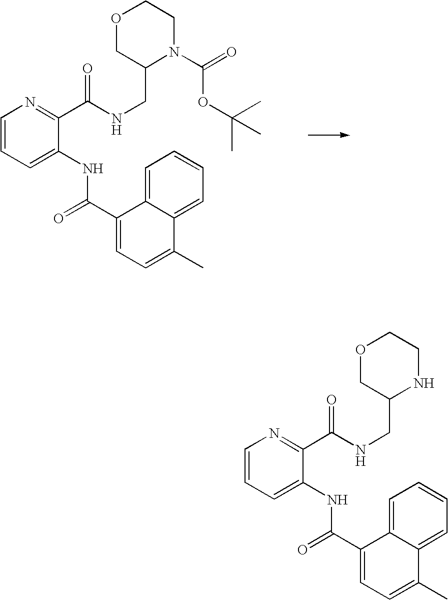

The crude tert-butyl 3-{[({3-[(4-methyl-1-naphthoyl)amino]pyridin-2-yl}carbonyl)amino]methyl}morpholine-4-carboxylate from Step B was treated with TFA in CH₂Cl₂ (1:1) for 1 hr at r.t. After evaporation, the residue was purified by reversed-phase HPLC to provide the title compound as its TFA salt (29 mg, 16% for two steps). ¹H NMR (400 MHz, CD₃OD) δ 2.68 (s, 3H), 3.02 (m, 1H), 3.21 (m, 2H), 3.47 (m, 2H), 3.59 (m, 2H), 3.82 (m, 1H), 3.92 (m, 1H), 7.34 (d, J=8.0 Hz, 1H), 7.54 (m, 3H), 7.71 (d, J=8.0 Hz, 1H), 8.06 (d, J=8.0 Hz, 1H), 8.32 (m, 1H), 8.39 (d, J=8.0 Hz, 1H), 9.20 (d, J=8.0 Hz, 1H); MS (ESI) (M+H)⁺=405.2.

### Step B: tert-Butyl 3-{[({3-[(4-methyl-1-naphthoyl)amino]pyridin-2-yl}carbonyl)amino]methyl}morpholine-4-carboxylate

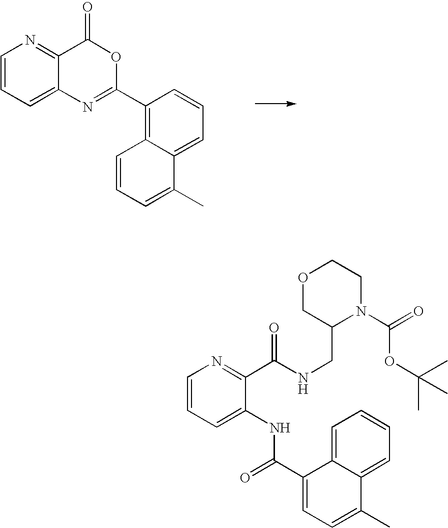

Following the procedure for Step A in Example 1, using 2-(4-methyl-1-naphthyl)-4H-pyrido[3,2-d][1,3]oxazin-4-one (100 mg, 0.35 mmol), and tert-butyl 3-(aminomethyl)morpholine-4-carboxylate (216 mg, 1.0 mmol) provided crude tert-butyl 3-{[({3-[(4-methyl-1-naphthoyl)amino]pyridin-2-yl}carbonyl)amino]methyl}morpholine-4-carboxylate, which was used directly in Step A.

### Example 103

### N-cyclopentyl-3-[(1-naphthalenylcarbonyl)amino]-2-pyridinecarboxamide

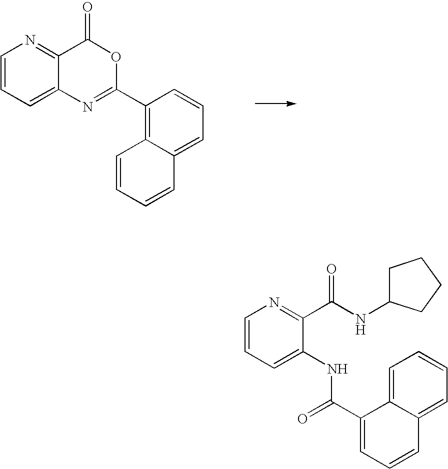

A solution of 2-(1-naphthyl)-4H-pyrido[3,2-d][1,3]oxazin-4-one (100 mg, 0.365 mmol) in DMF (1 mL) was treated with cyclopentylamine (0.22 mL, 2.16 mmol) at room temperature. The mixture was stirred for 3 h at room temperature. After evaporation of the solvent, the residue was purified by reversed-phase HPLC (40-95% CH₃CN in H₂O) to provide the title compound as its TFA salt (22.1 mg, 13%). ¹H NMR (400 MHz, CDCl₃) δ 1.52-1.66 (m, 4H), 1.70-1.80 (m, 2H), 1.94-2.02 (m, 2H), 4.18-4.25 (m, 1H), 7.54-7.62 (m, 4H), 7.89-7.91 (m, 1H), 7.93-7.97 (m, 1H), 8.05-8.07 (m, 1H), 8.34 (dd, J=4.49, 1.37 Hz, 1H), 8.42-8.45 (m, 1H), 9.28 (dd, J=8.59, 1.37 Hz, 1H); MS (ESI) (M+H)⁺ 360.0; Anal. (C, H, N) calcd for C₂₂H₂₁N₃O₂+0.40CH₃OH: C, 72.28; H, 6.12; N, 11.29. found C, 72.23; H, 6.03; N, 11.13.

### Example 104

### N-butyl-3-[[[4-(1H-1,2,3-triazol-1-ylmethyl)-1-naphthalenyl]carbonyl]amino]-2-pyridinecarboxamide

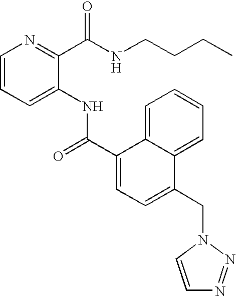

### Step A. N-butyl-3-[[[4-(1H-1,2,3-triazol-1-ylmethyl)-1-naphthalenyl]carbonyl]amino]-2-pyridinecarboxamide

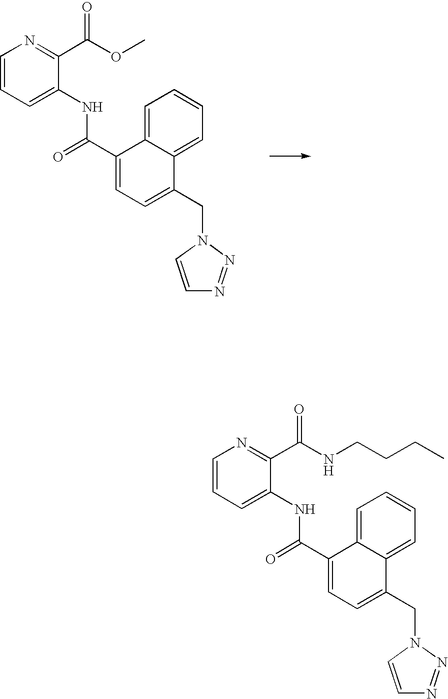

To a solution of methyl 3-{[4-(1H-1,2,3-triazol-1-ylmethyl)-1-naphthoyl]amino}pyridine-2-carboxylate (100 mg, 0.26 mmol) in DMF (1.7 mL) was added butylamine (0.15 mL, 1.51 mmol) at room temperature. The solution was heated at 80° C. for 2 hours and cooled to room temperature. Evaporation of the solvent and purification by reversed-phase HPLC (40-95% CH₃CN in H₂O) afforded the title compound as its TFA salt (16.5 mg, 3%). ¹H NMR (400 MHz, CDCl₃) δ 0.95 (t, J=7.32 Hz, 3H), 1.36-1.46 (m, 2H), 1.57-1.64 (m, 2H), 3.39 (q, J=7.03 Hz, 2H), 6.07 (s, 2H), 7.45 (d, J=7.22 Hz, 1H), 7.53 (dd, J=8.59, 4.49 Hz, 1H), 7.57-7.63 (m, 2H), 7.74 (br.s, 1H), 7.88 (d, J=7.22 Hz, 1H), 8.00-8.02 (m, 1H), 8.30 (dd, J=4.49, 1.46 Hz, 1H), 8.45-8.51 (m, 1H), 8.54-8.57 (m, 1H), 9.39 (dd, J=8.59, 1.46 Hz, 1H), 12.95 (s, 1H); MS (ESI) (M+H)⁺ 429.0; Anal. (C, H, N) calcd for C₂₄H₂₄N₆O₂: C, 67.27; H, 5.65; N, 19.61. found C, 68.29; H, 5.74; N, 19.50.

### Step B. Methyl 3-{[4-(bromomethyl)-1-naphthoyl]amino}pyridine-2-carboxylate

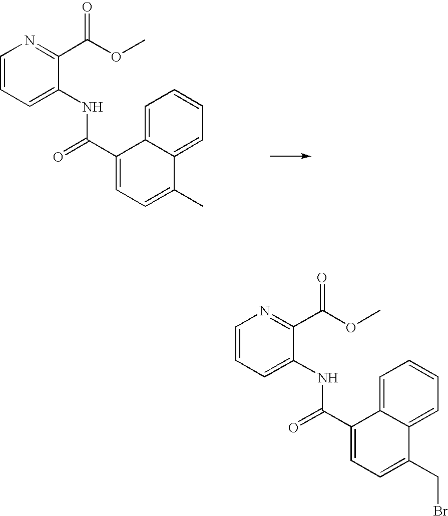

To a solution of methyl 3-[(4-methyl-1-naphthoyl)amino]pyridine-2-carboxylate (700 mg, 2.2 mmol) and NBS (979 mg, 5.5 mmol) in DCE (44 mL) at room temperature, was added 1,1′-azobis(cyclohexanecarbonitrile) (30 mg, 0.12 mmol), in one portion. The solution was heated at 110° C. for 2 hours, and then cooled to room temperature. The solution was concentrated, and the residue was used directly in Step C. MS (ESI) (M+H)⁺ 400.92.

### Step C. Methyl 3-{[4-(1H-1,2,3-triazol-1-ylmethyl)-1-naphthoyl]amino}pyridine-2-carboxylate

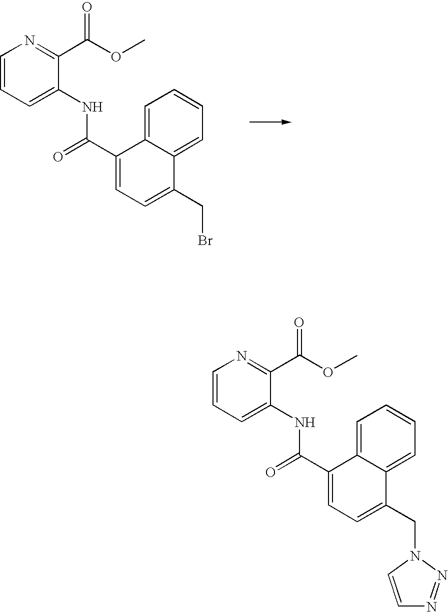

To a solution of methyl 3-{[4-(bromomethyl)-1-naphthoyl]amino}pyridine-2-carboxylate (410 mg, 1.05 mmol) in DMF (20 mL) at room temperature, was added 1,2,3-triazole (1.8 mL, 31.2 mmol), in one portion. The solution was heated at 100° C. for 1 hour, and then cooled to room temperature. The solution was concentrated, and the residue was used directly in Step A. MS (ESI) (M+H)⁺ 387.95.

### Example 105

### N-(cyclopropylmethyl)-3-[[[4-(1H-1,2,3-triazol-1-ylmethyl)-1-naphthalenyl]carbonyl]amino]-2-pyridinecarboxamide

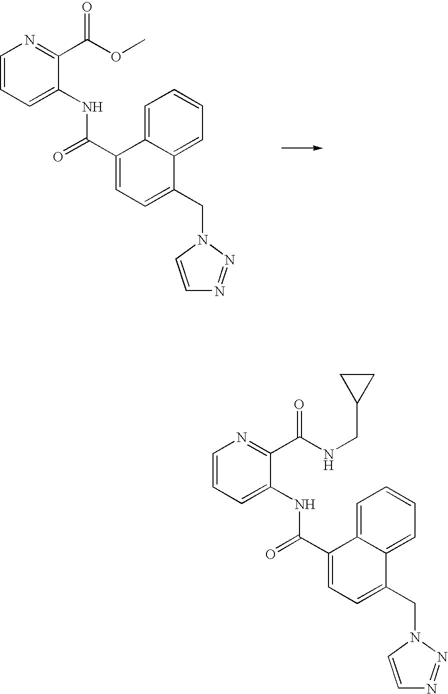

Following the procedure for Step A in Example 104, using methyl 3-{[4-(1H-1,2,3-triazol-1-ylmethyl)-1-naphthoyl]amino}pyridine-2-carboxylate (200 mg, 0.52 mmol) and cyclopropanemethylamine (0.27 mL, 3.12 mmol) provided the title compound as its TFA salt (42.2 mg, 15%) following purification by reversed-phase HPLC (40-95% CH₃CN in H₂O). ¹H NMR (400 MHz, CDCl₃) δ 0.26-0.30 (m, 2H), 0.55-0.60 (m, 2H), 1.01-1.11 (m, 1H), 3.26 (dd, J=7.03, 5.86 Hz, 2H), 6.08 (s, 2H), 7.43 (s, 1H), 7.47 (d, J=7.42 Hz, 1H), 7.55 (dd, J=8.59, 4.49 Hz, 1H), 7.57-7.64 (m, 2H), 7.75 (s, 1H), 7.88 (d, J=7.42 Hz, 1H), 7.98-7.80 (m, 1H), 8.33 (dd, J=4.49, 1.56 Hz, 1H), 8.55-8.57 (m, 2H), 9.39 (dd, J=8.59, 1.56 Hz, 1H), 12.94 (s, 1H); MS (ESI) (M+H)⁺ 427.0; Anal. (C, H, N) calcd for C₂₄H₂₂N₆O₂+0.10CF₃COOH+0.10CH₃OH: C, 66.17; H, 5.14; N, 19.05. found C, 66.26; H, 5.24; N, 19.10.

### Example 106

### N-(cyclopentylmethyl)-3-[[[4-(1H-1,2,3-triazol-1-ylmethyl)-1-naphthalenyl]carbonyl]amino]-2-pyridinecarboxamide

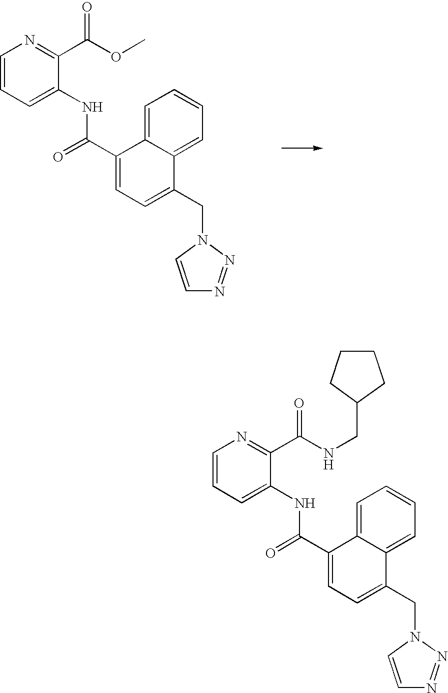

Following the procedure for Step A in Example 104, using methyl 3-{[4-(1H-1,2,3-triazol-1-ylmethyl)-1-naphthoyl]amino}pyridine-2-carboxylate (200 mg, 0.52 mmol) and cyclopentanemethylamine (0.92 mL, 3.12 mmol, 3.4 M in MeOH) provided the title compound as its TFA salt (16.3 mg, 6%) following purification by reversed-phase HPLC (50-95% CH₃CN in H₂O). ¹H NMR (400 MHz, CDCl₃) δ 1.22-1.30 (m, 2H), 1.53-1.67 (m, 4H), 1.76-1.85 (m, 2H), 2.12-2.21 (m, 1H), 3.32-3.35 (m, 2H), 3.49 (s, 1H), 6.07 (s, 2H), 7.40 (s, 1H), 7.45 (d, J=7.42 Hz, 1H), 7.54 (dd, J=8.59, 4.49 Hz, 1H), 7.57-7.63 (m, 2H), 7.70 (s, 1H), 7.88 (d, J=7.42 Hz, 1H), 8.00-8.02 (m, 1H), 8.30 (dd, J=4.49, 1.37 Hz, 1H), 8.51-8.57 (m, 1H), 9.39 (dd, J=8.59, 1.37 Hz, 1H), 12.95 (s, 1H); MS (ESI) (M+H)⁺ 455.0; Anal. (C, H, N) calcd for C₂₆H₂₆N₆O₂+0.10CF₃COOH+0.40CH₃OH: C, 66.73; H, 5.83; N, 17.55. found C, 66.85; H, 5.70; N, 17.43.

### Example 107

### N-hexyl-3-[[[4-(1H-1,2,3-triazol-1-ylmethyl)-1-naphthalenyl]carbonyl]amino]-2-pyridinecarboxamide

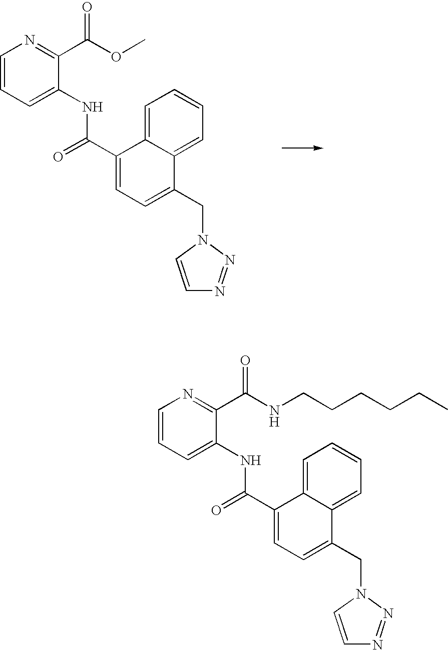

Following the procedure for Step A in Example 104, using methyl 3-{[4-(1H-1,2,3-triazol-1-ylmethyl)-1-naplhthoyl]amino}pyridine-2-carboxylate (100 mg, 0.26 mmol) in DMF (1 mL) and hexylamine (0.2 mL, 1.51 mmol) provided the title compound as its TFA salt (27.6 mg, 18%) following purification by reversed-phase HPLC (40-95% CH₃CN in H₂O). ¹H NMR (400 MHz, CDCl₃) δ 0.86-0.90 (m, 3H), 1.28-1.41 (m, 6H), 1.58-1.65 (m, 2H), 3.36-3.41 (m, 2H), 6.07 (s, 2H), 7.40 (s, 1H), 7.44 (d, J=7.42 Hz, 1H), 7.53 (dd, J=8.59, 4.49 Hz, 1H), 7.56-7.63 (m, 2H), 7.70 (s, 1H), 7.88 (d, J=7.42 Hz, 1H), 8.00-8.02 (m, 1H), 8.30 (dd, J=4.49, 1.46 Hz, 1H), 8.47-8.50 (m, 1H), 8.55-8.57 (m, 1H), 9.39 (dd, J=8.59, 1.46 Hz, 1H), 12.95 (s, 1H); MS (ESI) (M+H)⁺ 457.0; Anal. (C, H, N) calcd for C₂₆H₂₈N₆O₂+1.80H₂O: C, 63.87; H, 6.51; N, 17.19. found C, 63.36; H, 5.77; N, 18.92.

### Example 108

### N-[3-(dimethylamino)propyl]-3-[[[4-(1H-1,2,3-triazol-1-ylmethyl)-1-naphthalenyl]carbonyl]amino]-2-pyridinecarboxamide

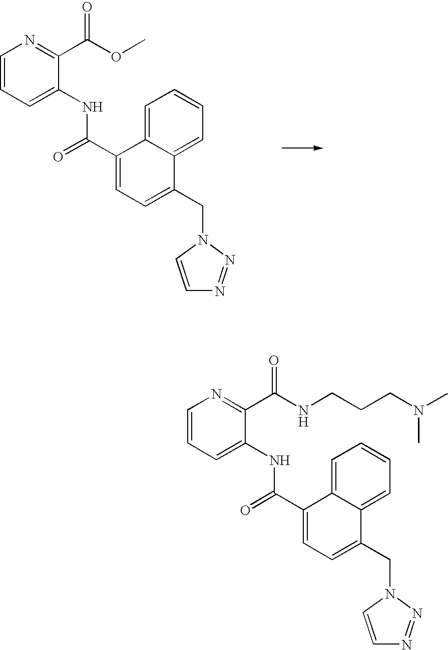

Following the procedure for Step A in Example 104, using methyl 3-{[4-(1H-1,2,3-triazol-1-ylmethyl)-1-naphthoyl]amino}pyridine-2-carboxylate (100 mg, 0.26 idol) in DMF (1 mL) and N,N-dimethyl-1,3-propanediamine (0.2 mL, 1.51 mmol) provided the title compound as its TFA salt (83.7 mg, 56%) following purification by reversed-phase HPLC (20-50% CH₃CN in H₂O). ¹H NMR (400 MHz, CDCl₃) δ 2.06-2.10 (m, 2H), 2.80 (s, 6H), 3.07-3.11 (m, 2H), 3.46-3.51 (q, 2H), 6.07 (s, 2H), 7.39 (d, J=7.22 Hz, 1H), 7.49 (s, 1H), 7.54 (dd, J=8.59, 4.49 Hz, 1H), 7.57-7.64 (m, 2H), 7.72 (s, 1H), 7.84 (d, J=7.22 Hz, 1H), 8.01-8.03 (m, 1H), 8.30 (dd, J=4.49, 1.37 Hz, 1H), 8.54-8.57 (m, 1H), 8.75-8.78 (m, 1H), 9.36 (dd, J=8.59, 1.37 Hz, 1H), 12.68 (s, 1H); MS (ESI) (M+H)⁺ 458.0; Anal. (C, H, N) calcd for C₂₅H₂₇N₇O₂+1.60CF₃COOH+0.70H₂O: C, 51.90; H, 4.62; N, 15.10. found C, 51.89; H, 4.63; N, 15.02.

### Example 109

### N-[2-(4-morpholinyl)ethyl]-3-[[[4-(1H-1,2,3-triazol-1-ylmethyl)-1-naphthalenyl]carbonyl]amino]-2-pyridinecarboxamide

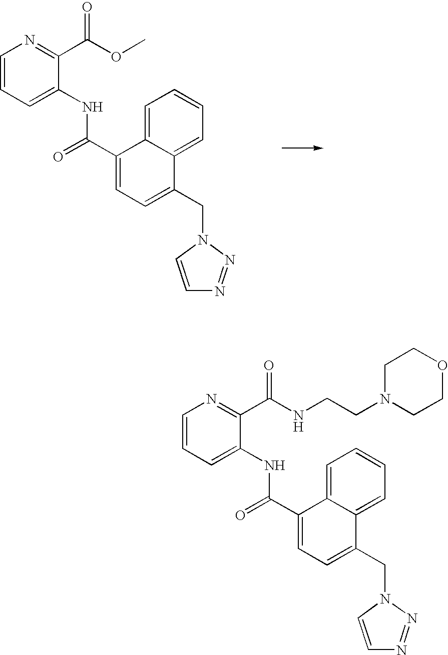

Following the procedure for Step A in Example 104, using methyl 3-{[4-(1H-1,2,3-triazol-1-ylmethyl)-1-naphthoyl]amino}pyridine-2-carboxylate (100 mg, 0.26 mmol) in DMF (1 mL) and 4(2-aminoethyl)morpholine (0.2 mL, 1.51 mmol) provided the title compound as its TFA salt (66.4 mg, 42%) following purification by reversed-phase HPLC (10-95% CH₃CN in H₂O). ¹H NMR (400 MHz, CDCl₃) δ 2.68-3.00 (m, 2H), 3.33-3.36 (m, 2H), 3.66-3.70 (m, 2H), 3.78-4.03 (m, 6H), 6.07 (s, 2H), 7.38 (d, J=7.42 Hz, 1H), 7.52 (dd, J=8.59, 4.49 Hz, 1H), 7.58-7.65 (m, 2H), 7.80 (m, 1H), 7.84 (d, J=7.42 Hz, 1H), 7.96-7.80 (m, 1H), 8.13-8.14 (m, 1H), 8.27 (dd, J=4.49, 1.37 Hz, 1H), 8.51-8.55 (m, 1H), 9.02-9.05 (m, 1H), 9.32 (dd, J=8.59, 1.37 Hz, 1H), 12.50 (s, 1H); MS (ESI) (M+H)⁺ 486.0.

### Examples 110 & 111

### N-(Cyclohexylmethyl)-3-{[4-(1H-1,2,3-triazol-1-ylmethyl)-1-naphthoyl]amino}pyridine-2-carboxamide and N-(cyclohexylmethyl)-3-{[4-(2H-1,2,3-triazol-2-ylmethyl)-1-naphthoyl]amino}pyridine-2-carboxamide

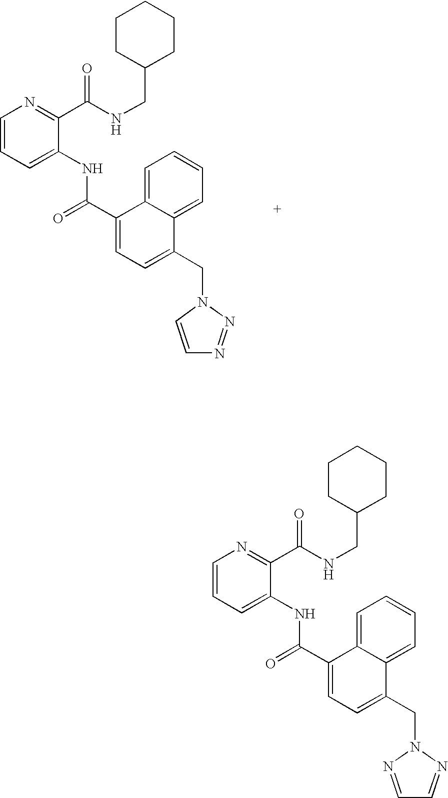

### Step A. N-(Cyclohexylmethyl)-3-{[4-(1H-1,2,3-triazol-1-ylmethyl)-1-naphthoyl]amino}pyridine-2-carboxamide and N-(cyclohexylmethyl)-3-{[4-(2H-1,2,3-triazol-2-ylmethyl)-1-naphthoyl]amino}pyridine-2-carboxamide

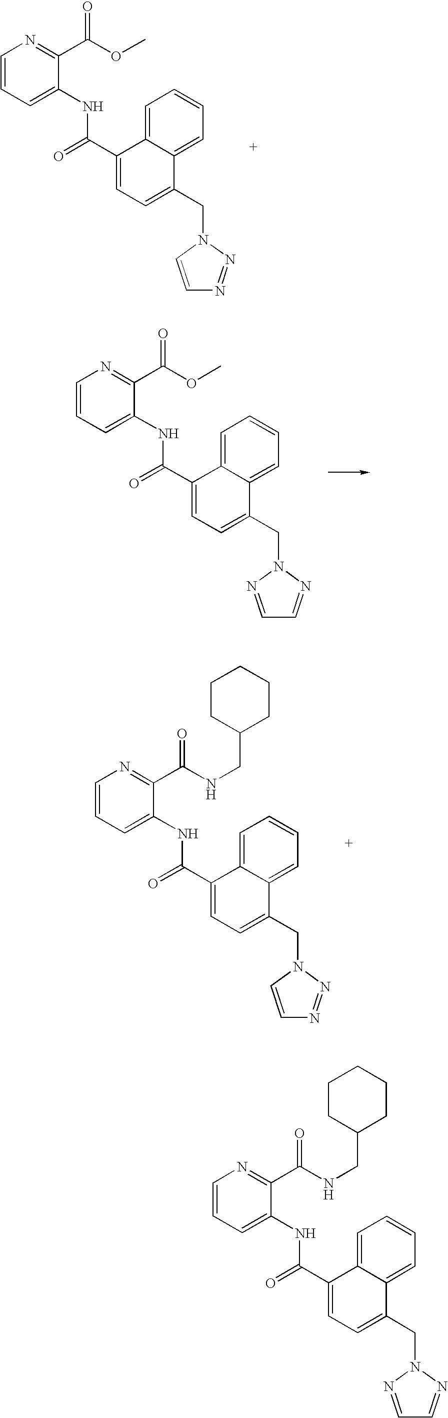

Following the procedure for Step A in Example 104, using the crude products from Step C (116 mg, 0.3 mmol) and (cyclohexylmethyl)amine (170 mg, 1.5 mmol) provided N-(cyclohexylmethyl)-3-{[4-(1H-1,2,3-triazol-1-ylmethyl)-1-naphthoyl]amino}pyridine-2-carboxamide as its TFA salt after purification by reversed-phase HPLC (59 mg, 34%). ¹H NMR (400 MHz, CDCl₃) δ 0.90 (m, 2H), 1.16 (m, 3H), 1.66 (m, 6H), 3.12 (d, J=6.8 Hz, 2H), 6.15 (s, 2H), 7.41 (d, J=8.0 Hz, 1H), 7.56 (dd, J=8.6, 4.5 Hz, 1H), 7.59 (m, 2H), 7.75 (brs, 1H), 7.84 (d, J=8.0 Hz, 1H), 7.95 (brs, 1H), 8.17 (m, 1H), 8.32 (dd, J=4.5, 1.3 Hz, 1H), 8.46 (m, 1H), 9.23 (dd, J=8.6, 1.3 Hz, 1H); MS (ESI) (M+H)⁺ 469.0;

And N-(cyclohexylmethyl)-3-{[4-(2H-1,2,3-triazol-2-ylmethyl)-1-naphthoyl]amino}pyridine-2-carboxamide as its TFA salt (59 mg, 34%). ¹H NMR (400 MHz, CDCl₃) δ 0.93 (m, 2H), 1.17 (m, 3H), 1.68 (m, 6H), 3.12 (d, J=6.8 Hz, 2H), 6.14 (s, 2H), 7.33 (d, J=8.0 Hz, 1H), 7.56 (m, 3H), 7.71 (s, 2H), 7.83 (d, J=8.0 Hz, 1H), 8.24 (m, 1H), 8.32 (m, 1H), 8.46 (m, 1H), 9.23 (d, J=8.0 Hz, 1H); MS (ESI) (M+H)⁺ 469.2.

### Step B. Methyl 3-{[4-(bromomethyl)-1-naphthoyl]amino}pyridine-2-carboxylate

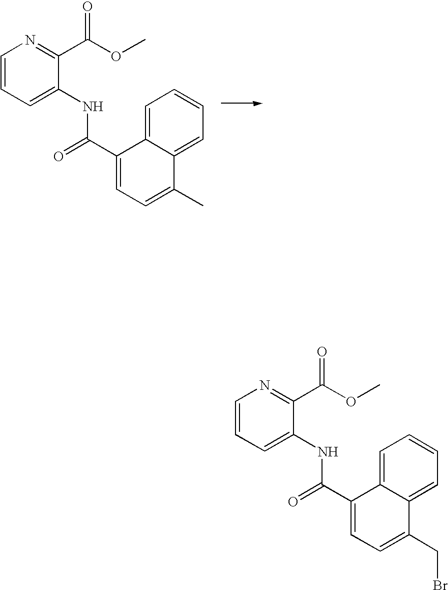

To a solution of methyl 3-[(4-methyl-1-naphthoyl)amino]pyridine-2-carboxylate (96 mg, 0.3 mmol) and NBS (107 mg, 0.6 mmol) in DCE (20 mL) at room temperature, was added 1,1′-azobis(cyclohexanecarbonitrile) (5 mg), in one portion. The solution was heated at 110° C. for 2 hours, and then cooled to room temperature. The solution was concentrated, and the residue was used directly in Step C. MS (ESI) (M+H)⁺ 400.92.

### Step C. Methyl 3-{[4-(1H-1,2,3-triazol-1-ylmethyl)-1-naphthoyl]amino}pyridine-2-carboxylate and methyl 3-{[4-(2H-1,2,3-triazol-2-ylmethyl)-1-naphthoyl]amino}pyridine-2-carboxylate

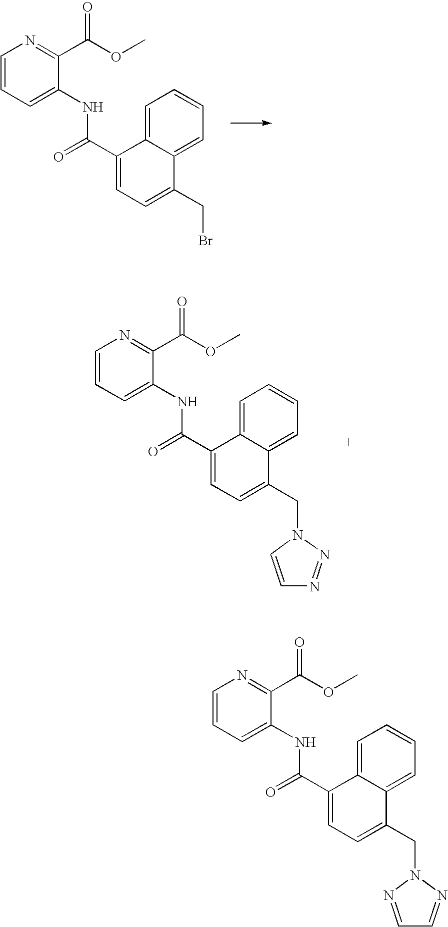

To a solution of crude methyl 3-{[4-(bromomethyl)-1-naphthoyl]amino}pyridine-2-carboxylate from Step C (0.3 mmol) in DMF (5 mL) at room temperature, was added 1,2,3-triazole (138 mg, 2.0 mmol), in one portion. The solution was heated at 100° C. for 1 hour, and then cooled to room temperature. The solution was concentrated, and the residue was used directly in Step A.

### Example 112

### N-Pentyl-3-{[4-(1H-1,2,3-triazol-1-ylmethyl)-1-naphthoyl]amino}pyridine-2-carboxamide

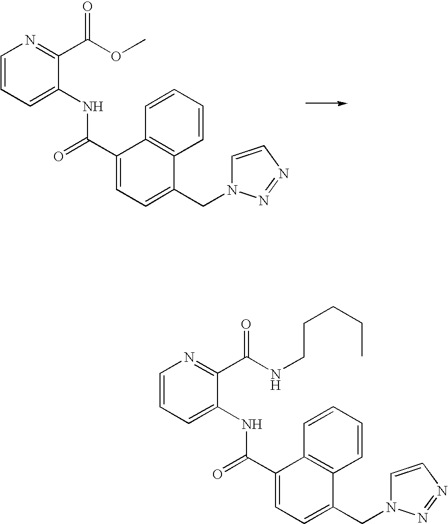

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and pentan-1-amine (130 mg, 1.5 mmol) provided the title compound as its TFA salt after purification by reversed-phase HPLC (55 mg, 33%). ¹H NMR (400 MHz, CDCl₃) δ AZM1229-49; MS (ESI) (M+H)⁺ 443.0.

### Example 113

### N-[2-(Tetrahydro-2H-pyran-4-yl)ethyl]-3-{[4-(1H-1,2,3-triazol-1-ylmethyl)-1-naphthoyl]amino}pyridine-2-carboxamide

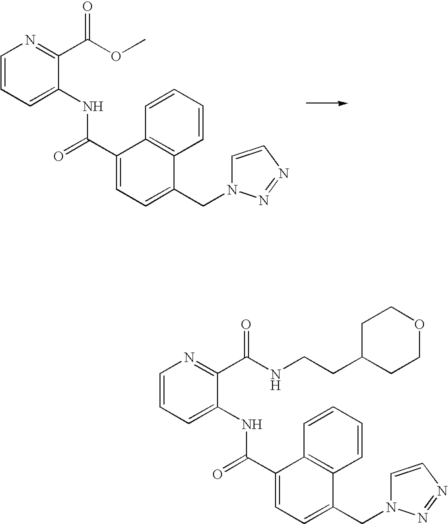

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and 2-(tetrahydro-2H-pyran-4-yl)ethanamine (194 mg, 1.5 mmol) provided the title compound as its TFA salt after purification by reversed-phase HPLC (118 mg, 66%). ¹H NMR (400 MHz, CD₃OD) δ 1.21 (m, 2H), 1.49 (m, 3H), 1.60 (m, 2H), 3.30 (m, 4H), 3.84 (m, 2H), 6.15 (s, 2H), 7.39 (d, J=8.0 Hz, 1H), 7.55 (dd, J=8.6, 4.5 Hz, 1H), 7.59 (m, 2H), 7.74 (brs, 1H), 7.84 (d, J=8.0 Hz, 1H), 7.95 (brs, 1H), 8.18 (m, 1H), 8.31 (dd, J=4.5, 1.3 Hz, 1H), 8.46 (m, 1H), 9.22 (dd, J=8.6, 1.3 Hz, 1H); MS (ESI) (M+H)⁺ 448.0.

### Example 114

### N-[2-(1H-Pyrrol-1-yl)ethyl]-3-{[4-(1H-1,2,3-triazol-1-ylmethyl)-1-naphthoyl]amino}pyridine-2-carboxamide

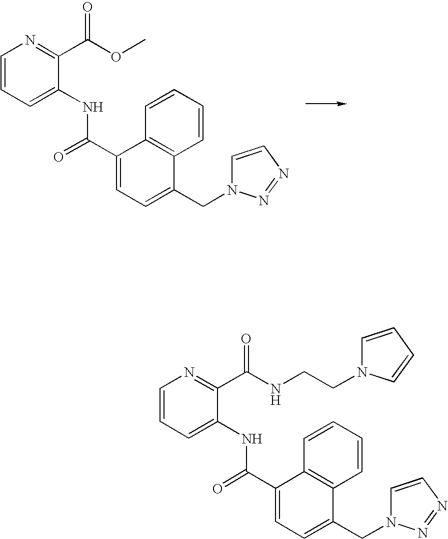

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and [2-(1H-pyrrol-1-yl)ethyl]amine (165 mg, 1.5 mmol) provided the title compound as its TFA salt after purification by reversed-phase HPLC (39 mg, 22%). ¹H NMR (400 MHz, CD₃OD) δ 3.58 (d, J=6.4 Hz, 2H), 4.02 (d, J=6.4 Hz, 2H), 5.98 (brs, 2H), 6.15 (s, 2H), 6.63 (brs, 2H), 7.38 (d, J=8.0 Hz, 1H), 7.53 (dd, J=8.6, 4.5 Hz, 1H), 7.59 (m, 2H), 7.74 (brs, 1H), 7.82 (d, J=8.0 Hz, 1H), 7.95 (brs, 1H), 8.17 (m, 1H), 8.28 (dd, J=4.5, 1.3 Hz, 1H), 8.46 (m, 1H), 9.20 (dd, J=8.6, 1.3 Hz, 1H); MS (ESI) (M+H)⁺ 466.0.

### Example 115

### N-[3-(1H-Imidazol-1-yl)propyl]-3-{[4-(1H-1,2,3-triazol-1-ylmethyl)-1-naphthoyl]amino}pyridine-2-carboxamide

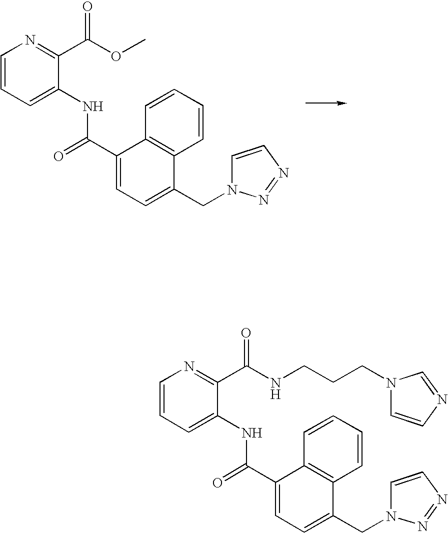

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and [3-(1H-imidazol-1-yl)propyl]amine (188 mg, 1.5 mmol) provided the title compound as its TFA salt after purification by reversed-phase HPLC (92 mg, 52%). ¹H NMR (400 MHz, CD₃OD) δ 2.15 (m, 2H), 3.39 (m, 2H), 4.26 (m, 2H), 6.17 (s, 2H), 7.37 (d, J=8.0 Hz, 1H), 7.46 (m, 1H), 7.59 (m, 4H), 7.75 (brs, 1H), 7.84 (d, J=8.0 Hz, 1H), 8.0 (brs, 1H), 8.19 (m, 1H), 8.36 (dd, J=4.5, 1.3 Hz, 1H), 8.46 (m, 1H), 8.90 (s, 1H), 9.22 (dd, J=8.6, 1.3 Hz, 1H); MS (ESI) (M+H)⁺ 481.0.

### Example 116

### N-[3-(1H-Pyrazol-1-yl)propyl]-3-{[4-(1H-1,2,3-triazol-1-ylmethyl)-1-naphthoyl]amino}pyridine-2-carboxamide

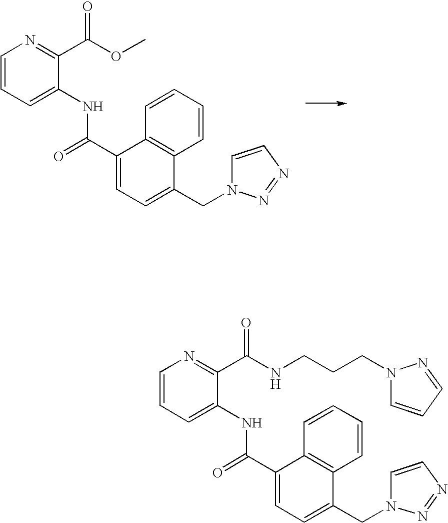

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and [3-(1H-pyrazol-1-yl)propyl]amine (188 mg, 1.5 mmol) provided the title compound as its TFA salt after purification by reversed-phase HPLC (62 mg, 35%). ¹H NMR (400 MHz, CD₃OD) δ 2.01 (m, 2H), 3.23 (m, 2H), 4.13 (m, 2H), 6.04 (s, 2H), 6.18 (s, 1H), 7.23 (d, J=8.0 Hz, 1H), 7.43 (m, 1H), 7.47 (m, 3H), 7.58 (brs, 1H), 7.69 (m, 1H), 7.74 (d, J=8.0 Hz, 1H), 7.88 (brs, 1H), 8.06 (m, 1H), 8.21 (d, J=4.5 Hz, 1H), 8.41 (m, 1H), 9.11 (d, J=8.0 Hz, 1H); MS (ESI) (M+H)⁺ 481.0.

### Example 117

### N-[2-(1H-Imidazol-1-yl)ethyl]-3-{[4-(1H-1,2,3-triazol-1-ylmethyl)-1-naphthoyl]amino}pyridine-2-carboxamide

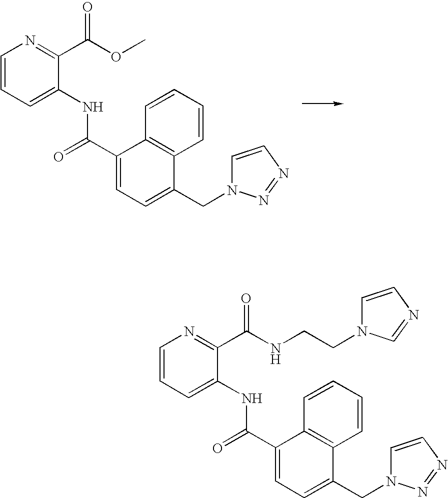

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and [2-(1H-imidazol-1-yl)ethyl]amine (167 mg, 1.5 mmol) provided the title compound as its TFA salt after purification by reversed-phase HPLC (48 mg, 28%). ¹H NMR (400 MHz, CD₃OD) δ 3.77 (m, 2H), 4.40 (m, 2H), 6.14 (s, 2H), 7.33 (d, J=8.0 Hz, 1H), 7.44 (s, 1H), 7.57 (m, 4H), 7.74 (brs, 1H), 7.77 (d, J=8.0 Hz, 1H), 7.98 (brs, 1H), 8.17 (d, J=8.0 Hz, 1H), 8.30 (m, 1H), 8.43 (d, J=8.0 Hz, 1H), 8.90 (s, 1H), 9.17 (dd, J=8.0 Hz, 1H); MS (ESI) (M+H)⁺ 467.0.

### Example 118

### N-[2-(1H-1,2,4-Triazol-1-yl)ethyl]-3-{[4-(1H-1,2,3-triazol-1-ylmethyl)-1-naphthoyl]amino}pyridine-2-carboxamide

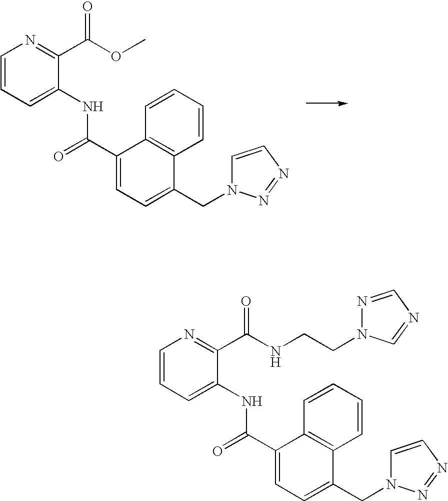

Following the procedure for Step A in Example 104, using methyl 3-{[4-(1H-1,2,3-triazol-1-ylmethyl)-1-naphthoyl]amino}pyridine-2-carboxylate (116 mg, 0.3 mmol) and 2-(1H-1,2,4-triazol-1-yl)ethanamine (168 mg, 1.5 mmol) provided the title compound as its TFA salt after purification by reversed-phase HPLC (46 mg, 26%). ¹H NMR (400 MHz, CD₃OD) δ 3.78 (m, 2H), 4.46 (m, 2H), 6.19 (s, 2H), 7.42 (d, J=8.0 Hz, 1H), 7.59 (m, 3H), 7.75 (brs, 1H), 7.83 (d, J=8.0 Hz, 1H), 7.98 (brs, 1H), 8.17 (s, 1H), 8.21 (m, 1H), 8.32 (m, 1H), 8.45 (d, J=8.0 Hz, 1H), 8.77 (s, 1H), 9.23 (d, J=8.0 Hz, 1H); MS (ESI) (M+H)⁺ 468.0.

### Example 119

### N-(2-Methoxyethyl)-3-{[4-(1H-1,2,3-triazol-1-ylmethyl)-1-naphthoyl]amino}pyridine-2-carboxamide

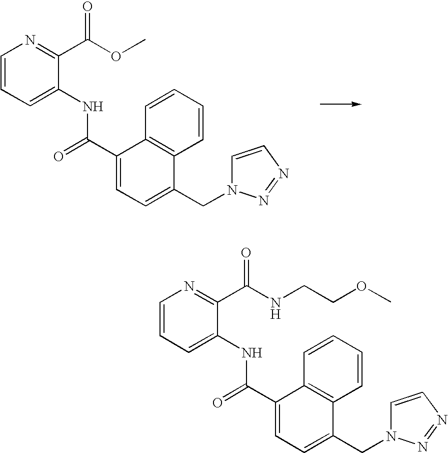

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and (2-methoxyethyl)amine (75 mg, 1.0 mmol) provided the title compound as its TFA salt after purification by reversed-phase HPLC (42 mg, 51%). ¹H NMR (400 MHz, CD₃OD) δ 3.35 (s, 3H), 3.52 (m, 4H), 6.21 (s, 2H), 7.46 (d, J=8.0 Hz, 1H), 7.63 (m, 3H), 7.75 (brs, 1H), 7.89 (d, J=8.0 Hz, 1H), 7.97 (brs, 1H), 8.23 (m, 1H), 8.37 (dd, J=8.0, 1.3 Hz, 1H), 8.48 (m, 1H), 9.28 (dd, J=8.0, 1.4 Hz, 1H); MS (ESI) (M+H)⁺ 431.0.

### Example 120

### N-(2-Ethoxyethyl)-3-{[4-(1H-1,2,3-triazol-1-ylmethyl)-1-naphthoyl]amino}pyridine-2-carboxamide

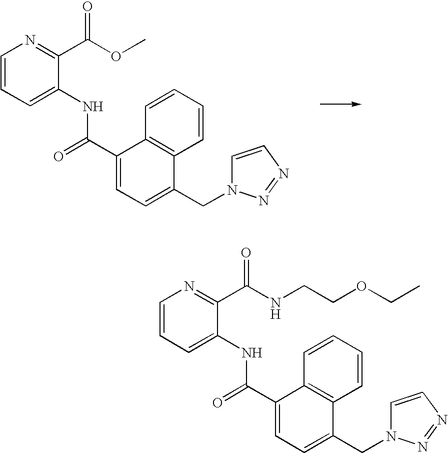

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and (2-ethoxyethyl)amine (89 mg, 1.0 mmol) provided the title compound as its TFA salt after purification by reversed-phase HPLC (22 mg, 26%). ¹H NMR (400 MHz, CD₃OD) δ 1.16 (t, J=6.8 Hz, 3H), 3.51 (m, 4H), 3.56 (m, 2H), 6.19 (s, 2H), 7.44 (d, J=8.0 Hz, 1H), 7.61 (m, 3H), 7.75 (brs, 1H), 7.87 (d, J=8.0 Hz, 1H), 7.96 (brs, 1H), 8.21 (m, 1H), 8.35 (dd, J=8.0, 1.3 Hz, 1H), 8.48 (m, 1H), 9.27 (dd, J=8.0, 1.4 Hz, 1H); MS (ESI) (M+H)⁺ 445.0.

### Example 121

### N-(2-Propoxyethyl)-3-{[4-(1H-1,2,3-triazol-1-ylmethyl)-1-naphthoyl]amino}pyridine-2-carboxamide

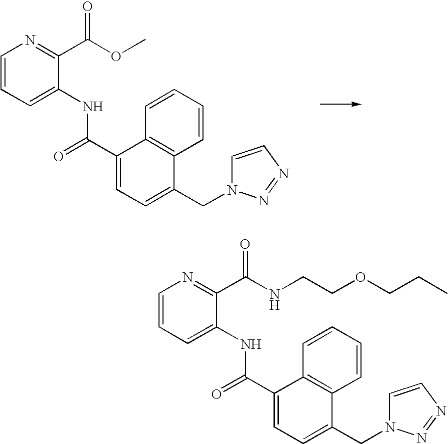

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and (2-propoxyethyl)amine (103 mg, 1.0 mmol) provided the title compound as its TFA salt after purification by reversed-phase HPLC (43 mg, 51%). ¹H NMR (400 MHz, CD₃OD) δ 0.86 (t, J=7.4 Hz, 3H), 1.53 (m, 2H), 3.39 (m, 2H), 3.49 (m, 2H), 3.53 (m, 2H), 6.15 (s, 2H), 7.39 (d, J=8.0 Hz, 1H), 7.58 (m, 3H), 7.75 (brs, 1H), 7.84 (d, J=8.0 Hz, 1H), 7.97 (brs, 1H), 8.17 (m, 1H), 8.31 (m, 1H), 8.46 (m, 1H), 9.22 (d, J=8.0 Hz, 1H); MS (ESI) (M+H)⁺ 459.0.

### Example 122

### N-(3-Methoxypropyl)-3-{[4-(1H-1,2,3-triazol-1-ylmethyl)-1-naphthoyl]amino}pyridine-2-carboxamide

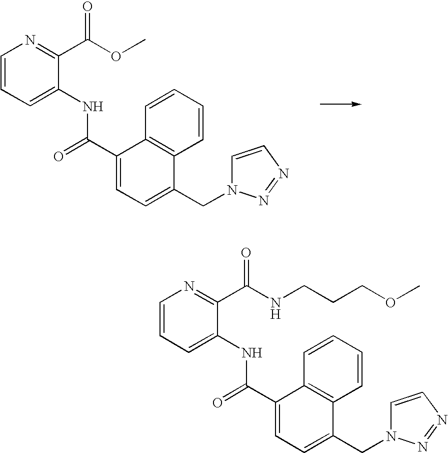

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and (3-methoxypropyl)amine (89 mg, 1.0 mmol) provided the title compound as its TFA salt after purification by reversed-phase HPLC (39 mg, 46%). ¹H NMR (400 MHz, CD₃OD) δ 1.81 (m, 2H), 3.29 (s, 3H), 3.42 (m, 2H), 3.44 (m, 2H), 6.16 (s, 2H), 7.41 (d, J=8.0 Hz, 1H), 7.58 (m, 3H), 7.73 (brs, 1H), 7.85 (d, J=8.0 Hz, 1H), 7.94 (brs, 1H), 8.19 (m, 1H), 8.33 (dd, J=4.5, 1.4 Hz, 1H), 8.46 (m, 1H), 9.23 (dd, J=8.0, 1.4 Hz, 1H); MS (ESI) (M+H)⁺ 445.0.

### Example 123

### N-(3-Ethoxypropyl)-3-{[4-(1H-1,2,3-triazol-1-ylmethyl)-1-naphthoyl]amino}pyridine-2-carboxamide

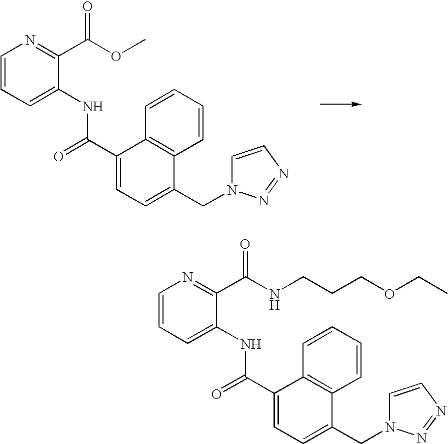

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and (3-ethoxypropyl)amine (103 mg, 1.0 mmol) provided the title compound as its TFA salt after purification by reversed-phase HPLC (38 mg, 44%). ¹H NMR (400 MHz, CD₃OD) δ 1.18 (t, J=7.0 Hz, 1H), 1.83 (m, 2H), 3.50 (m, 4H), 3.52 (m, 2H), 6.21 (s, 2H), 7.47 (d, J=8.0 Hz, 1H), 7.63 (m, 3H), 7.76 (brs, 1H), 7.88 (d, J=8.0 Hz, 1H), 7.98 (brs, 1H), 8.22 (m, 1H), 8.36 (dd, J=4.5, 1.4 Hz, 1H), 8.48 (m, 1H), 9.27 (dd, J=8.0, 1.4 Hz, 1H); MS (ESI) (M+H)⁺ 459.0.

### Example 124

### N-Allyl-3-{[4-(1H-1,2,3-triazol-1-ylmethyl)-1-naphthoyl]amino}pyridine-2-carboxamide

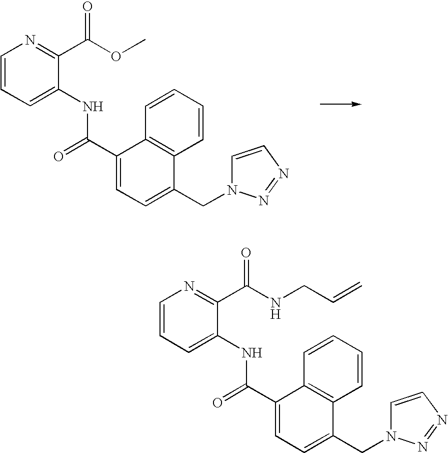

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and allylamine (57 mg, 1.0 mmol) provided the title compound as its TFA salt after purification by reversed-phase HPLC (42 mg, 53%). ¹H NMR (400 MHz, CD₃OD) δ 3.92 (d, J=5.5 Hz, 2H), 5.08 (m, 1H), 5.19 (m, 1H), 5.85 (m, 1H), 6.13 (s, 2H), 7.37 (d, J=8.0 Hz, 1H), 7.56 (m, 3H), 7.72 (brs, 1H), 7.82 (d, J=8.0 Hz, 1H), 7.92 (brs, 1H), 8.16 (m, 1H), 8.31 (dd, J=4.5, 1.4 Hz, 1H), 8.46 (m, 1H), 9.22 (dd, J=8.0, 1.4 Hz, 1H); MS (ESI) (M+H)⁺ 413.0.

### Example 125

### N-Propyl-3-{[4-(1H-1,2,3-triazol-1-ylmethyl)-1-naphthoyl]amino}pyridine-2-carboxamide

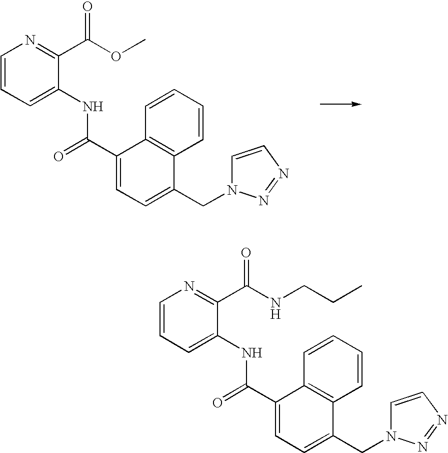

Following the procedure for Step A in Example 104, using methyl 3-{[4-(1H-1,2,3-triazol-1-ylmethyl)-1-naphthoyl]amino}pyridine-2-carboxylate (58 mg, 0.15 mmol) and propylamine (59 mg, 1.0 mmol) provided the title compound as its TFA salt after purification by reversed-phase HPLC (32 mg, 40%). ¹H NMR (400 MHz, CD₃OD) δ 0.89 (t, J=7.4 Hz, 3H), 1.56 (m, 2H), 3.24 (m, 2H), 6.13 (s, 2H), 7.37 (d, J=8.0 Hz, 1H), 7.56 (m, 3H), 7.72 (brs, 1H), 7.83 (d, J=8.0 Hz, 1H), 7.93 (brs, 1H), 8.16 (m, 1H), 8.31 (dd, J=4.5, 1.4 Hz, 1H), 8.46 (m, 1H), 9.21 (dd, J=8.0, 1.4 Hz, 1H); MS (ESI) (M+H)⁺ 415.0.

### Example 128

### N-[(tetrahydro-2H-pyran-4-yl)methyl]-3-[[[4-(1H-1,2,3-triazol-1-ylmethyl)-1-naphthalenyl]carbonyl]amino]-2-pyridinecarboxamide

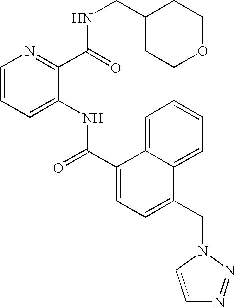

### Step A. N-[(tetrahydro-2H-pyran-4-yl)methyl]-3-[[[4-(1H-1,2,3-triazol-1-ylmethyl)-1-naphthalenyl]carbonyl]amino]-2-pyridinecarboxamide

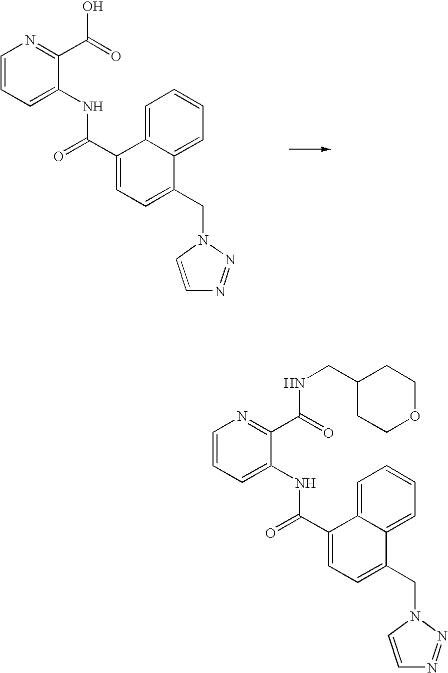

To a solution of 3-{[4-(1H-1,2,3-triazol-1-ylmethyl)-1-naphthoyl]amino}pyridine-2-carboxylic acid (20 mg, 0.054 mmol, see Step D for its preparation) and DIPEA (60 μL, 0.324 mmol) at room temperature in DMF (1 mL), was added HATU (14 mg, 0.12 mmol) in one portion. The solution was heated at 50° C. for 1 hour, cooled to room temperature and 4-tetrahydropyranmethylamine was added. The solution was heated at 50° C. for 30 minutes. After evaporation of the solvent, the residue was purified by reversed-phase HPLC (15-95% CH₃CN in H₂O) to provide the title compound as its TFA salt (8.18 mg, 32%). ¹H NMR (400 MHz, CDCl₃) δ 1.37-1.51 (m, 2H), 1.66-1.70 (m, J=12.69 Hz, 2H), 1.81-1.92 (m, 1H), 3.31 (t, J=6.64 Hz, 2H), 3.36-3.42 (m, 2H), 3.98-4.02 (m, 2H), 6.09 (s, 2H), 7.44-7.46 (m, 1H), 7.48 (d, J=7.22 Hz, 1H), 7.56 (dd, J=8.59, 4.49 Hz, 1H), 7.61-7.65 (m, 2H), 7.79 (s, 1H), 7.88 (d, J=7.23 Hz, 1H), 7.98-7.80 (m, 1H), 8.31 (dd, J=4.49, 1.37 Hz, 1H), 8.54-8.56 (m, 1H), 8.60-8.64 (m, 1H), 9.40 (dd, J=8.59, 1.56 Hz, 1H), 12.86 (s, 1H); MS (ESI) (M+H)⁺ 471.0; Anal. (C, H, N) calcd for C₂₆H₂₆N₆O₃+0.20CF₃COOH+0.20CH₃OH: C, 60.93; H, 6.26; N, 14.91. found C, 61.17; H, 5.69; N, 14.25.

### Step B. 4-(bromomethyl)-1-naphthoyl chloride

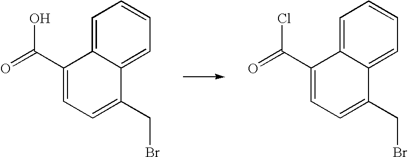

To a suspension of 4-(bromomethyl)-1-naphthoic acid (112 mg, 0.42 mmol) in CH₂Cl₂ (5 mL) at room temperature, was added oxalyl chloride (0.63 mL, 1.26 mmol) drop wise. The solution was stirred at room temperature for 10 minutes, and then heated at 50° C. for 30 minutes. The solution was concentrated, and the residue was used directly in Step C.

### Step C. 3-{[4-(bromomethyl)-1-naphthoyl]amino}pyridine-2-carboxylic acid

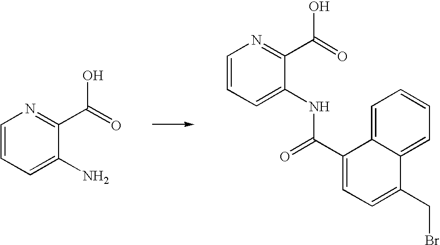

To a suspension of 3-aminopyridine-2-carboxylic acid (48.4 mg, 0.35 mmol) and DIPEA (0.12 mL, 0.7 mmol) in DMF (4.5 mL) at 0° C., was added 4-(bromomethyl)-1-naphthoyl chloride (119 mg, 0.42 mmol, see Step D for its preparation). The solution was stirred at room temperature overnight. The solution was concentrated, and the residue was used directly in Step D. MS (ESI) (M+H)⁺ 385.79.

### Step D. 3-{[4-(1H-1,2,3-triazol-1-ylmethyl)-1-naphthoyl]amino}pyridine-2-carboxylic acid

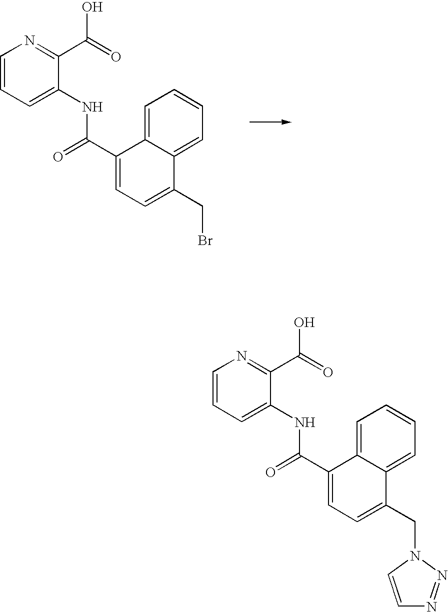

To a solution of 3-{[4-(bromomethyl)-1-naphthoyl]amino}pyridine-2-carboxylic acid (134.8 mg, 0.35 mmol, see Step C for its preparation) in DMF (1 mL) at room temperature, was added 1,2,3-triazole (200 mg, excess), in one portion. The solution was heated at 90° C. for 1 hour, concentrated and the residue was used directly in Step A. MS (ESI) (M+H)⁺ 373.94.

### Examples 129 & 130

### N-[(tetrahydro-2H-pyran-4-yl)methyl]-3-[[[4-(4H-1,2,4-triazol-4-ylmethyl)-1-naphthalenyl]carbonyl]amino]-2-pyridinecarboxamide

### and N-[(tetrahydro-2H-pyran-4-yl)methyl]-3-[[[4-(1H-1,2,4-triazol-1-ylmethyl)-1-naphthalenyl]carbonyl]amino]-2-pyridinecarboxamide

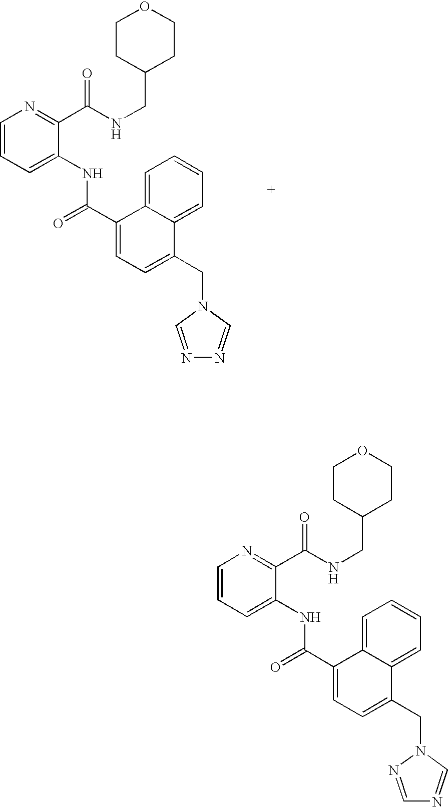

### Step A. N-[(tetrahydro-2H-pyran-4-yl)methyl]-3-[[[4-(4H-1,2,4-triazol-4-ylmethyl)-1-naphthalenyl]carbonyl]amino]-2-pyridinecarboxamide

### and N-[(tetrahydro-2H-pyran-4-yl)methyl]-3-[[[4-(1H-1,2,4-triazol-1-ylmethyl)-1-naphthalenyl]carbonyl]amino]-2-pyridinecarboxamide

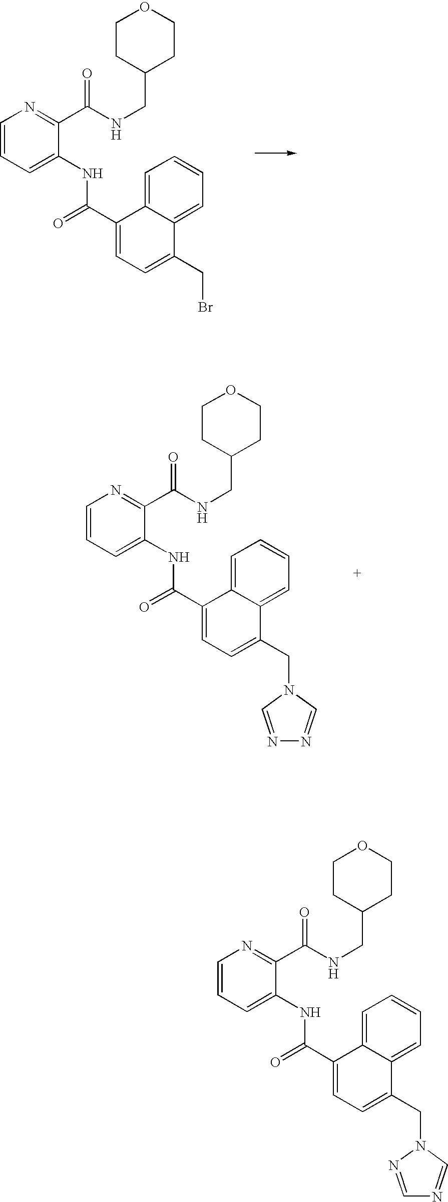

To a solution of 1,2,4-triazole (116.1 mg, 1.68 mmol) at room temperature in DMF (1 mL) was added 3-{[4-(bromomethyl)-1-naphthoyl]amino}-N-(tetrahydro-2H-pyran-4-ylmethyl)pyridine-2-carboxamide (100 mg, 0.21 mmol, see Step B for its preparation). The solution was heated at 90° C. for 30 minutes and cooled to room temperature. After evaporation of the solvent, the residue was purified by reversed-phase HPLC (20-50% CH₃CN in H₂O) to provide isomer 1 as its TFA salt (14.27 mg, 29%). ¹H NMR (400 MHz, CDCl₃) δ 1.33-1.44 (m, 2H), 1.66-1.69 (m, 2H), 1.80-1.92 (m, 1H), 3.31 (t, J=6.64 Hz, 2H), 3.35-3.41 (m, 2H), 3.99 (dd, J=11.33, 3.52 Hz, 2H), 5.77 (s, 2H), 7.40 (d, J=7.23 Hz, 1H), 7.56 (dd, J=8.59, 4.49 Hz, 1H), 7.68 (m, 2H), 7.86 (m, 2H), 8.31 (dd, J=4.49, 1.17 Hz, 1H), 8.51 (br. s., 1H), 8.61 (m, 2H), 9.39 (dd, J=8.59, 1.17 Hz, 1H), 12.93 (s, 1H); MS (ESI) (M+H)⁺ 471.0; Anal. (C, H, N) calcd for C₂₆H₂₆N₆O₃+1.50CF₃COOH+0.20H₂O: C, 53.99; H, 4.36; N, 13.03. found C, 53.94; H, 4.33; N, 12.99; and isomer 2 as its TFA salt (13.16 mg, 27%). ¹H NMR (400 MHz, CDCl₃) δ 1.33-1.44 (m, 2H), 1.63-1.69 (m, 2H), 1.80-1.92 (m, 1H), 3.31 (t, J=6.54 Hz, 2H), 3.35-3.41 (m, 2H), 3.97-4.01 (m, 2H), 5.89 (s, 2H), 7.46 (d, J=7.23 Hz, 1H), 7.55 (dd, J=8.59, 4.49 Hz, 1H), 7.61-7.66 (m, 2H), 7.89 (d, J=7.23 Hz, 1H), 7.96-7.99 (m, 1H), 8.14 (s, 1H), 8.19 (s, 1H), 8.30 (dd, J=4.49, 1.17 Hz, 1H), 8.58 (m, 2H), 9.40 (dd, J=8.59, 1.17 Hz, 1H), 12.88 (s, 1H); MS (ESI) (M+H)⁺ 471.0; Anal. (C, H, N) calcd for C₂₆H₂₆N₆O₃+0.20CH₃CN+1.60CF₃COOH+0.10H₂O: C, 53.63; H, 4.32; N, 13.10. found C, 53.56; H, 4.28; N, 13.14.

### Step B. 3-{[4-(bromomethyl)-1-naphthoyl]amino}-N-(tetrahydro-2H-pyran-4-ylmethyl)pyridine-2-carboxamide

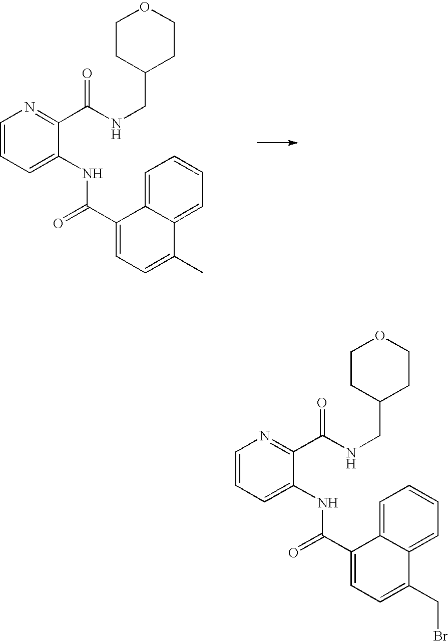

To a solution of 3-[(4-methyl-1-naphthoyl)amino]-N-(tetrahydro-2H-pyran-4-ylmethyl)pyridine-2-carboxamide (400 mg, 0.99 mmol) and NBS (356 mg, 2 mmol) in 1,2-C₂H₂Cl₂ (20 mL) at room temperature, was added 1,1′-azobis(cyclohexanecarbonitrile) (15 mg, 0.06 mmol), in one portion. The solution was heated at 80° C. for 2.5 hours, then cooled to room temperature, concentrated and the residue was used directly in Step A. MS (ESI) (M+H)⁺ 483.87.

### Example 131

### 3-[[[4-(1-pyrrolidinylmethyl)-1-naphthalenyl]carbonyl]amino]-N-[(tetrahydro-2H-pyran-4-yl)methyl]-2-pyridinecarboxamide

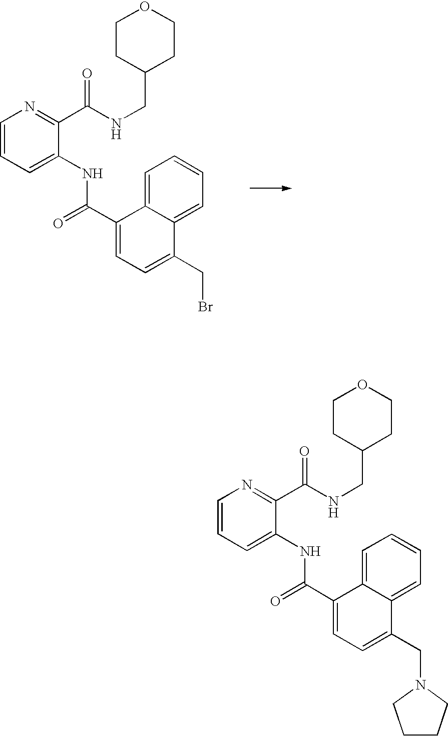

Following the procedure for Step A in Example 129/130, using 3-{[4-(bromomethyl)-1-naphthoyl]amino}-N-(tetrahydro-2H-pyran-4-ylmethyl)pyridine-2-carboxamide (100 mg, 0.21 mmol), and pyrrolidine (0.14 mL, 2.16 mmol) provided the title compound as its TFA salt (13.9 mg, 14%) following purification by reversed-phase HPLC (15-95% CH₃CN in H₂O). ¹H NMR (400 MHz, CDCl₃) δ 1.33-1.44 (m, 2H), 1.68 (d, J=12.89 Hz, 2H), 1.83-1.91 (m, 1H), 2.04-2.18 (m, 4H), 2.88-3.00 (m, 2H), 3.31 (t, J=6.64 Hz, 2H), 3.35-3.41 (m, 2H), 3.72-3.86 (m, 2H), 3.99 (dd, J=111.23, 3.42 Hz, 2H), 4.83 (s, 2H), 7.56 (dd, J=8.59, 4.49 Hz, 1H), 7.64-7.72 (m, 2H), 7.76 (d, J=7.23 Hz, 1H), 7.90 (d, J=7.42 Hz, 1H), 8.17 (d, J=8.01 Hz, 1H), 8.31 (dd, J=4.49, 1.17 Hz, 1H), 8.55-8.62 (m, 2H), 9.39 (dd, J=8.59, 1.17 Hz, 1H), 12.90 (s, 1H); MS (ESI) (M+H)⁺ 473.2. Anal. (C, H, N) calcd for C₂₈H₃₂N₄O₃+1.70CF₃COOH: C, 56.59; H, 5.10; N, 8.41. found C, 56.67; H, 5.14; N, 8.43.

### Example 132

### 3-[[[4-(1H-pyrazol-1-ylmethyl)-1-naphthalenyl]carbonyl]amino]-N-[(tetrahydro-2H-pyran-4-yl)methyl]-2-pyridinecarboxamide

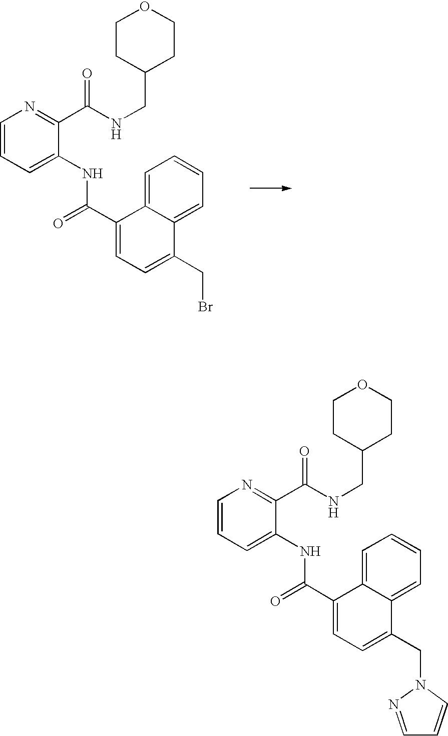

Following the procedure for Step A in Example 129/130, using 3-{[4-(bromomethyl)-1-naphthoyl]amino}-N-(tetrahydro-2H-pyran-4-ylmethyl)pyridine-2-carboxamide (100 mg, 0.21 mmol), and pyrazole (114.4 mg, 1.68 mmol) provided the title compound as its TFA is salt (25.9 mg, 26%) following purification by reversed-phase HPLC (30-60% CH₃CN in H₂O). ¹H NMR (400 MHz, CDCl₃) δ 1.32-1.43 (m, 2H), 1.65-1.69 (m, 2H), 1.81-1.90 (m, 1H), 3.30 (t, J=6.64 Hz, 2H), 3.35-3.40 (m, 2H), 3.98 (dd, J=11.33, 3.52 Hz, 2H), 5.85 (s, 2H), 6.29-6.30 (m, 1H), 7.24 (d, J=7.42 Hz, 1H), 7.34 (d, J=2.15 Hz, 1H), 7.54 (dd, J=8.49, 4.49 Hz, 1H), 7.58-7.62 (m, 2H), 7.85 (d, J=7.42 Hz, 1H), 8.02-8.05 (m, 1H), 8.28 (dd, J=4.49, 1.27 Hz, 1H), 8.56-8.59 (m, 2H), 9.40 (dd, J=8.49, 1.27 Hz, 1H), 12.82 (s, 1H); MS (ESI) (M+H)⁺ 470.0.

### Example 133

### N-[(tetrahydro-2H-pyran-4-yl)methyl]-3-[[[4-(2H-tetrazol-2-ylmethyl)-1-naphthalenyl]carbonyl]amino]-2-pyridinecarboxamide

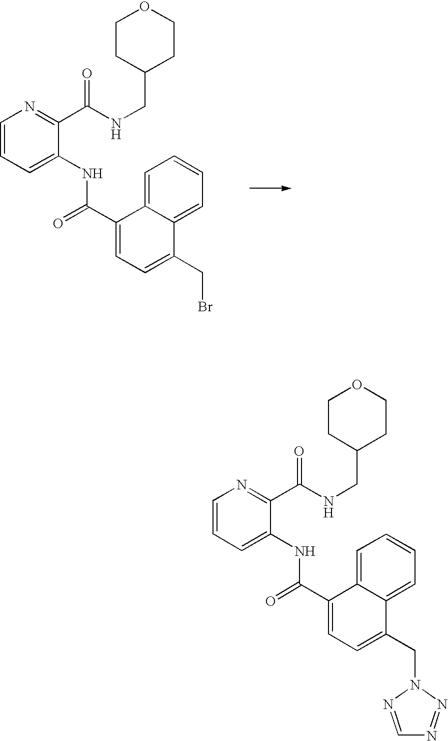

Following the procedure for Step A in Example 129/130, using 3-{[4-(bromomethyl)-1-naphthoyl]amino}-N-(tetrahydro-2H-pyran-4-ylmethyl)pyridine-2-carboxamide (100 mg, 0.21 mmol), and tetrazole (117.7 mg, 1.68 mmol) provided the title compound as its TFA salt (17.4 mg, 3%) following purification by reversed-phase HPLC (40-95% CH₃CN in H₂O). ¹H NMR (400 MHz, CDCl₃) δ 1.38 (m, 1H) 1.67 (m, J=12.89 Hz, 3H) 1.86 (m, 1H) 3.31 (t, J=6.64 Hz, 2H) 3.37 (m, 2H) 3.98 (dd, J=11.42, 3.42 Hz, 1H) 6.10 (s, 2H) 7.52 (d, J=7.42 Hz, 1H) 7.56 (dd, J=8.59, 4.49 Hz, 1H) 7.64 (m, 2H) 7.90 (d, J=7.23 Hz, 1H) 7.93 (m, 1H) 8.31 (dd, J=4.49, 1.37 Hz, 1H) 8.42 (s, 1H) 8.59 (m, 2H) 9.39 (dd, J=8.59, 1.17 Hz, 1H) 12.91 (s, 1H); MS (ESI) (M+H)⁺ 472.0; Anal. (C, H₁₁N) calcd for C₂₅H₂₅N₇O₃+0.30CH₃CN+0.10CF₃COOH: C, 62.18; H, 5.38; N, 19.91. found C, 62.20; H, 5.29; N, 19.74.

### Example 134

### N-(Tetrahydro-2H-pyran-4-yl)-3-[[[4-(1H-1,2,3-triazol-1-ylmethyl)-1-naphthalenyl]carbonyl]amino]-2-pyridinecarboxamide

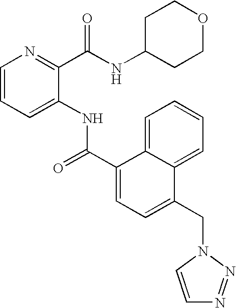

### Step A. N-(Tetrahydro-2H-pyran-4-yl)-3-[[[4-(1H-1,2,3-triazol-1-ylmethyl)-1-naphthalenyl]carbonyl]amino]-2-pyridinecarboxamide

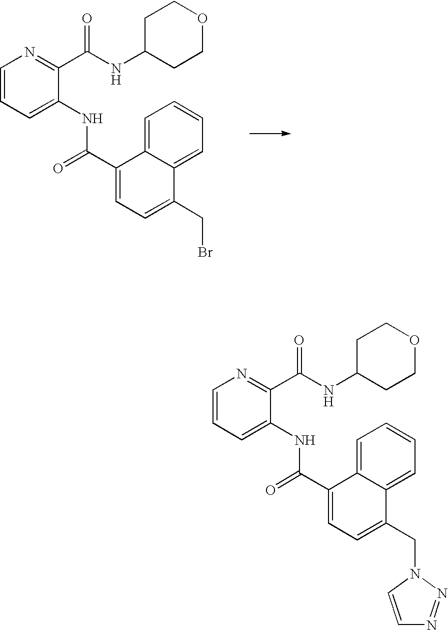

To a solution of 3-{[4-(bromomethyl)-1-naphthoyl]amino}-N-(tetrahydro-2H-pyran-4-yl)pyridine-2-carboxamide (100 mg, 0.214 mmol, see Step B for its preparation) at room temperature in DMF (1.07 mL) was added 1,2,3-triazole (0.1 mL, 1.712 mmol). The solution was heated at 90° C. for 30 minutes and cooled to room temperature. After evaporation of the solvent, the residue was purified by reversed-phase HPLC (30-90% CH₃CN in H₂O) to provide the title compound as its TFA salt (18.6 mg, 19%). ¹H NMR (400 MHz, CDCl₃) δ 1.63-1.73 (m, 2H), 1.82-1.83 (m, 2H), 3.44-3.50 (m, 2H), 3.92-3.95 (m, 2H), 3.98-4.04 (m, 1H), 6.20 (s, 2H), 7.45 (d, J=7.42 Hz, 1H), 7.60-7.66 (m, 3H), 7.73-7.79 (br.s, 1H), 7.88 (br.s, 1H), 7.94-8.03 (m, 1H), 8.22-8.24 (m, 1H), 8.37-8.38 (m, 1H), 8.47-8.49 (m, 1H), 9.26-9.28(m, 1H); MS (ESI) (M+H)⁺ 457.0; Anal. (C, H, N) calcd for C₂₅H₂₄N₆O₃+0.20CF₃COOH: C, 63.65; H, 5.09; N, 17.53. found C, 63.60; H, 5.11; N, 17.37.

### Step B. 3-{[4-(bromomethyl)-1-naphthoyl]amino}-N-(tetrahydro-2H-pyran-4-yl)pyridine-2-carboxamide

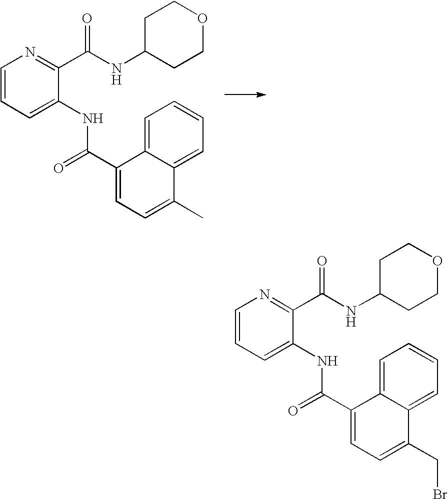

To a solution of 3-[(4-methyl-1-naphthoyl)amino]-N-(tetrahydro-2H-pyran-4-yl)pyridine-2-carboxamide (410 mg, 1.05 mmol) and NBS (374 mg, 2.1 mmol) in 1,2-C₂H₂Cl₂ (21 mL) at room temperature, was added 1,1′-azobis(cyclohexanecarbonitrile) (15 mg, 0.06 mmol), in one portion. The solution was heated at 90° C. for 2 hours, and then cooled to room temperature. The solution was concentrated, and the residue was used directly in Step A. MS (ESI) (M+H)⁺ 469.88.

### Example 135

### 3-[[[4-(1H-imidazol-1-ylmethyl)-1-naphthalenyl]carbonyl]amino]-N-(tetrahydro-2H-pyran-4-yl)-2-pyridinecarboxamide

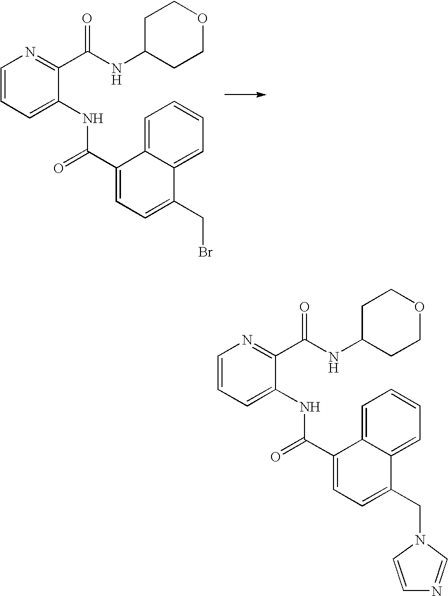

Following the procedure for Step A in Example 134, using 3-{[4-(bromomethyl)-1-naphthoyl]amino}-N-(tetrahydro-2H-pyran-4-ylmethyl)pyridine-2-carboxamide (100 mg, 0.21 mmol), and imidazole (116 mg, 1.71 mmol) provided the title compound as its TFA salt (30.8 mg, 25%) following purification by reversed-phase HPLC (10-90% CH₃CN in H₂S). ¹H NMR (400 MHz, CDCl₃) δ 1.64-1.74 (m, 2H), 1.82-1.85 (m, 2H), 3.43-3.49 (m, 2H), 3.93-3.96 (m, 2H), 3.96-4.04 (m, 1H), 6.04 (s, 2H), 7.58-7.63 (m, 3H), 7.66-7.72 (m, 3H), 7.93 (d, J=7.42 Hz, 1H), 8.13-8.15 (m, 1H), 8.39 (d, J=3.51 Hz, 1H), 8.49-8.53 (m, 1H), 9.05 (s, 1H), 9.28 (dd, J=8.59, 0.98 Hz, 1H); MS (ESI) (M+H)⁺ 456.0; Anal. (C, H, N) calcd for C₂₆H₂₅N₅O₃+1.40CF₃COOH+0.20H₂O: C, 55.91; H, 4.37; N, 11.32. found C, 55.89; H, 4.24; N, 11.25.

### Example 136

### 3-[[[4-(1H-pyrazol-1-ylmethyl)-1-naphthalenyl]carbonyl]amino]-N-(tetrahydro-2H-pyran-4-yl)-2-pyridinecarboxamide

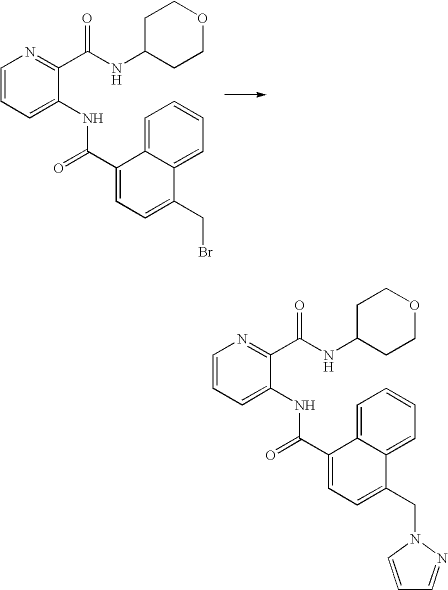

Following the procedure for Step A in Example 134, using 3-{[4-(bromomethyl)-1-naphthoyl]amino}-N-(tetrahydro-2H-pyran-4-ylmethyl)pyridine-2-carboxamide (100 mg, 0.21 mmol), and pyrazole (116 mg, 1.71 mmol) provided the title compound as its TFA salt (20.5 mg, 21%) following purification by reversed-phase HPLC (30-90% CH₃CN in H₂O). ¹H NMR (400 MHz, CDCl₃) δ 1.60-1.70 (m, 2H), 1.93-1.96 (m, 2H), 3.48-3.54 (m, 2H), 3.99-4.02 (m, 2H), 4.04-4.12 (m, 1H), 5.89 (s, 2H), 6.32-6.33 (m, 1H), 7.28-7.32 (m, 2H), 7.55 (dd, J=8.59, 4.49 Hz, 1H), 7.58-7.63 (m, 2H), 7.69-7.69 (m, 1H), 7.85 (d, J=7.23 Hz, 1H), 7.98-8.01 (m, 1H), 8.30 (dd, J=4.49, 1.17 Hz, 1H), 8.41-8.43 (m, 1H), 8.55-8.58 (m, 1H), 9.40 (dd, J=8.59, 1.17 Hz, 1H), 12.81 (br.s, 1H); MS (ESI) (M+H)⁺ 456.0; Anal. (C, H, N) calcd for C₂₆H₂₅N₅O₃+0.50CF₃COOH+0.50CH₃CN+0.10CH₃OH: C, 62.94; H, 5.15; N, 14.37. found C, 62.89; H, 4.89; N, 14.45.

### Example 137

### 3-[[[4-(methoxymethyl)-1-naphthalenyl]carbonyl]amino]-N-[(tetrahydro-2H-pyran-4-yl)methyl]-2-pyridinecarboxamide

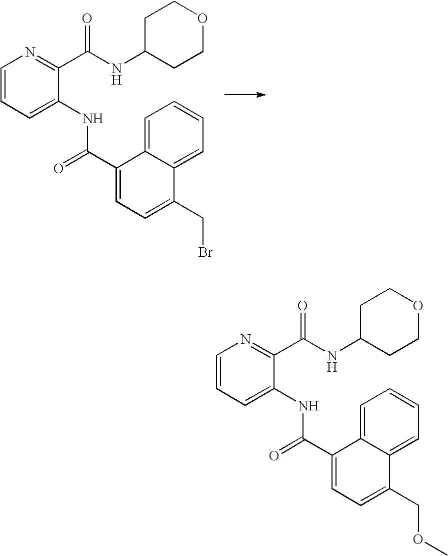

Following the procedure for Step A in Example 134, using 3-{[4-(bromomethyl)-1-naphthoyl]amino}-N-(tetrahydro-2H-pyran-4-ylmethyl)pyridine-2-carboxamide (100 mg, 0.21 mmol), methanol (3 mL, 0.07 mmol) and NaOMe (1 mL, excess, 25-30% solution in MeOH) provided the title compound as its TFA salt (16 mg, 14%) following purification by reversed-phase HPLC (30-90% CH₃CN in H₂O). ¹H NMR (400 MHz, CDCl₃) δ 1.50 (t, J=7.52 Hz, 3H), 1.61-1.71 (m, 2H), 1.92-1.95 (m, 2H), 3.16 (q, J=7.42 Hz, 2H), 3.47-3.53 (m, 2H), 3.98-4.01 (m, 2H), 4.03-4.12 (m, 1H), 5.70 (s, 2H), 6.88 (d, J=1.17 Hz, 1H), 7.05 (d, J=7.23 Hz, 1H), 7.43 (d, J=11.17 Hz, 1H), 7.57 (dd, J=8.59, 4.49 Hz, 1H), 7.69-7.71 (m, 2H), 7.79-7.81 (m, 1H), 7.85-7.87 (m, 1H), 8.33 (dd, J=4.49, 1.27 Hz, 1H), 8.44-8.46 (m, 1H), 8.61-8.63 (m, 1H), 9.38 (dd, J=8.59, 1.27 Hz, 1H), 12.90 (s, 1H); MS (ESI) (M+H)⁺ 484.0; Anal. (C, H, N) calcd for C₂H₂₉N₅O₃+1.60CF₃COOH+0.10H₂O: C, 56.12; H, 4.65; N, 10.49. found C, 56.10; H, 4.70; N, 10.45.

### Example 138

### 3-[[[4-(methoxymethyl)-1-naphthalenyl]carbonyl]amino]-N-[(tetrahydro-2H-pyran-4-yl)methyl]-2-pyridinecarboxamide

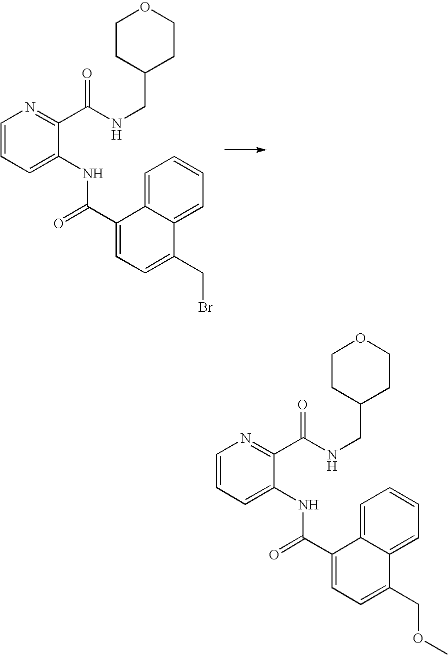

Following the procedure for Step A in Example 134, using 3-{[4-(bromomethyl)-1-naphthoyl]amino}-N-(tetrahydro-2H-pyran-4-ylmethyl)pyridine-2-carboxamide (100 mg, 0.21 mmol), methanol (3 mL, 0.07 mmol) and NaOMe (1 mL, excess, 25-30% solution in MeOH) provided the title compound as its TFA salt (32.2 mg, 28%) following purification by reversed-phase HPLC (30-90% CH₃CN in H₂O). ¹H NMR (400 MHz, CDCl₃) δ 1.37 (m, 2H) 1.66 (m, J=12.89, 1.76 Hz, 2H) 1.84 (m, 1H) 3.35 (m, 4H) 3.48 (s, 3H) 3.98 (m, 2H) 4.95 (s, 2H) 7.53 (dd, J=8.59, 4.49 Hz, 1H) 7.59 (m, 3H) 7.87 (d, J=7.22 Hz, 1H) 8.14 (m, 1H) 8.27 (dd, J=4.49, 1.37 Hz, 1H) 8.56 (m, 2H) 9.41 (dd, J=8.59, 1.37 Hz, 1H) 12.79 (s, 1H); MS (ESI) (M+H)⁺ 434.0; Anal. (C, H, N) calcd for C₂₅H₂₇N₃O₄+0.20CH₃CN: C, 69.07; H, 6.30; N, 10.15. found C, 69.16; H, 6.39; N, 10.25.

### Example 139

### 3-[(4-benzyl-1-naphthoyl)amino]-N-(tetrahydro-2H-pyran-4-ylmethyl)pyridine-2-carboxamide

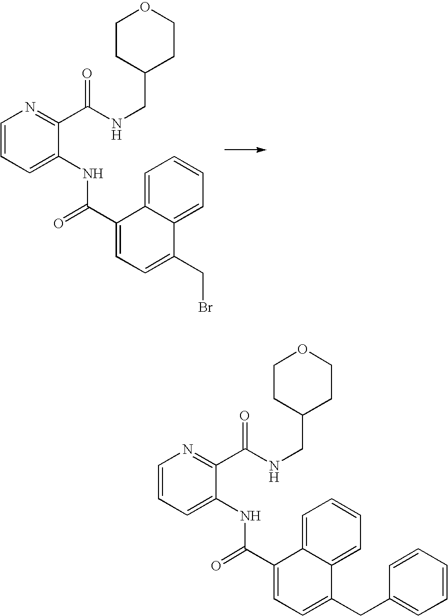

To a solution of 3-{[4-(bromomethyl)-1-naphthoyl]amino}-N-(tetrahydro-2H-pyran-4-ylmethyl)pyridine-2-carboxamide (150 mg, 0.31 mmol) and phenyl boronic acid (61 mg, 0.5 mmol) in THF (4 mL) was added 2M Na₂CO₃ aq (0.39 mL, 0.78 mmol) at room temperature. The solution was degassed by bubbling N₂ through it for 20 minutes and tetrakis(triphenylphosphine)palladium (35.8 mg, 0.031 mmol) was added in one portion at room temperature. The suspension was heated at reflux for 4 hours and cooled to room temperature. After evaporation of the solvent, the residue was purified by MPLC (0-100% EtOAc in Hexanes) followed by reversed-phase HPLC (30-95% CH₃CN in H₂O) to provide the title compound as its TFA salt (27.6 mg, 15%). ¹H NMR (400 MHz, CDCl₃) δ 1.33-1.43 (m, 2H), 1.65-1.69 (m, 2H), 1.81-1.92 (m, 1H), 3.31 (t, J=6.64 Hz, 2H), 3.34-3.41 (m, 2H), 3.96-3.40 (m, 2H), 4.49 (s, 2H), 7.20-7.24 (m, 3H), 7.28-7.35 (m, 3H), 7.49-7.57 (m, 3H), 7.84 (d, J=7.42 Hz, 1H), 8.06-8.08 (m, 1H), 8.27 (dd, J=4.49, 1.37 Hz, 1H), 8.57-8.59 (m, 2H), 9.42 (dd, J=8.59, 1.37 Hz, 1H), 12.77 (br.s, 1H); MS (ESI) (M+H)⁺ 480.0; Anal. (C, H, N) calcd for C₃₀H₂₉N₃O₃+0.10CH₃OH+0.20H₂O: C, 74.33; H, 6.18; N, 8.64. found C, 74.43; H, 6.03; N, 8.63.

### Example 140

### 3-[[[4-(3-furanylmethyl)-1-naphthalenyl]carbonyl]amino]-N-[(tetrahydro-2H-pyran-4-yl)methyl]-2-pyridinecarboxamide

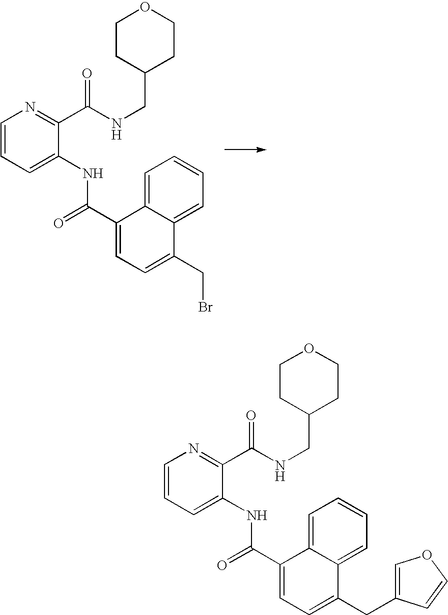

To a solution of 3-{[4-(bromomethyl)-1-naphthoyl]amino}-N-(tetrahydro-2H-pyran-4-ylmethyl)pyridine-2-carboxamide (100 mg, 0.21 mmol), and 3-furanboronic acid (37.6 mL, 0.34 mmol) in DME (2.8 mL) was added 2M Na₂CO₃ aq (0.27 mL, 0.53 mmol) at room temperature. The solution was degassed by bubbling N₂ through it for 20 minutes and tetrakis(triphenylphosphine)palladium (24.3 mg, 0.021 mmol) was added in one portion at room temperature. The suspension was heated at reflux for 3.5 hours and cooled to room temperature. After evaporation of the solvent, the residue was redissolved in CH₂Cl₂. Extraction with CH₂Cl₂ (2×), washing with brine (1×), drying (Na₂SO₄), filtration and concentration of the solvent provided the title compound as its TFA salt (25.7 mg, 21%) following purification by reversed-phase HPLC (40-90% CH₃CN in H₂O). ¹H NMR (400 MHz, CDCl₃) δ 1.38 (m, 2H), 1.67 (m, 2H), 1.86 (m, 1H) 3.31 (m, 2H), 3.37 (m, 2H), 3.98 (m, 2H), 4.27 (s, 2H), 6.29 (s, 1H), 7.18 (s, 1H), 7.41 (m, 2H), 7.55 (m, 3H), 7.84 (d, J=7.22 Hz, 1H), 8.11 (m, 1H), 8.27 (dd, J=4.59, 1.27 Hz, 1H), 8.58 (m, 2H), 9.41 (dd, J=8.59, 1.27 Hz, 1H), 12.77 (br.s, 1H); MS (ESI) (M+H)⁺ 470.0.

### Example 141

### 3-[[[4-(2-furanylmethyl)-1-naphthalenyl]carbonyl]amino]-N-[(tetrahydro-2H-pyran-4-yl)methyl]-2-pyridinecarboxamide

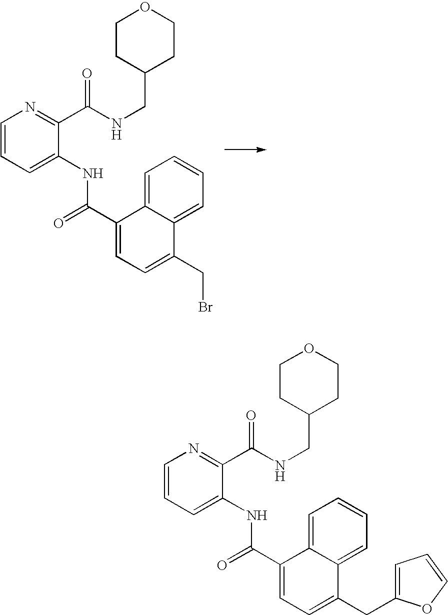

Following the procedure in Example 140, using 3-{[4-(bromomethyl)-1-naphthoyl]amino}-N-(tetrahydro-2H-pyran-4-ylmethyl)pyridine-2-carboxamide (100 mg, 0.21 mmol), 2-furanylboronic acid (37.6 mg, 0.34 mmol), toluene (2.8 mL) and ethanol (0.56 mL) instead of DME, 2M Na₂CO₃ aq (0.27 mL, 0.53 mmol) and tetrakis(triphenylphosphine)palladium (24.3 mg, 0.021 mmol) provided the title compound as its TFA salt (33.1 mg, 27%) following purification by reversed-phase HPLC (40-90% CH₃CN in H₂O). ¹H NMR (400 MHz, CDCl₃) δ 1.67 (m, 4H), 1.85 (m, 1H), 3.31 (m, 2H), 3.38 (m, 2H), 3.98 (m, 2H), 4.47 (s, 2H), 5.98 (m, 1H), 6.30 (m, 1H), 7.36 (m, 1H), 7.40 (d, J=7.23 Hz, 1H), 7.53 (dd, J=8.69, 4.59 Hz, 1H), 7.57 (m, 2H), 7.84 (d, J=7.23 Hz, 1H), 8.11 (m, 1H), 8.27 (dd, J=4.39, 1.46 Hz, 1H), 9.41 (dd, J=8.69, 1.27 Hz, 1H), 12.77 (s, 1H); MS (ESI) (M+H)⁺ 470.0; Anal. (C, H, N) calcd for C₂₈H₂₇N₃O₄+0.20CH₃CN+0.20CF₃COOH: C, 61.11; H, 5.60; N, 8.95. found C, 69.20; H, 5.68; N, 9.00.

### Example 142

### N-[(tetrahydro-2H-pyran-4-yl)methyl]-3-[[[4-(2-thienylmethyl)-1-naphthalenyl]carbonyl]amino]-2-pyridinecarboxamide

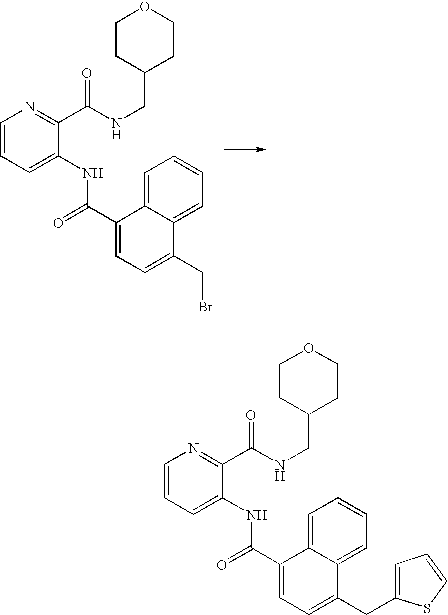

Following the procedure in Example 140, using 3-{[4-(bromomethyl)-1-naphthoyl]amino}-N-(tetrahydro-2H-pyran-4-ylmethyl)pyridine-2-carboxamide (100 mg, 0.21 mmol), 2-thiopheneboronic acid (43.5 mg, 0.34 mmol), 2M Na₂CO₃ aq (0.27 mL, 0.53 mmol) and tetrakis(triphenylphosphine)palladium (24.3 mg, 0.021 mmol) provided the title compound as its TFA salt (13.9 mg, 11%) following purification by reversed-phase HPLC (30-90% CH₃CN in H₂O). ¹H NMR (400 MHz, CDCl₃) δ 1.38 (m, 2H), 1.64 (m, 2H), 1.86 (m, 1H), 3.31 (m, 2H), 3.37 (m, 2H), 3.98 (m, 2H), 4.65 (s, 2H), 6.78 (dd, J=3.51, 1.17 Hz, 1H), 6.92 (dd, J=5.08, 3.51 Hz, 1H), 7.16 (dd, J=5.08, 1.17 Hz, 1H), 7.45 (d, J=7.42 Hz, 1H), 7.53 (dd, J=8.59, 4.49 Hz, 1H), 7.57 (m, 2H), 7.85 (d, J=7.42 Hz, 1H), 8.12 (m, 1H), 8.27 (dd, J=4.49, 1.37 Hz, 1H), 8.58 (m, 2H), 9.41 (dd, J=8.59, 1.37 Hz, 1H), 12.78 (s, 1H); MS (ESI) (M+H)⁺ 486.0; Anal. (C, H, N) calcd for C₂₈H₂₇N₃O₃S+0.10CF₃COOH+0.30H₂O: C, 67.42; H, 5.56; N, 8.36. found C, 67.40; H, 5.39; N, 8.42.

### Example 143

### N-[(tetrahydro-2H-pyran-4-yl)methyl]-3-[[[4-(3-thienylmethyl)-1-naphthalenyl]carbonyl]amino]-2-pyridinecarboxamide

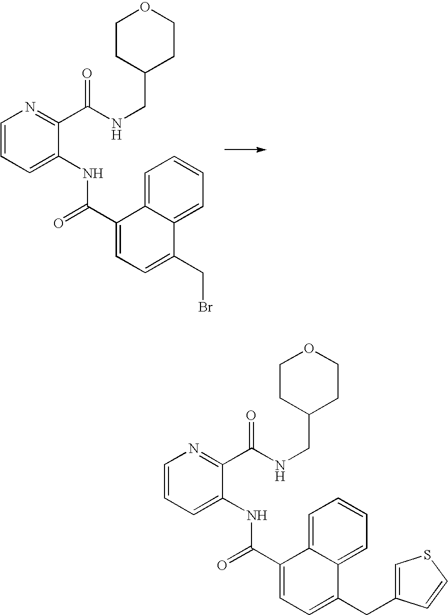

Following the procedure in Example 140, using 3-{[4-(bromomethyl)-1-naphthoyl]amino}-N-(tetrahydro-2H-pyran-4-ylmethyl)pyridine-2-carboxamide (100 mg, 0.21 mmol), 3-thiopheneboronic acid (43.5 mg, 0.34 mmol), 2M Na₂CO₃ aq (0.27 mL, 0.53 mmol) and tetrakis(triphenylphosphine)palladium (24.3 mg, 0.021 mmol) provided the title compound as its TFA salt (22.7 mg, 18%) following purification by reversed-phase HPLC (50-90% CH₃CN in H₂O). ¹H NMR (400 MHz, CDCl₃) δ 1.34-1.43 (m, 2H), 1.65-1.69 (m, J=13.47, 2.54 Hz, 2H), 1.80-1.92 (m, 1H), 3.30-3.33 (m, 2H), 3.35-3.41 (m, 2H), 3.95-4.01 (m, 2H), 4.48 (s, 2H), 6.90-6.91 (m, 1H), 6.97 (dd, J=4.98, 1.27 Hz, 1H), 7.28 (dd, J=4.98, 2.93 Hz, 1H), 7.38 (d, J=7.23 Hz, 1H), 7.51-7.58 (m, 3H), 7.84 (d, J=7.42 Hz, 1H), 8.07-8.09 (m, 1H), 8.27 (dd, J=4.49, 1.37 Hz, 1H), 8.57-8.59 (m, 2H), 9.42 (dd, J=8.59, 1.37 Hz, 1H), 12.77 (s, 1H); MS (ESI) (M+H)⁺ 486.0; Anal. (C, H, N) calcd for C₂₈H₂₇N₃O₃S+0.20CF₃COOH+0.10CH₃CN+0.10CH₃OH: C, 66.84; H, 5.45; N, 8.42. found C, 66.90; H, 5.26; N, 8.41.

### Example 144

### N-(2-methylcyclohexyl)-3-[(1-naphthalenylcarbonyl)amino]-2-pyridinecarboxamide

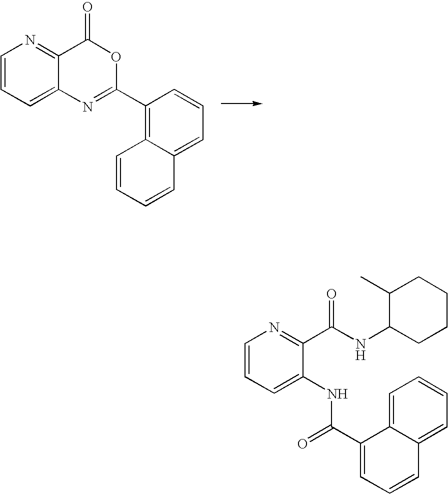

Following the procedure for Step A in Example 1, using 2-(1-naphthalenyl)-4H-pyrido[3,2-d][1,3]oxazin-4-one (100 mg, 0.36 mmol), and 2-methylcyclohexylamine (0.30 mL, 2.16 mmol) provided the title compound as its TFA salt (19.8 mg, 11%) following purification by reversed-phase HPLC (45-95% CH₃CN in H₂O). ¹H NMR (400 MHz, CDCl₃) δ 0.90 (d, J=6.44 Hz, 3H), 1.05-1.14 (m, 1H), 1.27-1.41 (m, 3H), 1.48-1.57 (m, 1H), 1.66-1.90 (m, 4H), 3.44-3.50 (m, 1H), 7.55-7.63 (m, 4H), 7.90-7.92 (m, 1H), 7.95-7.98 (m, 1H), 8.06-8.08 (m, 1H), 8.38 (dd, J=4.49, 1.37 Hz, 1H), 8.43-8.45 (m, 1H), 9.31 (dd, J=8.59, 1.37 Hz, 1H); MS (ESI) (M+H)⁺ 388.0; Anal. (C, H, N) calcd for C₂₄H₂₅N₃O₂+0.20CH₃OH: C, 73.79; H, 6.60; N, 10.67. found C, 73.86; H, 6.53; N, 10.61.

### Example 145

### 3-[(1-naphthalenylcarbonyl)amino]-N-[2-(1-pyrrolidinyl)ethyl]-2-pyridinecarboxamide

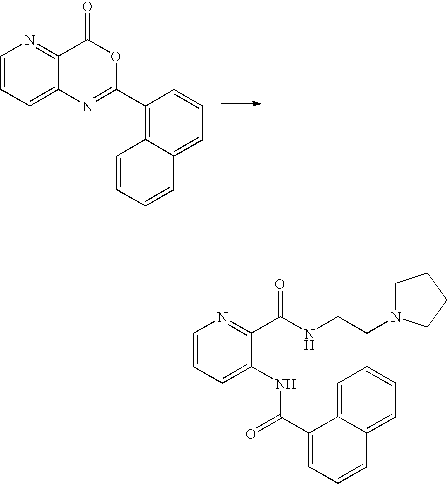

Following the procedure for Step A in Example 1, using 2-(1-naphthalenyl)-4H-pyrido[3,2-d][1,3]oxazin-4-one (100 mg, 0.36 mmol), and 1-(2-aminoethyl)pyrrolidine (0.30 mL, 2.16 mmol) provided the title compound as its TFA salt (26.3 mg, 15%) following purification by reversed-phase HPLC (20-50% CH₃CN in H₂O). ¹H NMR (400 MHz, CDCl₃) δ 1.82-1.94 (m, 2H), 2.01-2.12 (m, 2H), 3.01-3.11 (m, 2H), 3.39 (t, J=5.86 Hz, 2H), 3.67-3.74 (m, 4H), 7.54-7.60 (m, 3H), 7.63 (dd, J=8.59, 4.49 Hz, 1H), 7.88-7.90 (m, 1H), 7.93-7.98 (m, 1H), 8.06-8.08 (m, 1H), 8.39 (dd, J=4.49, 1.37 Hz, 1H), 8.40-8.43 (m, 1H), 9.24 (dd, J=8.59, 1.37 Hz, 1H); MS (ESI) (M+H)⁺ 389.0; Anal. (C, H, N) calcd for C₂₃H₂₄N₄O₂+1.50CF₃COOH+0.20H₂O: C, 55.46; H, 4.64; N, 9.95. found C, 55.43; H, 4.62; N, 9.91.

### Example 146

### N-(cyclobutylmethyl)-3-[[2-(4-morpholinyl)benzoyl]amino]-2-pyridinecarboxamide

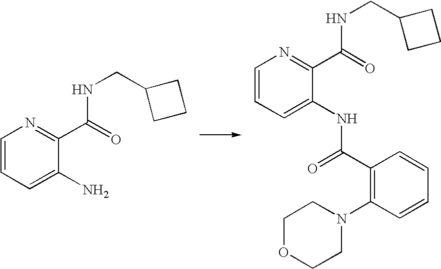

To a solution of 3-amino-N-(cyclobutylmethyl)pyridine-2-carboxamide (100 mg, 0.49 mmol), DIPEA (0.17 mL, 0.97 mmol) and 2-morpholino benzoic acid (203 mg, 0.97 mmol) at room temperature in DMF (1.6 mL), was added HATU (369 mg, 0.97 mmol) in one portion at room temperature. The solution was heated at 100° C. over night. After evaporation of the solvent, the residue was purified by reversed-phase HPLC (30-95% CH₃CN in H₂O) to provide the title compound as its TFA salt (50.8 mg, 20%). ¹H NMR (400 MHz, DMSO-D₆) δ 1.66-1.83 (m, 4H), 1.91-1.99 (m, 2H), 2.58-2.52 (m, 1H), 2.95-2.97 (m, 4H), 3.29-3.32 (m, 2H), 3.61-3.63 (m, 4H), 7.13-7.19 (m, 2H), 7.48-7.53 (m, 1H), 7.62-7.66 (m, 2H), 8.35 (dd, J=4.49, 1.56 Hz, 1H), 9.09-9.12 (m, 1H), 9.19-9.21 (m, 1H), 13.02 (s, 1H); MS (ESI) (M+H)⁺ 395.2; Anal. (C, H, N) calcd for C₂₂H₂₆N₄O₃+0.10H₂O: C, 66.68; H, 6.66; N, 14.14. found C, 66.60; H, 6.74; N, 14.10.

### Example 147

### N-(Tetrahydro-2H-pyran-4-ylmethyl)-3-({4-[(3H-[1,2,3]triazolo[4,5-b]pyridin-3-yloxy)methyl]-1-naphthoyl}amino)pyridine-2-carboxamide

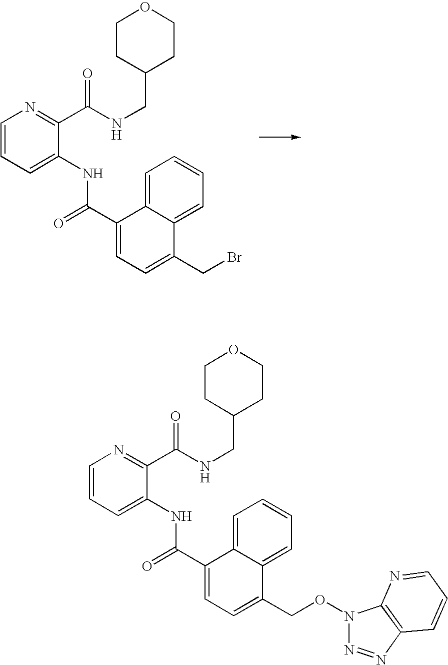

Following the procedure for Step A in Example 129/130, using 3-{[4-(bromomethyl)-1-naphthoyl]amino}-N-(tetrahydro-2H-pyran-4-ylmethyl)pyridine-2-carboxamide (47 mg, 0.1 mmol), and 3H-[1,2,3]triazolo[4,5-b]pyridin-3-ol (136 mg, 1.0 mmol) provided the title compound (25 mg, 38%). ¹H NMR (400 MHz, CDCl₃) δ 1.40 (m, 2H), 1.67 (m, 2H), 1.87(m, 1H), 3.31 (m, 2H), 3.40(m, 2H), 3.99(m, 2H), 6.13 (s, 2H), 7.45 (dd, J=8.0, 4.0 Hz, 1H), 7.56 (dd, J=8.0, 4.0 Hz, 1H), 7.67 (t, J=8.0 Hz, 1H), 7.70 (d, J=8.0 Hz, 1H), 7.77 (t, J=8.0 Hz, 1H), 7.81 (d, J=8.0 Hz, 1H), 8.31 (d, J=4.0 Hz, 1H), 8.42 (d, J=8.0 Hz, 1H), 8.54 (d, J=8.0 Hz, 1H), 8.60 (m, 1H), 8.63 (d, J=8.0 Hz, 1H), 8.76 (d, J=4.0 Hz, 1H), 9.40 (d, J=8.0 Hz, 1H), 12.84 (s, 1H); MS (ESI) (M+H)⁺=438.0.

### Example 148

### 3-(1-Naphthoylamino)-N-(pyrrolidin-2-ylmethyl)pyridine-2-carboxamide

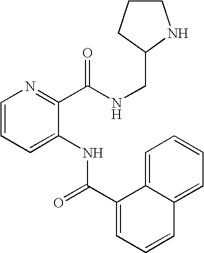

### Step A. 3-(1-Naphthoylamino)-N-(pyrrolidin-2-ylmethyl)pyridine-2-carboxamide

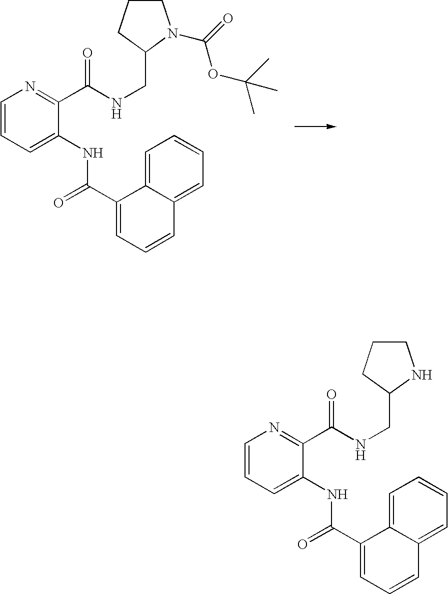

The crude tert-butyl 2-[({[3-(1-naphthoylamino)pyridin-2-yl]carbonyl}amino)methyl]pyrrolidine-1-carboxylate from Step B was treated with 4 N HCl in dioxane for 2 hrs at r.t. Removal of solvents gave a residue which was purified by reversed-phase HPLC to provide the title compound as its TFA salt (54 mg, 31%). ¹H NMR (400 MHz, CD₃OD) δ 1.80 (m, 1H), 2.03 (m, 2H), 2.21 (m, 1H), 3.20 (m, 1H), 3.28 (m, 1H), 3.68 (m, 3H), 7.60 (m, 3H), 7.68 (m 1H), 7.91 (d, J=8.0 Hz, 1H), 7.98 (d, J=8.0 Hz, 1H), 8.09 (d, J=8.0 Hz, 1H), 8.42 (m, 2H), 9.31 (d, J=8.0 Hz, 1H); MS (ESI) (M+H)⁺ 375.2.

### Step B. tert-Butyl 2-[({[3-(1-naphthoylamino)pyridin-2-yl]carbonyl}amino)methyl]pyrrolidine-1-carboxylate

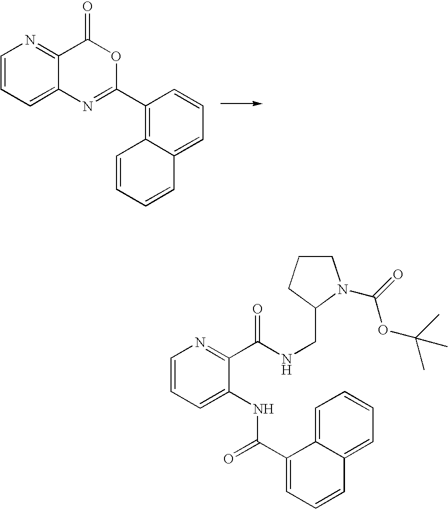

Following the procedure for Step A in Example 1, using 2-(1-naphthalenyl)-4H-pyrido[3,2-d][1,3]oxazin-4-one (100 mg, 0.36 mmol), and tert-butyl 2-(aminomethyl)pyrrolidine-1-carboxylate (300 mg, 1.5 mmol) provided a crude product, which was used in Step A directly.

### Example 149

### N-[(1-Methylpyrrolidin-2-yl)methyl]-3-(1-naphthoylamino)pyridine-2-carboxamide

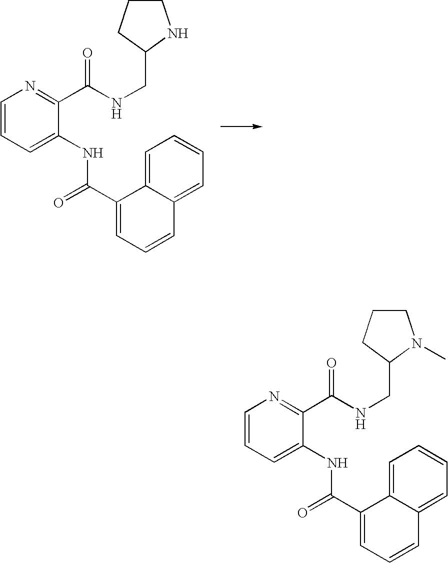

Following the procedure in Example 89, using 3-(1-naphthoylamino)-N-(pyrrolidin-2-ylmethyl)pyridine-2-carboxamide (TFA salt, 30 mg) and formaldehyde (37% in H₂O, 100 mg) provided the title compound as its TFA salt after purification by reversed-phase HPLC. ¹H NMR (400 MHz, CD₃OD) δ 1.96 (m, 2H), 2.08 (m, 1H), 2.28 (m, 1H), 2.97 (s, 3H), 3.12 (m, 1H), 3.67 (m, 3H), 3.88 (m, 1H), 7.59 (m, 3H), 7.61 (m 1H), 7.91 (d, J=8.0 Hz, 1H), 7.98 (d, J=8.0 Hz, 1H), 8.09 (d, J=8.0 Hz, 1H), 8.42 (m, 2H), 9.28 (d, J=8.0 Hz, 1H); MS (ESI) (M+H)⁺ 389.2.

### Example 150

### N-[(1-Methylpiperidin-2-yl)methyl]-3-(1-naphthoylamino)pyridine-2-carboxamide

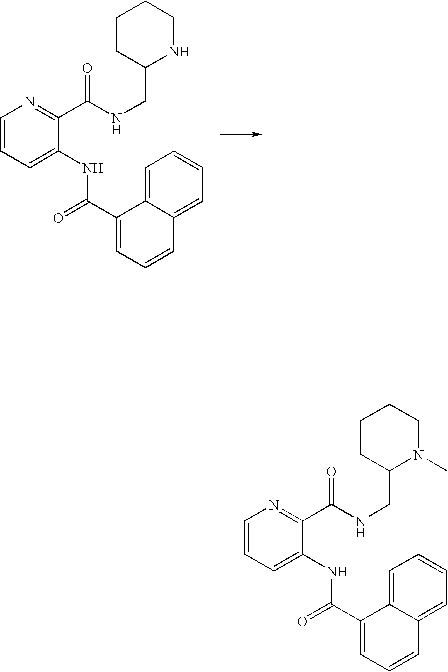

Following the procedure in Example 89, using 3-(1-naphthoylamino)-N-(piperidin-2-ylmethyl)pyridine-2-carboxamide (TFA salt, 100 mg) and formaldehyde (37% in H₂O, 100 mg) provided the title compound as its TFA salt after purification by reversed-phase HPLC (52 mg, 51%). ¹H NMR (400 MHz, CD₃OD) δ 1.67 (m, 3H), 1.86 (m, 2H), 2.05 (m, 1H), 3.02 (s, 3H), 3.03 (m, 1H), 3.25 (m, 1H), 3.44 (m, 1H), 3.60 (m, 1H), 3.96 (m, 1H), 7.58 (m, 3H), 7.61 (m 1H), 7.91 (d, J=7.2 Hz, 1H), 7.98 (d, J=8.0 Hz, 1H), 8.09 (d, J=8.0 Hz, 1H), 8.42 (m, 2H), 9.29 (d, J=8.0 Hz, 1H); MS (ESI) (M+H)⁺ 403.3.

### Example 151

### N-[(1-Acetylpiperidin-2-yl)methyl]-3-(1-naphthoylamino)pyridine-2-carboxamide

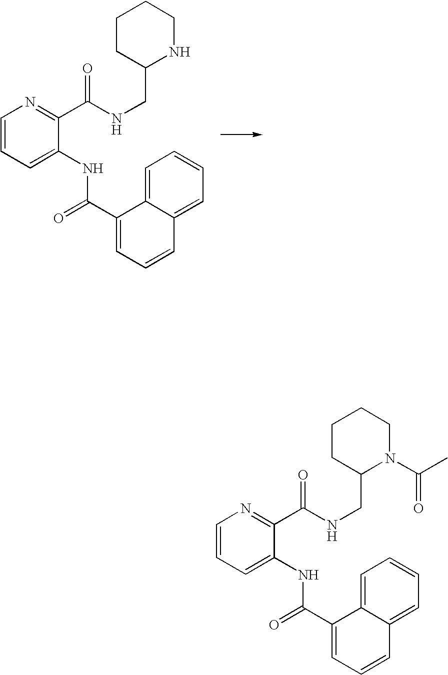

To a solution of 3-(1-naphthoylamino)-N-(piperidin-2-ylmethyl)pyridine-2-carboxamide (100 mg, 0.26 mmol) and DIPEA (129 mg, 1.0 mmol) in CH₂Cl₂ (10 mL) was added acetyl chloride (78 mg, 1.0 mmol) at r.t. After 1 hr, the reaction mixture was condensed. The residue was purified by reversed-phase HPLC to provide the title compound as its TFA salt. ¹H NMR (400 MHz, CD₃OD) δ 1.34 (m, 1H), 1.65 (m, 5H), 2.02 & 1.98 (s, 3H), 2.85 (m, 1H), 3.37 (m, 2H), 3.55-3.95 (m, 1H), 4.10-4.50 (m, 1H), 7.57 (m, 4H), 7.90 (d, J=8.0 Hz, 1H), 7.97 (m, 1H), 8.05 (d, J=8.0 Hz, 1H), 8.36 (m, 1H), 8.42 (d, J=8.0 Hz, 1H), 9.29 (d, J=8.0 Hz, 1H); MS (ESI) (M+H)⁺ 431.0.

### Example 152

### Methyl 2-[({[3-(1-naphthoylamino)pyridin-2-yl]carbonyl}amino)methyl]piperidine-1-carboxylate

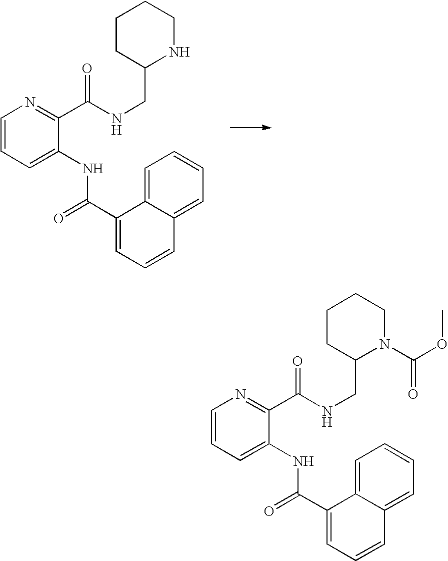

Following the procedure in Example 151, using 3-(1-naphthoylamino)-N-(piperidin-2-ylmethyl)pyridine-2-carboxamide (100 mg, 0.26 mmol) and methyl chloroformate (94 mg, 1.0 mmol) provided the title compound as its TFA salt after purification by reversed-phase HPLC. ¹H NMR (400 MHz, CD₃OD) δ 1.34 (m, 1H), 1.58 (m, 5H), 2.99 (m, 1H), 3.28 (m, 1H), 3.45 (s, 3H), 3.79 (m, 1H), 3.89 (m, 1H), 4.47 (m, 1H), 7.56 (m, 4H), 7.91 (m, 2H), 8.04 (d, J=8.0 Hz, 1H), 8.31 (brs, 1H), 8.43 (d, J=8.0 Hz, 1H), 9.25 (d, J=8.0 Hz, 1H); MS (ESI) (M+H)⁺ 447.0.

### Example 153

### N-(Cyclopentylmethyl)-4-(1-naphthoylamino)nicotinamide

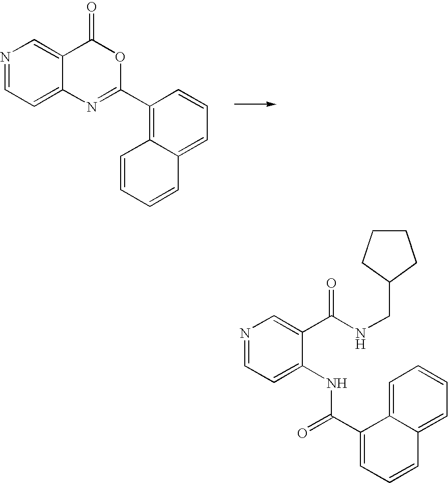

Following the procedure for Step A in Example 1, using 2-(1-naphthyl)-4H-pyrido[4,3-d][1,3]oxazin-4-one (55 mg, 0.2 mmol) and (cyclopentylmethyl)amine (99 mg, 1.0 mmol) provided the title compound as its TFA salt after purification by reversed-phase HPLC (36 mg, 37%). ¹H NMR (400 MHz, CD₃OD) δ 1.29 (m, 2H), 1.58 (m, 2H), 1.65 (m, 2H), 1.80 (m, 2H), 2.21 (m, 1H), 3.32(m, 2H), 7.65 (m, 3H), 8.01 (m, 2H),), 8.17 (d, J=8.0 Hz, 1H), 8.54 (m, 1H),), 8.77 (m, 1H), 9.07 (s, 1H), 9.28 (d, J=8.0 Hz, 1H); MS (ESI) (M+H)⁺ 374.2.

### Example 154

### N-Cyclopentyl-4-(1-naphthoylamino)nicotinamide

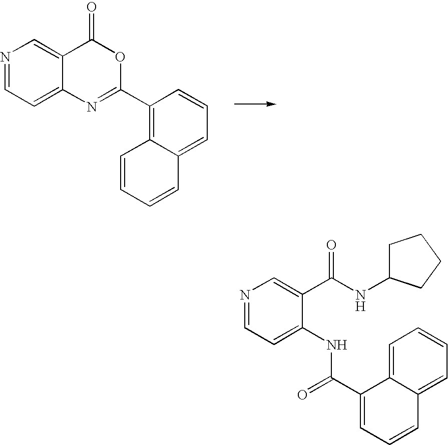

Following the procedure for Step A in Example 1, using 2-(1-naphthyl)-4H-pyrido[4,3-d][1,3]oxazin-4-one (55 mg, 0.2 mmol) and cyclopentylamine (85 mg, 1.0 mmol) provided the title compound as its TFA salt after purification by reversed-phase HPLC (62 mg, 66%). ¹H NMR (400 MHz, CD₃OD) δ 1.63 (m, 4H), 1.78 (m, 2H), 2.03 (m, 2H), 4.31 (m, 1H), 7.63 (m, 3H), 8.01 (m, 2H),), 8.16 (d, J=8.0 Hz, 1H), 8.54 (m, 1H),), 8.76 (m, 1H), 9.09 (s, 1H), 9.25 (d, J=8.0 Hz, 1H); MS (ESI) (M+H)⁺ 360.3.

### Example 155

### N-(Cyclopropylmethyl)-4-(1-naphthoylamino)nicotinamide

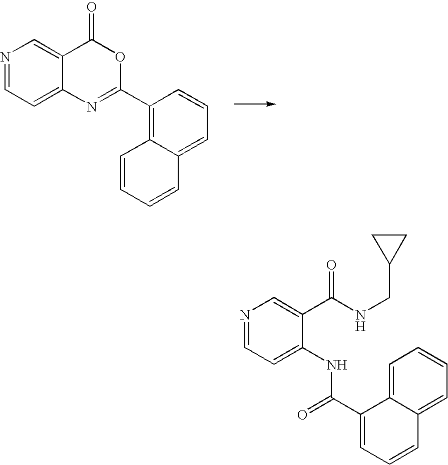

Following the procedure for Step A in Example 1, using 2-(1-naphthyl)-4H-pyrido[4,3-d][1,3]oxazin-4-one (55 mg, 0.2 mmol) and cyclopropylmethylamine (71 mg, 1.0 mmol) provided the title compound as its TFA salt after purification by reversed-phase HPLC (9 mg, 10%). ¹H NMR (400 MHz, CD₃OD) δ 10.02 (m, 2H), 0.28 (m, 2H), 0.85 (m, 1H), 2.98 (d, J=7.2 Hz, 1H), 7.36 (m, 3H), 7.74 (m, 2H),), 7.89 (d, J=8.0 Hz, 1H), 8.27 (m, 1H),), 8.49 (m, 1H), 8.83 (s, 1H), 8.98 (m, 1H); MS (ESI) (M+H)⁺ 346.3.

### Example 156

### N-Isobutyl-4-(1-naphthoylamino)nicotinamide

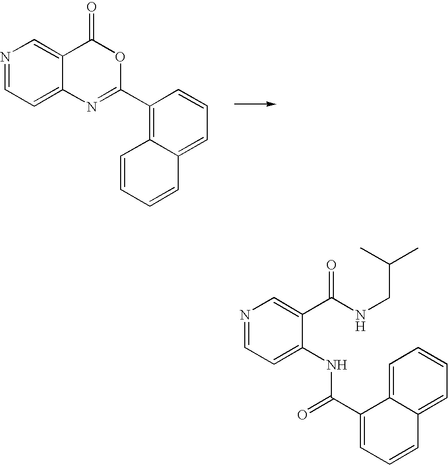

Following the procedure for Step A in Example 1, using 2-(1-naphthyl)-4H-pyrido[4,3-d][1,3]oxazin-4-one (55 mg, 0.2 mmol) and isobutylamine (73 mg, 1.0 mmol) provided the title compound as its TFA salt after purification by reversed-phase HPLC (9 mg, 10%). ¹H NMR (400 MHz, CD₃OD) δ 0.97 (d, J=6.6 Hz, 6H), 1.93 (m, 1H), 3.22 (d, J=7.0 Hz, 1H), 7.63 (m, 3H), 8.01 (m, 2H),), 8.17 (d, J=8.0 Hz, 1H), 8.54 (m, 1H),), 8.78 (m, 1H), 9.10 (s, 1H), 9.32 (d, J=8.0 Hz, 1H); MS (ESI) (M+H)⁺ 348.3.

### Example 157

### N-(Cyclobutylmethyl)-4-[(4-methyl-1-naphthoyl)amino]nicotinamide

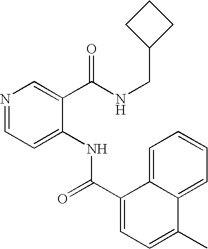

### Step A. N-(Cyclobutylmethyl)-4-[(4-methyl-1-naphthoyl)amino]nicotinamide

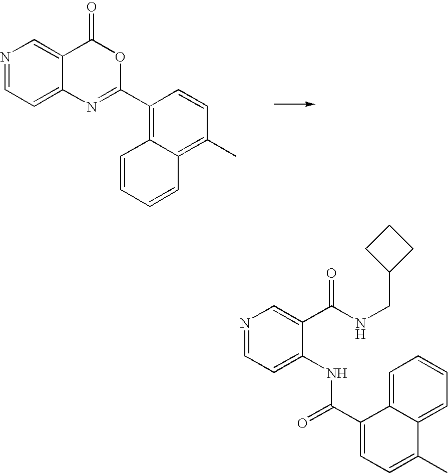

Following the procedure for Step A in Example 1, using 2-(4-methyl-1-naphthyl)-4H-pyrido[4,3-d][1,3]oxazin-4-one (58 mg, 0.2 mmol) and (cyclobutylmethyl)amine (85 mg, 1.0 mmol) provided the title compound as its TFA salt after purification by reversed-phase HPLC (28 mg, 29%). ¹H NMR (400 MHz, CD₃OD) δ 1.79 (m, 2H), 1.89 (m, 2H), 2.09 (m, 2H), 2.62 (m, 1H), 2.77 (s, 3H), 3.41 (d, J=7.4 Hz, 2H), 7.48 (d, J=7.4 Hz, 1H), 7.64 (m, 2H), 7.89(d, J=7.4 Hz, 1H)), 8.14 (d, J=8.0 Hz, 1H), 8.57 (m, 1H),), 8.71 (m, 1H), 9.04 (s, 1H), 9.23 (m, 1H); MS (ESI) (M+H)⁺ 374.2.

### Step B. 2-(4-Methyl-1-naphthyl)-4H-pyrido[4,3-d][1,3]oxazin-4-one

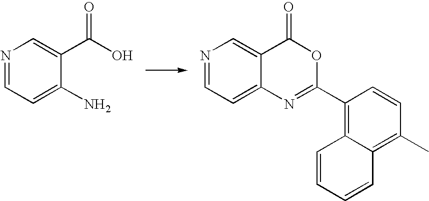

Following the procedure for Step B in Example 1, using 4-aminonicotinic acid (55 mg, 0.4 mmol), 4-methyl-1-naphthalenecarbonyl chloride (102 mg, 0.5 mmol), DIPEA (284 mg, 2.2 mmol), and then HATU (419 mg, 1.1 mmol) provided the title compound as a DMF (6 mL) solution which was used directly in Step A. MS (ESI) (M+H)⁺ 288.8.

### Example 158

### N-(Cyclopentylmethyl)-4-[(4-methyl-1-naphthoyl)amino]nicotinamide

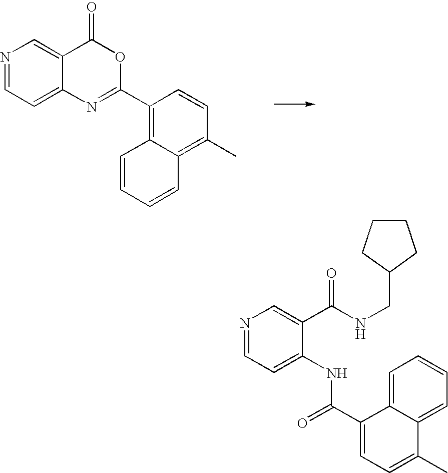

Following the procedure for Step A in Example 1, using 2-(4-methyl-1-naphthyl)-4H-pyrido[4,3-d][1,3]oxazin-4-one (58 mg, 0.2 mmol) and (cyclopentylmethyl)amine (99 mg, 1.0 mmol) provided the title compound as its TFA salt after purification by reversed-phase HPLC (18 mg, 18%). ¹H NMR (400 MHz, CD₃OD) δ 1.29 (m, 2H), 1.58 (m, 2H), 1.65 (m, 2H), 1.80 (m, 2H), 2.22 (m, 1H), 2.78 (s, 3H), 3.32 (m, 2H), 7.50 (d, J=7.4 Hz, 1H), 7.64 (m, 2H), 7.91 (d, J=7.4 Hz, 1H),), 8.16 (d, J=8.0 Hz, 1H), 8.59 (m, 1H),), 8.75 (m, 1H), 9.06 (s, 1H), 9.26 (m, 1H); MS (ESI) (M+H)⁺ 388.3.

### Example 159

### 3-{[4-(Hydroxymethyl)-1-naphthoyl]amino}-N-(tetrahydro-2H-pyran-4-ylmethyl)pyridine-2-carboxamide

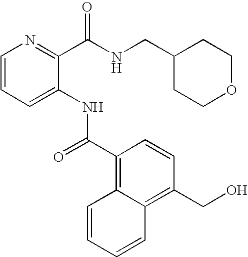

### Step A. 3-{[4-(Hydroxymethyl)-1-naphthoyl]amino}-N-(tetrahydro-2H-pyran-4-ylmethyl)pyridine-2-carboxamide

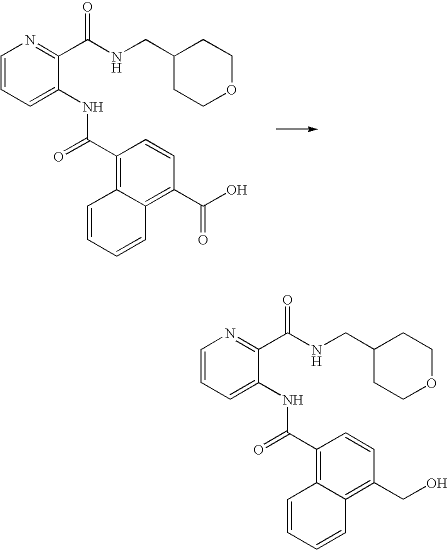

Oxalyl chloride (0.011 mL, 0.115 mmol) was added to a mixture of 4-{[(2-{[(Tetrahydro-2H-pyran-4-ylmethyl)amino]carbonyl}pyridin-3-yl)amino]carbonyl}-1-naphthoic acid (50 mg, 0.11 mmol) and DCE (20 mL) at 0° C. The reaction mixture was allowed to warm to ambient temperature and oxalyl chloride (0.005 mL, 0.057 mmol) was added. The reaction mixture was heated to 70° C., stirred for 1 hr and cooled to 0° C. NaBH₄ (22 mg, 0.57 mmol) and iodine (one crystal) were added. The reaction mixture was stirred for 1 hr. at 0° C. and quenched with MeOH (5 mL). The solvent was concentrated and the product was purified by preparative reverse-phase HPLC to provide the TFA salt of the title compound as white powder (41 mg, 67%); ¹H NMR (400 MHz, CHLOROFORM-D) δ 1.31-1.45 (m, 2H), 1.67 (dt, J=13.03, 1.78 Hz, 2H), 1.78-1.96 (m, 3H), 3.31 (t, J=6.64 Hz, 2H), 3.37 (td, J=11.77, 2.05 Hz, 2H), 3.98 (dd, J=11.52, 3.71 Hz, 2H), 5.20 (d, J=0.59 Hz, 2H), 7.54 (dd, J=8.59, 4.49 Hz, 1H), 7.57-7.62 (m, 2H), 7.64 (d, J=7.42 Hz, 1H), 7.87 (d, J=7.23 Hz, 1H), 8.09-8.16 (m, 1H), 8.28 (dd, J=4.49, 1.37 Hz, 1H), 8.52-8.61 (m, 1H), 9.40 (dd, J=8.59, 1.37 Hz, 1H), 12.80 (s, 1H); MS (ESI) (M+H)⁺ 420.0: Anal. Calcd. for C₂₄H₂₅N₃O₄+0.10TFA+0.20H₂O: C, 67.15; H, 5.94; N, 9.71. Found: C, 67.09; H, 5.78; N, 9.58.

### Step B. 3-Amino-N-(tetrahydro-2H-pyran-4-ylmethyl)pyridine-2-carboxamide

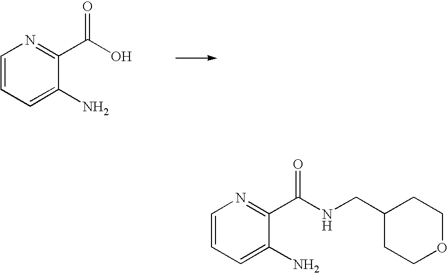

HATU (2.63 g, 6.93 mmol) and 4-aminomethyltetrahydropyran (0.80 g, 6.94 mmol) were added to a solution of 3-amino-2-pyridine carboxylic acid (0.91 g, 6.60 mmol) and DIPEA (1.26 mL, 7.26 mmol) in DMF (120 mL) at 0° C. The reaction mixture was allowed to warm to ambient temperature and heated to 50° C. for 3 hrs. The solvent was concentrated and the residue was recovered in EtOAc (300 mL). The solution washed with water, saturated NaHCO₃ solution, brine and dried over anhydrous Na₂SO₄. The solvent was concentrated and the product was purified on silica gel by flash chromatography using Et₃N 0.1%, MeOH 3% and Acetone 5% in DCM to provide the title compound as white solid (1.40 g, 90%).

### Step C. Naphthalene-1,4-dicarbonyl dichloride

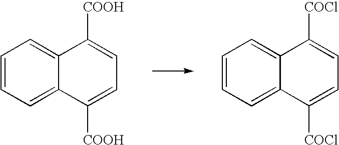

Naphtalene 1,4-dicarboxylic acid (0.25 g, 1.15 mmol) was added to SOCl₂ (10 mL). The reaction mixture was heated to reflux and stirred for 3 hrs. The resulting solution was cooled to ambient temperature and the solvent was concentrated. The residue was dried under vacuum. The crude product was used for the next step without further purification.

### Step D. 4-{[(2-{[(Tetrahydro-2H-pyran-4-ylmethyl)amino]carbonyl}pyridin-3-yl)amino]carbonyl}-1-naphthoic acid

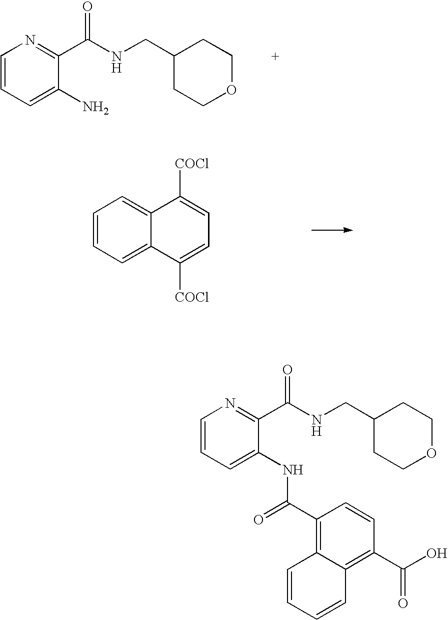

A solution of 3-Amino-N-(tetrahydro-2H-pyran-4-ylmethyl)pyridine-2-carboxamide (67 mg, 0.28 mmol) and DIPEA (1 mL, 5.74 mmol) in DCE (2 mL) was added to a solution of naphthalene-1,4-dicarbonyl dichloride (example 1, step C) in DCE (20 mL). The reaction mixture was stirred for 3 hrs. at ambient temperature and quenched with water (20 mL). The organic layer was separated and dried over anhydrous Na₂SO₄. The solvent was concentrated and the product was purified by preparative reverse-phase HPLC to provide the TFA salt of the title compound as white powder (20 mg, 16%). ¹H NMR (400 MHz, DMSO-D6) δ 1.49 (dd, J=12.89, 2.15 Hz, 2H), 2.07 (d, J=3.91 Hz, 2H), 3.12 (m, 2H), 3.19 (m, 2H), 3.32 (s, 2H), 3.78 (dd, J=10.74, 3.32 Hz, 2H), 3.89 (s, 1H), 7.67 (t, J=7.71 Hz, 1H), 7.73 (dd, J=8.59, 4.69 Hz, 2H), 7.92 (d, J=7.42 Hz, 1H), 8.19 (d, J=7.62 Hz, 1H), 8.35 (d, J=8.20 Hz, 1H), 8.42 (dd, J=4.49, 1.37 Hz, 1H), 8.85 (d, J=8.40 Hz, 1H), 9.20 (dd, J=8.49, 1.27 Hz, 1H), 12.96 (s, 1H); MS (ESI) (M+H)⁺ 434.0; Anal. Calcd. for C₂₄H₂₃N₃O₅+0.20TFA+0.10H₂O: C, 63.98; H, 5.15; N, 9.17. Found: C, 64.09; H, 5.15; N, 9.02.

### Example 160

### 3-{[4-(Piperidin-1-ylmethyl)-1-naphthoyl]amino}-N-(tetrahydro-2H-pyran-4-ylmethyl)pyridine-2-carboxamide

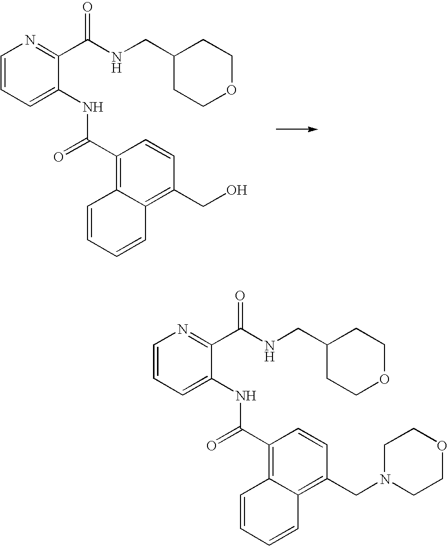

Methane sulfonyl chloride (0.011 mL, 0.14 mmol) was added to a solution of 3-{[4-(Hydroxymethyl)-1-naphthoyl]amino}-N-(tetrahydro-2H-pyran-4-ylmethyl)pyridine-2-carboxamide (50 mg, 0.11 mmol) and Et₃N (0.032 mL, 0.17 mmol) in DCM (20 mL) at 0° C. The reaction mixture was allowed to warm to ambient temperature and stirred for 4 hrs. The solvent was concentrated and the product was recovered in DMF (10 mL). Morpholine (0.10 mL, 1.19 mmol) and KI (69 mg, 0.41 mmol) were added to the resulting solution. The reaction mixture was heated to 80° C. for 2 hrs. The solvent was concentrated and the product was purified by preparative reverse-phase HPLC to provide the TFA salt of the title compound as white powder (49 mg, 68%); ¹H NMR (400 MHz, CHLOROFORM-D) δ 1.30-1.47 (m, 2H), 1.67 (dd, J=13.86, 2.73 Hz, 2H), 1.79-1.96 (m, 1H), 3.31 (t, J=6.64 Hz, 2H), 3.38 (td, J=11.81, 2.15 Hz, 2H), 3.91-4.05 (m, 8H), 4.74-4.81 (m, 2H), 7.56 (dd, J=8.59, 4.49 Hz, 1H), 7.61-7.75 (m, 2H), 7.82 (d, J=7.42 Hz, 1H), 7.91 (d, J=7.22 Hz, 1H), 8.17 (d, J=7.81 Hz, 1H), 8.31 (dd, J=4.49, 1.37 Hz, 1H), 8.54-8.58 (m, 1H), 8.60 (t, J=6.44 Hz, 1H), 9.39 (dd, J=8.59, 1.37 Hz, 1H), 12.91 (s, 1H); MS (ESI) (M+H)⁺ 489.2; Anal. Calcd. for C₂₈H₃₂N₄O₄+1.10TFA+1.60H₂O+0.50MeCN: C, 57.56; H, 5.58; N, 8.89. Found: C, 57.62; H, 5.55; N, 8.86.

### Example 161

### 3-[(4-{[(2-Hydroxyethyl)amino]methyl}-1-naphthoyl)amino]-N-(tetrahydro-2H-pyran-4-ylmethyl)pyridine-2-carboxamide

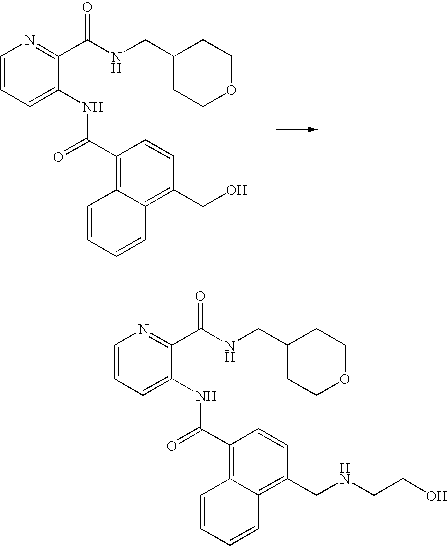

Following the procedure in Example 160, using ethanolamine (0.072 mL, 1.19 mmol) provided the title compound as its TFA salt after purification by reversed-phase HPLC (44 mg, 64%). ¹H NMR (400 MHz, CHLOROFORM-D) δ 1.30-1.47 (m, 2H), 1.67 (dd, J=12.99, 1.86 Hz, 2H), 1.78-1.96 (m, 1H), 2.20-2.34 (m, 1H), 2.65-2.83 (m, 1H), 3.31 (t, J=6.64 Hz, 2H), 3.38 (td, J=11.77, 2.05 Hz, 2H), 3.91 (q, J=9.24 Hz, 2H), 3.99 (dd, J=11.23, 3.22 Hz, 2H), 4.27-4.40 (m, 2H), 4.71-4.79 (m, 2H), 7.56 (dd, J=8.59, 4.49 Hz, 1H), 7.61-7.76 (m, 3H), 7.91 (d, J=7.22 Hz, 1H), 8.08 (d, J=7.81 Hz, 1H), 8.31 (dd, J=4.49, 1.56 Hz, 1H), 8.56 (dd, J=8.20, 1.37 Hz, 1H), 8.61 (t, J=6.15 Hz, 1H), 9.39 (dd, J=8.59, 1.37 Hz, 1H), 12.90 (s, 1H); MS (ESI) (M+H)⁺ 463.0; Anal. Calcd. for C₂₆H₃₀N₄O₄+1.80TFA+1.60H₂O+0.50MeCN: C, 51.25; H, 5.13; N, 8.79. Found: C, 51.30; H, 5.09; N, 8.81.

### Example 162

### 3-({4-[(Dimethylamino)methyl]-1-naphthoyl}amino)-N-(tetrahydro-2H-pyran-4-ylmethyl)pyridine-2-carboxamide

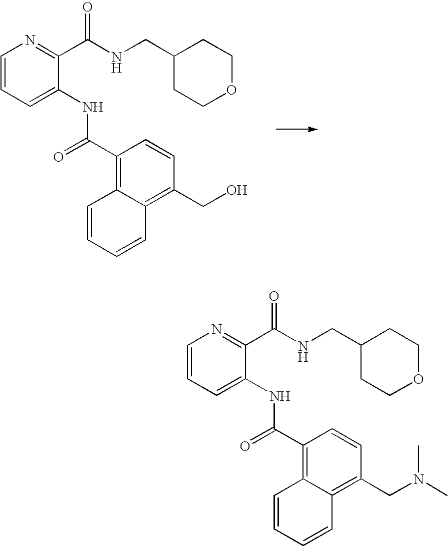

Following the procedure in Example 160, using dimethylamine hydrochloride (89 mg, 1.07 mmol) provided the title compound as its TFA salt after purification by reversed-phase HPLC (30 mg, 44%). ¹H NMR (400 MHz, CHLOROFORM-D) δ 1.30-1.47 (m, 2H), 1.62-1.73 (m, 2H), 1.78-1.96 (m, 1H), 2.87 (s, 6H), 3.31 (t, J=6.64 Hz, 2H), 3.38 (td, J=11.81, 1.95 Hz, 2H), 3.99 (dd, J=11.13, 3.71 Hz, 2H), 4.73-4.82 (m, 2H), 7.56 (dd, J=8.59, 4.49 Hz, 1H), 7.63-7.74 (m, 2H), 7.78 (d, J=7.42 Hz, 1H), 7.92 (d, J=7.42 Hz, 1H), 8.16 (d, J=7.81 Hz, 1H), 8.31 (dd, J=4.49, 1.37 Hz, 1H), 8.57 (d, J=8.20 Hz, 1H), 8.60 (t, J=6.54 Hz, 1H), 9.39 (dd, J=8.59, 1.37 Hz, 1H), 12.91 (s, 1H); MS (ESI) (M+H)⁺ 447.0; Anal. Calcd. for C₂₆H₃₀N₄O₃+1.60TFA+0.90H₂O: C, 54.36; H, 5.22; N, 8.68. Found: C, 54.37; H, 5.24; N, 8.48.

### Example 163

### 3-{[4-(1H-Imidazol-1-ylmethyl)-1-naphthoyl]amino}-N-(tetrahydro-2H-pyran-4-ylmethyl)pyridine-2-carboxamide

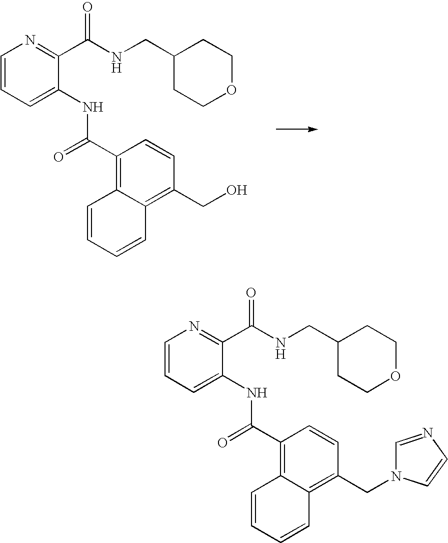

Following the procedure in Example 160, using imidazole (81 mg, 1.19 mmol, after imidazole addition, the reaction mixture was heated to 80° C. and stirred overnight) provided the title compound as its TFA salt after purification by reversed-phase HPLC (20 mg, 28%). ¹H NMR (400 MHz, CHLOROFORM-D) δ 1.29-1.48 (m, 2H), 1.67 (d, J=12.89 Hz, 2H), 1.79-1.93 (m, 1H), 3.31 (t, J=6.64 Hz, 2H), 3.37 (td, J=11.77, 1.86 Hz, 2H), 3.98 (dd, J=11.13, 4.10 Hz, 2H), 5.81 (s, 2H), 7.06 (s, 1H), 7.38 (s, 1H), 7.47 (d, J=7.42 Hz, 1H), 7.56 (dd, J=8.59, 4.49 Hz, 1H), 7.62-7.71 (m, 2H), 7.82-7.88 (m, 1H), 7.90 (d, J=7.23 Hz, 1H), 8.31 (dd, J=4.59, 1.46 Hz, 1H), 8.54-8.66 (m, 2H), 8.85 (s, 1H), 9.39 (dd, J=8.59, 1.37 Hz, 1H), 12.92 (s, 1H); MS (ESI) (M+H)⁺ 470.0.

### Example 164

### 3-{[4-(Azetidin-1-ylmethyl)-1-naphthoyl]amino}-N-(tetrahydro-2H-pyran-4-ylmethyl)pyridine-2-carboxamide

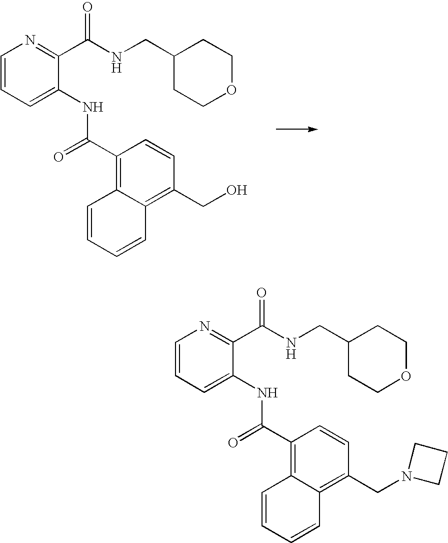

Following the procedure in Example 160, using azetidine (68 mg, 1.19 mmol, after azetidine addition, the reaction mixture was heated to 80° C. and stirred overnight) provided the title compound as its TFA salt after purification by reversed-phase HPLC (42 mg, 61%). ¹H NMR (400 MHz, CHLOROFORM-D) δ 1.30-1.47 (m, 2H), 1.67 (dd, J=12.99, 1.86 Hz, 2H), 1.78-1.96 (m, 1H), 2.20-2.34 (m, 1H), 2.65-2.83 (m, 1H), 3.31 (t, J=6.64 Hz, 2H), 3.38 (td, J=11.77, 2.05 Hz, 2H), 3.91 (q, J=9.24 Hz, 2H), 3.99 (dd, J=11.23, 3.22 Hz, 2H), 4.27-4.40 (m, 2H), 4.71-4.79 (m, 2H), 7.56 (dd, J=8.59, 4.49 Hz, 1H), 7.61-7.76 (m, 3H), 7.91 (d, J=7.22 Hz, 1H), 8.08 (d, J=7.81 Hz, 1H), 8.31 (dd, J=4.49, 1.56 Hz, 1H), 8.56 (dd, J=8.20, 1.37 Hz, 1H), 8.61 (t, J=6.15 Hz, 1H), 9.39 (dd, J=8.59, 1.37 Hz, 1H), 12.90 (s, 1H); MS (ESI) (M+H)⁺ 459.2; Anal. Calcd. for C₂₇H₃₀N₄O₃+1.60TFA+0.80H₂O: C, 55.34; H, 5.11; N, 8.55. Found: C, 55.29; H, 5.14; N, 8.50.

### Example 165

### Methyl 4-{[(2-{[(tetrahydro-2H-pyran-4-ylmethyl)amino]carbonyl}pyridin-3-yl)amino]carbonyl}-1-naphthoate

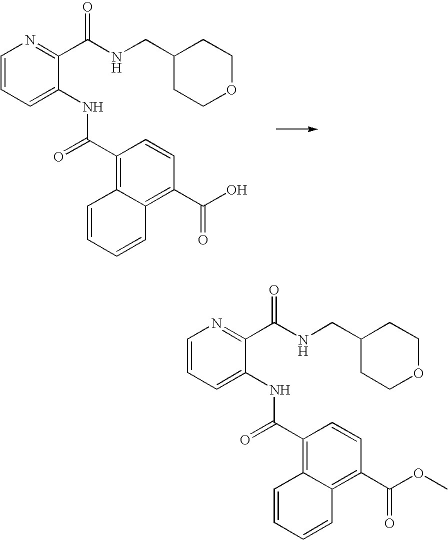

Oxalyl chloride (0.011 mL, 0.115 mmol) was added to a mixture of 4-{[(2-{[(Tetrahydro-2H-pyran-4-ylmethyl)amino]carbonyl}pyridin-3-yl)amino]carbonyl}-1-naphthoic acid (50 mg, 0.11 mmol) and DCE (20 mL) at 0° C. The reaction mixture was allowed to warm to ambient temperature and oxalyl chloride (0.005 mL, 0.057 mmol) was added. The reaction mixture was heated to 70° C., stirred for 1 hr., cooled to 0° C. and quenched with MeOH (5 mL). The solvent was concentrated and the product was purified by preparative reverse-phase HPLC to provide the TFA salt of the title compound as white powder (20 mg, 30%). ¹H NMR (400 MHz, CHLOROFORM-D) δ 1.30-1.46 (m, 2H), 1.66 (dd, J=12.89, 1.76 Hz, 2H), 1.77-1.92 (m, 1H), 3.29 (t, J=6.64 Hz, 2H), 3.37 (td, J=11.81, 1.95 Hz, 2H), 3.97 (dd, J=11.13, 3.51 Hz, 2H), 4.03 (s, 3H), 7.54 (dd, J=8.59, 4.49 Hz, 1H), 7.58-7.70 (m, 2H), 7.87 (d, J=7.42 Hz, 1H), 8.20 (d, J=7.42 Hz, 1H), 8.29 (dd, J=4.49, 1.37 Hz, 1H), 8.49 (d, J=8.01 Hz, 1H), 8.57 (t, J=6.05 Hz, 1H), 8.90 (d, J=8.20 Hz, 1H), 9.40 (dd, J=8.59, 1.37 Hz, 1H), 12.88 (s, 1H); MS (ESI) (M+H)⁺ 448.0; Anal. Calcd. for C₂₅H₂₅N₃O₅+0.30H₂O: C, 66.30; H, 5.70; N, 9.28. Found: C, 66.38; H, 5.67; N, 8.97.

### Example 166

### N,N-Dimethyl-N′-(2-{[(tetrahydro-2H-pyran-4-ylmethyl)amino]carbonyl}pyridin-3-yl)naphthalene-1,4-dicarboxamide

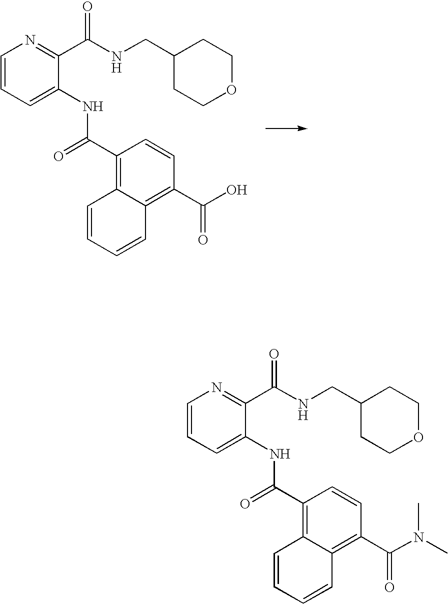

Following the procedure for Example 165, using dimethylamine hydrochloride (75 mg, 0.91 mmol) provided the title compound as its TFA salt after purification by reversed-phase HPLC (30 mg, 43%); ¹H NMR (400 MHz, CHLOROFORM-D) δ 1.32-1.47 (m, 2H), 1.62-1.73 (m, 2H), 1.80-1.95 (m, 1H), 2.74 (s, 3H), 2.85 (s, 2H), 3.27-3.34 (m, 4H), 3.39 (td, J=11.81, 1.95 Hz, 2H), 4.00 (dd, J=11.23, 3.42 Hz, 2H), 7.51 (d, J=7.23 Hz, 1H), 7.55 (dd, J=8.59, 4.49 Hz, 1H), 7.57-7.65 (m, 2H), 7.78-7.85 (m, 1H), 7.92 (d, J=7.22 Hz, 1H), 8.29 (dd, J=4.59, 1.46 Hz, 1H), 8.52-8.63 (m, 1H), 9.40 (dd, J=8.59, 1.37 Hz, 1H), 12.86 (s, 1H); MS (ESI) (M+H)⁺ 461.0; Anal. Calcd. for C₂₆H₂₈N₄O₄+0.50TFA: C, 62.66; H, 5.55; N, 10.83. Found: C, 62.80; H, 5.59; N, 10.64.

### Example 167

### 2-Hydroxyethyl 4-{[(2-{[(tetrahydro-2H-pyran-4-ylmethyl)amino]carbonyl}pyridin-3-yl)amino]carbonyl}-1-naphthoate

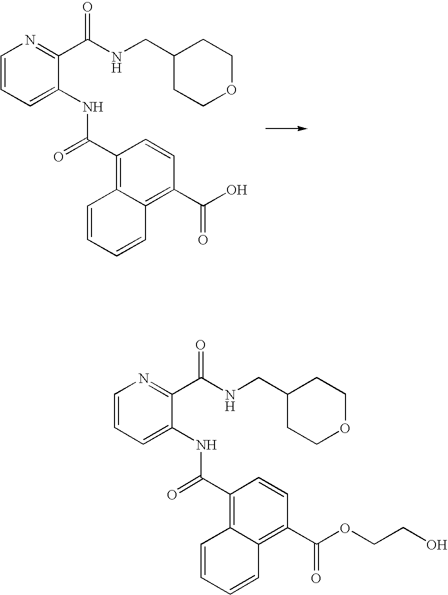

Following the procedure for Example 165, using ethylene glycol (171 mg, 2.76 mmol) provided the title compound as its TFA salt after purification by reversed-phase HPLC (20 mg, 12%); ¹H NMR (400 MHz, CHLOROFORM-D) δ 1.30-1.46 (m, 2H), 1.66 (dd, J=12.89, 1.95 Hz, 2H), 1.75-1.94 (m, 1H), 3.15 (s, 1H), 3.37 (td, J=11.81, 1.95 Hz, 2H), 3.93-4.07 (m, 4H), 4.51-4.61 (m, 2H), 7.54 (dd, J=8.59, 4.49 Hz, 1H), 7.58-7.72 (m, 2H), 7.87 (d, J=7.42 Hz, 1H), 8.23 (d, J=7.62 Hz, 1H), 8.29 (dd, J=4.49, 1.56 Hz, 1H), 8.50 (dd, J=8.20, 0.98 Hz, 1H), 8.58 (t, J=6.15 Hz, 1H), 8.88 (d, J=7.62 Hz, 1H), 9.39 (dd, J=8.59, 1.37 Hz, 1H), 12.89 (s, 1H); MS (ESI) (M+H)⁺ 478.0; Anal. Calcd. for C₂₆H₂₇N₃O₆+0.30TFA+0.20H₂O: C, 62.00; H, 5.42; N, 8.15. Found: C, 61.93; H, 5.27; N, 8.15.

### Example 168

### 3-[(1-Benzofuran-2-ylcarbonyl)amino]-N-(tetrahydro-2H-pyran-4-ylmethyl)pyridine-2-carboxamide

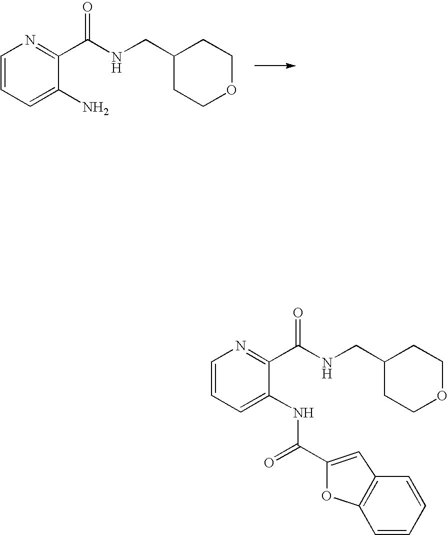

Following the procedure for Step A in Example 30, using 2-benzofurancarboxylic acid (172 mg, 1.06 mmol) and 3-Amino-N-(tetrahydro-2H-pyran-4-ylmethyl)pyridine-2-carboxamide (250 mg, 1.06 mmol) provided the title compound as its TFA salt after purification by reversed-phase HPLC (100 mg, 19%); ¹H NMR (400 MHz, DMSO-D6) δ 1.23 (m, 2H), 1.59 (dd, J=12.89, 1.76 Hz, 2H), 3.26 (m, 4H), 3.84 (dd, J=11.52, 2.54 Hz, 2H), 7.38 (m, 1H), 7.53 (td, J=7.81, 1.37 Hz, 1H), 7.68 (dd, J=8.59, 4.49 Hz, 1H), 7.74 (d, J=0.98 Hz, 1H), 7.80 (dd, J=34.47, 8.49 Hz, 2H), 8.40 (dd, J=4.39, 1.47 Hz, 1H), 9.11 (dd, J=8.59, 1.56 Hz, 1H), 9.31 (t, J=6.25 Hz, 1H), 13.39 (s, 1H); MS (ESI) (M+H)⁺ 380.2; Anal. Calcd. for C₂₁H₂₁N₃O₄+0.20H₂O: C, 65.85; H, 5.63; N, 10.97. Found: C, 65.79; H, 5.57; N, 11.09.

### Example 169

### N-(Cyclohexylmethyl)-3-[(4-iodo-1-naphthoyl)amino]pyridine-2-carboxamide

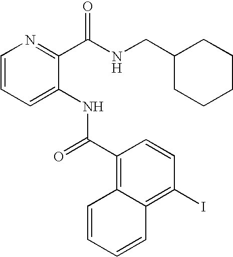

### Step A. N-(Cyclohexylmethyl)-3-[(4-iodo-1-naphthoyl)amino]pyridine-2-carboxamide

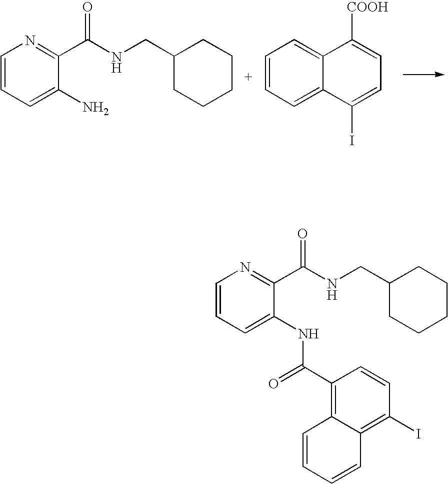

Oxalyl chloride (0.26 mL, 3.0 mmol) was added to a solution of 4-Iodo-1-naphthoic acid (580 mg, 1.85 mmol) in DCE (100 mL) at 0° C. DMF (1 drop) was added and the reaction mixture was stirred for 1 hr. at 0° C. A solution of 3-Amino-N-(cyclohexylmethyl)pyridine-2-carboxamide (465 mg, 1.9 mmol) and DIPEA (0.65 mL, 3.7 mmol) in DCE (20 mL) was added. The reaction mixture was heated to 70° C. and stirred overnight. The solvent was concentrated and the product was purified on silica gel by flash chromatography to provide the title compound as white solid (810 mg, 84%); ¹H NMR (400 MHz, CHLOROFORM-D) δ 1.00 (m, 2H), 1.20 (m, 3H), 1.56 (m, 2H), 1.74 (m, 3H), 3.23 (t, J=6.64 Hz, 2H), 7.52 (dd, J=8.59, 4.49 Hz, 1H), 7.57 (d, J=7.62 Hz, 1H), 7.61 (m, 2H), 8.18 (m, 2H), 8.29 (dd, J=4.49, 1.56 Hz, 1H), 8.46 (m, 1H), 9.37 (dd, J=8.59, 1.37 Hz, 1H), 12.94 (s, 1H); MS (ESI) (M+H)⁺ 514.0.

### Step B. 4-Iodo-1-naphthonitrile

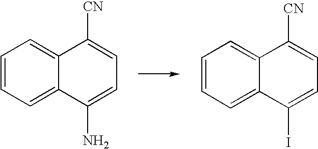

A solution of NaNO₂ (0.83 g, 12.1 mmol) in water (10 mL) was added over 30 min. to a mixture of 4-amino-1-naphthonitrile (1.94 g, 11.5 mmol), concentrated HCl (12 mL) and glacial acetic acid (25 mL) at 0° C. The reaction mixture was stirred for 1.5 hr. and cold water (25 mL) was added. A solution of KI (2.29 g, 13.8 mmol) and iodine (1.75 g, 6.9 mmol) in water (15 mL) was added. The reaction mixture was stirred for 2 hrs. at 0° C. and allowed to warm to ambient temperature. The product was extracted with EtOAc, washed with water and brine, and dried over anhydrous Na₂SO₄. The solvent was concentrated and the product was purified on silica gel by flash chromatography to provide the title compound as white solid (2.21 g, 67%).

### Step C. 4-Iodo-1-naphthoic acid

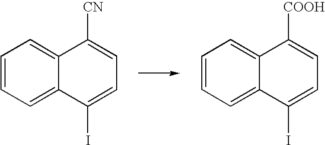

4-iodo-1-naphthonitrile (2.21 g, 7.92 mmol), concentrated HCl (20 mL) and glacial acetic acid (10 mL) were mixed together and heated to 130° C. overnight in a closed reaction vessel. The reaction mixture was cooled to ambient temperature and filtered. The residue was recovered in EtAOc and dried over anhydrous Na₂SO₄. The solvent was concentrated to provide the title compound as white solid (1.59 g, 67%)

### Example 170

### N-(Cyclohexylmethyl)-3-[(4-piperidin-1-yl-1-naphthoyl)amino]pyridine-2-carboxamide

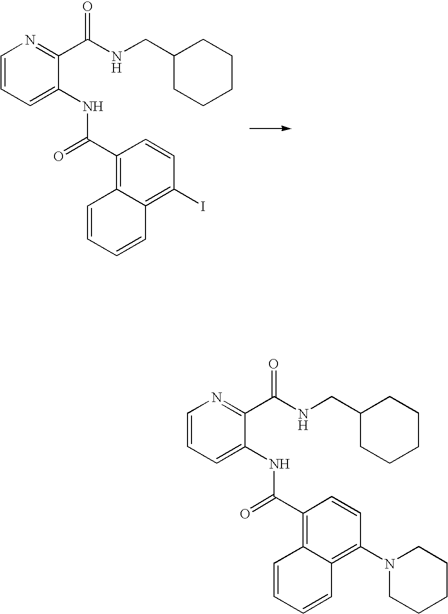

An oven dried reaction flask was loaded with Pd₂(dba)₃ (3.5 mg, 0.0038 mmol), N-(Cyclohexylmethyl)-3-[(4-iodo-1-naphthoyl)amino]pyridine-2-carboxamide (100 mg, 0.19 mmol), 2-dicyclohexylphosphino-2′-(N,N-dimethylamino)biphenyl (3.1 mg, 0.0078 mmol), 1.0 M solution LiHMDS in THF (0.62 mL, 0.62 mmol), piperidine (0.023 mL, 0.23 mmol) and anhydrous THF (1.5 mL) under nitrogen atmosphere. The reaction mixture was heated to 65° C. and stirred overnight. The reaction was cooled to ambient temperature and filtered. The solvent was concentrated and the product was purified on silica gel by MPLC to provide the title compound as white solid (36 mg, 39%); ¹H NMR (400 MHz, CHLOROFORM-D) δ 0.87-1.08 (m, 2H), 1.09-1.34 (m, 3H), 1.52-1.63 (m, 2H), 1.63-1.82 (m, 7H), 1.82-1.92 (m, 3H), 2.98-3.17 (m, J=5.47, 3.32 Hz, 2H), 3.20-3.30 (m, 2H), 7.07 (d, J=7.81 Hz, 1H), 7.44-7.61 (m, 3H), 7.84-7.95 (m, 1H), 8.18-8.30 (m, 2H), 8.49-8.57 (m, J=6.64 Hz, 1H), 8.58-8.63 (m, 1H), 9.36-9.43 (m, 1H), 12.77 (s, 1H); MS (ESI) (M+H)⁺ 471.3; Anal. Calcd. for C₂₆H₃₀N₄O₂+0.10H₂O: C, 73.73; H, 7.30; N, 11.86. Found: C, 73.66; H, 7.24; N, 11.87.

### Example 171

### 3-[(4-Azetidin-1-yl-1-naphthoyl)amino]-N-(cyclohexylmethyl)pyridine-2-carboxamide

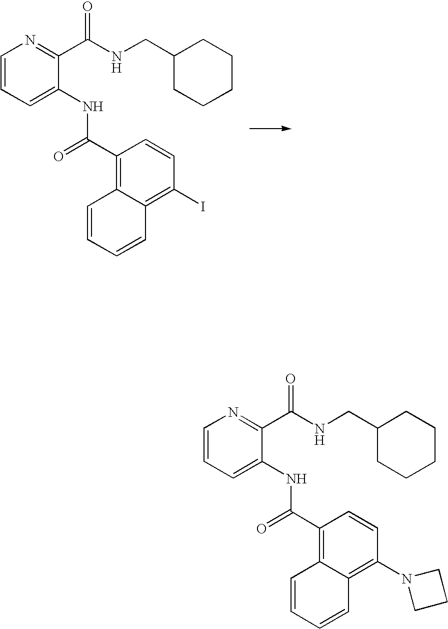

Following the procedure for example 170 (heating at 65° C. for 3 days and re-crystallizing in MeOH after flash chromatography) using azetidine (18 mg, 0.35 mmol) provided the title compound as a white solid (75 mg, 58%); ¹H NMR (400 MHz, CHLOROFORM-D) δ 0.92-1.05 (m, 2H), 1.10-1.31 (m, 2H), 1.52-1.62 (m, 2H), 1.62-1.70 (m, 1H), 1.70-1.84 (m, 4H), 2.40-2.50 (m, 2H), 3.26 (t, J=6.64 Hz, 2H), 4.24-4.31 (m, 4H), 6.49 (d, J=8.01 Hz, 1H), 7.37-7.44 (m, 1H), 7.48 (dd, J=8.59, 4.49 Hz, 1H), 7.50-7.55 (m, 1H), 7.88 (d, J=8.20 Hz, 1H), 7.99 (d, J=8.59 Hz, 1H), 8.23 (dd, J=4.49, 1.56 Hz, 1H), 8.52 (t, J=6.05 Hz, 1H), 8.72 (dd, J=8.59, 0.78 Hz, 1H), 9.37 (dd, J=8.59, 1.56 Hz, 1H), 12.72 (s, 1H); MS (ESI) (M+H)⁺ 443.1; Anal. Calcd. for C₂₇H₃₀N₄O₂: C, 73.28; H, 6.83; N, 12.66. Found: C, 73.25; H, 6.88; N, 12.69.

### Example 172

### N-(Cyclohexylmethyl)-3-({4-[ethyl(methyl)amino]-1-naphthoyl}amino)pyridine-2-carboxamide

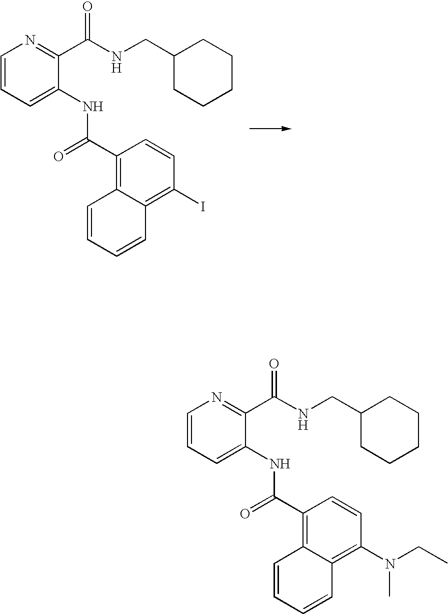

Following the procedure for example 170, using ethylmethylamine (0.05 mL, 0.58 mmol) and purifying by reverse-phase preparative HPLC provided the TFA salt of the title compound as a white solid (68 mg, 31%); ¹H NMR (400 MHz, CHLOROFORM-D) δ 0.91-1.06 (m, 2H), 1.12-1.32 (m, 4H), 1.52-1.63 (m, 1H), 1.63-1.84 (m, 4H), 2.18 (s, 3H), 3.09 (s, 3H), 3.21-3.28 (m, 2H), 3.44 (q, J=6.90 Hz, 2H), 7.30 (d, J=7.81 Hz, 1H), 7.52 (dd, J=8.59, 4.49 Hz, 1H), 7.57-7.64 (m, 2H), 7.90 (d, J=7.81 Hz, 1H), 8.28 (dd, J=4.49, 1.37 Hz, 1H), 8.30-8.38 (m, 1H), 8.56 (t, J=6.35 Hz, 1H), 8.58-8.64 (m, 1H), 9.39 (dd, J=8.59, 1.37 Hz, 1H), 12.87 (s, 1H); MS (ESI) (M+H)⁺ 445.0; Anal. Calcd. for C₂₇H₃₂N₄O₂+0.70TFA+0.10H₂O+0.10MeCN: C, 64.78; H, 6.31; N, 10.83. Found: C, 64.81; H, 6.07; N, 10.90.

### Example 173

### N-(Cyclohexylmethyl)-3-[(4-pyrrolidin-1-yl-1-naphthoyl)amino]pyridine-2-carboxamide

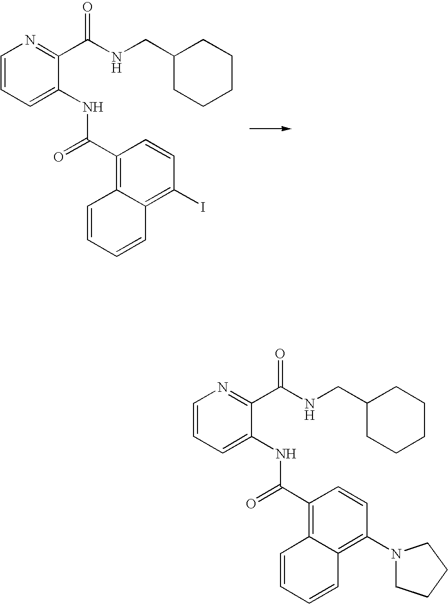

Following the procedure for example 170, using pyrrolidine (0.02 mL, 0.23 mmol) provided the title compound as a white solid (25 mg, 28%); ¹H NMR (400 MHz, CHLOROFORM-D) δ 0.88-1.07 (m, 2H), 1.09-1.33 (m, 3H), 1.54-1.62 (m, 3H), 1.67 (d, J=11.72 Hz, 1H), 1.70-1.83 (m, 2H), 1.98-2.08 (m, 4H), 3.26 (t, J=6.64 Hz, 2H), 3.45-3.55 (m, 4H), 6.89 (d, J=8.20 Hz, 1H), 7.39-7.46 (m, 1H), 7.46-7.55 (m, 2H), 7.87 (d, J=8.01 Hz, 1H), 8.19-8.25 (m, 2H), 8.53 (t, J=5.86 Hz, 1H), 8.68 (dd, J=8.59, 0.78 Hz, 1H), 9.38 (dd, J=8.59, 1.37 Hz, 1H), 12.73 (s, 1H); MS (ESI) (M+H)⁺ 457.2; Anal. Calcd. for C₂₈H₃₂N₄O₂+0.20H₂O: C, 73.08; H, 7.10; N, 12.17. Found: C, 73.08; H, 7.18; N, 11.90.

### Example 174

### N-(Cyclohexylmethyl)-3-{[4-(4-isopropylpiperazin-1-yl)-1-naphthoyl]amino}pyridine-2-carboxamide

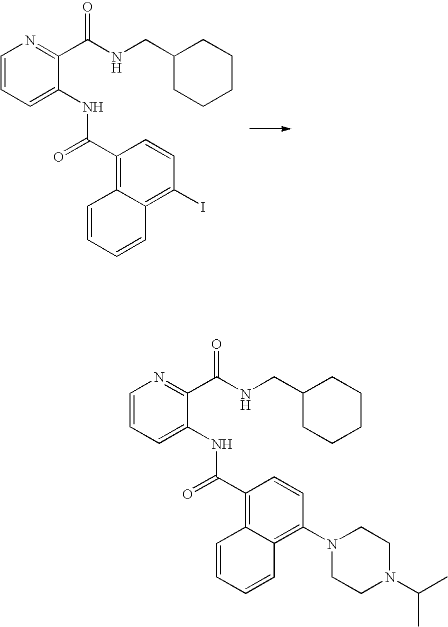

Following the procedure for example 170, using N-isopropylpiperazine (30 mg, 0.23 mmol) and purifying by reverse-phase preparative HPLC provided the TFA salt of the title compound as a white solid (33 mg, 27%); ¹H NMR (400 MHz, CHLOROFORM-D) δ 0.91-1.07 (m, 1H), 1.12-1.33 (m, 2H), 1.48 (d, J=6.64 Hz, 5H), 1.52-1.62 (m, 1H), 1.66 (d, J=13.67 Hz, 1H), 1.70-1.83 (m, 3H), 1.88 (s, 6H), 3.25 (t, J=6.54 Hz, 3H), 3.52 (d, J=6.83 Hz, 3H), 3.68 (d, J=9.76 Hz, 2H), 7.21 (d, J=7.62 Hz, 1H), 7.48-7.62 (m, 2H), 7.89 (d, J=7.62 Hz, 1H), 8.06-8.12 (m, 1H), 8.28 (dd, J=4.49, 1.56 Hz, 1H), 8.54 (t, J=6.15 Hz, 1H), 8.57-8.62 (m, 1H), 9.37 (dd, J=8.59, 1.37 Hz, 1H), 12.89 (s, 1H); MS (ESI) (M+H)⁺ 514.2; Anal. Calcd. for C₃₁H₃₉N₅O₂+1.50TFA+0.20H₂O: C, 59.33; H, 5.99; N, 10.17. Found: C, 59.40; H, 5.97; N, 9.94.

### Example 175

### N-(Cyclohexylmethyl)-3-({4-[3-(diethylamino)pyrrolidin-1-yl]-1-naphthoyl}amino)pyridine-2-carboxamide

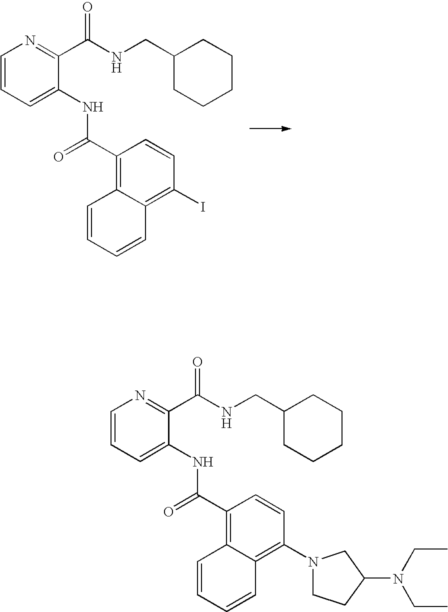

Following the procedure for example 170 using N,N-diethylpyrrolidin-3-amine (33 mg, 0.23 mmol) and purifying by reverse-phase preparative HPLC provided the TFA salt of the title compound as a white solid (37 mg, 29%); ¹H NMR (400 MHz, CHLOROFORM-D) δ 0.91-1.07 (m, 2H), 1.12-1.32 (m, 3H), 1.42 (q, J=6.90 Hz, 6H), 1.52-1.63 (m, 1H), 1.67 (d, J=11.13 Hz, 1H), 1.70-1.85 (m, 4H), 2.40-2.66 (m, 4H), 3.11-3.22 (m, 1H), 3.25 (t, J=6.64 Hz, 2H), 3.33-3.46 (m, 2H), 3.47-3.62 (m, 2H), 3.84 (dd, J=10.25, 6.35 Hz, 1H), 3.97-4.09 (m, 1H), 7.07 (d, J=8.01 Hz, 1H), 7.47-7.60 (m, 3H), 7.86 (d, J=7.81 Hz, 1H), 8.10 (d, J=7.81 Hz, 1H), 8.26 (dd, J=4.49, 1.37 Hz, 1H), 8.54 (t, J=6.54 Hz, 1H), 8.61 (dd, J=8.30, 1.27 Hz, 1H), 9.37 (dd, J=8.59, 1.37 Hz, 1H), 12.84 (s, 1H); MS (ESI) (M+H)⁺ 528.3; Anal. Calcd. for C₃₂H₄₁N₅O₂+1.50TFA: C, 60.16; H, 6.13; N, 10.02. Found: C, 60.14; H, 6.07; N, 9.85.

### Example 176

### N′-(2-{[(Cyclohexylmethyl)amino]carbonyl}pyridin-3-yl)-N,N-dimethylnaphthalene-1,4-dicarboxamide

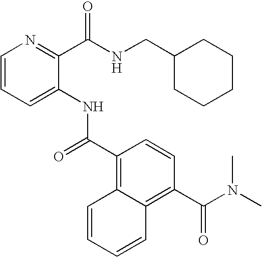

### Step A. N′-(2-{[(Cyclohexylmethyl)amino]carbonyl}pyridin-3-yl)-N,N-dimethylnaphthalene-1,4-dicarboxamide

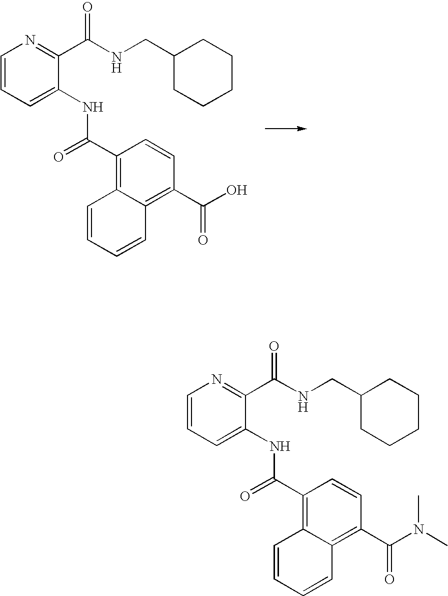

Following the procedure for Step A in Example 30 (correct??), using 4-{[(2-{[(Cyclohexylmethyl)amino]carbonyl}pyridin-3-yl)amino]carbonyl}-1-naphthoic acid (100 mg, 0.23 mmol), dimethylamine hydrochloride (187 mg, 2.31 mmol) and Et₃N (0.48 mL, 3.47 mmol) and purifying by reverse-phase preparative HPLC provided the TFA salt of the title compound as white powder (30 mg, 43%); ¹H NMR (400 MHz, CHLOROFORM-D) δ 0.91-1.06 (m, 2H), 1.09-1.33 (m, 3H), 1.51-1.62 (m, 1H), 1.63-1.84 (m, 5H), 2.84 (s, 3H), 3.24 (t, J=6.54 Hz, 2H), 3.27-3.30 (m, 3H), 7.47-7.55 (m, 2H), 7.55-7.65 (m, 2H), 7.78-7.86 (m, 1H), 7.92 (d, J=7.23 Hz, 1H), 8.29 (dd, J=4.49, 1.56 Hz, 1H), 8.49-8.60 (m, 2H), 9.39 (dd, J=8.49, 1.46 Hz, 1H), 12.94 (s, 1H); MS (ESI) (M+H)⁺ 459.0; Anal. Calcd. for C₂₇H₃₀N₄O₃+0.40TFA+0.10H₂O: C, 65.99; H, 6.10; N, 11.07. Found: C, 65.90; H, 6.00; N, 11.04.

### Step B. 4-{[(2-{[(Cyclohexylmethyl)amino]carbonyl}pyridin-3-yl)amino]carbonyl}-1-naphthoic acid

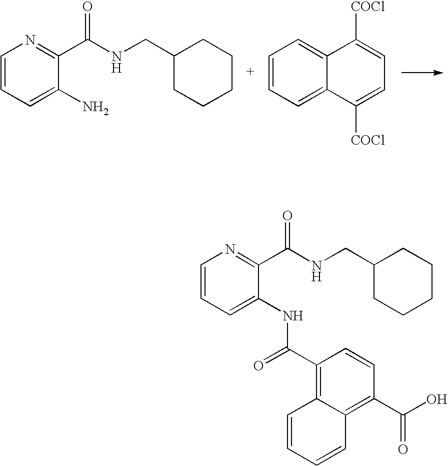

A solution of 3-Amino-N-(cyclohexylmethyl)pyridine-2-carboxamide (500 mg, 2.14 mmol, see step B. of example 82 for its preparation) and DIPEA (0.37 mL, 2.14 mmol) in THF (2 mL) was added to a solution of naphthalene-1,4-dicarbonyl dichloride (1.6 g, 6.4 mmol, see step C. of example 159 for its preparation) in THF (300 mL) at 0° C. The reaction mixture was allowed to warm to ambient temperature and NaOH 0.1 M (10 drops) and MeCN (50 mL) were added. The reaction mixture was stirred for 2 hrs. and the solvent volume was reduced. The resulting precipitate was filtered, washed with small portions of cold THF and air dried to provide the pure title compound as a white solid (600 mg, 64%).

### Example 177

### N-(Cyclohexylmethyl)-3-{[4-(methoxymethyl)-1-naphthoyl]amino}pyridine-2-carboxamide

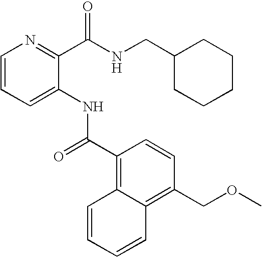

### Step A. N-(Cyclohexylmethyl)-3-{[4-(methoxymethyl)-1-naphthoyl]amino}pyridine-2-carboxamide

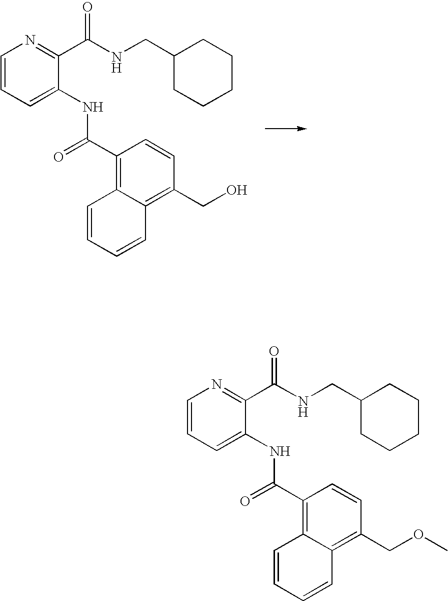

Following the procedure for Example 160, using N-(cyclohexylmethyl)-3-{[4-(hydroxymethyl)-1-naphthoyl]amino}pyridine-2-carboxamide (103 mg, 0.24 mmol) and NaOMe 25% in MeOH (10 mL) and purifying by reverse-phase preparative HPLC provided the TFA salt of title compound as white powder (30 mg, 22%); ¹H NMR (400 MHz, CHLOROFORM-D) δ 0.91-1.05 (m, 2H), 1.10-1.32 (m, 3H), 1.52-1.62 (m, 1H), 1.61-1.70 (m, 1H), 1.70-1.83 (m, 4H), 3.24 (t, J=6.54 Hz, 2H), 3.44-3.50 (m, 3H), 4.95 (s, 2H), 7.52 (dd, J=8.59, 4.49 Hz, 1H), 7.55-7.63 (m, 3H), 7.87 (d, J=7.42 Hz, 1H), 8.10-8.17 (m, 1H), 8.28 (d, J=3.91 Hz, 1H), 8.47-8.59 (m, 2H), 9.40 (d, J=8.40 Hz, 1H), 12.87 (s, 1H); MS (ESI) (M+H)⁺ 432.0.

### Step B. N-(Cyclohexylmethyl)-3-{[4-(hydroxymethyl)-1-naphthoyl]amino}pyridine-2-carboxamide

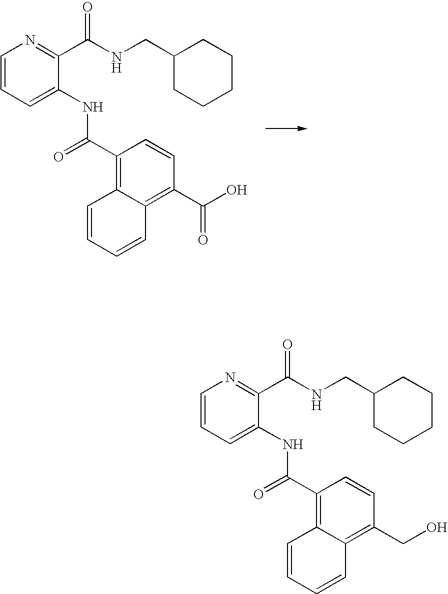

Following the procedure for Step A in Example 159, using 4-{[(2-{[(Cyclohexylmethyl)amino]carbonyl}pyridin-3-yl)amino]carbonyl}-1-naphthoic acid (600 mg, 1.39 mmol, see step B. of example 27 (not correct!!) for its preparation) and purifying on silica gel by flash chromatography provided the title compound as a white solid (307 mg, 52%).

### Example 178

### N-(Cyclohexylmethyl)-3-({4-[(dimethylamino)methyl]-1-naphthoyl}amino)pyridine-2-carboxamide

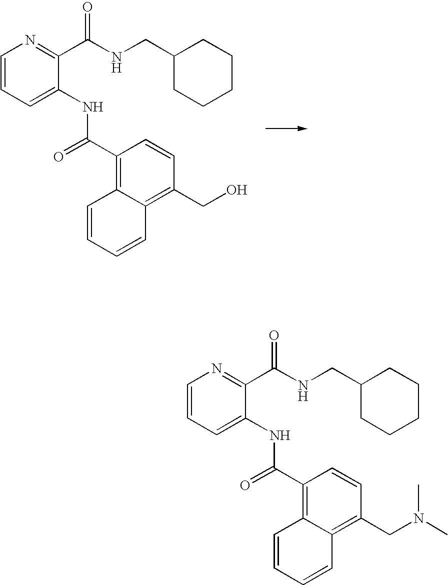

Following the procedure for example 160, using N-(cyclohexylmethyl)-3-{[4-(hydroxymethyl)-1-naphthoyl]amino}pyridine-2-carboxamide (103 mg, 0.24 mmol) provided the TFA salt of the title compound as a white solid (20 mg, 14%); ¹H NMR (400 MHz, CHLOROFORM-D) δ 0.90-1.07 (m, 2H), 1.11-1.30 (m, 2H), 1.66-1.82 (m, 4H), 2.84 (s, 6H), 3.23 (t, J=6.64 Hz, 2H), 4.76 (s, 2H), 7.54 (dd, J=8.49, 4.59 Hz, 1H), 7.61-7.74 (m, 2H), 7.84 (dd, J=59.56, 7.42 Hz, 2H), 8.17 (d, J=7.42 Hz, 1H), 8.31 (dd, J=4.49, 1.37 Hz, 1H), 8.56 (dd, J=8.20, 0.98 Hz, 2H), 9.38 (dd, J=8.59, 1.37 Hz, 1H), 12.99 (s, 1H); MS (ESI) (M+H)⁺ 445.2; Anal. Calcd. for C₂₇H₃₂N₄O₂+1.40TFA: C, 59.24; H, 5.57; N, 9.27. Found: C, 59.64; H, 4.51; N, 9.29.

### Example 179

### N-(Cyclobutylmethyl)-3-{[4-(1H-pyrrol-1-ylmethyl)-1-naphthoyl]amino}pyridine-2-carboxamide

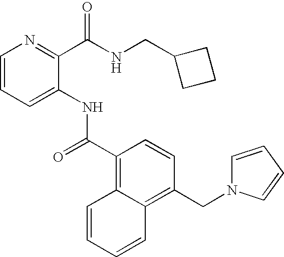

### Step A. N-(Cyclobutylmethyl)-3-{[4-(1H-pyrrol-1-ylmethyl)-1-naphthoyl]amino}pyridine-2-carboxamide

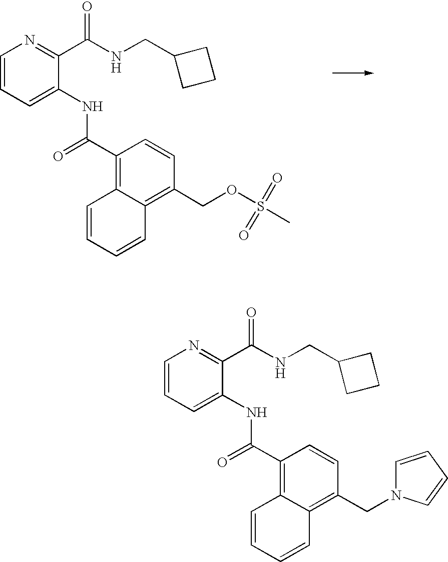

(4-{[(2-{[(Cyclobutylmethyl)amino]carbonyl}pyridin-3-yl)amino]carbonyl}-1-naphthyl)methyl methanesulfonate (85 mg, 0.18 mmol) from step D, pyrrole (624 mg, 9.30 mmol), KI (33 mg, 0.20 mmol) and DMF (2 mL) were mixed together and heated to 80° C. for 1 hrs. The solvent was concentrated and the residue was recovered in EtOAc. The solution washed with saturated NaHCO₃ solution, water, brine and dried over anhydrous Na₂SO₄. The solvent was concentrated and the product was purified by preparative reverse-phase HPLC to provide the TFA salt of the title compound as a white powder (29 mg, 28%); ¹H NMR (400 MHz, CHLOROFORM-D) δ 1.67-1.84 (m, 3H), 1.85-1.97 (m, 2H), 2.04-2.17 (m, 2H), 2.52-2.64 (m, 1H), 3.42 (dd, J=7.13, 6.15 Hz, 2H), 4.45-4.50 (m, 2H), 6.06-6.11 (m, 1H), 6.18 (q, J=2.73 Hz, 1H), 6.62-6.68 (m, 1H), 7.38 (d, J=7.42 Hz, 1H), 7.48-7.61 (m, 3H), 7.84 (d, J=7.23 Hz, 1H), 8.09-8.15 (m, 1H), 8.28 (dd, J=4.49, 1.56 Hz, 1H), 8.45 (t, J=5.76 Hz, 1H), 8.54-8.59 (m, 1H), 9.40 (dd, J=8.59, 1.56 Hz, 1H), 12.86 (s, 1H); MS (ESI) (M+H)⁺ 439.0; Anal. Calcd. for C₂₇H₂₆N₄O₂+5.10TFA+7.00MeCN+5.10H₂O: C, 43.95; H, 4.49; N, 11.01. Found: C, 44.13; H, 4.14; N, 10.93.

### Step B. 4-{[(2-{[(Cyclobutylmethyl)amino]carbonyl}pyridin-3-yl)amino]carbonyl}-1-naphthoic acid

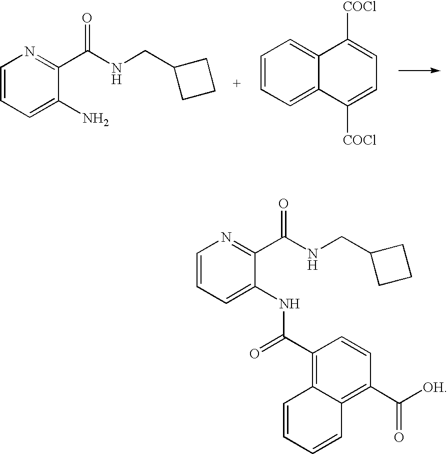

A solution of 3-Amino-N-(cyclobutylmethyl)pyridine-2-carboxamide (3.0 g, 14.6 mmol) and Et₃N (2.6 mL, 14.6 mmol) in MeCN (50 mL) was added to a solution of naphthalene-1,4-dicarbonyl dichloride (4.7 g, 18.5 mmol, see step C. of example 159 for its preparation) in MeCN (700 mL) at 0° C. The reaction mixture was stirred for 2 hrs. and NaOH 0.1 M solution (0.44 mL) was added. The reaction mixture was stirred for 1 extra hrs. and NaOH 0.1 M solution (excess) was added. The solvent was concentrated and water was added to the residue. The precipitate was filtered and the filtrate was acidified with concentrated HCl. The resulting precipitate was filtered. The precipitates were recovered in DCM, combined and dried over anhydrous Na₂SO₄. The solvent was concentrated to provide the pure title compound as beige solid (5.43 g, 92%).

### Step C. N-(Cyclobutylmethyl)-3-{[4-(hydroxymethyl)-1-naphthoyl]amino}pyridine-2-carboxamide

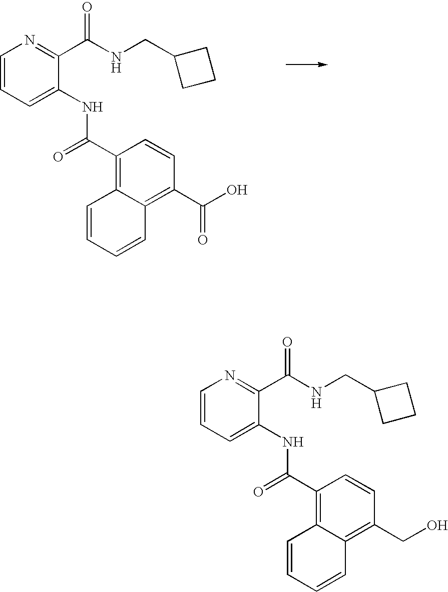

Following the procedure for Step A in Example 159, using 4-{[(2-{[(Cyclobutylmethyl)amino]carbonyl}pyridin-3-yl)amino]carbonyl}-1-naphthoic acid (1.33 g, 3.30 mmol) from step B and performing a work-up in EtOAc provided the pure title compound as pale yellow oil (1.01 g, 78%).

### Step D. (4-{[(2-{[(Cyclobutylmethyl)amino]carbonyl}pyridin-3-yl)amino]carbonyl}-1-naphthyl)methyl methanesulfonate

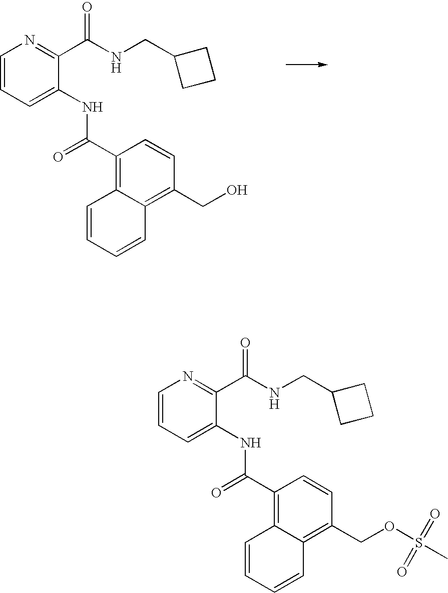

Methane sulfonyl chloride (0.24 mL, 3.11 mmol) was added to a solution of N—(Cyclobutylmethyl)-3-{[4-(hydroxymethyl)-1-naphthoyl]amino}pyridine-2-carboxamide (1.01 g, 2.59 mmol) from step C and Et₃N (0.45 mL, 3.23 mmol) in DCM (150 mL) at 0° C. The reaction mixture was allowed to warm to ambient temperature and stirred for 3 hrs. The reaction mixture washed with NaHCO₃ saturated solution, water, brine and dried over anhydrous Na₂SO₄. The solvent was concentrated and the product was purified on silica gel by flash chromatography to provide the title compound as colorless oil (342 mg, 28%).

### Example 180

### N-(Cyclobutylmethyl)-3-{[4-(1H-1,2,3-triazol-1-ylmethyl)-1-naphthoyl]amino}pyridine-2-carboxamide

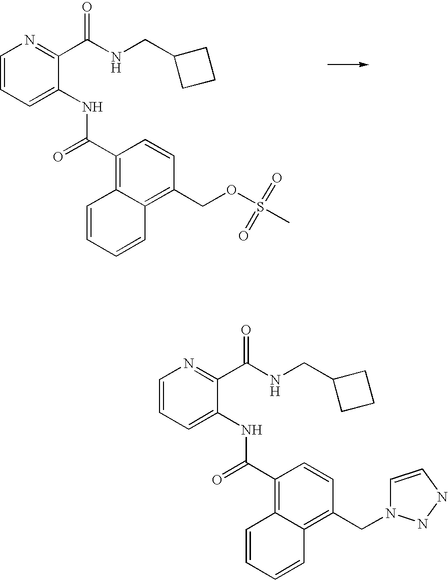

Following the procedure for example 179, using 1,2,3-triazole (0.64 g, 9.30 mmol) provided the TFA salt of the title compound as white powder (63 mg, 64%); ¹H NMR (400 MHz, CHLOROFORM-D) δ 1.68-1.81 (m, 2H), 1.85-1.98 (m, 2H), 2.05-2.16 (m, 2H), 2.52-2.65 (m, 1H), 3.42 (dd, J=7.13, 6.15 Hz, 2H), 6.08 (s, 2H), 7.43 (s, 1H), 7.48 (d, J=7.23 Hz, 1H), 7.54 (dd, J=8.59, 4.49 Hz, 1H), 7.57-7.66 (m, 2H), 7.76 (s, 1H), 7.88 (d, J=7.42 Hz, 1H), 7.95-8.02 (m, 1H), 8.30 (dd, J=4.49, 1.37 Hz, 1H), 8.48 (t, J=5.76 Hz, 1H), 8.52-8.59 (m, 1H), 9.39 (dd, J=8.59, 1.56 Hz, 1H), 12.95 (s, 1H); MS (ESI) (M+H)⁺ 441.0; Anal. Calcd. for C₂₅H₂₄N₆O₂+0.30TFA: C, 64.77; H, 5.16; N, 17.70. Found: C, 64.75; H, 5.04; N, 17.30.

### Example 181

### N-(Cyclobutylmethyl)-3-{[4-(1H-pyrazol-1-ylmethyl)-1-naphthoyl]amino}pyridine-2-carboxamide

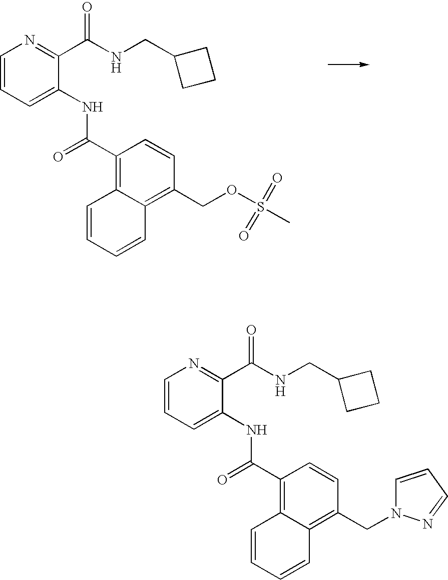

Following the procedure for example 179, using pyrazole (0.72 g, 10.5 mmol) provided the TFA salt of the title compound as white powder (33 mg, 32%); ¹H NMR (400 MHz, CHLOROFORM-D) δ 1.67-1.81 (m, 2H), 1.84-1.98 (m, 2H), 2.04-2.16 (m, 2H), 2.52-2.64 (m, 1H), 3.42 (dd, J=7.22, 6.25 Hz, 2H), 5.85 (s, 2H), 6.30 (s, 1H), 7.22-7.28 (m, 1H), 7.33 (s, 1H), 7.52 (dd, J=8.59, 4.49 Hz, 1H), 7.56-7.61 (m, 2H), 7.61-7.65 (m, 1H), 7.85 (d, J=7.42 Hz, 1H), 7.98-8.06 (m, 1H), 8.28 (dd, J=4.49, 1.56 Hz, 1H), 8.44 (t, J=5.76 Hz, 1H), 8.53-8.61 (m, 1H), 9.39 (dd, J=8.59, 1.37 Hz, 1H), 12.90 (s, 1H); MS (ESI) (M+H)⁺ 440.0; Anal. Calcd. for C₂₆H₂₅N₅O₂+0.70TFA+0.10H₂O+0.80MeCN: C, 62.88; H, 5.15; N, 14.66. Found: C, 62.89; H, 4.86; N, 14.66.

### Example 182

### N-(Cyclobutylmethyl)-3-[(4-{[ethyl(methyl)amino]methyl}-1-naphthoyl)amino]pyridine-2-carboxamide

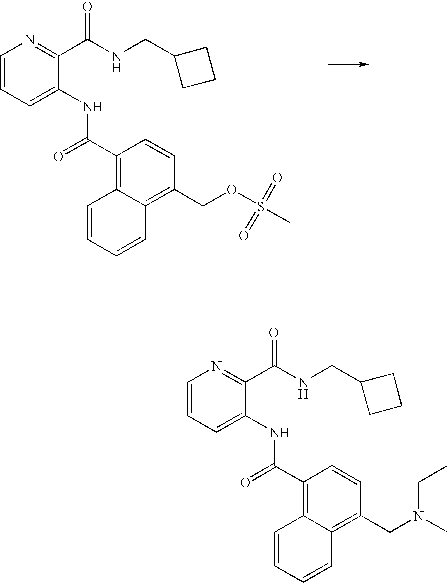

Following the procedure for example 179, using ethylmethylamine (0.55 g, 9.30 mmol) provided the TFA salt of the title compound as white powder (95 mg, 96%); ¹H NMR (400 MHz, CHLOROFORM-D) δ 1.43 (t, J=7.23 Hz, 3H), 1.67-1.82 (m, 2H), 1.84-1.99 (m, 2H), 2.05-2.16 (m, 2H), 2.52-2.65 (m, 1H), 2.75 (s, 3H), 2.96-3.10 (m, 1 H), 3.42 (dd, J=7.03, 6.25 Hz, 3H), 4.65-4.92 (m, 2H), 7.54 (dd, J=8.59, 4.49 Hz, 1H), 7.61-7.74 (m, 2H), 7.85 (dd, J=49.79, 7.42 Hz, 2H), 8.16 (d, J=8.20 Hz, 1H), 8.31 (dd, J=4.49, 1.56 Hz, 1H), 8.47 (t, J=5.76 Hz, 1H), 8.55 (dd, J=8.40, 1.17 Hz, 1H), 9.38 (dd, J=8.59, 1.37 Hz, 1H), 12.98 (s, 1H); MS (ESI) (M+H)⁺ 431.3; Anal. Calcd. for C₂₆H₃₀N₄O₂+1.90TFA+1.00H₂O+0.60MeCN: C, 53.97; H, 5.22; N, 9.34. Found: C, 53.93; H, 5.19; N, 9.40.

### Example 183

### N-(Cyclobutylmethyl)-3-{[4-(1H-imidazol-1-ylmethyl)-1-naphthoyl]amino}pyridine-2-carboxamide

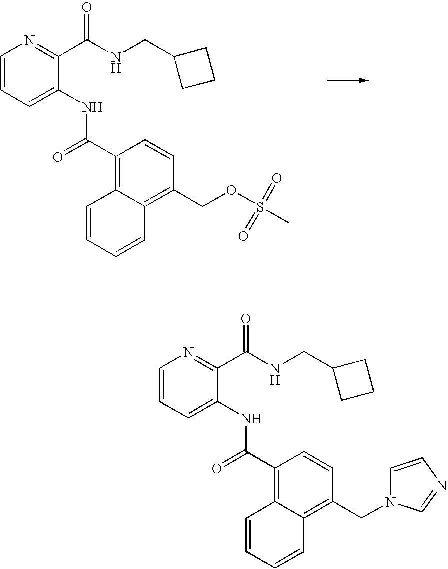

Following the procedure for example 179, using imidazole (0.33 g, 4.84 mmol) provided the TFA salt of the title compound as white powder (50 mg, 18%); ¹H NMR (400 MHz, CHLOROFORM-D) δ 1.68-1.81 (m, 2H), 1.83-1.99 (m, 2H), 2.04-2.16 (m, 2H), 2.52-2.64 (m, 1H), 3.37-3.45 (m, 2H), 5.83 (s, 2H), 7.04 (s, 1H), 7.36 (s, 1H), 7.46 (d, J=7.42 Hz, 1H), 7.54 (dd, J=8.59, 4.49 Hz, 1H), 7.60-7.69 (m, 2H), 7.82-7.92 (m, 2H), 8.31 (dd, J=4.49, 1.37 Hz, 1H), 8.47 (t, J=5.96 Hz, 1H), 8.55-8.62 (m, 1H), 8.98 (s, 1H), 9.38 (dd, J=8.59, 1.37 Hz, 1H), 13.00 (s, 1H), MS (ESI) (M+H)⁺ 440.0; Anal. Calcd. for C₂₆H₂₅N₅O₂+1.20TFA+0.10H₂O: C, 59.00; H, 4.60; N, 12.11. Found: C, 59.05; H, 4.72; N, 12.04.

### Example 184

### N-(Cyclobutylmethyl)-3-({4-[(dimethylamino)methyl]-1-naphthoyl}amino)pyridine-2-carboxamide

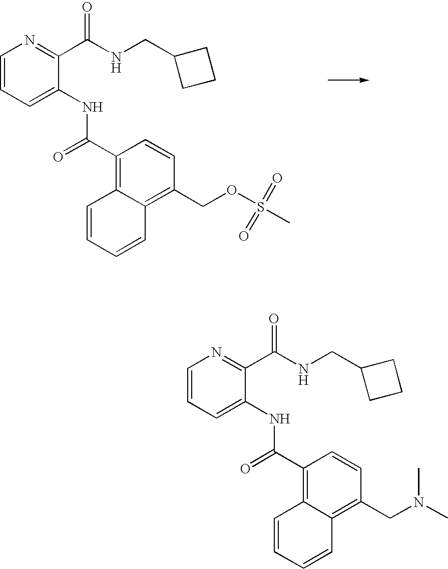

Following the procedure for example 179, using dimethylamine hydrochloride (0.20 g, 2.45 mmol) provided the TFA salt of the title compound as white powder (30 mg, 44%); ¹H NMR (400 MHz, CHLOROFORM-D) δ 1.69-1.80 (m, 2H), 1.85-1.98 (m, 2H), 2.05-2.16 (m, 2H), 2.53-2.64 (m, 1H), 2.84 (s, 6H), 3.38-3.45 (m, 2H), 4.73-4.79 (m, 2H), 7.55 (dd, J=8.49, 4.59 Hz, 1H), 7.63-7.74 (m, 2H), 7.85 (dd, J=60.44, 7.32 Hz, 2H), 8.17 (d, J=7.81 Hz, 1H), 8.31 (dd, J=4.49, 1.37 Hz, 1H), 8.46 (t, J=5.66 Hz, 1H), 8.56 (dd, J=8.40, 1.17 Hz, 1H), 9.38 (dd, J=8.59, 1.56 Hz, 1H), 12.99 (s, 1H), MS (ESI) (M+H)⁺ 417.3; Anal. Calcd. for C₂₅H₂₈N₄O₂+1.30TFA+0.70H₂O: C, 57.42; H, 5.36; N, 9.70. Found: C, 57.50; H, 5.31; N, 9.65.

### Example 185

### N-(Cyclobutylmethyl)-3-{[4-(methoxymethyl)-1-naphthoyl]amino}pyridine-2-carboxamide

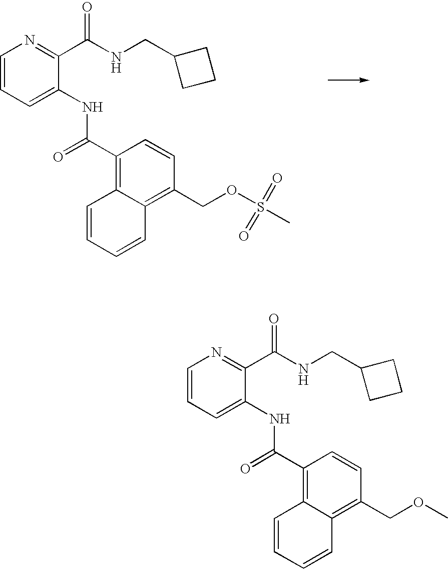

Following the procedure for example 179, using NaOMe 20% in MeOH (15 mL) provided the TFA salt of the title compound as white powder (30 mg, 44%); ¹H NMR (400 MHz, CHLOROFORM-D) δ 1.68-1.81 (m, 2H), 1.83-1.99 (m, 2H), 2.03-2.16 (m, 2H), 2.52-2.64 (m, 1H), 3.42 (t, J=6.05 Hz, 2H), 3.47 (s, 3H), 4.92-4.99 (m, 2H), 7.52 (dd, J=3.12, 1.37 Hz, 1H), 7.59 (dd, J=6.64, 2.73 Hz, 3H), 7.87 (d, J=7.23 Hz, 1H), 8.14 (dd, J=6.64, 2.93 Hz, 1H), 8.28 (s, 1H), 8.43 (s, 1H), 8.56 (dd, J=6.64, 2.93 Hz, 1H), 9.40 (d, J=8.20 Hz, 1H), 12.87 (s, 1H); MS (ESI) (M+H)⁺ 404.0; Anal. Calcd. for C₂₄H₂₅N₃O₃+0.10H₂O: C, 71.13; H, 6.27; N, 10.37. Found: C, 71.07; H, 6.53; N, 9.91.

### Example 186

### N-(Cyclobutylmethyl)-3-{[4-(ethoxymethyl)-1-naphthoyl]amino}pyridine-2-carboxamide

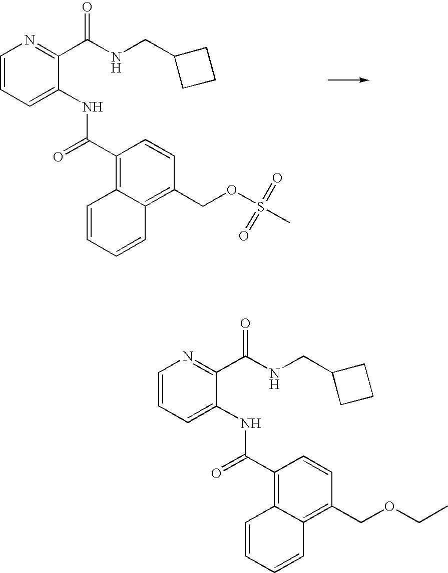

NaH 60% suspension in oil (0.20 g, 5.00 mmol) was slowly added to EtOH (20 mL) at 0° C. The reaction mixture was allowed to warm to ambient temperature and stirred for 1 hr. The solution was cooled to 0° C. and a solution of (4-{[(2-{[(Cyclobutylmethyl)amino]carbonyl}pyridin-3-yl)amino]carbonyl}-1-naphthyl)methyl methanesulfonate (60 mg, 0.12 mmol) in EtOH (2 mL) was added. The reaction mixture was allowed to warm to ambient temperature, heated to 70° C. and stirred for 3 hrs. The solvent was concentrated and the product was purified by preparative reverse-phase HPLC to provide the TFA salt of the title compound as white solid (40 mg, 58%); ¹H NMR (400 MHz, CHLOROFORM-D) δ 1.29 (t, J=6.93 Hz, 3H), 1.67-1.82 (m, 2H), 1.84-1.97 (m, 2H), 2.03-2.16 (m, 2H), 2.53-2.64 (m, 1H), 3.37-3.48 (m, J=3.51 Hz, 2H), 3.64 (q, J=7.03 Hz, 2H), 5.00 (s, 2H), 7.48-7.55 (m, 1H), 7.55-7.64 (m, 3H), 7.87 (d, J=6.83 Hz, 1H), 8.15 (dd, J=6.44, 3.12 Hz, 1H), 8.27 (s, 1H), 8.45 (s, 1H), 8.56 (dd, J=6.35, 2.83 Hz, 1H), 9.42 (d, J=6.64 Hz, 1H), 12.87 (s, 1H); MS (ESI) (M+H)⁺ 418.0; Anal. Calcd. for C₂₅H₂₇N₃O₃: C, 71.92; H, 6.52; N, 10.06. Found: C, 71.94; H, 6.18; N, 9.64.

### Example 187

### N′-(2-{[(Cyclobutylmethyl)amino]carbonyl}pyridin-3-yl)-N,N-dimethylnaphthalene-1,4-dicarboxamide

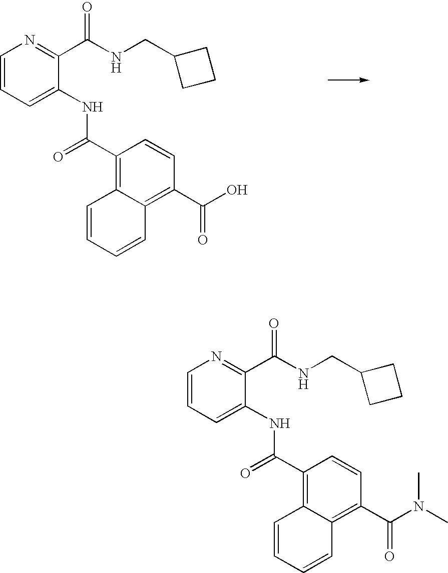

Following the procedure for Step A in Example 30 (correct??), using 4-{[(2-{[(Cyclobutylmethyl)amino]carbonyl}pyridin-3-yl)amino]carbonyl}-1-naphthoic acid (50 mg, 0.12 mmol), dimethylamine hydrochloride (100 mg, 1.23 mmol) and Et₃N (0.20 mL, 1.23 mmol) and purifying by preparative reversed-phase HPLC provided the TFA salt of the title compound as a white powder (25 mg, 37%); ¹H NMR (400 MHz, CHLOROFORM-D) δ 1.69-1.81 (m, 2H), 1.84-1.97 (m, 2H), 2.05-2.21 (m, 2H), 2.54-2.64 (m, 1H), 2.84 (s, 3H), 3.29 (s, 3H), 3.40-3.45 (m, 2H), 7.50 (d, J=7.22 Hz, 1H), 7.53 (dd, J=8.59, 4.49 Hz, 1H), 7.56-7.64 (m, 2H), 7.80-7.85 (m, 1H), 7.92 (d, J=7.42 Hz, 1H), 8.29 (dd, J=4.49, 1.17 Hz, 1H), 8.45 (t, J=5.57 Hz, 1H), 8.57 (dd, J=7.81, 1.56 Hz, 1H), 9.40 (dd, J=8.49, 1.46 Hz, 1H), 12.94 (s, 1H); MS (ESI) (M+H)⁺ 431.0; Anal. Calcd. for C₂₅H₂₆N₄O₃+0.30H₂O: C, 68.88; H, 6.15; N, 12.85. Found: C, 68.89; H, 5.99; N, 12.75.

### Example 188

### N-(Cyclohexylmethyl)-3-{[4-(dimethylamino)-1-naphthoyl]amino}pyrazine-2-carboxamide

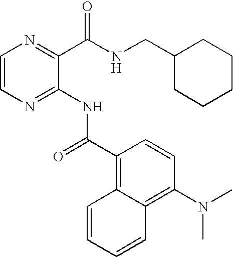

### Step A. N-(Cyclohexylmethyl)-3-{[4-(dimethylamino)-1-naphthoyl]amino}pyrazine-2-carboxamide

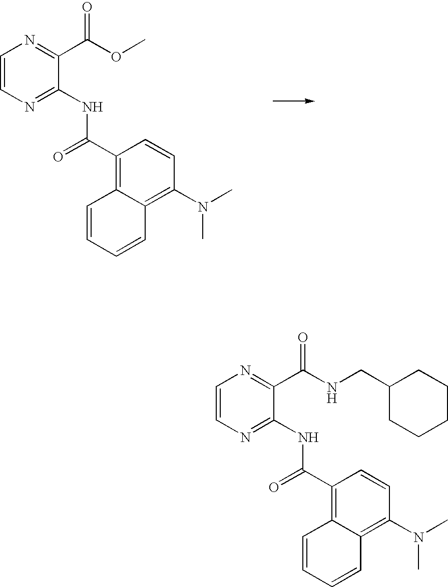

Methyl 3-{[4-(dimethylamino)-1-naphthoyl]amino}pyrazine-2-carboxylate (100 mg, 0.28 mmol) and cyclohexylmethylamine (0.18 mL, 1.42 mmol) in EtOH (25 mL) were heated to 90° C. for 2 days. The solvent was concentrated and the product was purified by reverse-phase HPLC to provide the TFA salt of the title compound as a yellow solid (105 mg, 67%); ¹H NMR (400 MHz, CHLOROFORM-D) δ 0.83-0.89 (m, J=7.03 Hz, 1H), 0.92-1.06 (m, 2H), 1.12-1.32 (m, 3H), 1.52-1.64 (m, 1H), 1.64-1.82 (m, 4H), 3.07 (s, 6H), 3.27 (t, J=6.64 Hz, 2H), 7.21 (d, J=8.01 Hz, 1H), 7.54-7.62 (m, 2H), 7.94 (d, J=7.81° Hz, 1H), 8.22-8.32 (m, 3H), 8.68-8.75 (m, 2H), 12.71 (s, 1H); MS (ESI) (M+H)⁺ 432.0; Anal. Calcd. for C₂₅H₂₉N₅O₂+0.60TFA+0.10H₂O: C, 62.72; H, 5.99; N, 13.96. Found: C, 62.91; H, 6.06; N, 13.06.

### Step B. Methyl 3-{[4-(dimethylamino)-1-naphthoyl]amino}pyrazine-2-carboxylate

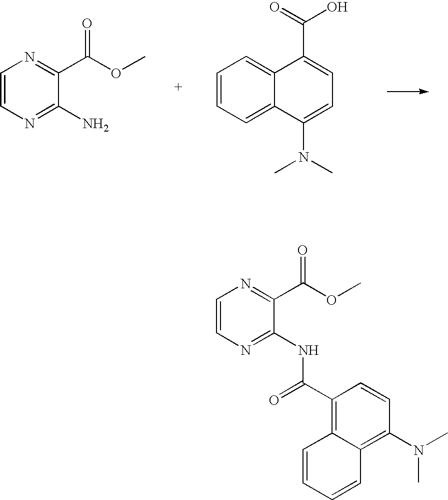

Oxalyl chloride (1.70 mL, 19.5 mmol) was added to a solution of 4-(dimethylamino)-1-naphthoic acid (2.63 g, 12.2 mmol) in DCE (125 mL) at 0° C. The reaction mixture was allowed to warm to ambient temperature, heated to 85° C. and stirred for 10 min. The reaction mixture was evaporated to dryness and the red residue was suspended in DCE (30 mL). The resulting suspension was added drop wise via a pump syringe over 7 hrs. to a solution of methyl 3-aminopyrazine-2-carboxylate (1.25 g, 8.16 mmol) and pyridine (4.75 mL, 58.7 mmol) in DCE (125 mL) at 80° C. The reaction mixture was stirred for 10 hrs at 80° C., cooled to ambient temperature and washed with 0.1M HCl solution. The solvent was concentrated and the product was purified on silica gel by MPLC to provide the title compound as white solid (1.24 g, 43%)

### Example 189

### N-(Cyclohexylmethyl)-3-{[5-(dimethylamino)-1-naphthoyl]amino}pyridine-2-carboxamide

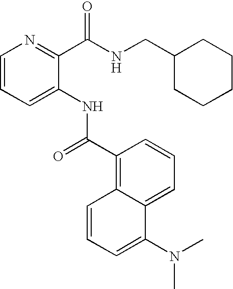

### Step A. N-(Cyclohexylmethyl)-3-{[5-(dimethylamino)-1-naphthoyl]amino}pyridine-2-carboxamide

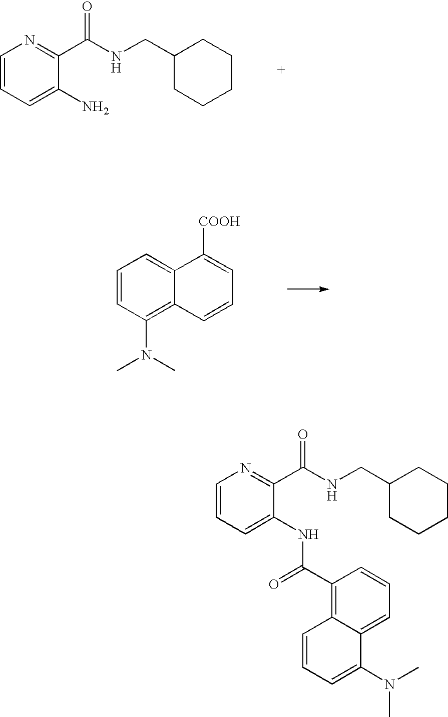

Following the procedure for Step B in Example 188, using 3-Amino-N-(cyclohexylmethyl)pyridine-2-carboxamide (279 mg, 1.19 mmol) and 5-(Dimethylamino)-1-naphthoic acid (387 mg, 1.79 mmol) and purifying the product by preparative reverse-phase HPLC provide the TFA salt of the title compound as yellow solid (30 mg, 4%); ¹H NMR (400 MHz, CHLOROFORM-D) δ 0.82-0.89 (m, 1H), 0.92-1.05 (m, 2H), 1.12-1.31 (m, 3H), 1.52-1.62 (m, 1H), 1.63-1.83 (m, 4H), 3.21-3.25 (m, 2H), 3.25-3.30 (m, 6H), 7.47-7.51 (m, 1H), 7.53 (dd, J=8.59, 4.49 Hz, 1H), 7.55-7.61 (m, 1H), 7.73 (dd, J=8.59, 7.23 Hz, 1H), 7.98 (d, J=7.03 Hz, 1H), 8.30 (dd, J=4.49, 1.37 Hz, 1H), 8.45-8.58 (m, 3H), 9.37 (dd, J=8.59, 1.37 Hz, 1H), 12.96 (s, 1H); MS (ESI) (M+H)⁺ 431.0; Anal. Calcd. for C₂₆H₃₀N₄O₂+0.30TFA: C, 68.74; H, 6.57; N, 12.05. Found: C, 69.20; H, 6.01; N, 10.06.

### Step B-C-D-E. 5-(Dimethylamino)-1-naphthoic acid

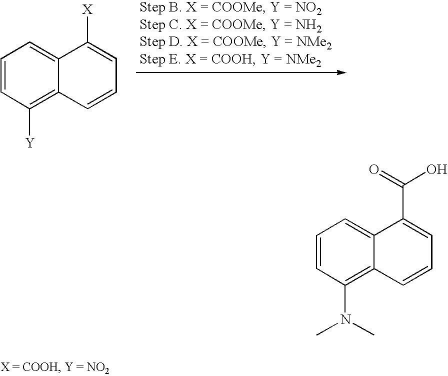

A 3 M solution of diazomethane in Et₂O (25 mL) was added to a solution of 5-nitro-1-naphthoic acid (2.40 g, 11.0 mmol) in THF (150 mL) at 0° C. The reaction mixture was allowed to warm to ambient temperature and stirred overnight. The solvent was concentrated and the product recovered in EtOAc (150 mL). The resulting solution was shaken overnight with 10% Pd/C in a Parr apparatus under 50 PSI hydrogen. The mixture was filtered on a celite pad and the solvent was concentrated. The residue, K₂CO₃ (7.64 g, 55.2 mmol) and MeI (4.69 g, 33.1 mmol) in THF were heated to 72° C. for 3 days. The solvent was concentrated. The product was recovered in EtOAc, washed with saturated NaHCO₃ solution, water, brine and dried over anhydrous Na₂SO₄. The solvent was concentrated and the product was purified on silica gel by MPLC using EtOAc in heptane 10 to 20% to provide colorless oil. The oil was mixed with 2 M NaOH (100 mL). The mixture was heated to 95° C. and stirred overnight. The reaction mixture was cooled to 0° C. and acidified with concentrated HCl (18 mL). The product was extracted with Et₂O, EtOAc and DCM. The organic phases were combined and dried with anhydrous Na₂SO₄. The solvent was concentrated to provide the pure title compound as yellow solid. Yield: 1.36 g (56%).

### Example 190

### 3-{[4-(Dimethylamino)-1-naphthoyl]amino}-N-(piperidin-2-ylmethyl)pyridine-2-carboxamide

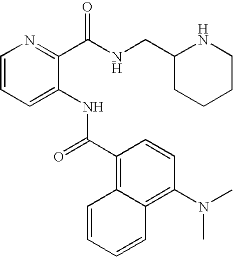

### Step A. 3-{[4-(Dimethylamino)-1-naphthoyl]amino}-N-(piperidin-2-ylmethyl)pyridine-2-carboxamide

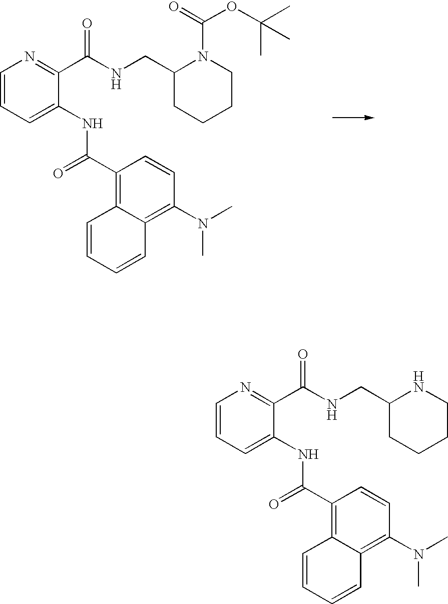

The TFA salt of tert-Butyl 2-({[(3-{[4-(dimethylamino)-1-naphthoyl]amino}pyridin-2-yl)carbonyl]amino}methyl)piperidine-1-carboxylate (56 mg, 0.086 mmol) was added to TFA (5 mL) at 0° C. The reaction mixture was allowed to warm to ambient temperature and stirred for 3 hrs. The solvent was concentrated and the product was purified by preparative reverse-phase HPLC to provide the TFA salt of the title compound as a white solid (30 mg, 64%); ¹H NMR (400 MHz, CHLOROFORM-D) δ 1.40 (t, J=13.08 Hz, 1H), 1.48-1.74 (m, 3H), 1.74-1.89 (m, 2H), 2.65-2.80 (m, 1H), 3.16 (s, 6H), 3.28 (d, J=12.89 Hz, 1H), 3.42-3.63 (m, 2H), 7.36 (d, J=7.81 Hz, 1H), 7.47 (dd, 3-8.59, 4.49 Hz, 1H), 7.57-7.67 (m, 2H), 7.85 (d, 3-7.81 Hz, 1H), 8.21 (d, J=3.71 Hz, 1H), 8.25-8.32 (m, 1H), 8.53-8.61 (m, 1H), 8.93 (t, J=6.15 Hz, 1H), 9.27 (dd, J=8.59, 0.98 Hz, 2H), 12.46 (s, 1H); MS (ESI) (M+H)⁺ 432.2; Anal. Calcd. for C₂₅H₂₉N₅O₂+2.50TFA+0.20H₂O: C, 50.03; H, 4.46; N, 9.72. Found: C, 50.00; H, 4.47; N, 9.78.

### Step B. tert-Butyl 2-({[(3-aminopyridin-2-yl)carbonyl]amino}methyl)piperidine-1-carboxylate

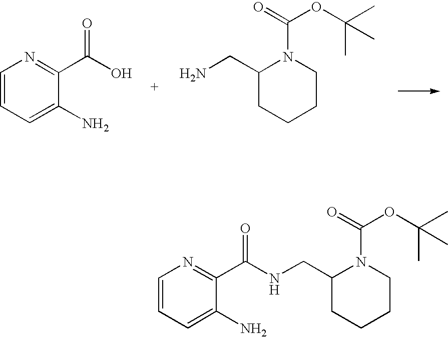

Following the procedure for Step B in Example 30, using tert-butyl 2-(aminomethyl)piperidine-1-carboxylate (0.49 g, 2.30 mmol) and purifying on silica gel by flash chromatography provided the title compound as colorless oil (477 mg, 92%).

### Step C. tert-Butyl 2-({[(3-{[4-(dimethylamino)-1-naphthoyl]amino}pyridin-2-yl)carbonyl]amino}methyl)piperidine-1-carboxylate

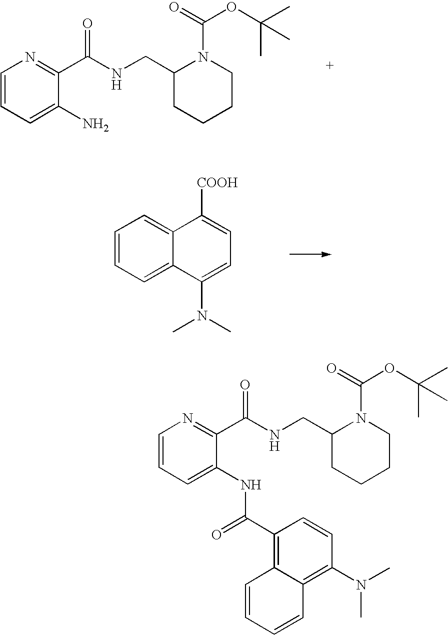

Oxalyl chloride (0.10 mL, 1.24 mmol) was added to a solution of 4-(dimethylamino)-1-naphthoic acid (0.17 g, 0.82 mmol) in DCM (40 mL) at 0° C. The reaction mixture was allowed to warm to ambient temperature and stirred for 1 hr. The solvent was concentrated. The product was purified by preparative reverse-phase HPLC to provide the TFA salt of the title compound as a white solid (56 mg, 10%).

### Example 191

### 3-{[4-(dimethylamino)-1-naphthoyl]amino}-N-pentylpyridine-2-carboxamide

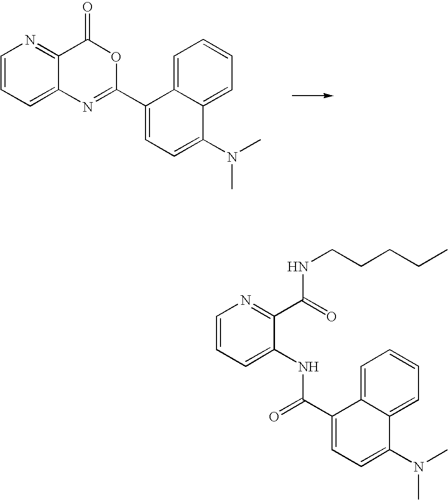

Following the procedure for Step A in Example 1, using 2-[4-(dimethylamino)-1-naphthyl]-4H-pyrido[3,2-d][1,3]oxazin-4-one (0.47 mmol) and amylamine (0.27 mL, 2.36 mmol) provided the title compound (26 mg, 14%). ¹H NMR (400 MHz, CDCl₃) δ ppm 0.89 (t, J=6.93 Hz, 3H), 1.26-1.41 (m, 4H), 1.53-1.68 (m, 2H), 2.95 (s, 6H), 3.38 (q, J=6.96 Hz, 2H), 7.08 (d, J=7.42 Hz, 1H), 7.43-7.59 (m, 3H), 7.87 (d, J=7.81 Hz, 1H), 8.18-8.32 (m, 2H), 8.45 (t, J=4.78 Hz, 1H), 8.57-8.65 (m, 1H), 9.38 (dd, J=8.59, 1.17 Hz, 1H), 12.78 (s, 1H). found: C, 69.01; H, 6.87; N, 13.05. C₂₄H₂₈N₄O₂×0.3HCl×0.1H₂O has C, 69.09; H, 6.88; N, 13.43%; MS (ESI) (M+H)⁺ 405.0

### Example 192

### 3-{[4-(dimethylamino)-1-naphthoyl]amino}-N-hexylpyridine-2-carboxamide

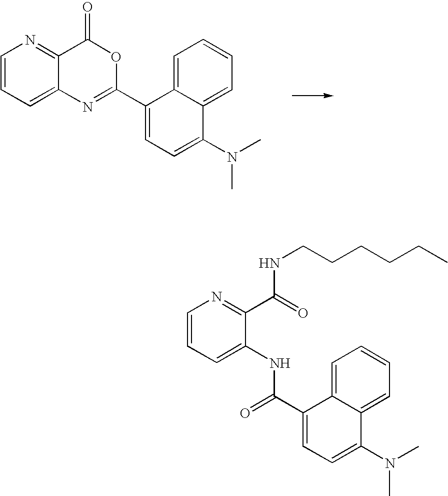

Following the procedure for Step A in Example 1, using 2-[4-(dimethylamino)-1-naphthyl]-4H-pyrido[3,2-d][1,3]oxazin-4-one (0.57 mmol) and hexylamine (0.38 mL, 2.85 mmol) provided the title compound. ¹H NMR (400 MHz, CD₃OD) δ ppm 0.81 (t, J=7.42 Hz, 6H), 1.21-1.33 (m, 4H), 1.38-1.50 (m, 1H), 2.85 (s, 6H), 3.17-3.24 (m, 2H), 7.05 (d, J=7.81 Hz, 1H), 7.40-7.52 (m, 3H), 7.76 (d, J=7.81 Hz, 1H), 8.14-8.20 (m, 1H), 8.24 (dd, J=4.49, 1.37 Hz, 1H), 8.37-8.45 (m, 1H), 8.89 (t, J=5.27 Hz, 1H), 9.17 (dd, J=8.59, 1.37 Hz, 1H), 12.77 (s, 1H); MS (ESI) (M+H)⁺ 419.0

### Example 193

### 3-{[4-(dimethylamino)-1-naphthoyl]amino}-N-[3-(dimethylamino)propyl]pyridine-2-carboxamide

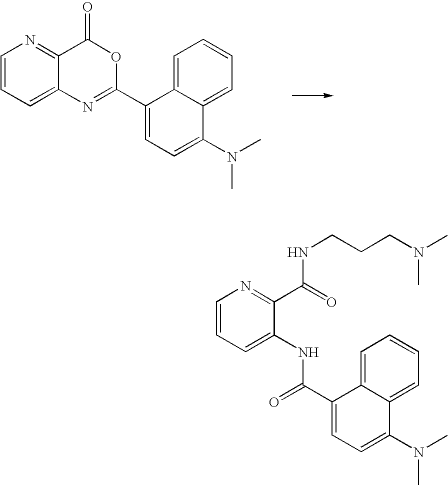

Following the procedure for Step A in Example 1, using 2-[4-(dimethylamino)-1-naphthyl]-4H-pyrido[3,2-d][1,3]oxazin-4-one (0.57 mmol) and N,N-dimethylpropane-1,3-diamine (0.36 mL; 2.85 mmol) provided the title compound. MS (ESI) (M+H)⁺ 419.0

### Example 194

### 3-{[4-(dimethylamino)-1-naphthoyl]amino}-N-propylpyridine-2-carboxamide

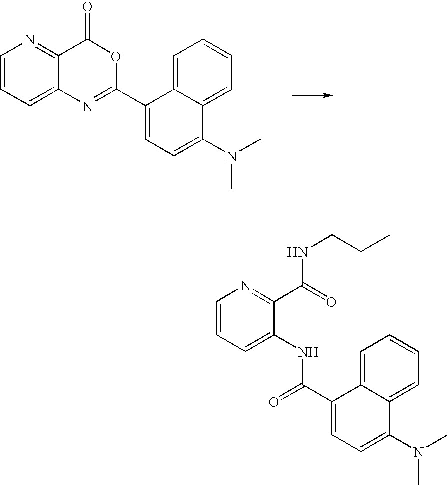

Following the procedure for Step A in Example 1, using 2-[4-(dimethylamino)-1-naphthyl]-4H-pyrido[3,2-d][1,3]oxazin-4-one (0.57 mmol) and propylamine (0.93 mL; 11.40 mmol) provided the title compound. ¹H NMR (400 MHz, CD₃OD) 5 ppm 0.85 (t, J=7.42 Hz, 3H), 1.52 (sext, 2H), 2.92 (s, 6H), 3.17-3.25 (m, 2H), 7.16 (d, J=8.01 Hz, 1H), 7.45-7.53 (m, 3H), 7.79 (d, J=8.01 Hz, 1H), 8.14-8.20 (m, 1H), 8.25 (dd, J=4.49, 1.56 Hz, 1H), 8.39-8.45 (m, 1H), 9.17 (dd, J=8.59, 1.56 Hz, 1H); MS (ESI) (M+H)⁺ 377.0

### Example 195

### 3-{[4-(dimethylamino)-1-naphthoyl]amino}-N-(2-ethylbutyl)pyridine-2-carboxamide

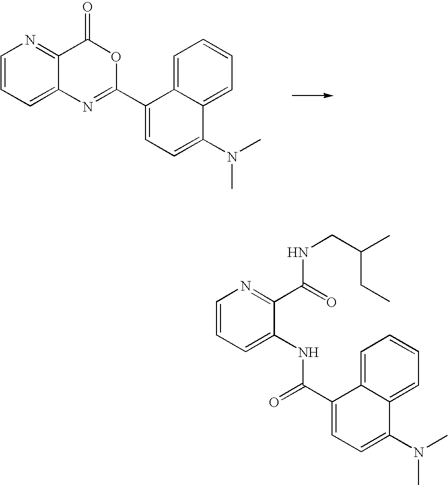

Following the procedure for Step A in Example 1, using 2-[4-(dimethylamino)-1-naphthyl]-4H-pyrido[3,2-d][1,3]oxazin-4-one (0.57 mmol) and (2-ethylbutyl)amine (0.37 mL; 2.85 mmol) provided the title compound. ¹H NMR (400 MHz, CDCl₃) δ ppm 0.72-1.00 (m, 3H), 1.14-1.49 (m, 6H), 1.49-1.73 (m, 2H), 2.96 (s, 6H), 3.39 (q, J=6.51 Hz, 2H), 6.97-7.21 (m, 1H), 7.38-7.68 (m, 3H), 7.87 (d, J=6.44 Hz, 1H), 8.14-8.37 (m, 2H), 8.46 (s, 1H), 8.61 (d, J=7.62 Hz, 1H), 9.38 (d, J=8.01 Hz, 1H), 12.79 (s, 1H). found: C, 68.43; H, 6.93; N, 12.18. C₂₅H₃₀N₄O₂×0.6HCl has C, 68.18; H, 7.00; N, 12.72%; MS (ESI) (M+H)⁺ 419.0

### Example 196

### N-(cyclohexylmethyl)-3-{[(5-phenyl-1,3-oxazol-4-yl)carbonyl]amino}pyridine-2-carboxamide

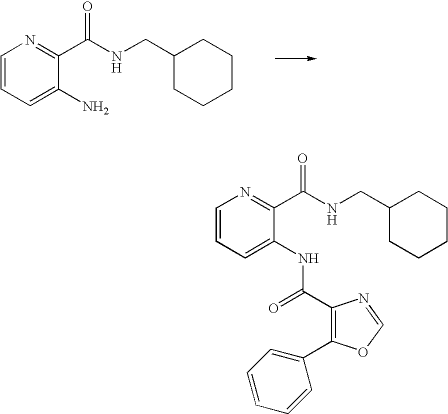

To a stock solution of 3-amino-N-(cyclohexylmethyl)pyridine-2-carboxamide in dimethylformamide (1.02 mmol) was added more dimethylformamide (3 mL), diisopropylethyl amine (0.81 mL; 4.65 mmol) followed by 5-phenyl-1,3-oxazole-4-carbonyl chloride (193 mg; 0.93 mmol). The reaction mixture was stirred over weekend, then was heated to 100° C. and stirred for 3 days. The reaction mixture was concentrated under reduced pressure. The residue was taken in ethyl acetate and washed with water. The organic layer was dried over anhydrous sodium sulphate, filtered and concentrated under reduced pressure to give the crude material. The crude material was suspended in acetonitrile and filtered to give the title compound. ¹H NMR (400 MHz, CDCl₃) δ ppm 0.93-1.10 (m, 2H), 1.09-1.36 (m, 3H), 1.46-1.91 (m, 6H), 3.35 (t, J=6.54 Hz, 2H), 7.38-7.55 (m, 4H), 8.01 (s, 1H), 8.18-8.30 (m, 3H), 8.53 (t, J=5.37 Hz, 1H), 9.30 (dd, J=8.59, 1.37 Hz, 1H), 13.50 (s, 1H); MS (ESI) (M+H)⁺ 405.0

### Example 197

### N-butyl-3-{[4-(dimethylamino)-1-naphthoyl]amino}pyridine-2-carboxamide

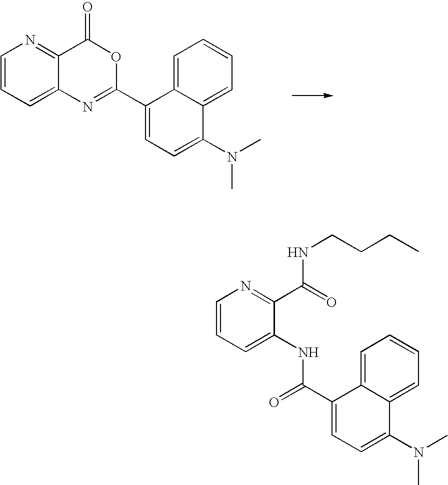

Following the procedure for Step A in Example 1, using 2-[4-(dimethylamino)-1-naphthyl]-4H-pyrido[3,2-d][1,3]oxazin-4-one (0.47 mmol) and N-butylamine (0.23 mL; 2.33 mmol) provided the title compound (16%). ¹H NMR (400 MHz, CDCl₃) δ ppm 0.93 (t, J=7.32 Hz, 3H), 1.40 (sext, J=7.61 Hz, 2H), 1.59 (quint, J=7.37 Hz, 2H), 3.02 (s, 6H), 3.39 (q, J=7.03 Hz, 2H), 7.10-7.24 (br. s, 1H), 7.49 (dd, J=8.59, 4.49 Hz, 1H), 7.56 (dd, J=6.44, 3.12 Hz, 2H), 7.87 (d, J=7.81 Hz, 1H), 8.24 (dd, J=4.49, 1.56 Hz, 1H), 8.31-8.43 (br. s, 1H), 8.46 (t, J=5.37 Hz, 1H), 8.56-8.65 (m, 1H), 9.37 (dd, J=8.59, 1.37 Hz, 1H), 12.82 (s, 1H); MS (ESI) (M+H)⁺ 391.0

### Example 198

### 3-{[(5-phenyl-1,3-oxazol-4-yl)carbonyl]amino}-N-(tetrahydro-2H-pyran-4-ylmethyl)pyridine-2-carboxamide

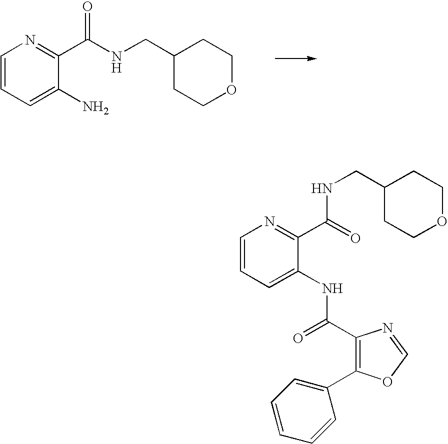

To a room temperature solution of 3-amino-N-(tetrahydro-2H-pyran-4-ylmethyl)pyridine-2-carboxamide (200 mg; 0.85 mmol) in dimethylformamide (2.6 mL) was added diisopropylethyl amine (0.67 mL; 3.86 mmol) followed by 5-phenyl-1,3-oxazole-4-carbonyl chloride (160 mg; 0.77 mmol). The reaction mixture was stirred over weekend, then was heated to 100° C. and stirred for 3 days. The reaction mixture was concentrated under reduced pressure. The residue was taken in ethyl acetate and washed with water. The organic layer was dried over anhydrous sodium sulphate, filtered and concentrated under reduced pressure to give the crude material. The crude material was suspended in acetonitrile and filtered to give the title compound (24%). ¹H NMR (400 MHz, CDCl₃) δ ppm 1.41 (dq, J=12.10, 4.49 Hz, 2H), 1.70 (d, J=12.89, 2H), 1.85-1.99 (m, 1H), 3.34-3.46 (m, 4H), 3.99 (dd, J=11.42, 4.20 Hz, 2H), 7.41-7.53 (m, 4H), 8.02 (s, 1H), 8.20-8.27 (m, 3H), 8.57 (t, J=5.96 Hz, 1H), 9.31 (dd, J=8.69, 1.27 Hz, 1H), 13.42 (s, 1H); MS (ESI) (M+H)⁺ 407.0

### Example 199

### 3-{[4-(dimethylamino)-1-naphthoyl]amino}-N-[3-(1H-imidazol-1-yl)propyl]pyridine-2-carboxamide

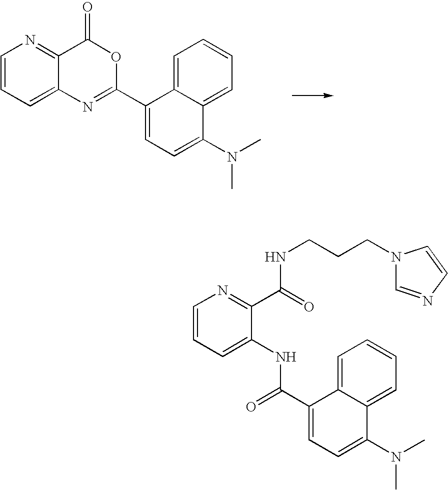

Following the procedure for Step A in Example 1, using 2-[4-(dimethylamino)-1-s naphthyl]-4H-pyrido[3,2-d][1,3]oxazin-4-one (0.58 mmol) and 1-(3-aminopropyl)imidazole (0.35 mL; 2.91 mmol) provided the title compound (15%). ¹H NMR (400 MHz, CD₃OD) 5 ppm 2.10 (quint., J=6.83 Hz, 2H), 3.34 (t, J=6.44 Hz, 2H), 3.44 (s, 6H), 4.21 (t, J=7.13 Hz, 2H), 7.43 (t, J=1.66 Hz, 1H), 7.53-7.61 (m, 2H), 7.71 (dt, J=7.71, 1.17 Hz, 1H), 7.80 (dt, J=7.03, 1.36 Hz, 1H), 7.99 (q, J=7.88 Hz, 2H), 8.28 (d, J=8.59 Hz, 1H), 8.33 (dd, J=4.49, 1.37 Hz, 1H), 8.49 (d, J=8.40 Hz, 1H), 8.86 (t, J=1.27 Hz, 1H), 9.18 (dd, J=8.59, 1.37 Hz, 1H). found: C, 50.62; H, 5.16; N, 13.84. C₂₅H₂₆N₆O₂×4.1HCl×0.1H₂O has C, 50.57; H, 5.14; N, 14.15%; MS (ESI) (M+H)⁺ 443.0.

### Example 200

### N-(4,4-difluorocyclohexyl)-3-(1-naphthoylamino)pyridine-2-carboxamide (IUPAC name)

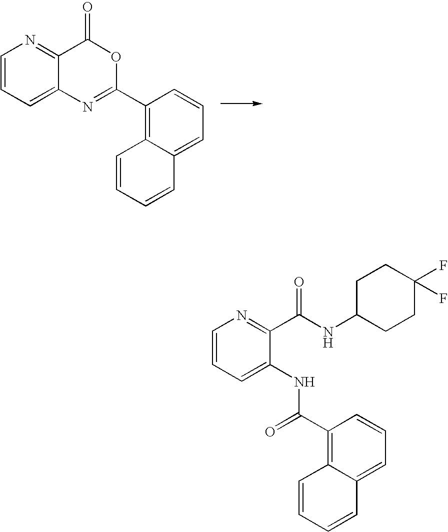

Following the procedure for Step A in Example 1, using 2-(1-naphthalenyl)-4H-pyrido[3,2-d][1,3]oxazin-4-one (274 mg, 1.00 mmol), and 4,4-difluorocyclohexanamine (405 mg, 3.00 mmol) provided the title compound (109 mg, 27%) after purification by flash column chromatography (heptan/EtOAc 2:1). ¹H NMR (400 MHz, CDCl₃) δ 1.64-1.74 (m, 2H), 1.79-1.85 (m, 2H), 2.00-2.14 (m, 2H), 3.93-4.02 (m, 1H), 7.50-7.58 (m, 4H), 7.86-7.90 (m, 2H), 7.97 (d, J=8.3 Hz, 1H), 8.26 (dd, J=4.4, 1.2 Hz, 1H), 8.40 (d, J=7.9 Hz, 1H), 8.51 (d, J=8.3 Hz, 1H), 9.39 (d, J=8.7 Hz, 1H), 12.70 (br s, 1H); MS (ESI) (M+H)⁺ 410.1

### Example 201

### N-(3,5-difluorobenzyl)-3-(1-naphthoylamino)pyridine-2-carboxamide (IUPAC name)

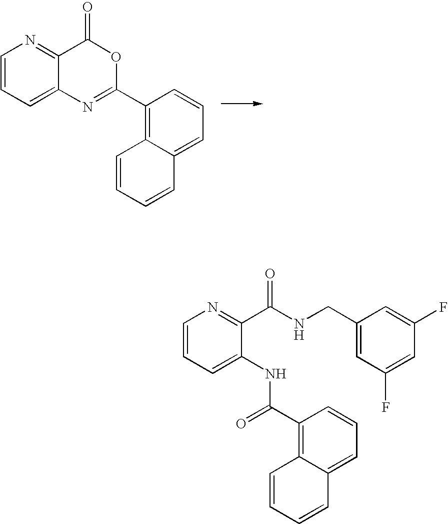

Following the procedure for Step A in Example 1, using 2-(1-naphthalenyl)-4H-pyrido[3,2-d][1,3]oxazin-4-one (137 mg, 0.50 mmol), and 3,5-difluorobenzylamine (215 mg, 1.50 mmol) provided the title compound (16 mg, 8%) after purification by flash column chromatography (heptan/EtOAc 5:2). ¹H NMR (400 MHz, CDCl₃) δ 4.56 (d, J=8.5 Hz, 2H), 6.66-6.72 (m, 1H), 6.80-6.85 (m, 2H), 7.50-7.58 (m, 4H), 7.86-7.90 (m, 2H), 7.97 (d, J=8.1 Hz, 1H), 8.27 (d, J=4.4 Hz, 1H), 8.52 (d, J=8.5 Hz, 1H), 8.83 (br s, 1H), 9.42 (d, J=8.5 Hz, 1H), 12.62 (br s, 1H); MS (ESI) (M−H)-416.0

### Example 202

### N-(4-morpholin-4-ylbenzy)-3-(1-naphthoylamino)pyridine-2-carboxamide (IUPAC name)

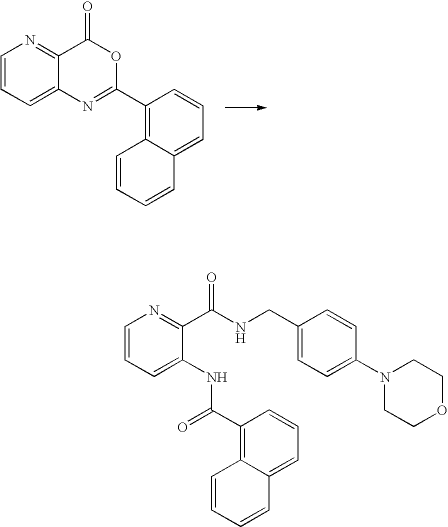

Following the procedure for Step A in Example 1, using 2-(1-naphthalenyl)-4H-pyrido[3,2-d][1,3]oxazin-4-one (137 mg, 0.50 mmol), and 1-(4-morpholin-4-ylphenyl)methanamine (288 mg, 1.50 mmol) provided the title compound (44 mg, 19%) after purification by flash column chromatography (heptan/EtOAc 1:1 and CH₂Cl₂:Et₂O 20:1). ¹H NMR (400 MHz, CDCl₃) δ 3.10-3.13 (m, 4H), 3.81-3.84 (m, 4H), 4.49 (d, J=5.8 Hz, 2H), 6.84-6.88 (m, 2H), 7.21-7.24 (m, 2H), 7.48-7.58 (m, 4H), 7.87-7.92 (m, 2H), 7.97 (d, J=8.2 Hz, 1H), 8.27 (dd, J=4.4, 1.2 Hz, 1H), 8.53 (d, J=8.3 Hz, 1H), 8.65 (br s, 1H), 9.39 (dd, J=8.5, 1.0 Hz, 1H), 12.80 (br s, 1H); MS (ESI) (M+H)⁺ 467.2

### Example 203

### 6-Methoxy-3-[(4-[1,2,3]triazol-1-ylmethyl-naphthalene-1-carbonyl)-amino]-pyridine-2-carboxylic acid cyclohexylmethyl-amide (IUPAC name)

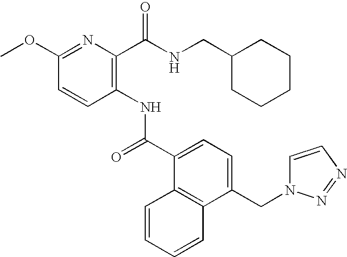

### Step A. 3-Amino-6-methoxy-pyridine-2-carboxylic acid

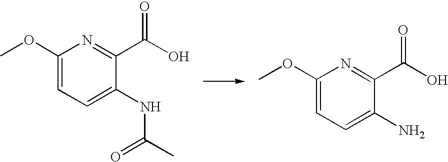

3-Acetylamino-6-methoxy-pyridine-2-carboxylic acid [Besly; Goldberg, JCSOA9; J. Chem. Soc.; 2448, 2455] (7.96 g, 37.88 mmol) was boiled for 80 min with 2.5 N NaOH_{(aq, sat)} (80 ml). The solution was adjusted to pH 4 with 4 N HCl_{(aq)} at 0° C. The formed precipitate was collected, washed with cold water and air dried to give 5.65 g (89%) of 3-Amino-6-methoxy-pyridine-2-carboxylic acid. MS (ESI) (M+H)⁺ 169.14.

### Step B. 6-Methoxy-3-[(4-methyl-naphthalene-1-carbonyl)-amino]-pyridine-2-carboxylic acid methyl ester

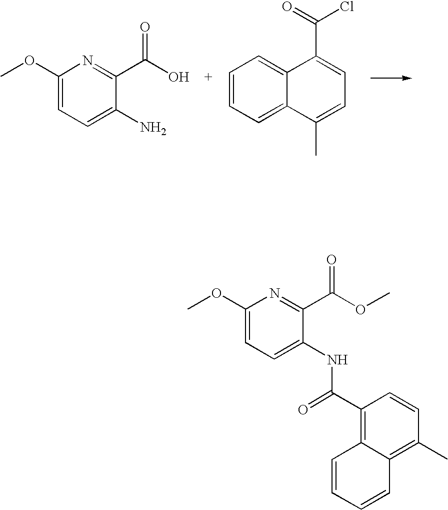

To a solution of 3-Amino-6-methoxy-pyridine-2-carboxylic acid (1.78 g, 10.6 mmol) from step A in anhydrous DMF (30 ml) was added DIPEA (11.07 ml, 63.6 mmol) and 4-methyl-1-naphthalenecarbonyl chloride (2.65 g, 12.95 mmol) under nitrogen. After stirred for 1 h at r.t., and for 1 h at 50° C., K₂CO₃ (2.2 g, 15.9 mmol) was added into the reaction mixture followed by addition of MeI (3.3 ml, 53 mmol) in portions at r.t. After stirred overnight, the reaction mixture was condensed, and the residue was suspended in water, and the crystals filtered, washed with water, ethanol and air dried. The crude product (2.7 g) was suspended in ethyl acetate/methanol, and the crystals filtered, washed with methanol, ether and air dried to give 2 g (54%) of 6-Methoxy-3-[(4-methyl-naphthalene-1-carbonyl)-amino]-pyridine-2-carboxylic acid methyl ester. MS (ESI) (M+H)⁺ 351.10.

### Step C. 6-Methoxy-3-[(4-[1,2,3]triazol-1-ylmethyl-naphthalene-1-carbonyl)-amino]-pyridine-2-carboxylic acid methyl ester

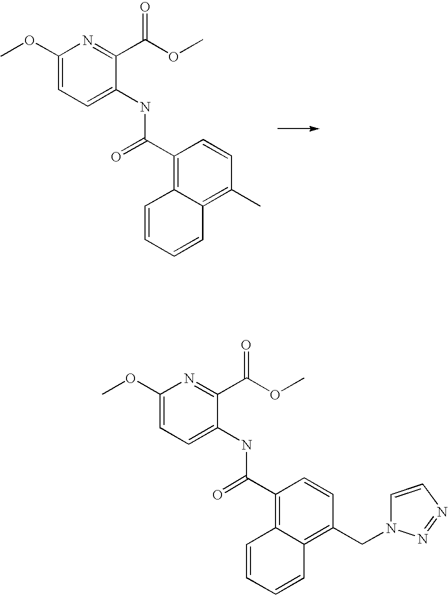

To a mixture of 6-Methoxy-3-[(4-methyl-naphthalene-1-carbonyl)-amino]-pyridine-2-carboxylic acid methyl ester (1.8 g, 5.14 mmol) from step B in CCl₄ (100 ml) was added NBS (0.96 g, 5.39 mmol) and benzoyl peroxide (0.125 g, 0.51 mmol). The reaction mixture was refluxed for 1.5 h under nitrogen. DMF (2.5 ml) and 1,2,3-triazole (2.98 ml, 51.4 mmol) was added, and the reaction mixture was refluxed overnight. After removal of solvents, the residue was suspended in cold water. The formed precipitate was collected, washed with water, air dried and purified by column chromatography on silica gel using first CH₂Cl₂ and then CH₂Cl₂/MeOH (100:1) as eluent to give 1.55 g (72%) of 6-Methoxy-3-[(4-[1,2,3]triazol-1-ylmethyl-naphthalene-1-carbonyl)-amino]-pyridine-2-carboxylic acid methyl ester. MS (ESI) (+H)⁺ 418.13.

### Step D. 6-Methoxy-3-[(4-[1,2,3]triazol-1-ylmethyl-naphthalene-1-carbonyl)-amino]-pyridine-2-carboxylic acid cyclohexylmethyl-amide

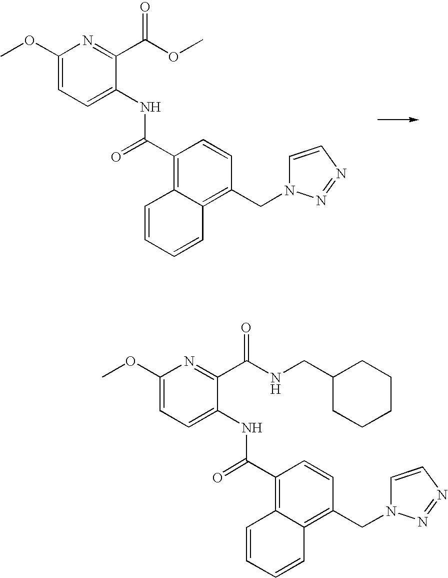

A solution of 6-Methoxy-3-[(4-[1,2,3]triazol-1-ylmethyl-naphthalene-1-carbonyl)-amino]-pyridine-2-carboxylic acid methyl ester (0.5 g, 1.2 mmol) from step C and cyclohexanemethylamine (0.41 g, 3.6 mmol) in DMF (3 ml) was heated at 80° C. for 40 min. The solution was evaporated under reduced pressure, and the residue was dissolved in dichloromethane. After addition of water (50 ml) and 2 N HCl_{(aq)} (13 ml), the organic phase was separated, washed with NaHCO_{3(aq sat)}, brine, dried and evaporated under reduced pressure. The residue was purified by preparative HPLC using acetonitrile and ammonium acetate buffer (30:70 to 95:5) as eluent to give 517 mg (86%) of 6-Methoxy-3-[(4-[1,2,3]triazol-1-ylmethyl-naphthalene-1-carbonyl)-amino]-pyridine-2-carboxylic acid cyclohexylmethyl-amide.

¹H-NMR (600 MHz, CDCl₃): 0.93-1.02 (m, 2H), 1.09-1.27 (m, 3H), 1.50-1.58 (m, 1H), 1.62-1.78 (m, 5H), 3.22 (t, J=6.66 Hz, 2H), 3.94 (s, 3H), 6.04 (s, 2H), 7.01 (d, J=9.1 Hz, 1H), 7.36 (s, 1H), 7.41 (d, J=7.18 Hz, 1H), 7.53-7.60 (m, 2H), 7.66 (s, 1H), 7.83 (d, J=7.17 Hz, 1H), 7.98 (d, J=7.82 Hz, 1H), 8.23 (t, J=6.5 Hz, 1H), 8.53 (d, J=8.52 Hz, 1H), 9.31 (d, J=9.1 Hz, 1H), 12.62 (s, 1H). MS (ESI) (M+H)⁺ 499.12.

### Example 204

### 6-Hydroxy-3-[(4-[1,2,3]triazol-1-ylmethyl-naphthalene-1-carbonyl)-amino]-pyridine-2-carboxylic acid cyclohexylmethyl-amide (IUPAC name)

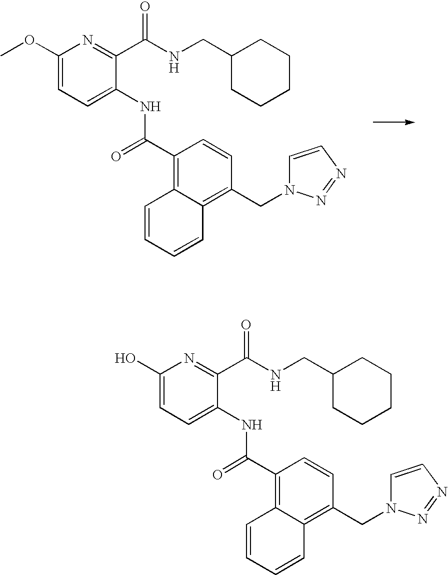

A mixture of 6-Methoxy-3-[(4-[1,2,3]triazol-1-ylmethyl-naphthalene-1-carbonyl)-amino]-pyridine-2-carboxylic acid cyclohexylmethyl-amide (0.29 g, 0.58 mmol) and pyridine hydrochloride (7.3 g, 63.17 mmol) was heated at 150° C. for 25 min. Water was added at r.t. The formed precipitate was collected, washed with water, dried and purified by preparative HPLC using acetonitrile and ammonium acetate buffer (25:75 to 95:5) to give the title compound (193 mg, 69%).

¹H-NMR (500 MHz, CD₃OD): 0.92-1.02 (m, 2H), 1.12-1.30 (m, 3H), 1.50-1.60 (m, 1H), 1.62-1.78 (m, 5H), 3.15 (d, J=7.04 Hz, 2H), 6.19 (s, 2H), 6.96 (d, J=8.91 Hz, 1H), 7.47 (d, J=7.04 Hz, 1H), 7.60-7.66 (m, 2H), 7.73 (d, J=0.94 Hz, 1H), 7.84 (d, J=7.04 Hz, 1H), 7.94 (d, J=0.94 Hz, 1H), 8.19-8.24 (m, 1H), 8.43-8.48 (m, 1H), 9.12 (d, J=8.92 Hz, 1H). MS (ESI) (M+H)⁺ 485.15.

### Example 205

### 6-Methoxy-3-[(4-[1,2,3]triazol-1-ylmethyl-naphthalene-1-carbonyl)-amino]-pyridine-2-carboxylic acid (tetrahydro-pyran-4-ylmethyl)-amide (IUPAC name)

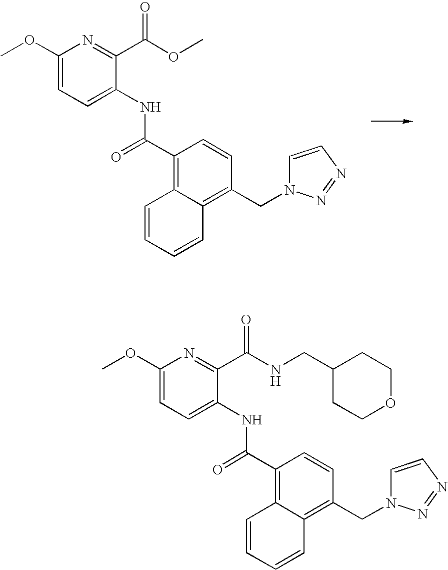

A solution of 6-Methoxy-3-[(4-[1,2,3]triazol-1-ylmethyl-naphthalene-1-carbonyl)-amino]-pyridine-2-carboxylic acid methyl ester (0.5 g, 1.2 mmol) and 4-tetrahydropyramethyl amine (0.395 g, 3.42 mmol) in DMF (3 ml) was heated at 80° C. for 3 h. The solution was evaporated under reduced pressure. The residue was purified by preparative HPLC using acetonitrile and ammonium acetate buffer (20:80 to 90:10) to give the title compound (473 mg, 79%).

¹NMR (300 MHz, CDCl₃): 1.30-1.41 (m, 2H), 1.60-1.70 (m, 2H), 1.80-1.94 (m, 1H), 3.26-3.43 (m, 4H), 3.96 (s, 3H), 3.96-4.02 (m, 2H), 6.06 (s, 2H), 7.04 (d, J=9.23 Hz, 1H), 7.39 (d, J=0.84 Hz, 1H), 7.43 (d, J=7.22 Hz, 1H), 7.54-7.64 (m, 2H), 7.69 (d, J=0.84 Hz, 1H), 7.85 (d, J=7.21 Hz, 1H), 7.96-8.04 (m, 1H), 8.27 (t, J=6.21 Hz, 1H), 8.51-8.59 (m, 1H), 9.33 (d, J=9.07 Hz, 1H), 12.55 (s, 1H). MS (ESI) (M+H)⁺ 501.12.

### Example 206

### 6-Hydroxy-3-[(4-[1,2,3]triazol-1-ylmethyl-naphthalene-1-carbonyl)-amino]-pyridine-2-carboxylic acid (tetrahydro-pyran-4-ylmethyl)-amide (IUPAC name)

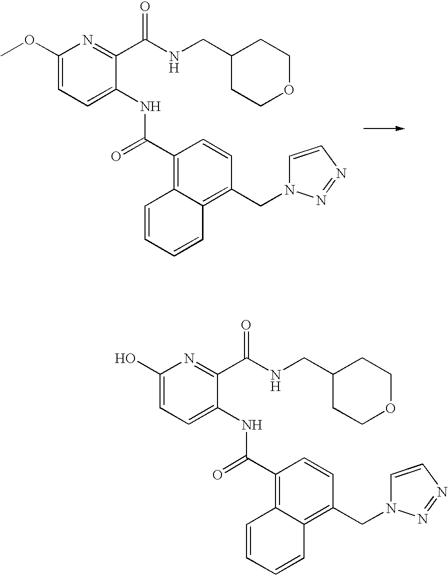

The compound was prepared according to the procedure for 6-Hydroxy-3-[(4-[1,2,3]triazol-1-ylmethyl-naphtlialene-1-carbonyl)-amino]-pyridine-2-carboxylic acid cyclohexylmethyl-amide in 80% isolated yield.

¹H-NMR (300 MHz, CD₃OD): 1.22-1.40 (m, 2H), 1.57-1.69 (m, 2H), 1.74-1.92 (m, 1H), 3-20-3.42 (m, 4H), 3.85-3.96 (m, 2H), 6.20 (s, 2H), 6.96 (d, J=9.07 Hz, 1H), 7.46 (d, J=7.39 Hz, 1H), 7.58-7.69 (m, 2H), 7.74 (s, 1H), 7.85 (d, J=7.22 Hz, 1H), 7.95 (s, 1H), 8.18-8.27 (m, 1H), 8.41-8.50 (m, 1H), 9.12 (d, J=9.07 Hz, 1H). MS (ESI) (M+H)⁺ 487.12.

### Example 207

### 6-Propoxy-3-[(4-[1,2,3]triazol-1-ylmethyl-naphthalene-1-carbonyl)-amino]-pyridine-2-carboxylic acid cyclohexylmethyl-amide (IUPAC name)

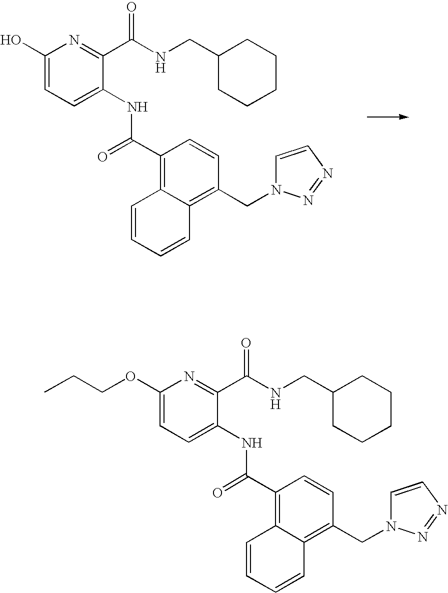

A mixture of 6-Hydroxy-3-[(4-[1,2,3]triazol-1-ylmethyl-naphthalene-1-carbonyl)-amino]-pyridine-2-carboxylic acid cyclohexylmethyl-amide (7 mg, 0.014 mmol), silver carbonate (50 mg, 0.18 mmol) and 4 drops of 1-iodopropane in acetonitrile (1.5 ml) was refluxed for 1 h. Dichloromethane and water were added at r.t. The organic layer was separated, washed with NaHCO₃(aq, sat), water, brine, dried and evaporated under reduced pressure. The residue was purified by column chromatography on silica gel using CH₂Cl₂/MeOH (100:2.5) as eluent to give the title compound (4.5 mg, 59%).

¹H-NMR (500 MHz, CDCl₃): 0.93-1.04 (m, 2H), 1.07 (t, J=7.51 Hz, 3H), 1.11-1.32 (m, 3H), 1.52-1.62 (m, 1H), 1.64-1.80 (m, 6H), 1.81-1.90 (m, 2H), 3.24 (t, J=6.58 Hz, 2H), 4.24 (t, J=6.57 Hz, 2H), 6.06 (s, 2H), 7.02 (d, J=8.92 Hz, 1H), 7.38 (d, J=0.94 Hz, 1H), 7.44 (d, J=7.51 Hz, 1H), 7.55-7.62 (m, 2H), 7.69 (d, J=0.94 Hz, 1H), 7.85 (d, J=7.04 Hz, 1H), 8.0 (dd, J=7.98, 1.41 Hz, 1H), 8.23 (t, J=6.11 Hz, 1H), 8.55 (dd, J=7.51, 1.87 Hz, 1H), 8.32 (d, J=9.39 Hz, 1H), 12.63 (s, 1H). MS (ESI) (M+H)⁺ 527.31.

1. A compound of formula I, a diasteriomer or enantiomer of the compound, a pharmaceutically acceptable salt of the compound diasteriomer, or enantiomer, or mixtures thereof: 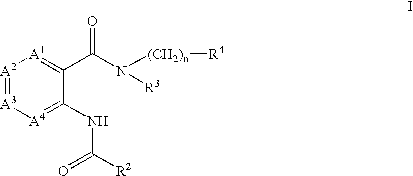
wherein:
one of A¹, A², A³ or A⁴ is N and the remaining are CR¹;
each R¹ is independently selected from the group consisting of hydrogen, halogen, cyano, amino, acetylamino, hydroxyl, alkoxy, alkyl, halogenated alkoxy, alkylene, halogenated alkyl, halogenated alkenyl, and NR⁵R⁶;
R² is selected from the group consisting of: 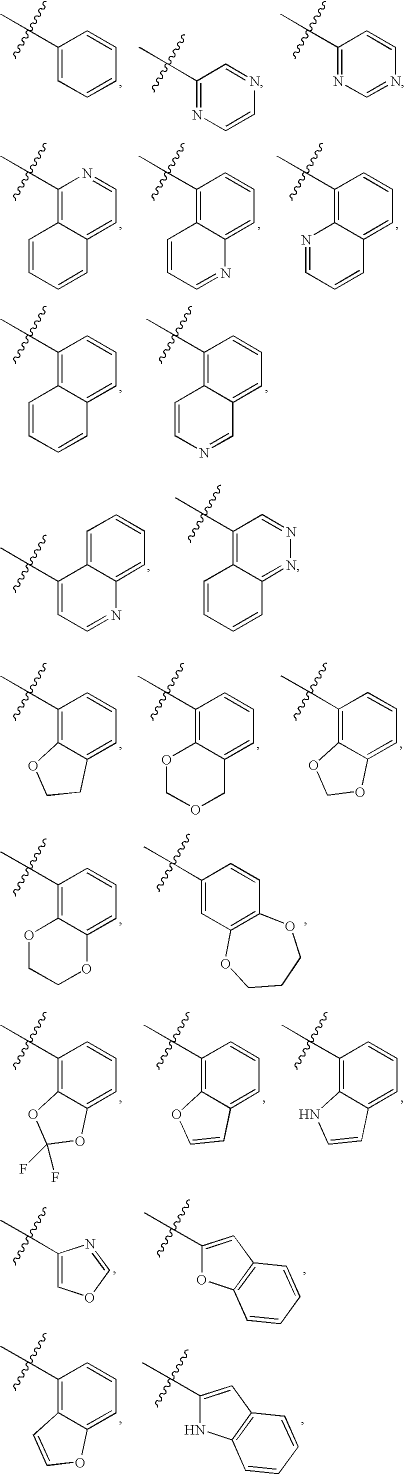 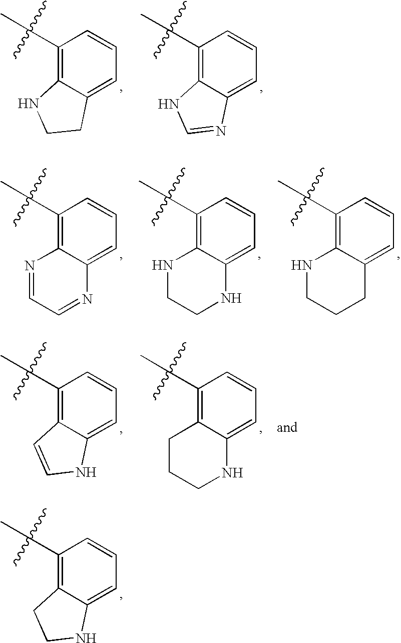
and is unsubstituted or substituted by one or more substituents selected from the group consisting of halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl, aryl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents selected from the group consisting of halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or substituted by one or more substituents selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₆alkylcarbonyl, C₃₋₆heterocyclyl, and C₃₋₆heterocyclyl-C₁₋₆alkyl and are unsubstituted or substituted by one or more substituents independently selected from the group consisting of halogen, cyano, nitro, C₁₋₆ alkoxy, C₁₋₆ alkyl, and hydroxy;
with a proviso that when n=0, then R⁴ is not thiazolyl or 5-chloropyridinyl;
with a further proviso that when R² is phenyl, then n=0 and R⁴ is not unsubstituted methyl, C₃ alkyl, or unsubstituted C₄ alkyl; and
with a further proviso that the compound of formula I is not any one of:
3-(benzoylamino)-N-benzylpyridine-2-carboxamide;
3-(benzoylamino)-N-pyridin-3-ylpyridine-2-carboxamide;
3-(benzoylamino)-N-phenylpyridine-2-carboxamide;
3-(benzoylamino)-N-(3-nitrophenyl)pyridine-2-carboxamide;
3-(benzoylamino)-N-(4-methoxyphenyl)pyridine-2-carboxamide;
3-(benzoylamino)-N-[4-(dimethylamino)phenyl]pyridine-2-carboxamide;
N-(2-hydroxyethyl)-4-(2-naphthoylamino)nicotinamide;
4-(benzoylamino)-N-(2-hydroxyethyl)nicotinamide;
3-(benzoylamino)-2,6-dimethyl-N-phenylisonicotinamide;
3-(benzoylamino)-2,6-dimethyl-N-(3-nitrophenyl)isonicotinamide;
2-(benzoylamino)-N-[cyano(2-thienyl)methyl]nicotinamide; and
2-(benzoylamino)-N-[cyano(phenyl)methyl]nicotinamide.

2. The compound according to claim 1, wherein:
each R¹ is independently selected from the group consisting of hydrogen, halogen, hydroxyl, alkoxy, alkyl, halogenated alkoxy, and halogenated alkyl;
R² is selected from the group consisting of: 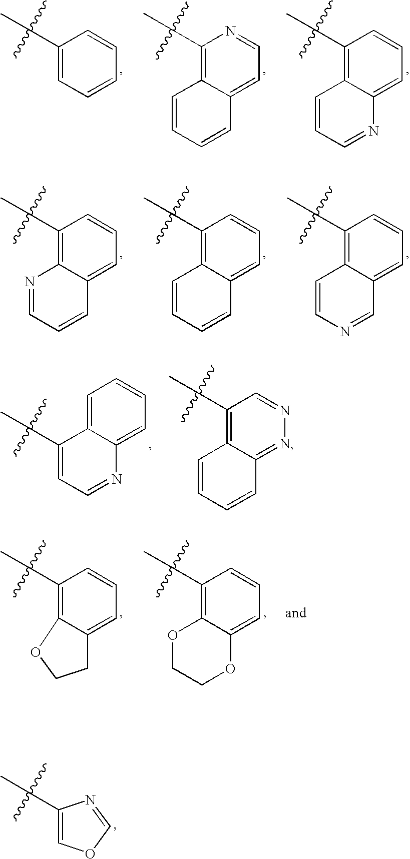
and is unsubstituted or substituted by one or more substituents selected from the group consisting of halogen, halogenated alkyl, alkyl, halogenated alkoxy, cyano, nitro, alkyl-alkoxy, hydroxy-alkyl, alkoxy, alkoxyalkyl, alkylamino, amino-alkyl, alkyl-amino-carbonyl, heterocyclyl, heteroaryl, heteroarylalkyl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkyl-carbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₆alkylcarbonyl, C₃₋₆heterocyclyl and C₃₋₆heterocyclyl-C₁₋₆alkyl and are each independently unsubstituted or substituted by one or more substituents independently selected from the group consisting of halogen, cyano, nitro, C₁₋₆ alkoxy, C₁₋₆ alkyl and hydroxy.

3. The compound according to claim 1, wherein:
each R¹ is independently selected from the group consisting of hydrogen, fluoro, chloro, hydroxyl, alkoxy, alkyl, halogenated alkoxy, and halogenated alkyl;
R² is selected from the group consisting of: 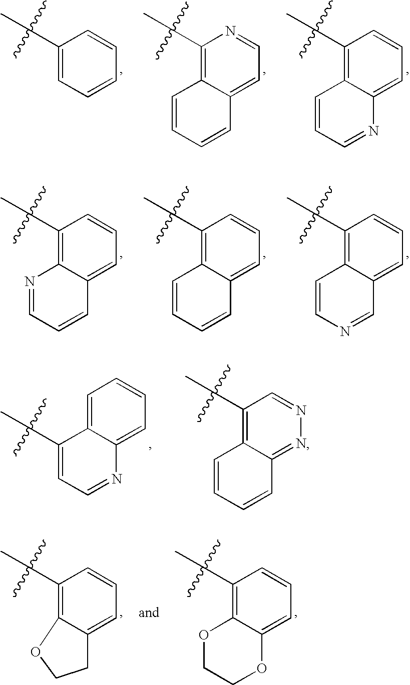
and is unsubstituted or substituted by one or more substituents independently selected from halogen, halogenated alkyl, alkyl, halogenated alkoxy, alkyl-alkoxy, hydroxy-alkyl, alkoxy, alkoxyalkyl, alkylamino, amino-alkyl, alkyl-amino-carbonyl, heterocyclyl, heteroaryl, heteroarylalkyl, and —NR⁵R⁶;
R³ is hydrogen or alkyl;
R⁴ is selected from alkyl, alkenyl, cycloalkyl, cycloalkenyl, alkoxy, aryl, heteroaryl and heterocyclyl and is unsubstituted or substituted by one or more substituents independently selected from the group consisting of halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₁₆alkylcarbonyl, C₃₋₆heterocyclyl, and C₃₋₆heterocyclyl-C₁₋₁₆alkyl and are independently unsubstituted or substituted by one or more substituents independently selected from the group consisting of halogen, C₁₋₆ alkoxy, C₁₋₆ alkyl, and hydroxy.

4. A compound of formula IB, a diasteriomer or enantiomer of the compound, a pharmaceutically acceptable salt of the compound, diasteriomer, or enantiomer, or mixtures thereof: 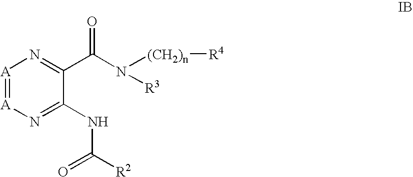
wherein:
A is CR¹;
each R¹ is independently selected from the group consisting of hydrogen, halogen, cyano, amino, acetylamino, hydroxyl, alkoxy, alkyl, halogenated alkoxy, alkylene, halogenated alkyl, halogenated alkenyl, and NR⁵R⁶;
R² is selected from the group consisting of 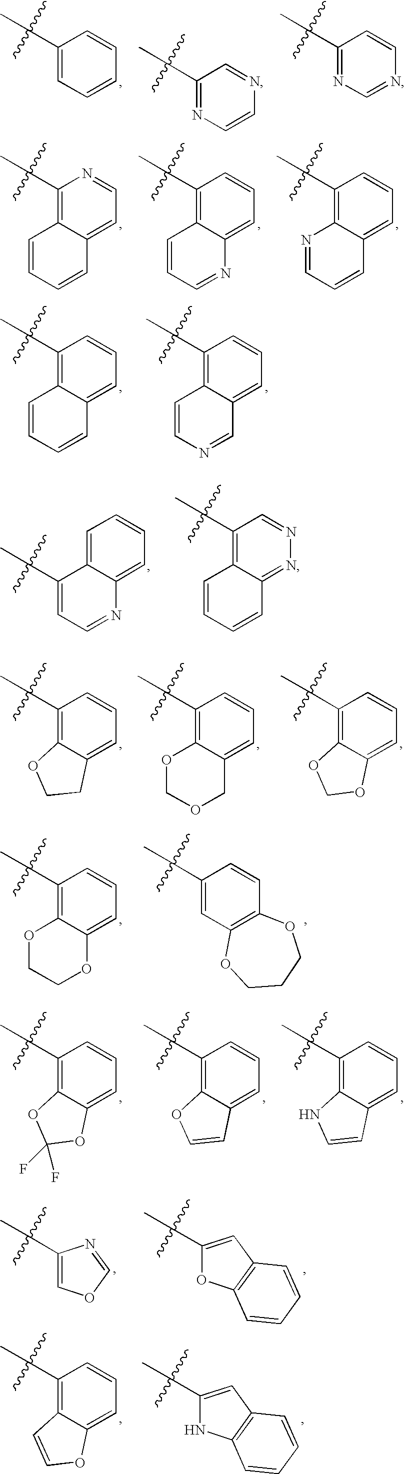 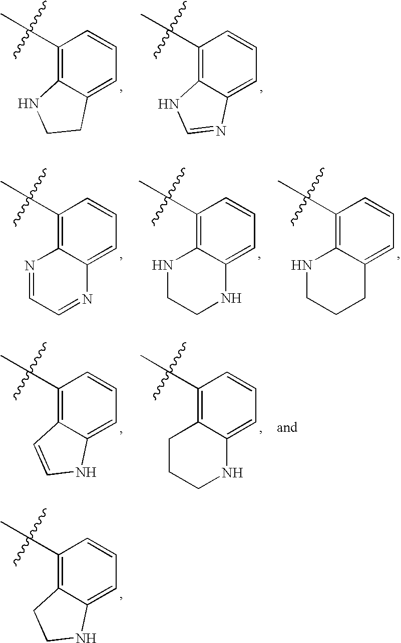
and is unsubstituted or substituted by one or more substituents independently selected from the group consisting of halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl, and C₃₋₆heterocyclyl-C₁₋₆alkyl and are each independently unsubstituted or substituted by one or more substituents independently selected from the group consisting of halogen, cyano, nitro, C₁₋₆ alkoxy, C₁₋₆ alkyl, and hydroxy;
with a proviso that said compound of formula IB is not any one of:
3-[(4-tert-butylbenzoyl)amino]-N-(5-chloro-pyridin-2-yl)pyrazine-2-carboxamide;
N-[2-(1H-imidazol-2-yl)ethyl]-3-[[4-(1,1-dimethylethyl)benzoyl]amino]-2-pyrazine-carboxamide; and
3-(benzoylamino)-N-(methoxycarbonylmethyl)pyrazine-2-carboxamide.

5. The compound according to claim 4, wherein:
A is CR¹;
each R¹ is independently selected from the group consisting of hydrogen, halogen, hydroxyl, alkoxy, alkyl, halogenated alkoxy, and halogenated alkyl;
R² is selected from the group consisting of: 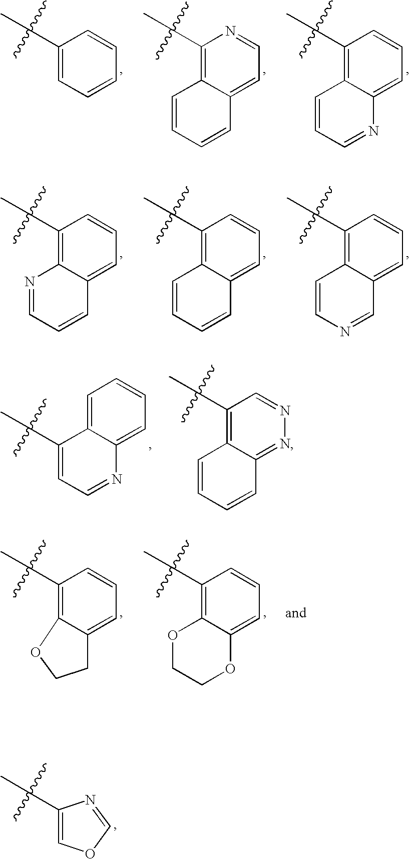
and is unsubstituted or substituted by one or more substituents independently selected from the group consisting of halogen, halogenated alkyl, alkyl, halogenated alkoxy, cyano, nitro, alkyl-alkoxy, hydroxy-alkyl, alkoxy, alkoxyalkyl, alkylamino, amino-alkyl, alkyl-amino-carbonyl, heterocyclyl, heteroaryl, heteroarylalkyl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkyl-carbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₆alkylcarbonyl, C₃₋₆heterocyclyl and C₃₋₆heterocyclyl-C₁₋₆alkyl and are each independently unsubstituted or substituted by one or more substituents independently selected from the group consisting of halogen, cyano, nitro, C₁₋₆ alkoxy, C₁₋₆ alkyl, and hydroxy.

6. The compound according to claim 4, wherein:
A is CR¹;
each R¹ is independently selected from the group consisting of hydrogen, fluoro, chloro, hydroxyl, alkoxy, alkyl, halogenated alkoxy, and halogenated alkyl;
R² is selected from the group consisting of: 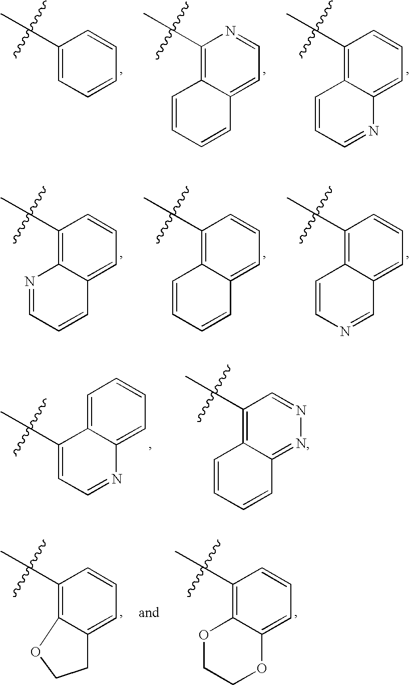
and is unsubstituted or substituted by one or more substituents independently selected from the group consisting of halogen, halogenated alkyl, alkyl, halogenated alkoxy, alkyl-alkoxy, hydroxy-alkyl, alkoxy, alkoxyalkyl, alkylamino, amino-alkyl, alkyl-amino-carbonyl, heterocyclyl, heteroaryl, heteroarylalkyl- and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₆alkylcarbonyl, C₃₋₆heterocyclyl, and C₃₋₆heterocyclyl-C₁₋₆alkyl and are each independently unsubstituted or substituted by one or more substituents independently selected from the group consisting of halogen, C₁₋₆ alkoxy, C₁₋₆ alkyl, and hydroxy.

7. A compound of formula IA, a diasteriomer or enantiomer of the compound, a pharmaceutically acceptable salt of the compound, diasteriomer, or enantiomer, or mixtures thereof: 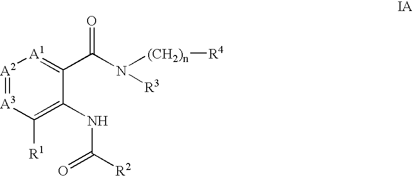
wherein:
one of A¹, A² or A is N and the remaining are CR¹;
each R¹ is independently selected from the group consisting of hydrogen, halogen, cyano, amino, acetylamino, hydroxyl, alkoxy, alkyl, halogenated alkoxy, alkylene, halogenated alkyl, halogenated alkenyl, and NR⁵R⁶;
R² is selected from the group consisting of: 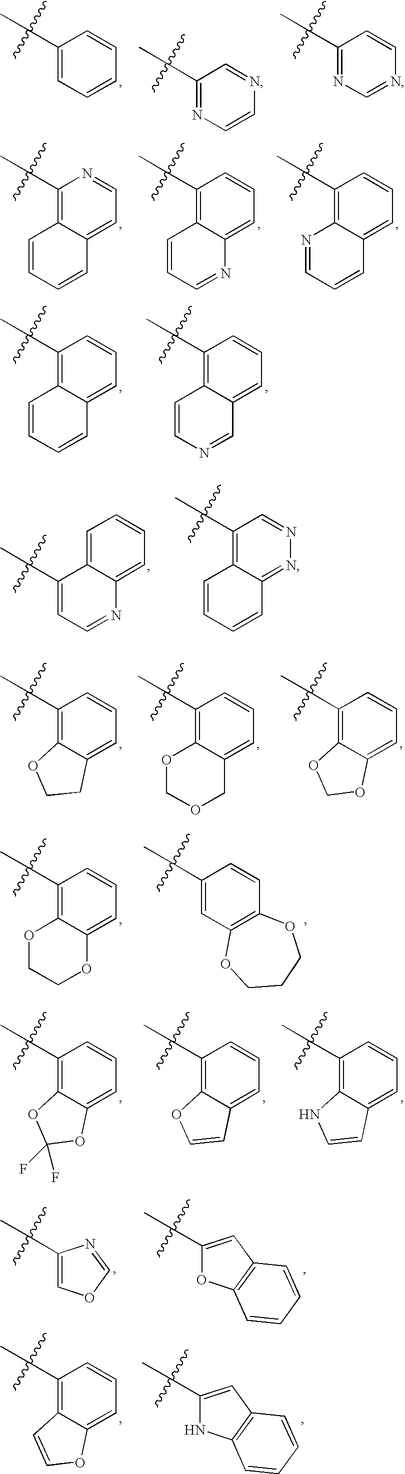 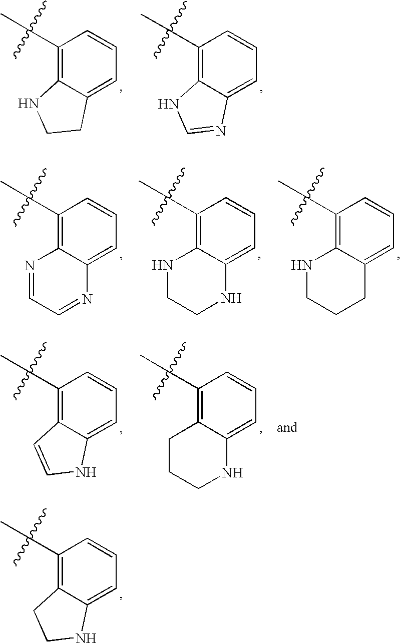
and is unsubstituted or substituted by one or more substituents independently selected from the group consisting of halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₁₆alkylcarbonyl, C₃₋₆heterocyclyl, and C₃₋₆heterocyclyl-C₁₋₁₆alkyl and are each independently unsubstituted or substituted by one or more substituents independently selected from the group consisting of halogen, cyano, nitro, C₁₋₆ alkoxy, C₁₋₆ alkyl, and hydroxy;
with a proviso that when n=0, then R⁴ is not thiazolyl or 5-chloropyridinyl;
with a further proviso that when R² is phenyl, then n=0 and R⁴ is not unsubstituted methyl, C₃ alkyl, or unsubstituted C₄ alkyl; and
with a further proviso that the compound of formula IA is not any one of:
3-(benzoylamino)-N-benzylpyridine-2-carboxamide;
3-(benzoylamino)-N-pyridin-3-ylpyridine-2-carboxamide;
3-(benzoylamino)-N-phenylpyridine-2-carboxamide;
3-(benzoylamino)-N-(3-nitrophenyl)pyridine-2-carboxamide;
3-(benzoylamino)-N-(4-methoxyphenyl)pyridine-2-carboxamide;
3-(benzoylamino)-N-[4-(dimethylamino)phenyl]pyridine-2-carboxamide;
N-(2-hydroxyethyl)-4-(2-naphthoylamino)nicotinamide;
4-(benzoylamino)-N-(2-hydroxyethyl)nicotinamide;
3-(benzoylamino)-2,6-dimethyl-N-phenylisonicotinamide; and
3-(benzoylamino)-2,6-dimethyl-N-(3-nitrophenyl)isonicotinamide.

8. The compound according to claim 7, wherein:
each R¹ is independently selected from the group consisting of hydrogen, halogen, hydroxyl, alkoxy, alkyl, halogenated alkoxy, and halogenated alkyl;
R² is selected from the group consisting of: 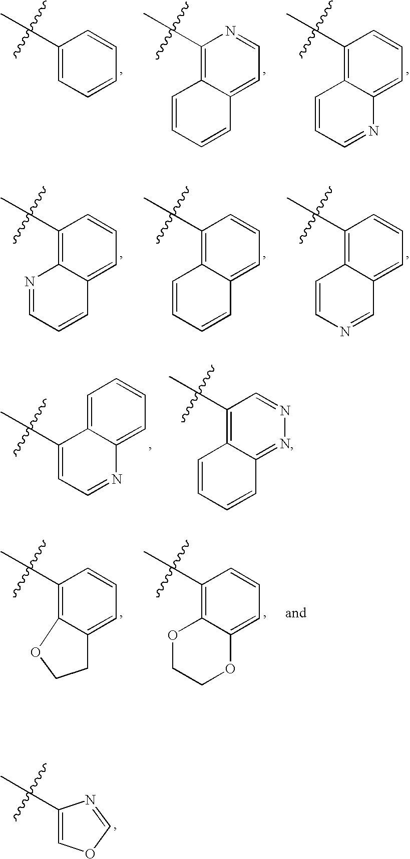
and is unsubstituted or substituted by one or more substituents independently selected from the group consisting of halogen, halogenated alkyl, alkyl, halogenated alkoxy, cyano, nitro, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl-, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl and heterocyclyl and is unsubstituted or substituted by one or more substituents independently selected from the group consisting of halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶;
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₁₆alkylcarbonyl, C₃₋₆heterocyclyl, and C₃₋₆heterocyclyl-C₁₋₁₆alkyl and are each independently unsubstituted or substituted by one or more substituents selected from the group consisting of halogen, cyano, nitro, C₁₋₆ alkoxy, C₁₋₆ alkyl, and hydroxy.

9. The compound according to claim 7, wherein:
each R¹ is independently selected from the group consisting of hydrogen, fluoro, chloro, hydroxyl, alkoxy, alkyl, halogenated alkoxy, and halogenated alkyl; and
R² is selected from the group consisting of: 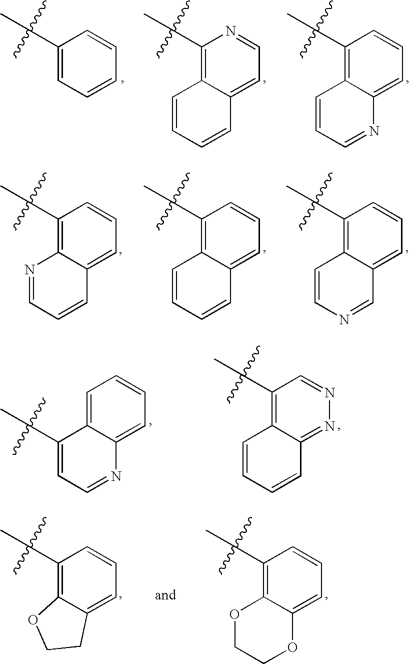
and is unsubstituted or substituted by one or more substituents independently selected from the group consisting of halogen, halogenated alkyl, alkyl, halogenated alkoxy, alkyl-alkoxy, hydroxy-alkyl, alkoxy, alkoxyalkyl, alkylamino, amino-alkyl, alkyl-amino-carbonyl, heterocyclyl, heteroaryl, -heteroarylalkyl- and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkyl-carbonyl, cyano, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or optionally substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₁₆alkyl, C₁₋₆alkylcarbonyl, C₃₋₆heterocyclyl, and C₃₋₆heterocyclyl-C₁₋₆alkyl and are each independently unsubstituted or substituted by one or more substituents independently selected from the group consisting of halogen, C₁₋₆ alkoxy, C₁₋₆ alkyl, and hydroxy.

10. A compound selected from the group consisting of:
N-(Cyclobutylmethyl)-3-[(1-naphthalenylcarbonyl)amino]-2-pyridinecarboxamide;
N-[2-(4-Morpholinyl)ethyl]-3-[(1-naphthalenylcarbonyl)amino]-2-pyridinecarboxamide;
N-4-morpholinyl-3-[(1-naphthalenylcarbonyl)amino]-2-pyridinecarboxamide;
3-[(1-Naphthalenylcarbonyl)amino]-N-[(tetrahydro-2H-pyran-4-yl)methyl]-2-pyridinecarboxamide;
N-Cyclohexyl-3-[(1-naphthalenylcarbonyl)amino]-2-pyridinecarboxamide;
N-(3-Methylcyclohexyl)-3-[(1-naphthalenylcarbonyl)amino]-2-pyridinecarboxamide;
N-Cyclobutyl-3-[(1-naphthalenylcarbonyl)amino]-2-pyridinecarboxamide;
N-(Cyclohexylmethyl)-3-[(1-naphthalenylcarbonyl)amino]-2-pyridinecarboxamide;
3-[(1-Naphthalenylcarbonyl)amino]-N-(tetrahydro-2H-pyran-4-yl)-2-pyridinecarboxamide;
3-[(1-Naphthalenylcarbonyl)amino]-N-[2-(1-piperidinyl)ethyl]-2-pyridinecarboxamide;
N-(2-Hydroxypropyl)-3-[(1-naphthalenylcarbonyl)amino]-2-pyridinecarboxamide;
N-(2-Hydroxybutyl)-3-[(1-naphthalenylcarbonyl)amino]-2-pyridinecarboxamide;
N-(Cyclopentylmethyl)-3-[(1-naphthalenylcarbonyl)amino]-2-pyridinecarboxamide;
3-[(1-Naphthalenylcarbonyl)amino]-N-(2-piperidinylmethyl)-2-pyridinecarboxamide;
N-(2,2-Dimethylpropyl)-3-(1-naphthoylamino)pyridine-2-carboxamide;
N-(2-Methoxy-1-methylethyl)-3-(1-naphthoylamino)pyridine-2-carboxamide;
N-[(1-Hydroxycyclohexyl)methyl]-3-(1-naphthoylamino)pyridine-2-carboxamide;
N-(Cyclobutylmethyl)-3-[[(4-methyl-1-naphthalenyl)carbonyl]amino]-2-pyridinecarboxamide;
3-[[(4-Methyl-1-naphthalenyl)carbonyl]amino]-N-[(tetrahydro-2H-pyran-4-yl)methyl]-2-pyridinecarboxamide;
3-[(4-Methyl-1-naphthoyl)amino]-N-(piperidin-2-ylmethyl)pyridine-2-carboxamide;
N-(Cyclobutylmethyl)-3-[[(4-methoxy-1-naphthalenyl)carbonyl]amino]-2-pyridinecarboxamide;
3-[(4-Methoxy-1-naphthoyl)amino]-N-(tetrahydro-2H-pyran-4-ylmethyl)pyridine-2-carboxamide;
N-(Cyclohexylmethyl)-3-[[[4-(dimethylamino)-1-naphthalenyl]carbonyl]amino]-2-pyridinecarboxamide;
3-[[[4-(Dimethylamino)-1-naphthalenyl]carbonyl]amino]-N-[(tetrahydro-2H-pyran-4-yl)methyl]-2-pyridinecarboxamide;
N-(Cyclobutylmethyl)-3-[[[4-(dimethylamino)-1-naphthalenyl]carbonyl]amino]-2-pyridinecarboxamide;
N-(Cyclobutyloxy)-3-[(1-naphthalenylcarbonyl)amino]-2-pyridinecarboxamide;
N-(Cyclopentyloxy)-3-[(1-naphthalenylcarbonyl)amino]-2-pyridinecarboxamide;
N-(Cyclohexyloxy)-3-[(1-naphthalenylcarbonyl)amino]-2-pyridinecarboxamide;
N-(Cyclohexyloxy)-3-[(4-methoxy-1-naphthalenylcarbonyl)amino]-2-pyridinecarboxamide;
N-(Cyclobutylmethyl)-3-[(2-methoxybenzoyl)amino]-2-pyridinecarboxamide;
N-[2-[[(Cyclobutylmethyl)amino]carbonyl]-3-pyridinyl]-4-quinolinecarboxamide;
N-[2-[[(Cyclobutylmethyl)amino]carbonyl]-3-pyridinyl]-5-isoquinolinecarboxamide;
N-(Cyclobutylmethyl)-3-[[(2,3-dihydro-1,4-benzodioxin-5-yl)carbonyl]amino]-2-pyridinecarboxamide;
N-(Cyclobutylmethyl)-3-[[(2,3-dihydro-7-benzofuranyl)carbonyl]amino]-2-pyridinecarboxamide;
N-(Cyclobutylmethyl)-3-[(3-methoxy-2-methylbenzoyl)amino]-2-pyridinecarboxamide;
N-(2-{[(Tetrahydro-2H-pyran-4-ylmethyl)amino]carbonyl}pyridin-3-yl)quinoline-4-carboxamide;
N-(2-{[(Tetrahydro-2H-pyran-4-ylmethyl)amino]carbonyl}pyridin-3-yl)isoquinoline-5-carboxamide;
N-(2-{[(Tetrahydro-2H-pyran-4-ylmethyl)amino]carbonyl}pyridin-3-yl)quinoline-5-carboxamide;
N-(Cyclohexylmethyl)-4-(1-naphthoylamino)nicotinamide;
N-(Cyclobutylmethyl)-4-(1-naphthoylamino)nicotinamide;
N-(Cyclohexylmethyl)-3-(1-naphthoylamino)isonicotinamide;
N-Cyclobutyl-3-(1-naphthoylamino)isonicotinamide;
3-(1-Naphthoylamino)-N-(tetrahydro-2H-pyran-4-ylmethyl)pyrazine-2-carboxamide;
N-(Cyclohexylmethyl)-3-(1-naphthoylamino)pyrazine-2-carboxamide;
N-(Cyclobutylmethyl)-3-(1-naphthoylamino)pyrazine-2-carboxamide;
N-(Cyclopentylmethyl)-3-(1-naphthoylamino)pyrazine-2-carboxamide;
N-(2-Cyclohexylethyl)-3-(1-naphthoylamino)pyrazine-2-carboxamide;
3-[(4-Methyl-1-naphthoyl)amino]-N-pentylpyrazine-2-carboxamide;
N-(3-Methylbutyl)-3-[(4-methyl-1-naphthoyl)amino]pyrazine-2-carboxamide;
N-(Cyclobutylmethyl)-3-[(4-methyl-1-naphthoyl)amino]pyrazine-2-carboxamide;
3-[(4-Methyl-1-naphthoyl)amino]-N-(tetrahydro-2H-pyran-4-ylmethyl)pyrazine-2-carboxamide;
N-(Cyclobutylmethyl)-3-[(4-ethyl-1-naphthoyl)amino]pyrazine-2-carboxamide;
N-(Cyclohexylmethyl)-3-[(4-ethyl-1-naphthoyl)amino]pyrazine-2-carboxamide;
3-[(4-Ethyl-1-naphthoyl)amino]-N-(tetrahydro-2H-pyran-4-ylmethyl)pyrazine-2-carboxamide;
N-(Cyclobutylmethyl)-3-{[4-(1H-1,2,3-triazol-1-ylmethyl)-1-naphthoyl]amino}pyrazine-2-carboxamide;
N-(Cyclohexylmethyl)-3-{[4-(1H-1,2,3-triazol-1-ylmethyl)-1-naphthoyl]amino}pyrazine-2-carboxamide;
N-(Tetrahydro-2H-pyran-4-ylmethyl)-3-{[4-(1H-1,2,3-triazol-1-ylmethyl)-1-naphthoyl]amino}pyrazine-2-carboxamide;
N-(3-Methylbutyl)-3-{[4-(1H-1,2,3-triazol-1-ylmethyl)-1-naphthoyl]amino}pyrazine-2-carboxamide;
3-{[4-(Methoxymethyl)-1-naphthoyl]amino}-N-(tetrahydro-2H-pyran-4-ylmethyl)pyrazine-2-carboxamide;
N-(Cyclobutylmethyl)-3-{[4-(methoxymethyl)-1-naphthoyl]amino}pyrazine-2-carboxamide;
N-(Cyclohexylmethyl)-3-[(4-methoxy-1-naphthoyl)amino]pyrazine-2-carboxamide;
3-{[5-Bromo-4-(1H-1,2,3-triazol-1-ylmethyl)-1-naphthoyl]amino}-N-(cyclohexylmethyl)pyrazine-2-carboxamide;
3-[(4-Methoxy-1-naphthoyl)amino]-N-(tetrahydrofuran-2-ylmethyl)pyridine-2-carboxamide;
N-(1,4-Dioxan-2-ylmethyl)-3-[(4-methoxy-1-naphthoyl)amino]pyridine-2-carboxamide;
3-[(4-Methoxy-1-naphthoyl)amino]-N-(tetrahydro-2H-pyran-4-yl)pyridine-2-carboxamide;
3-[(4-Methoxy-1-naphthoyl)amino]-N-[2-(tetrahydro-2H-pyran-4-yl)ethyl]pyridine-2-carboxamide;
3-[(4-Methoxy-1-naphthoyl)amino]-N-[(2R)-piperidin-2-ylmethyl]pyridine-2-carboxamide;
3-[(4-Methoxy-1-naphthoyl)amino]-N-(morpholin-3-ylmethyl)pyridine-2-carboxamide;
N-[(1-Hydroxycyclohexyl)methyl]-3-[(4-methoxy-1-naphthoyl)amino]pyridine-2-carboxamide;
N-(Cyclohexylmethyl)-3-[(4-ethoxy-1-naphthoyl)amino]pyridine-2-carboxamide;
3-[(4-Ethoxy-1-naphthoyl)amino]-N-pentylpyridine-2-carboxamide;
3-[(4-Ethoxy-1-naphthoyl)amino]-N-(tetrahydro-2H-pyran-4-ylmethyl)pyridine-2-carboxamide;
N-(Cyclopentylmethyl)-3-[(4-ethoxy-1-naphthoyl)amino]pyridine-2-carboxamide;
3-[(4-Ethoxy-1-naphthoyl)amino]-N-[2-(tetrahydro-2H-pyran-4-yl)ethyl]pyridine-2-carboxamide;
N-(Cyclobutylmethyl)-3-[(4-ethoxy-1-naphthoyl)amino]pyridine-2-carboxamide;
N-Cyclobutyl-3-[(5-methyl-1-naphthoyl)amino]pyridine-2-carboxamide;
3-(1-Naphthoylamino)-N-[(2R)-piperidin-2-ylmethyl]pyridine-2-carboxamide;
3-(1-Naphthoylamino)-N-[(2S)-piperidin-2-ylmethyl]pyridine-2-carboxamide;
3-(1-Naphthoylamino)-N-(pyridin-2-ylmethyl)pyridine-2-carboxamide;
3-(4-Methyl 1-naphthoylamino)-N-(pyridin-2-ylmethyl)pyridine-2-carboxamide;
3-[(4-Amino-1-naphthoyl)amino]-N-(cyclohexylmethyl)pyridine-2-carboxamide;
N-(Cyclohexylmethyl)-3-[(4-methyl-1-naphthalenylcarbonyl)amino]-2-pyridinecarboxamide;
N-(Cyclohexylmethyl)-3-[(2,2-dimethylbutanoyl)amino]pyridine-2-carboxamide;
3-[(4-Amino-1-naphthoyl)amino]-N-(tetrahydro-2H-pyran-4-ylmethyl)pyridine-2-carboxamide;
3-{[4-(Acetylamino)-1-naphthoyl]amino}-N-(tetrahydro-2H-pyran-4-ylmethyl)pyridine-2-carboxamide;
3-[(4-{[(Methylamino)carbonyl]amino}-1-naphthoyl)amino]-N-(tetrahydro-2H-pyran-4-ylmethyl)pyridine-2-carboxamide;
Methyl (4-{[(2-{[(tetrahydro-2H-pyran-4-ylmethyl)amino]carbonyl}pyridin-3-yl)amino]carbonyl}-1-naphthyl)carbamate;
N-(Cyclohexyloxy)-3-[(4-methyl-1-naphthoyl)amino]pyridine-2-carboxamide;
3-[(4-Methyl-1-naphthoyl)amino]-N-[(1-methylpiperidin-2-yl)methyl]pyridine-2-carboxamide;
3-[(4-Ethyl-1-naphthoyl)amino]-N-(tetrahydro-2H-pyran-4-ylmethyl)pyridine-2-carboxamide;
3-[(4-Ethyl-1-naphthoyl)amino]-N-(piperidin-2-ylmethyl)pyridine-2-carboxamide;
3-[(4-Isopropyl-1-naphthoyl)amino]-N-(tetrahydro-2H-pyran-4-ylmethyl)pyridine-2-carboxamide;
N-(2-Hydroxyethyl)-3-(1-naphthoylamino)pyridine-2-carboxamide;
3-[(4-Isopropyl-1-naphthoyl)amino]-N-(piperidin-2-ylmethyl)pyridine-2-carboxamide;
3-{[4-(Methoxymethyl)-1-naphthoyl]amino}-N-(piperidin-2-ylmethyl)pyridine-2-carboxamide;
3-{[4-(Ethoxymethyl)-1-naphthoyl]amino}-N-(piperidin-2-ylmethyl)pyridine-2-carboxamide;
N-(piperidin-2-ylmethyl)-3-{[4-(1H-1,2,4-triazol-1-ylmethyl)-1-naphthoyl]amino}pyridine-2-carboxamide;
N-(Piperidin-2-ylmethyl)-3-{[4-(1H-1,2,3-triazol-1-ylmethyl)-1-naphthoyl]amino}pyridine-2-carboxamide;
N-(Piperidin-2-ylmethyl)-3-{[4-(2H-1,2,3-triazol-2-ylmethyl)-1-naphthoyl]amino}pyridine-2-carboxamide;
3-[(4-Methyl-1-naphthoyl)amino]-N-[2-(tetrahydro-2H-pyran-4-yl)ethyl]pyridine-2-carboxamide;
3-{[4-(Methoxymethyl)-1-naphthoyl]amino}-N-[2-(tetrahydro-2H-pyran-4-yl)ethyl]pyridine-2-carboxamide;
3-[(4-Methyl-1-naphthoyl)amino]-N-(morpholin-3-ylmethyl)pyridine-2-carboxamide;
N-cyclopentyl-3-[(1-naphthalenylcarbonyl)amino]-2-pyridinecarboxamide;
N-butyl-3-[[[4-(1H-1,2,3-triazol-1-ylmethyl)-1-naphthalenyl]carbonyl]amino]-2-pyridinecarboxamide;
N-(cyclopropylmethyl)-3-[[[4-(1H-1,2,3-triazol-1-ylmethyl)-1-naphthalenyl]carbonyl]amino]-2-pyridinecarboxamide;
N-(cyclopentylmethyl)-3-[[[4-(1H-1,2,3-triazol-1-ylmethyl)-1-naphthalenyl]carbonyl]amino]-2-pyridinecarboxamide;
N-hexyl-3-[[[4-(1H-1,2,3-triazol-1-ylmethyl)-1-naphthalenyl]carbonyl]amino]-2-pyridinecarboxamide;
N-[3-(dimethylamino)propyl]-3-[[[4-(1H-1,2,3-triazol-1-ylmethyl)-1-naphthalenyl]carbonyl]amino]-2-pyridinecarboxamide;
N-[2-(4-morpholinyl)ethyl]-3-[[[4-(1H-1,2,3-triazol-1-ylmethyl)-1-naphthalenyl]carbonyl]amino]-2-pyridinecarboxamide;
N-(Cyclohexylmethyl)-3-{[4-(1H-1,2,3-triazol-1-ylmethyl)-1-naphthoyl]amino}pyridine-2-carboxamide;
N-(cyclohexylmethyl)-3-{[4-(2H-1,2,3-triazol-2-ylmethyl)-1-naphthoyl]amino}pyridine-2-carboxamide;
N-Pentyl-3-{[4-(1H-1,2,3-triazol-1-ylmethyl)-1-naphthoyl]amino}pyridine-2-carboxamide;
N-[2-(Tetrahydro-2H-pyran-4-yl)ethyl]-3-{[4-(1H-1,2,3-triazol-1-ylmethyl)-1-naphthoyl]amino}pyridine-2-carboxamide;
N-[2-(1H-Pyrrol-1-yl)ethyl]-3-{[4-(1H-1,2,3-triazol-1-ylmethyl)-1-naphthoyl]amino}pyridine-2-carboxamide;
N-[3-(1H-Imidazol-1-yl)propyl]-3-{[4-(1H-1,2,3-triazol-1-ylmethyl)-1-naphthoyl]amino}pyridine-2-carboxamide;
N-[3-(1H-Pyrazol-1-yl)propyl]-3-{[4-(1H-1,2,3-triazol-1-ylmethyl)-1-naphthoyl]amino}pyridine-2-carboxamide;
N-[2-(1H-Imidazol-1-yl)ethyl]-3-{[4-(1H-1,2,3-triazol-1-ylmethyl)-1-naphthoyl]amino}pyridine-2-carboxamide;
N-[2-(1H-1,2,4-Triazol-1-yl)ethyl]-3-{[4-(1H-1,2,3-triazol-1-ylmethyl)-1-naphthoyl]amino}pyridine-2-carboxamide;
N-(2-Methoxyethyl)-3-{[4-(1H-1,2,3-triazol-1-ylmethyl)-1-naphthoyl]amino}pyridine-2-carboxamide;
N-(2-Ethoxyethyl)-3-{[4-(1H-1,2,3-triazol-1-ylmethyl)-1-naphthoyl]amino}pyridine-2-carboxamide;
N-(2-Propoxyethyl)-3-{[4-(1H-1,2,3-triazol-1-ylmethyl)-1-naphthoyl]amino}pyridine-2-carboxamide;
N-(3-Methoxypropyl)-3-{[4-(1H-1,2,3-triazol-1-ylmethyl)-1-naphthoyl]amino}pyridine-2-carboxamide;
N-(3-Ethoxypropyl)-3-{[4-(1H-1,2,3-triazol-1-ylmethyl)-1-naphthoyl]amino}pyridine-2-carboxamide;
N-Allyl-3-{[4-(1H-1,2,3-triazol-1-ylmethyl)-1-naphthoyl]amino}pyridine-2-carboxamide;
N-Propyl-3-{[4-(1H-1,2,3-triazol-1-ylmethyl)-1-naphthoyl]amino}pyridine-2-carboxamide;
N-[(tetrahydro-2H-pyran-4-yl)methyl]-3-[[[4-(1H-1,2,3-triazol-1-ylmethyl)-1-naphthalenyl]carbonyl]amino]-2-pyridinecarboxamide;
N-[(tetrahydro-2H-pyran-4-yl)methyl]-3-[[[4-(4H-1,2,4-triazol-4-ylmethyl)-1-naphthalenyl]carbonyl]amino]-2-pyridinecarboxamide;
N-[(tetrahydro-2H-pyran-4-yl)methyl]-3-[[[4-(1H-1,2,4-triazol-1-ylmethyl)-1-naphthalenyl]carbonyl]amino]-2-pyridinecarboxamide;
3-[[[4-(1-pyrrolidinylmethyl)-1-naphthalenyl]carbonyl]amino]-N-[(tetrahydro-2H-pyran-4-yl)methyl]-2-pyridinecarboxamide;
3-[[[4-(1H-pyrazol-1-ylmethyl)-1-naphthalenyl]carbonyl]amino]-N-[(tetrahydro-2H-pyran-4-yl)methyl]-2-pyridinecarboxamide;
N-[(tetrahydro-2H-pyran-4-yl)methyl]-3-[[[4-(2H-tetrazol-2-ylmethyl)-1-naphthalenyl]carbonyl]amino]-2-pyridinecarboxamide;
N-(Tetrahydro-2H-pyran-4-yl)-3-[[[4-(1H-1,2,3-triazol-1-ylmethyl)-1-naphthalenyl]carbonyl]amino]-2-pyridinecarboxamide;
3-[[[4-(1H-imidazol-1-ylmethyl)-1-naphthalenyl]carbonyl]amino]-N-(tetrahydro-2H-pyran-4-yl)-2-pyridinecarboxamide;
3-[[[4-(1H-pyrazol-1-ylmethyl)-1-naphthalenyl]carbonyl]amino]-N-(tetrahydro-2H-pyran-4-yl)-2-pyridinecarboxamide;
3-[[[4-(methoxymethyl)-1-naphthalenyl]carbonyl]amino]-N-[(tetrahydro-2H-pyran-4-yl)methyl]-2-pyridinecarboxamide;
3-[[[4-(methoxymethyl)-1-naphthalenyl]carbonyl]amino]-N-[(tetrahydro-2H-pyran-4-yl)methyl]-2-pyridinecarboxamide;
3-[(4-benzyl-1-naphthoyl)amino]-N-(tetrahydro-2H-pyran-4-ylmethyl)pyridine-2-carboxamide;
3-[[[4-(3-furanylmethyl)-1-naphthalenyl]carbonyl]amino]-N-[(tetrahydro-2H-pyran-4-yl)methyl]-2-pyridinecarboxamide;
3-[[[4-(2-furanylmethyl)-1-naphthalenyl]carbonyl]amino]-N-[(tetrahydro-2H-pyran-4-yl)methyl]-2-pyridinecarboxamide;
N-[(tetrahydro-2H-pyran-4-yl)methyl]-3-[[[4-(2-thienylmethyl)-1-naphthalenyl]carbonyl]amino]-2-pyridinecarboxamide;
N-[(tetrahydro-2H-pyran-4-yl)methyl]-3-[[[4-(3-thienylmethyl)-1-naphthalenyl]carbonyl]amino]-2-pyridinecarboxamide;
N-(2-methylcyclohexyl)-3-[(1-naphthalenylcarbonyl)amino]-2-pyridinecarboxamide;
3-[(1-naphthalenylcarbonyl)amino]-N-[2-(1-pyrrolidinyl)ethyl]-2-pyridinecarboxamide;
N-(cyclobutylmethyl)-3-[[2-(4-morpholinyl)benzoyl]amino]-2-pyridinecarboxamide;
N-(Tetrahydro-2H-pyran-4-ylmethyl)-3-({4-[(3H-[1,2,3]triazolo[4,5-b]pyridin-3-yloxy)methyl]-1-naphthoyl}amino)pyridine-2-carboxamide;
3-(1-Naphthoylamino)-N-(pyrrolidin-2-ylmethyl)pyridine-2-carboxamide;
N-[(1-Methylpyrrolidin-2-yl)methyl]-3-(1-naphthoylamino)pyridine-2-carboxamide;
N-[(1-Methylpiperidin-2-yl)methyl]-3-(1-naphthoylamino)pyridine-2-carboxamide;
N-[(1-Acetylpiperidin-2-yl)methyl]-3-(1-naphthoylamino)pyridine-2-carboxamide;
Methyl 2-[({[3-(1-naphthoylamino)pyridin-2-yl]carbonyl}amino)methyl]piperidine-1-carboxylate;
N-(Cyclopentylmethyl)-4-(1-naphthoylamino)nicotinamide;
N-Cyclopentyl-4-(1-naphthoylamino)nicotinamide;
N-(Cyclopropylmethyl)-4-(1-naphthoylamino)nicotinamide;
N-Isobutyl-4-(1-naphthoylamino)nicotinamide;
N-(Cyclobutylmethyl)-4-[(4-methyl-1-naphthoyl)amino]nicotinamide;
N-(Cyclopentylmethyl)-4-[(4-methyl-1-naphthoyl)amino]nicotinamide;
3-{[4-(Hydroxymethyl)-1-naphthoyl]amino}-N-(tetrahydro-2H-pyran-4-ylmethyl)pyridine-2-carboxamide;
3-{[4-(Piperidin-1-ylmethyl)-1-naphthoyl]amino}-N-(tetrahydro-2H-pyran-4-ylmethyl)pyridine-2-carboxamide;
3-[(4-{[(2-Hydroxyethyl)amino]methyl}-1-naphthoyl)amino]-N-(tetrahydro-2H-pyran-4-ylmethyl)pyridine-2-carboxamide;
3-({4-[(Dimethylamino)methyl]-1-naphthoyl}amino)-N-(tetrahydro-2H-pyran-4-ylmethyl)pyridine-2-carboxamide;
3-{[4-(1H-Imidazol-1-ylmethyl)-1-naphthoyl]amino}-N-(tetrahydro-2H-pyran-4-ylmethyl)pyridine-2-carboxamide;
3-{[4-(Azetidin-1-ylmethyl)-1-naphthoyl]amino}-N-(tetrahydro-2H-pyran-4-ylmethyl)pyridine-2-carboxamide;
Methyl 4-{[(2-{[(tetrahydro-2H-pyran-4-ylmethyl)amino]carbonyl}pyridin-3-yl)amino]carbonyl}-1-naphthoate;
N,N-Dimethyl-N′-(2-{[(tetrahydro-2H-pyran-4-ylmethyl)amino]carbonyl}pyridin-3-yl)naphthalene-1,4-dicarboxamide;
2-Hydroxyethyl 4-{[(2-{[(tetrahydro-2H-pyran-4-ylmethyl)amino]carbonyl}pyridin-3-yl)amino]carbonyl}-1-naphthoate;
3-[(1-Benzofuran-2-ylcarbonyl)amino]-N-(tetrahydro-2H-pyran-4-ylmethyl)pyridine-2-carboxamide;
N-(Cyclohexylmethyl)-3-[(4-iodo-1-naphthoyl)amino]pyridine-2-carboxamide;
N-(Cyclohexylmethyl)-3-[(4-piperidin-1-yl-1-naphthoyl)amino]pyridine-2-carboxamide;
3-[(4-Azetidin-1-yl-1-naphthoyl)amino]-N-(cyclohexylmethyl)pyridine-2-carboxamide;
N-(Cyclohexylmethyl)-3-({4-[ethyl(methyl)amino]-1-naphthoyl}amino)pyridine-2-carboxamide;
N-(Cyclohexylmethyl)-3-[(4-pyrrolidin-1-yl-1-naphthoyl)amino]pyridine-2-carboxamide;
N-(Cyclohexylmethyl)-3-{[4-(4-isopropylpiperazin-1-yl)-1-naphthoyl]amino}pyridine-2-carboxamide;
N-(Cyclohexylmethyl)-3-({4-[3-(diethylamino)pyrrolidin-1-yl]-1-naphthoyl}amino)pyridine-2-carboxamide;
N′-(2-{[(Cyclohexylmethyl)amino]carbonyl}pyridin-3-yl)-N,N-dimethylnaphthalene-1,4-dicarboxamide;
N-(Cyclohexylmethyl)-3-{[4-(methoxymethyl)-1-naphthoyl]amino}pyridine-2-carboxamide;
N-(Cyclohexylmethyl)-3-({4-[(dimethylamino)methyl]-1-naphthoyl}amino)pyridine-2-carboxamide;
N-(Cyclobutylmethyl)-3-{[4-(1H-pyrrol-1-ylmethyl)-1-naphthoyl]amino}pyridine-2-carboxamide;
N-(Cyclobutylmethyl)-3-{[4-(1H-1,2,3-triazol-1-ylmethyl)-1-naphthoyl]amino}pyridine-2-carboxamide;
N-(Cyclobutylmethyl)-3-{[4-(1H-pyrazol-1-ylmethyl)-1-naphthoyl]amino}pyridine-2-carboxamide;
N-(Cyclobutylmethyl)-3-[(4-{[ethyl(methyl)amino]methyl}-1-naphthoyl)amino]pyridine-2-carboxamide;
N-(Cyclobutylmethyl)-3-{[4-(1H-imidazol-1-ylmethyl)-1-naphthoyl]amino}pyridine-2-carboxamide;
N-(Cyclobutylmethyl)-3-({4-[(dimethylamino)methyl]-1-naphthoyl}amino)pyridine-2-carboxamide;
N-(Cyclobutylmethyl)-3-{[4-(methoxymethyl)-1-naphthoyl]amino}pyridine-2-carboxamide;
N-(Cyclobutylmethyl)-3-{[4-(ethoxymethyl)-1-naphthoyl]amino}pyridine-2-carboxamide;
N′-(2-{[(Cyclobutylmethyl)amino]carbonyl}pyridin-3-yl)-N,N-dimethylnaphthalene-1,4-dicarboxamide;
N-(Cyclohexylmethyl)-3-{[4-(dimethylamino)-1-naphthoyl]amino}pyrazine-2-carboxamide;
N-(Cyclohexylmethyl)-3-{[5-(dimethylamino)-1-naphthoyl]amino}pyridine-2-carboxamide;
3-{[4-(Dimethylamino)-1-naphthoyl]amino}-N-(piperidin-2-ylmethyl)pyridine-2-carboxamide;
3-{[4-(dimethylamino)-1-naphthoyl]amino}-N-pentylpyridine-2-carboxamide;
3-{[4-(dimethylamino)-1-naphthoyl]amino}-N-hexylpyridine-2-carboxamide;
3-{[4-(dimethylamino)-1-naphthoyl]amino}-N-[3-(dimethylamino)propyl]pyridine-2-carboxamide;
3-{[4-(dimethylamino)-1-naphthoyl]amino}-N-propylpyridine-2-carboxamide;
3-{[4-(dimethylamino)-1-naphthoyl]amino}-N-(2-ethylbutyl)pyridine-2-carboxamide;
N-(cyclohexylmethyl)-3-{[(5-phenyl-1,3-oxazol-4-yl)carbonyl]amino}pyridine-2-carboxamide;
N-butyl-3-{[4-(dimethylamino)-1-naphthoyl]amino}pyridine-2-carboxamide;
3-{[(5-phenyl-1,3-oxazol-4-yl)carbonyl]amino}-N-(tetrahydro-2H-pyran-4-ylmethyl)pyridine-2-carboxamide;
3-{[4-(dimethylamino)-1-naphthoyl]amino}-N-[3-(1H-imidazol-1-yl)propyl]pyridine-2-carboxamide;
N-(4,4-difluorocyclohexyl)-3-(1-naphthoylamino)pyridine-2-carboxamide;
N-(3,5-difluorobenzyl)-3-(1-naphthoylamino)pyridine-2-carboxamide;
N-(4-morpholin-4-ylbenzyl)-3-(1-naphthoylamino)pyridine-2-carboxamide;
6-Methoxy-3-[(4-[1,2,3]triazol-1-ylmethyl-naphthalene-1-carbonyl)-amino]-pyridine-2-carboxylic acid cyclohexylmethyl-amide;
6-Hydroxy-3-[(4-[1,2,3]triazol-1-ylmethyl-naphthalene-1-carbonyl)-amino]-pyridine-2-carboxylic acid cyclohexylmethyl-amide;
6-Methoxy-3-[(4-[1,2,3]triazol-1-ylmethyl-naphthalene-1-carbonyl)-amino]-pyridine-2-carboxylic acid (tetrahydro-pyran-4-ylmethyl)-amide;
6-Hydroxy-3-[(4-[1,2,3]triazol-1-ylmethyl-naphthalene-1-carbonyl)-amino]-pyridine-2-carboxylic acid (tetrahydro-pyran-4-ylmethyl)-amide; and
6-Propoxy-3-[(4-[1,2,3]triazol-1-ylmethyl-naphthalene-1-carbonyl)-amino]-pyridine-2-carboxylic acid cyclohexylmethyl-amide;
and pharmaceutically acceptable salts thereof.

11. (canceled)

12. A method for the treatment of pain, the method comprising administering a therapeutically effective amount of a compound according to any one of claims 1, 4, 7, and 10 to a patient in need thereof.

13-14. (canceled)

15. A method for the treatment of a medical condition selected from the group consisting of anxiety, cancer, multiple sclerosis, Parkinson's disease, Huntington's chorea, Alzheimer's disease, and cardiavascular disorders, the method comprising administering a therapeutically effective amount of the compound according to any one of claims 1, 4, 7, and 10 to a patient in need thereof.

16. (canceled)

17. A pharmaceutical composition comprising a compound according to any one of claims 1, 4, 7, and 10 and a pharmaceutically acceptable carrier.

18. A method for the treatment of functional gastrointestinal disorders, the method comprising the step of administering a therapeutically effective amount of a compound according to any one of claims 1, 4, 7, and 10 to a patient in need thereof.

19. A method for the treatment of irritable bowel syndrome the method comprising the step of administering a therapeutically effective amount of a compound according to any one of claims 1, 4, 7, and 10 to a patient in need thereof.

20. A method for the treatment of gastroesophageal reflux disorder, the method comprising the step of administering a therapeutically effective amount of a compound according to any one of claims 1, 4, 7, and 10 to a patient in need thereof.

21. A method for preparing a compound of formula I, 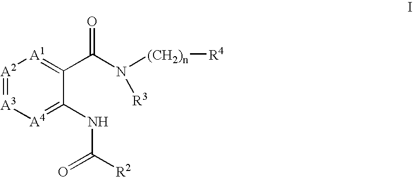
comprising the step of reacting a compound of formula II, 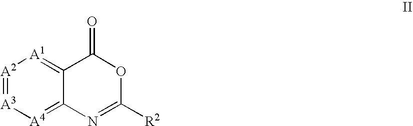
with a compound of formula R³(CH₂)ₙR⁴NH, in the presence of a base and a solvent,
wherein:
one of A¹, A², A³ or A⁴ is N and the remaining are CR¹;
each R¹ is independently selected from the group consisting of hydrogen, halogen, cyano, amino, acetylamino, hydroxyl, alkoxy, alkyl, halogenated alkoxy, alkylene, halogenated alkyl, halogenated alkenyl, and NR⁵R⁶;
R² is selected from the group consisting of: 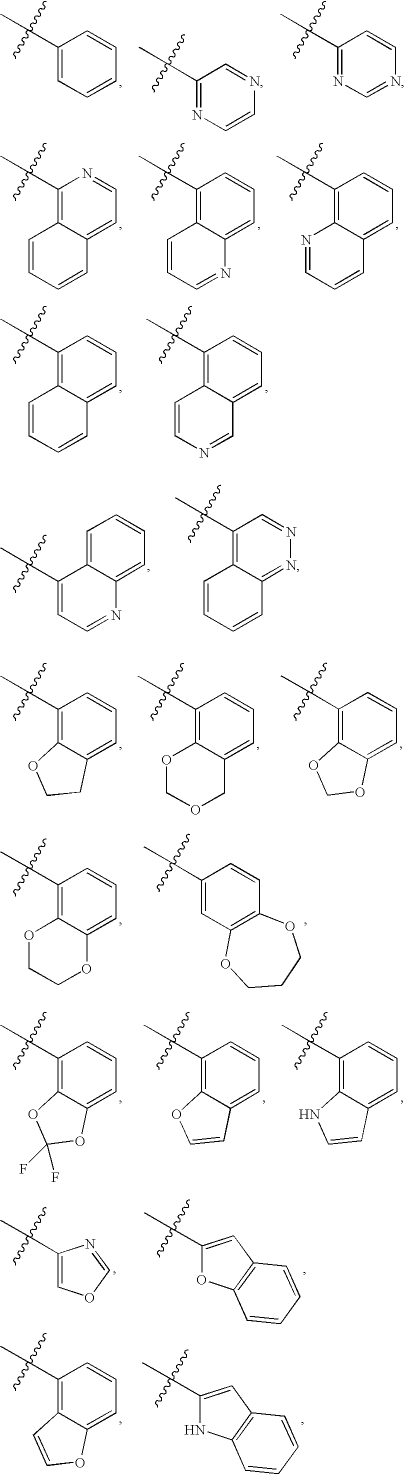 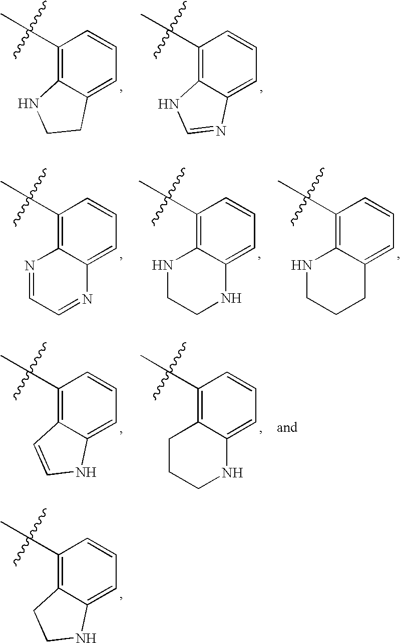
and is unsubstituted or substituted by one or more substituents independently selected from the group consisting of halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl, and heterocyclyl and is unsubstituted or substituted by one or more substituents independently selected from the group consisting of halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₁₆alkylcarbonyl, C₃₋₆heterocyclyl, and C₃₋₆heterocyclyl-C₁₋₁₆alkyl and are unsubstituted or substituted by one or more substituents independently selected from the group consisting of halogen, cyano, nitro, C₁₋₆ alkoxy, C₁₋₆ alkyl, and hydroxy;
with a proviso that when n=0, then R4 is not thiazolyl or 5-chloropyridinyl;
with a further proviso that when R2 is phenyl, then n=0 and R4 is not unsubstituted methyl, C₃ alkyl or unsubstituted C₄ alkyl; and
with a further proviso that the compound of formula I is not any one of
3-(benzoylamino)-N-benzylpyridine-2-carboxamide;
3-(benzoylamino)-N-pyridin-3-ylpyridine-2-carboxamide;
3-(benzoylamino)-N-phenylpyridine-2-carboxamide;
3-(benzoylamino)-N-(3-nitrophenyl)pyridine-2-carboxamide;
3-(benzoylamino)-N-(4-methoxyphenyl)pyridine-2-carboxamide;
3-(benzoylamino)-N-[4-(dimethylamino)phenyl]pyridine-2-carboxamide;
N-(2-hydroxyethyl)-4-(2-naphthoylamino)nicotinamide;
4-(benzoylamino)-N-(2-hydroxyethyl)nicotinamide;
3-(benzoylamino)-2,6-dimethyl-N-phenylisonicotinamide;
3-(benzoylamino)-2,6-dimethyl-N-(3-nitrophenyl)isonicotinamide;
2-(benzoylamino)-N-[cyano(2-thienyl)methyl]nicotinamide; and
2-(benzoylamino)-N-[cyano(phenyl)methyl]nicotinamide.

22. A method for preparing a compound of formula IB, 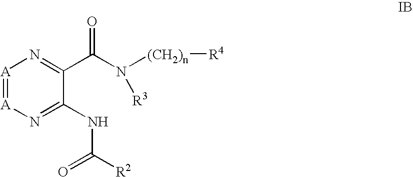
comprising the step of reacting a compound of formula IIB, 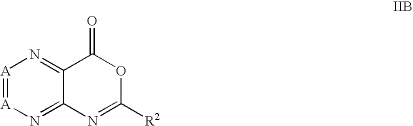
with a compound of formula R³(CH₂)ₙR⁴NH, in the presence of a base and a solvent,
wherein:
A is CR¹;
each R¹ is independently selected from the group consisting of hydrogen, halogen, cyano, amino, acetylamino, hydroxyl, alkoxy, alkyl, halogenated alkoxy, alkylene, halogenated alkyl, halogenated alkenyl, and NR⁵R⁶;
R² is selected from the group consisting of: 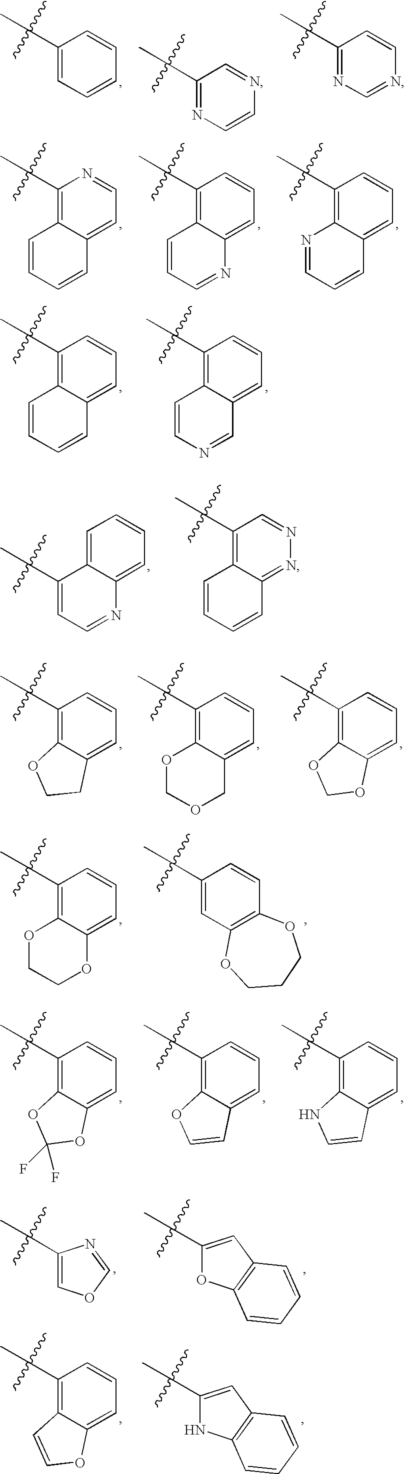 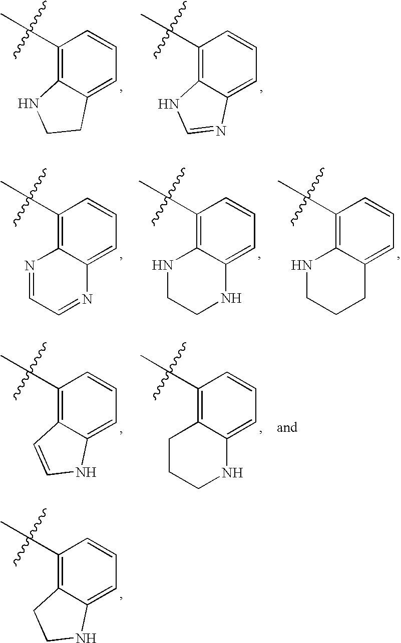
and is unsubstituted or substituted by one or more substituents independently selected from halogen, halogenated alkyl, alkyl, halogenated alkoxy, cyano, nitro, alkoxy, hydroxy, hydroxy-alkyl, amino, alkyl-aryl, alkoxy, alkoxy-alkyl, alkylcarbonyl, alkoxycarbonyl, alkylamino, amino-alkyl, alkyl-amino-carbonyl, heteroaryl-carbonyl, heterocyclyl-carbonyl, arylcarbonyl, heterocyclyl, cycloalkyl, heteroaryl, heteroarylalkyl-, aryl, aryl-alkyl, and —NR⁵R⁶;
R³ is hydrogen or alkyl;
R⁴ is selected from the group consisting of alkyl, alkenyl, cycloalkyl, cycloalkenyl, alkoxy, aryl, heteroaryl and heterocyclyl and is unsubstituted or substituted by one or more substituents independently selected from halogen, halogenated alkyl, alkyl, alkylcarbonyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-alkyl, and —NR⁵R⁶; and
n is 0, 1, 2, 3, 4, or 5; or
R³ and R⁴ together with the nitrogen atom to which they are attached form a heterocyclyl moiety which is optionally fused with a five or six membered ring containing one or more heteroatoms and is unsubstituted or substituted by one or more substituents independently selected from the group consisting of halogen, halogenated alkyl, alkyl, cyano, nitro, amino, amino-alkyl, alkoxy, halogenated alkoxy, hydroxy, alkoxy-alkyl, alkoxy-aryl, alkoxy-carbonyl, heterocyclic moiety, aryl, aryl-alkyl, heterocyclic-alkyl, hydroxy-alkyl, heteroaryl, alkyl-heteroaryl, aryl-C₁₋₆alkyl, and —NR⁵R⁶; and
R⁵ and R⁶ are each independently selected from the group consisting of hydrogen, C₁₋₆alkyl, C₂₋₆alkenyl, alkoxyC₁₋₆ alkyl, C₁₋₆ alkylcarbonyl, C₁₋₆ alkoxycarbonyl, hydroxyC₁₋₆ alkyl, alkoxy, C₃₋₆cycloalkyl, C₃₋₆cycloalkyl-C₁₋₆alkyl, C₁₋₁₆alkylcarbonyl, C₃₋₆heterocyclyl, and C₃₋₆heterocyclyl-C₁₋₁₆alkyl and are each independently unsubstituted or substituted by one or more substituents independently selected from the group consisting of halogen, cyano, nitro, C₁₋₆ alkoxy, C₁₋₆ alkyl, and hydroxy;
with a proviso that the compound of formula IB is not any one of:
3-[(4-tert-butylbenzoyl)amino]-N-(5-chloro-pyridin-2-yl)pyrazine-2-carboxamide;
N-[2-(1H-imidazol-2-yl)ethyl]-3-[[4-(1,1-dimethylethyl)benzoyl]amino]-2-pyrazine-carboxamide; and
3-(benzoylamino)-N-(methoxycarbonylmethyl)pyrazine-2-carboxamide.

23. The method according to any one of claims 20-22, wherein the base is DIPEA.

24. The method according to any one of claims 20-22, wherein the solvent is DMF.

25. A method for the inhibition of transient lower esophageal sphincter relaxations, the method comprising administering a therapeutically effective amount of a compound according to claim 1 to a patient in need thereof.

26. A method for the treatment of gastroesophageal reflux disorder (GERD), the method comprising administering a therapeutically effective amount of a compound according to claim 1 to a patient in need thereof.

27. A method for the treatment of reflux, the method comprising administering a therapeutically effective amount of a compound according to claim 1 to a patient in need thereof.
